(19) United States
(12) Patent Application Publication
(10) Pub. No.: US 2023/0106293 A1
(43) Pub. Date: Apr. 6, 2023

(54) ORGANIC ELECTROLUMINESCENT ELEMENT AND ELECTRONIC DEVICE

(71) Applicant: IDEMITSU KOSAN CO., LTD., Tokyo (JP)

(72) Inventors: Takayasu SADO, Tokyo (JP); Sayaka MIZUTANI, Tokyo (JP)

(73) Assignee: IDEMITSU KOSAN CO., LTD., Tokyo (JP)

(21) Appl. No.: 17/956,427

(22) Filed: Sep. 29, 2022

Related U.S. Application Data
(63) Continuation of application No. 14/653,722, filed on Jun. 18, 2015.

(30) Foreign Application Priority Data
Dec. 21, 2012 (JP) 2012-280306

(51) Int. Cl. H01L 51/00; C07D 403/14; C07D 405/14; C07D 471/04; C07F 7/08; C07D 487/04; C07D 405/10; C07D 409/14; C07D 401/14; C07D 401/10; C07D 498/06; C07D 491/048; C07D 519/00; C07D 471/06; C07D 487/16; C07D 409/10; C07D 491/22; C07D 403/10; C07D 487/06; C07D 251/24; C07D 513/06

(52) U.S. Cl. CPC H01L 51/0072; H01L 51/0052; C07D 403/14; C07D 405/14; C07D 471/04; C07F 7/0816; C07D 487/04; C07D 405/10; C07D 409/14; C07D 401/14; C07D 401/10; C07D 498/06; C07D 491/048; C07D 519/00; C07D 471/06; C07D 487/16; C07D 409/10; C07D 491/22; C07D 403/10; C07D 487/06; C07D 251/24; C07D 513/06; H01L 51/0067; H01L 51/0073; H01L 51/0074; H01L 51/0054; H01L 51/508

(57) ABSTRACT

An organic electroluminescence device includes: an anode; a cathode opposite the anode; and an organic layer interposed between the anode and the cathode, in which the organic layer includes: an emitting layer; and a first electron transporting layer and a second electron transporting layer which are provided on a side of the emitting layer near the cathode. The first electron transporting layer includes a compound represented by a formula (1) below, and the second electron transporting layer includes a compound represented by a formula (2) below.

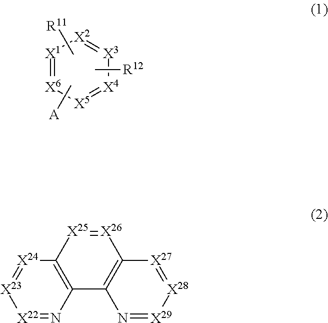

### CROSS-REFERENCE TO RELATED APPLICATIONS

The present application is a Divisional of U.S. patent application Ser. No. 14/653,722, filed on Jun. 18, 2015, which claims priority under 37 U.S.C. § 371 to International Patent Application No. PCT/JP2013/077342, filed Oct. 8, 2013, which claims priority to and the benefit of Japanese Patent Application No. 2012-280306, filed on Dec. 21, 2012. The contents of these applications are hereby incorporated by reference in their entireties.

### TECHNICAL FIELD

The present invention relates to an organic electroluminescence device and an electronic device.

### BACKGROUND ART

An organic electroluminescence device (hereinafter, occasionally abbreviated as organic EL device) using an organic substance is highly expected to be used as an inexpensive solid-emitting full-color display device having a large area and has been variously developed.

In general, an organic EL device includes a pair of opposing electrodes and an emitting layer between the pair of electrodes. When an electrical field is applied to the opposing electrodes of the organic EL device, electrons are injected from a cathode and holes are injected from an anode. The injected electrons and holes are recombined in the emitting layer to form excitons. Energy generated when the excitons in an excited state are returned to a ground state is irradiated as light. The organic EL device emits light in accordance with such a principle.

A typical organic EL device requires a higher drive voltage than an inorganic light-emitting diode. Moreover, since properties of the organic EL device are considerably deteriorated, the organic EL device is not in practical use. Although the organic EL device has been gradually improved in recent years, further voltage reduction and a higher efficiency have been demanded.

Patent Literature 1 discloses an organic EL device having an organic metal complex and an electron transporting layer that contains a compound having a phenanthroline skeleton or benzoquinoline skeleton.

### CITATION LIST

### Patent Literature(s)

Patent Literature 1: JP-A-2010-27761

### SUMMARY OF THE INVENTION

### Problems to be Solved by the Invention

In the organic EL device disclosed in Patent Literature 1, however, a drive voltage and an efficiency are substantially at the same level of those of a typical organic EL device although a lifetime is improved compared with that of the typical organic EL device. Accordingly, further voltage reduction and a higher efficiency have been demanded.

An object of the invention is to provide a highly efficient organic EL device driven at a low voltage. Another object of the invention is to provide an electronic device including the organic electroluminescence device of the invention.

### Means for Solving the Problems

According to an aspect of the invention, an organic electroluminescence device includes: an anode; a cathode opposite the anode; and an organic layer interposed between the anode and the cathode, in which the organic layer includes: an emitting layer; a first electron transporting layer interposed between the emitting layer and the cathode; and a second electron transporting layer interposed between the first electron transporting layer and the cathode, in which the first electron transporting layer includes a compound represented by a formula (1) below, and the second electron transporting layer includes a compound represented by a formula (2) below.

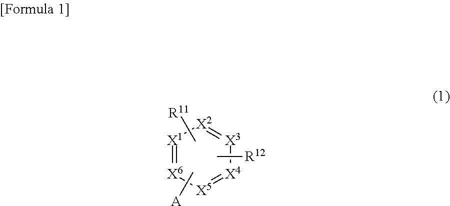

In the formula (1), X¹ to X⁶ each independently represent a nitrogen atom, CR, CA, CR¹¹ or CR¹². At least one of X¹ to X⁶ is a nitrogen atom.

R is each independently selected from the group consisting of a hydrogen atom, halogen atom, cyano group, nitro group, hydroxyl group, carboxyl group, sulfonyl group, mercapto group, substituted or unsubstituted boryl group, substituted or unsubstituted phosphino group, substituted or unsubstituted acyl group, substituted or unsubstituted amino group, substituted or unsubstituted silyl group, substituted or unsubstituted alkyl group having 1 to 30 carbon atoms, substituted or unsubstituted alkenyl group having 2 to 30 carbon atoms, substituted or unsubstituted alkynyl group having 2 to 30 carbon atoms, substituted or unsubstituted aralkyl group having 6 to 30 carbon atoms, substituted or unsubstituted alkoxy group having 1 to 30 carbon atoms, substituted or unsubstituted aryloxy group having 6 to 40 ring carbon atoms, substituted or unsubstituted heteroaryloxy group having 5 to 40 ring carbon atoms, substituted or unsubstituted alkylthio group having 1 to 30 carbon atoms, substituted or unsubstituted arylthio group having 6 to 40 ring carbon atoms, substituted or unsubstituted heteroarylthio group having 5 to 40 ring carbon atoms, substituted or unsubstituted alkoxycarbonyl group having 2 to 30 carbon atoms, substituted or unsubstituted aryloxycarbonyl group having 6 to 40 ring carbon atoms, substituted or unsubstituted heteroaryloxycarbonyl group having 5 to 40 ring carbon atoms, substituted or unsubstituted aromatic hydrocarbon group having 6 to 40 ring carbon atoms, and substituted or unsubstituted heterocyclic group having 5 to 40 ring atoms.

A is represented by a formula (11) below.

R¹¹ and R¹² are a hydrogen atom, a substituted or unsubstituted aromatic hydrocarbon group having 6 to 40 ring carbon atoms, a substituted or unsubstituted heterocyclic group having 5 to 40 ring atoms, or a group represented by the formula (11) below.

In the formula (1), when at least one of X¹ to X⁶ is a nitrogen atom and a triphenylenyl group is present in any one of A, R¹¹ and R¹² in the formula (1), the total number of the triphenylenyl group contained in A, R¹¹ and R¹² is not 1. In the formula (1), when two or three of X¹ to X⁶ are nitrogen atoms and the triphenylenyl group is present in any one of A, R¹¹ and R¹² in the formula (1), the total number of the triphenylenyl group contained in A, R¹¹ and R¹² is neither 1 nor 2.

A, R¹¹ and R¹² are respectively bonded to carbon atoms of any ones of X¹ to X⁶.

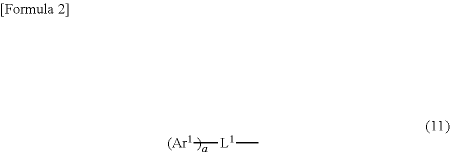

In the formula (11), a is an integer of 1 to 5. L¹ is a single bond or a linking group. The linking group in L¹ is a substituted or unsubstituted polyvalent linear, branched or cyclic aliphatic hydrocarbon group having 1 to 30 carbon atoms, a substituted or unsubstituted polyvalent amino group, a substituted or unsubstituted polyvalent aromatic hydrocarbon group having 6 to 40 ring carbon atoms, a substituted or unsubstituted polyvalent heterocyclic group having 5 to 40 ring atoms, or a polyvalent multiple linking group provided by bonding two or three groups selected from the aromatic hydrocarbon group and the heterocyclic group.

In the multiple linking group, the aromatic hydrocarbon group and the heterocyclic group forming the multiple linking group are mutually the same or different, and the aromatic hydrocarbon group and the heterocyclic group which are adjacent to each other are optionally mutually bonded to form a ring.

Ar¹ is a substituted or unsubstituted aromatic hydrocarbon group having 6 to 40 ring carbon atoms or a substituted or unsubstituted heterocyclic group having 5 to 40 ring atoms. When a is from 2 to 5, Ar¹ are mutually the same or different.

Ar¹ and L¹ are bonded to each other to form a cyclic structure or are not bonded,

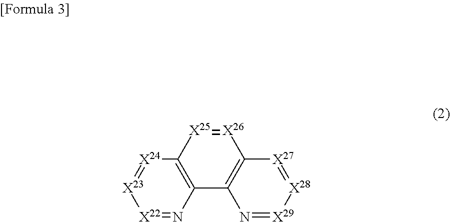

In the formula (2), X²² to X²⁹ each independently represent a nitrogen atom, CR²¹, or a carbon atom to be bonded to a group represented by a formula (21) below.

At least one of X²² to X²⁹ is a carbon atom to be bonded to a group represented by a formula (21) below. When a plurality of ones of X²² to X²⁹ are carbon atoms to be bonded to the group represented by the formula (21) below, a plurality of the group represented by the formula (21) are the same or different.

R²¹ represents the same as R of the formula (1). R²¹ of adjacent CR²¹ in X²² to X²⁹ are bonded to each other to form a cyclic structure or are not bonded.

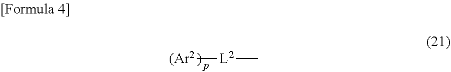

In the formula (21), p is an integer of 1 to 5.

Ar² is a substituted or unsubstituted aromatic hydrocarbon group having 6 to 40 ring carbon atoms or a substituted or unsubstituted heterocyclic group having 5 to 40 ring atoms. When p is from 2 to 5, a plurality of Ar² are mutually the same or different.

L² is a single bond or a linking group. The linking group in L² represents a substituted or unsubstituted polyvalent linear, branched or cyclic aliphatic hydrocarbon group having 1 to 30 carbon atoms, a substituted or unsubstituted polyvalent aromatic hydrocarbon group having 6 to 40 ring carbon atoms, a substituted or unsubstituted polyvalent heterocyclic group having 5 to 40 ring atoms, or a polyvalent multiple linking group provided by bonding two or three groups selected from the aromatic hydrocarbon group and the heterocyclic group.

In the multiple linking group, the aromatic hydrocarbon group and the heterocyclic group forming the multiple linking group are mutually the same or different, and the aromatic hydrocarbon group and the heterocyclic group which are adjacent to each other are optionally mutually bonded to form a ring.

The heterocyclic group having 5 to 40 ring atoms in Ar² of the formula (21) includes a substituted or unsubstituted group derived from the compound represented by the formula (2). However, the compound represented by the formula (2) contains at most 6 Ar² that is a substituted or unsubstituted group derived from the compound represented by the formula (2).

Adjacent Ar² and L² are bonded to each other to form a cyclic structure or are not bonded.

L² bonded to any one of carbon atoms in X²² to X²⁹ of the formula (2) and a carbon atom or R²¹ of CR²¹ in one of X²² to X²⁹ adjacent to the carbon atom bonded to L² are further bonded to each other to form a ring, or are not bonded.

According to another aspect of the invention, an electronic device includes the organic electroluminescence device according to the above aspect of the invention.

According to the above aspects of the invention, a highly efficient organic EL device driven at a low voltage can be provided. Moreover, an electronic device including the organic electroluminescence device according to the above aspect of the invention can be provided.

### BRIEF DESCRIPTION OF DRAWINGS

FIG. 1 shows an example of an organic electroluminescence device according to a first exemplary embodiment of the invention.

FIG. 2 shows an example of an organic electroluminescence device according to a second exemplary embodiment of the invention.

### DESCRIPTION OF EMBODIMENT(S)

### Organic EL Device

### Arrangement(s) of Organic EL Device

Typical device arrangements of an organic EL device include the following arrangements (a) to (e) and the like:

(a) anode/emitting layer/cathode;
(b) anode/hole injecting transporting layer/emitting layer/cathode;
(c) anode/emitting layer/electron injecting transporting layer/cathode;
(d) anode/hole injecting transporting layer/emitting layer/electron injecting transporting layer/cathode; and
(e) anode/hole injecting transporting layer/first emitting layer/intermediate layer/second emitting layer/electron injecting transporting layer/cathode.

While the arrangements (d) and (e) are preferably used among the above arrangements, the arrangement of the invention is not limited to the above arrangements.

It should be noted that the aforementioned “emitting layer” is an organic layer having an emission function and, when a doping system is employed, containing a host material and a dopant material. At this time, the host material has a function to mainly promote recombination of electrons and holes and trap excitons within the emitting layer while the dopant material has a function to promote an efficient emission from the excitons obtained by the recombination. In a phosphorescent device, the host material has a function of trapping the excitons, which are generated mainly in the dopant, within the emitting layer.

The “hole injecting/transporting layer (or hole injecting transporting layer)” means “at least one of a hole injecting layer and a hole transporting layer while the “electron injecting/transporting layer (or electron injecting transporting layer)” means “an electron transporting layer” or “an electron injecting layer and an electron transporting layer.” Herein, when the hole injecting layer and the hole transporting layer are provided, the hole injecting layer is preferably closer to the anode. When the electron injecting layer and the electron transporting layer are provided, the electron injecting layer is preferably closer to the cathode.

The intermediate layer (also referred to as an intermediate conductive layer, charge generating layer or CGL) is a layer including at least one of an intermediate conductive layer and a charge generating layer, or at least one of the intermediate conductive layer and the charge generation layer. The intermediate layer serves as a source for supplying electrons or holes to be injected in an emitting unit. In addition to charges injected from a pair of electrodes, charges supplied from the intermediate layer are injected into the emitting unit. Accordingly, by providing the intermediate layer, luminous efficiency (current efficiency) relative to injected current is improved.

In an exemplary embodiment of the invention, an organic EL device includes: a cathode; an anode; and an organic layer interposed between the cathode and the anode. The organic layer at lease includes an emitting layer, first electron transporting layer and second electron transporting layer. Further, the organic layer may include a layer employed in a known organic EL device such as a hole injecting layer, a hole transporting layer, an electron injecting layer, a hole blocking layer and an electron blocking layer. The organic layer may include an inorganic compound.

### First Exemplary Embodiment

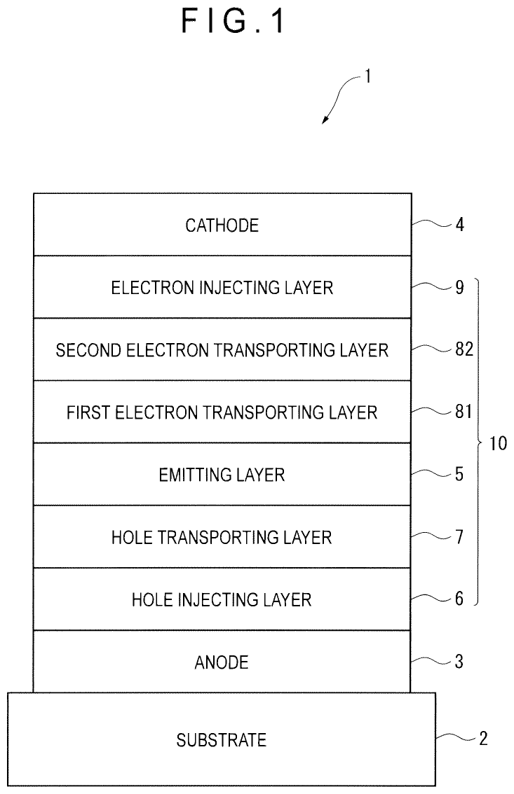

An organic EL device 1 in a first exemplary embodiment includes a light-transmissive substrate 2, an anode 3, a cathode 4 and an organic layer 10 interposed between the anode 3 and the cathode 4 as shown in FIG. 1.

The organic layer 10 includes a hole injecting layer 6, hole transporting layer 7, emitting layer 5, first electron transporting layer 81, second electron transporting layer 82, and electron injecting layer 9 sequentially from the anode 3.

### First Electron Transporting Layer

The first electron transporting layer of the organic EL device 1 of the first exemplary embodiment contains a compound represented by a formula (1) below.

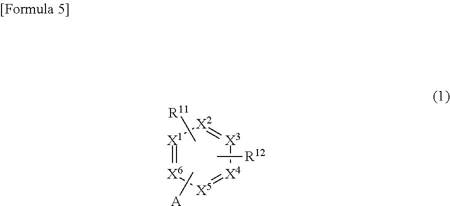

In the formula (1), X¹ to X⁶ are each independently a nitrogen atom, CR, CA, CR¹¹ or CR¹². However, at least one of X¹ to X⁶ is a nitrogen atom. R is each independently selected from the group consisting of a hydrogen atom, halogen atom, cyano group, nitro group, hydroxyl group, carboxyl group, sulfonyl group, mercapto group, substituted or unsubstituted boryl group, substituted or unsubstituted phosphino group, substituted or unsubstituted acyl group, substituted or unsubstituted amino group, substituted or unsubstituted silyl group, substituted or unsubstituted alkyl group having 1 to 30 carbon atoms, substituted or unsubstituted alkenyl group having 2 to 30 carbon atoms, substituted or unsubstituted alkynyl group having 2 to 30 carbon atoms, substituted or unsubstituted aralkyl group having 6 to 30 carbon atoms, substituted or unsubstituted alkoxy group having 1 to 30 carbon atoms, substituted or unsubstituted aryloxy group having 6 to 40 ring carbon atoms, substituted or unsubstituted heteroaryloxy group having 5 to 40 ring carbon atoms, substituted or unsubstituted alkylthio group having 1 to 30 carbon atoms, substituted or unsubstituted arylthio group having 6 to 40 ring carbon atoms, substituted or unsubstituted heteroarylthio group having 5 to 40 ring carbon atoms, substituted or unsubstituted alkoxycarbonyl group having 2 to 30 carbon atoms, substituted or unsubstituted aryloxycarbonyl group having 6 to 40 ring carbon atoms, substituted or unsubstituted heteroaryloxycarbonyl group having 5 to 40 ring carbon atoms, substituted or unsubstituted aromatic hydrocarbon group having 6 to 40 ring carbon atoms, and substituted or unsubstituted heterocyclic group having 5 to 40 ring atoms.

A is represented by a formula (11) below. R¹¹ and R¹² are a hydrogen atom, a substituted or unsubstituted aromatic hydrocarbon group having 6 to 40 ring carbon atoms, a substituted or unsubstituted heterocyclic group having 5 to 40 ring atoms, or a group represented by the formula (11) below.

However, when at least one of X¹ to X⁶ is a nitrogen atom and a triphenylenyl group is present in any one of A, R¹¹ and R¹² in the formula (1), the total number of the triphenylenyl group contained in A, R¹¹ and R¹² is not 1. Moreover, when two or three of X¹ to X⁶ are nitrogen atoms and the triphenylenyl group is present in any one of A, R¹¹ and R¹² in the formula (1), the total number of the triphenylenyl group contained in A, R¹¹ and R¹² is neither 1 nor 2.

A, R¹¹ and R¹² are respectively bonded to any ones of carbon atoms in X¹ to X⁶.

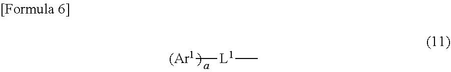

In the formula (11), a is an integer of 1 to 5, L¹ is a single bond or a linking group. The linking group in L¹ is a substituted or unsubstituted polyvalent linear, branched or cyclic aliphatic hydrocarbon group having 1 to 30 carbon atoms, a substituted or unsubstituted polyvalent amino group, a substituted or unsubstituted polyvalent aromatic hydrocarbon group having 6 to 40 ring carbon atoms, a substituted or unsubstituted polyvalent heterocyclic group having 5 to 40 ring atoms, or a polyvalent multiple linking group provided by bonding two or three groups selected from the aromatic hydrocarbon group and the heterocyclic group.

In the multiple linking group, the aromatic hydrocarbon group and the heterocyclic group forming the multiple linking group are mutually the same or different. The aromatic hydrocarbon group and the heterocyclic group which are adjacent to each other may be mutually bonded to form a ring.

Ar¹ is a substituted or unsubstituted aromatic hydrocarbon group having 6 to 40 ring carbon atoms or a substituted or unsubstituted heterocyclic group having 5 to 40 ring atoms. When a is from 2 to 5, Ar¹ are mutually the same or different.

Ar¹ and L¹ are bonded to each other to form a cyclic structure or are not bonded.

In the formula (11), a case where L¹ is a multiple linking group and the adjacent aromatic hydrocarbon group and heterocyclic group form a ring is exemplified by groups represented by formulae (11a) and (11b) below, in which three phenylene groups are provided in L¹.

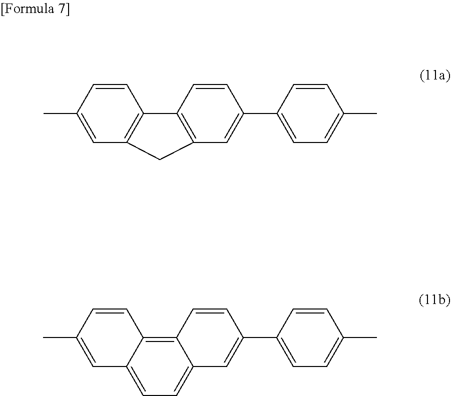

In the formula (11), Ar¹ is preferably represented by a formula (12) below.

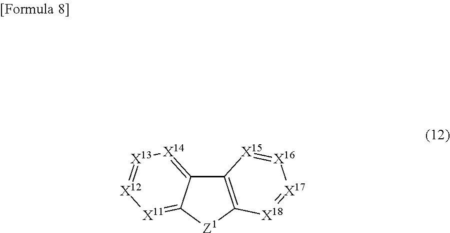

In the formula (12), X¹¹ to X¹⁸ are each independently a nitrogen atom or CR¹³. Z¹ is an oxygen atom, sulfur atom, NR¹⁴, CR¹⁵R¹⁶, or SiR¹⁷R¹⁸. R¹³ to R¹⁸ represent the same as R of the formula (1).

However, one of R¹³ to R¹⁸ is a single bond to be bonded with L¹ in the formula (11).

When the formula (12) includes a plurality of R¹³, the plurality of R¹³ are mutually the same or different.

R¹³ in adjacent CR¹³ may be bonded together to form a saturated or unsaturated ring. When Z¹ is one of NR¹⁴, CR¹⁵R¹⁶ and SiR¹⁷R¹⁸ and at least one of X¹¹ and X¹⁸ is CR¹³, one of R¹⁴ to R¹⁸ may be bonded to R¹³ to form a ring. Alternatively, Z¹ may be directly bonded to R¹³ to form a ring.

The rest of R¹³ to R¹⁸ except for a single bond to be bonded to L¹ may be further bonded to L¹ of the formula (11) to form a saturated or unsaturated ring.

When R¹³ of adjacent CR¹³ are bonded together to form a saturated or unsaturated ring in the formula (12), the ring is exemplarily represented by a formula (12A) or (12B) below.

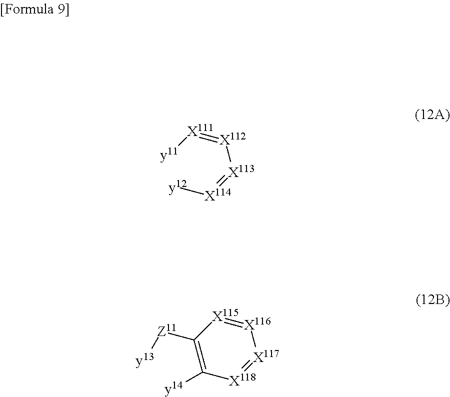

In the formula (12A), y¹¹ and y¹² indicate two bonding positions to the adjacent two CR¹³ in the formula (12).

In the formula (12B), y¹³ and y¹⁴ indicate two bonding positions to the adjacent two CR¹³ in the formula (12).

X¹¹¹ to X¹¹⁸ respectively represent the same as X¹¹ to X¹⁸ in the formula (12).

Z¹¹ represents the same as Z¹ in the formula (12).

In the formula (12), a case where R¹³ in adjacent CR¹³ are bonded together to form the ring represented by the formula (12A) or (12B) is exemplified by structures represented by formulae (12A-1) to (12A-3) and (12B-1) to (12B-6) below.

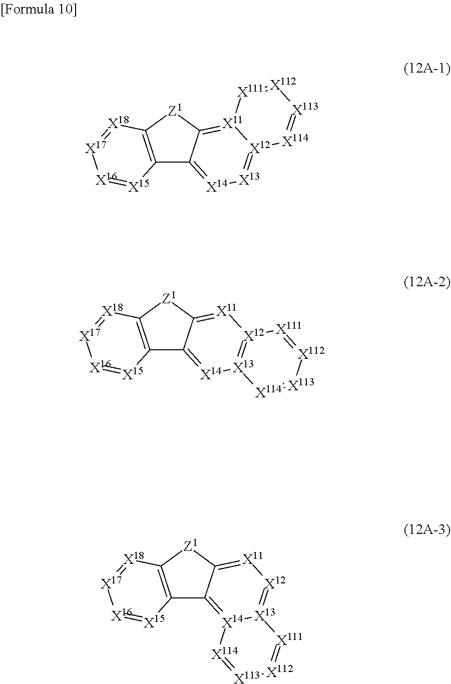

In the formulae (12A-1) to (12A-3), Z¹ and X¹¹ to X¹⁸ represent the same as Z¹ and X¹¹ to X¹⁸ in the formula (12) and X¹¹¹ to X¹¹⁴ represent the same as X¹¹ to X¹⁸ in the formula (12).

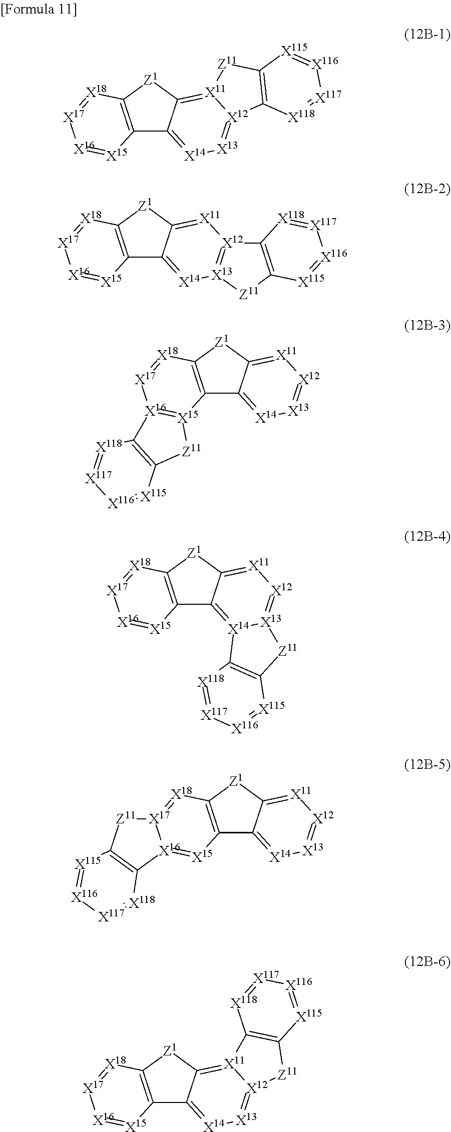

In the formulae (12B-1) to (12B-6), Z¹ and X¹¹ to X¹⁸ represent the same as Z and X¹¹ to X¹⁸ in the formula (12), Z¹¹ represents the same as Z¹ in the formula (12), and X¹¹⁵ to X¹¹¹ represent the same as X¹¹ to X¹⁸ in the formula (12).

Further, in the formula (12), it is preferable that X¹³ or X¹⁶ is CR¹³ and R¹³ is a single bond to be bonded to L¹.

In the formula (12), Z¹ is preferably an oxygen atom or NR¹⁴, more preferably NR¹⁴.

In the formula (12), it is preferable that Z¹ is NR¹⁴ and X¹¹ to X¹⁸ are CR¹³. In other words, in the formula (11), Ar¹ is preferably a substituted or unsubstituted carbazolyl group.

In the formula (12), it is also preferable that Z¹ is NR¹⁴ and X¹¹ to X¹⁸ are CR¹³ and R¹⁴ is a single bond to be bonded to L¹ of the formula (11).

In the formula (11), Ar¹ is a substituted or unsubstituted fused aromatic hydrocarbon group having 8 to 20 ring carbon atoms.

Examples of the fused aromatic hydrocarbon group having 8 to 20 ring carbon atoms include groups derived from naphthalene, anthracene, acephenanthrylene, aceanthrylene, benzanthracene, triphenylene, pyrene, chrysene, and naphthacene.

In the formula (11), Ar¹ is also preferably a group represented by a formula (13) below.

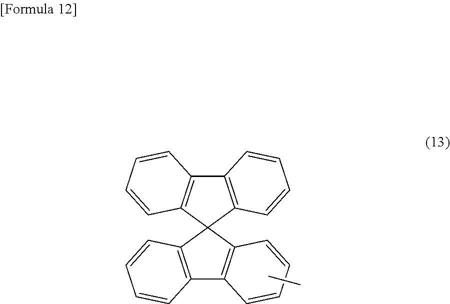

In the formula (13), a bond represents a single bond between one of carbon atoms and L¹ of the formula (11).

In the formula (11), Ar¹ is also preferably a group represented by a formula (14) below.

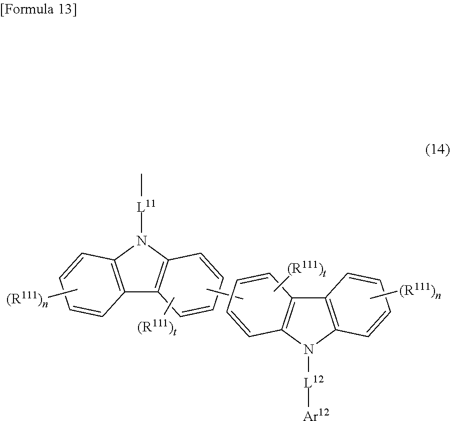

In the formula (14), t is an integer of 1 to 3. n is an integer of 1 to 4. R¹¹¹ represents the same as R of the formula (1). A plurality of R¹¹¹ are mutually the same or different. L¹¹ and L¹² represent the same as L¹ of the formula (11).

Ar¹² is a substituted or unsubstituted aromatic hydrocarbon group having 6 to 40 ring carbon atoms or a substituted or unsubstituted heterocyclic group having 5 to 40 ring atoms.

Note that L¹¹ is a single bond to be bonded to L¹ of the formula (11). The plurality of R¹¹¹ are bonded to any ones of carbon atoms in the carbazolyl group. Two carbazolyl groups are mutually bonded at any one of the respective positions 1 to 4.

In the formula (11), a is preferably an integer of 1 to 3, more preferably 1 or 2.

In the formula (11), it is preferable that a is 1 and L¹ is a linking group that is a substituted or unsubstituted divalent aromatic hydrocarbon group having 6 to 40 ring carbon atoms or a substituted or unsubstituted divalent heterocyclic group having 5 to 40 ring atoms.

In the formula (11), it is also preferable that a is 2 and L¹ is a linking group that is a substituted or unsubstituted trivalent aromatic hydrocarbon group having 6 to 40 ring carbon atoms or a substituted or unsubstituted trivalent heterocyclic group having 5 to 40 ring atoms.

In the formula (11), L¹ is further preferably a divalent or trivalent residue derived from one of benzene, biphenyl, terphenyl, naphthalene and phenanthrene.

The formula (1) is preferably represented by a formula (100) below.

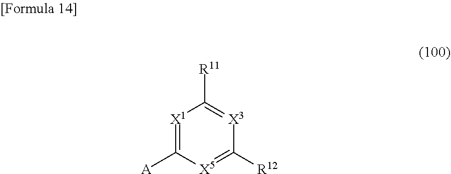

In the formula (100), X¹, X³ and X⁵ are each independently a nitrogen atom or CR. However, at least one of X¹, X³ and X⁵ is a nitrogen atom.

A, R, R¹¹ and R¹² respectively represent the same as A, R, R¹¹ and R¹² of the formula (1).

In the formula (1), two or three of X¹ to X⁶ are preferably nitrogen atoms. Among X¹ to X⁶ of the formula (1), two or three of X¹, X³ and X⁵ are preferably nitrogen atoms. In other words, two or three of X¹, X³ and X⁵ are preferably nitrogen atoms in the formula (11).

The formula (1) is particularly preferably represented by formulae (1A) to (1D) below. Specifically, pyrimidine in which X¹ and X³ are nitrogen atoms, pyrimidine in which X³ and X⁵ are nitrogen atoms, pyrimidine in which X¹ and X⁵ are nitrogen atoms, and triazine in which all of X¹, X³ and X⁵ are nitrogen atoms are preferable.

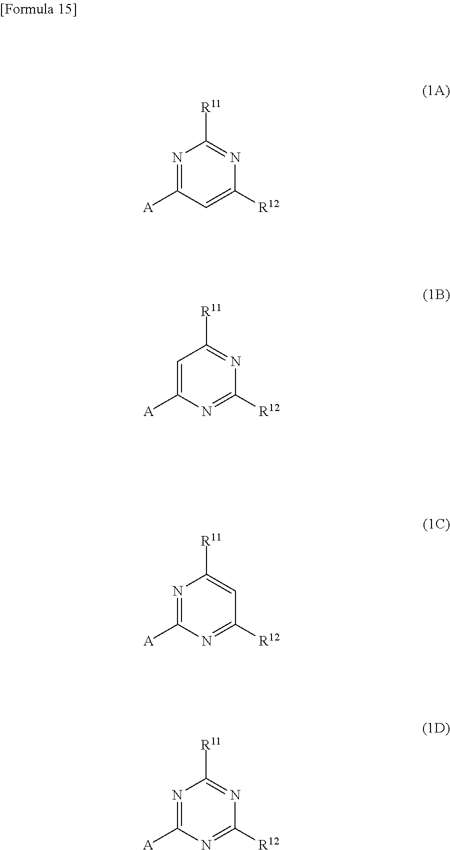

In the formula (1), R¹¹ or R¹² is a group selected from groups represented by formulae (1a) to (1p) below.

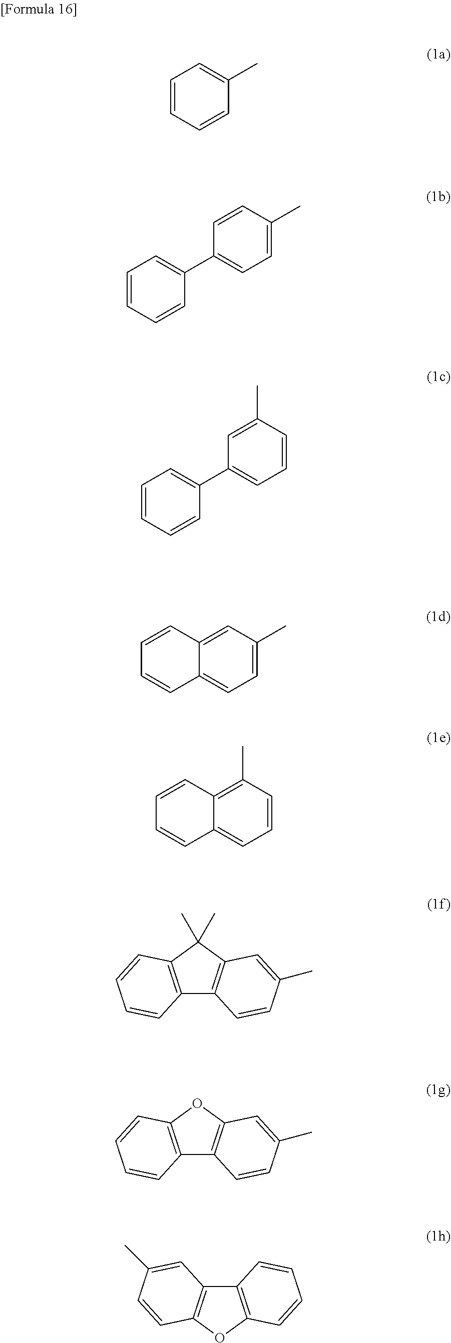

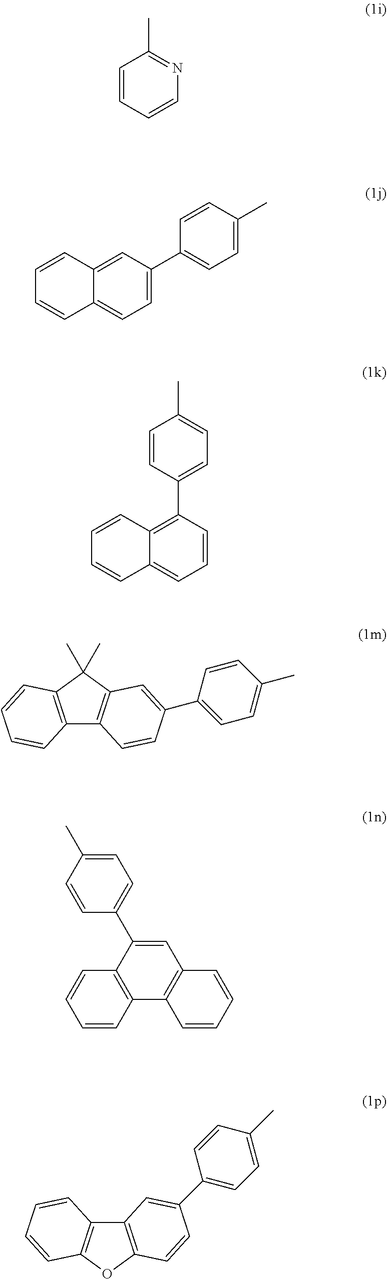

In the formula (1), it is preferable that X¹ and X³ are nitrogen atoms, X⁴ is CR¹², and R¹² is a group selected from groups represented by the formulae (1a) to (1p). In other words, it is preferable that the formula (1) is represented by the formula (1A) and R¹² is a group selected from the groups represented by the formulae (1a) to (1p).

The compound represented by the formula (1) is preferably a compound represented by a formula (1-1) below, more preferably by a formula (1-2) below. The compound represented by the formula (1) is further preferably a compound represented by a formula (1-3) below, particularly preferably by a formula (1-4) below.

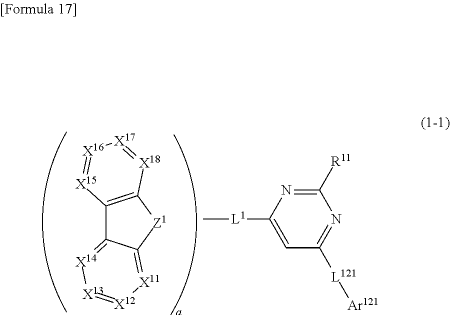

In the formula (1-1), R¹¹ represents the same as R¹¹ of the formula (1). L¹ and L¹²¹ represent the same as L¹ of the formula (11). a represents the same as a of the formula (11). Ar¹²¹ represents the same as Ar¹ of the formula (11). Z¹ and X¹¹ to X¹⁸ respectively represent the same as Z¹ and X¹¹ to X¹⁸ of the formula (12). Note that L¹ is bonded to one of Z¹ and X¹¹ to X¹⁸.

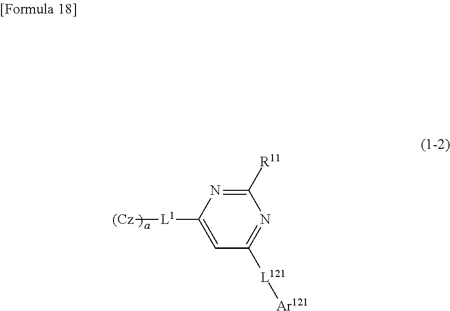

In the formula (1-2), R¹¹ represents the same as R¹¹ of the formula (1). L¹ and L¹²¹ represent the same as L¹ of the formula (11). a represents the same as a of the formula (11). Ar¹²¹ represents the same as Ar¹ of the formula (11). Cz is a group represented by a formula (1-21) below.

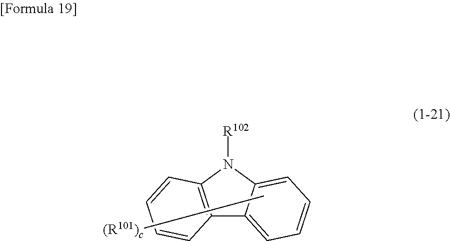

In the formula (1-21), R¹⁰¹ each independently represents the same as R¹³ of the formula (12) and R¹⁰² represents the same as R¹⁴ of the formula (12). c is an integer of 1 to 8. When c is 2 or more, a plurality of R¹⁰¹ are mutually the same or different.

However, at least one of R¹⁰¹ and R¹⁰² is a single bond to be bonded to L¹ in the formula (1-2). Note that R¹⁰¹ is bonded to any one of carbon atoms in the carbazolyl group.

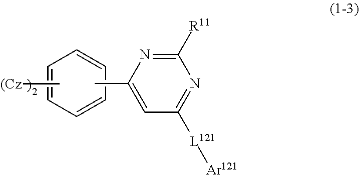

In the formula (1-3), R¹¹, L¹²¹, Ar¹²¹ and Cz respectively represent the same as R¹¹, L¹²¹, Ar¹²¹ and Cz of the formula (1-2). Note that Cz and a pyrimidine ring are bonded to any ones of carbon atoms in a benzene ring.

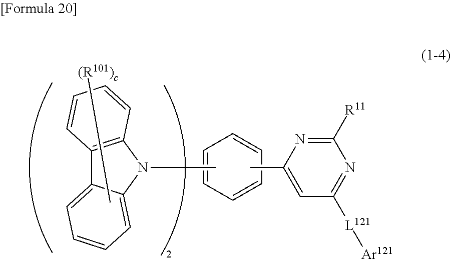

In the formula (1-4), R¹¹, L¹²¹, Ar¹²¹ and Cz respectively represent the same as R¹¹, L¹²¹, Ar¹²¹ and Cz of the formula (1-2). R¹⁰¹ and c respectively represent the same as R¹⁰¹ and c of the formula (1-21). Note that the carbazolyl group and the pyrimidine ring are bonded to any ones of carbon atoms in the benzene ring. Moreover, R¹⁰¹ is bonded to any one of carbon atoms of the carbazolyl group.

In the formula (1-4), it is more preferable that R¹⁰¹ is a hydrogen atom and R¹¹ is a substituted or unsubstituted aromatic hydrocarbon group having 6 to 40 ring carbon atoms. R¹¹ is further preferably a substituted or unsubstituted phenyl group, particularly preferably an unsubstituted phenyl group.

Moreover, in the formula (1-4), it is more preferable that -L¹²¹-Ar¹²¹ is represented by one of the formulae (1a) to (1p).

Next, each of the substituents described in the formulae (1), (1A) to (1D), (1-1) to (1-4), (1-21), (11), (12), (12A), (12B), (12A-1) to (12A-3), (12B-1) to (12B-6), (14) and (100) will be described below. Examples of the substituents described in the formulae (1), (1A) to (1D), (1-1) to (1-4), (1-21), (11), (12), (12A), (12B), (12A-1) to (12A-3), (12B-1) to (12B-6), (14) and (100) include a halogen atom, cyano group, nitro group, hydroxyl group, carboxyl group, sulfonyl group, mercapto group, substituted or unsubstituted boryl group, substituted or unsubstituted phosphino group, substituted or unsubstituted acyl group, substituted or unsubstituted amino group, substituted or unsubstituted silyl group, substituted or unsubstituted alkyl group having 1 to 30 carbon atoms, substituted or unsubstituted alkenyl group having 2 to 30 carbon atoms, substituted or unsubstituted alkynyl group having 2 to 30 carbon atoms, substituted or unsubstituted aralkyl group having 6 to 30 carbon atoms, substituted or unsubstituted alkoxy group having 1 to 30 carbon atoms, substituted or unsubstituted aryloxy group having 6 to 40 ring carbon atoms, substituted or unsubstituted heteroaryloxy group having 5 to 40 ring carbon atoms, substituted or unsubstituted alkylthio group having 1 to 30 carbon atoms, substituted or unsubstituted arylthio group having 6 to 40 ring carbon atoms, substituted or unsubstituted heteroarylthio group having 5 to 40 ring carbon atoms, substituted or unsubstituted alkoxycarbonyl group having 2 to 30 carbon atoms, substituted or unsubstituted aryloxycarbonyl group having 6 to 40 ring carbon atoms, substituted or unsubstituted heteroaryloxycarbonyl group having 5 to 40 ring carbon atoms, substituted or unsubstituted aromatic hydrocarbon group having 6 to 40 ring carbon atoms, and substituted or unsubstituted heterocyclic group having 5 to 40 ring atoms.

Examples of the halogen atom in the formulae (1), (1-1) to (1-4), (1-21), (11), (12), (12A), (12B), (12A-1) to (12A-3), (12B-1) to (12B-6), (14) and (100) include fluorine, chlorine, bromine, and iodine, among which a fluorine atom is preferable.

Examples of the substituted or unsubstituted boryl group in the formulae (1), (1-1) to (1-4), (1-21), (11), (12), (12A), (12B), (12A-1) to (12A-3), (12B-1) to (12B-6), (14) and (100) include a boryl group (—BH₂) and a group represented by —BR^{E}R^{E} that is obtained by substituting H of a boryl group (—BH₂) with R^{E},R^{E}.

Herein, when R^{E} is an alkyl group, the group represented by —BR^{E}R^{E} is an alkylboryl group, preferably a substituted or unsubstituted alkylboryl. The alkyl group as R^{E} is preferably an alkyl group having 1 to 30 carbon atoms below.

When R^{E} is an aryl group, the group represented by —BR^{E}R^{E} is an arylboryl group, preferably a substituted or unsubstituted arylboryl. The aryl group as R^{E} is preferably an aromatic hydrocarbon group having 6 to 40 ring carbon atoms below.

When R^{E} is a heteroaryl group, the group represented by —BR^{E}R^{E} is a heteroarylboryl group, preferably a substituted or unsubstituted heteroarylboryl. The heteroaryl group as R^{E} is preferably a hetercyclic group having 5 to 40 ring atoms below. In addition, a dihydroxyboryl group (—B(OH)₂) is also usable.

Examples of the substituted or unsubstituted phosphino group in the formulae (1), (1-1) to (1-4), (1-21), (11), (12), (12A), (12B), (12A-1) to (12A-3), (12B-1) to (12B-6), (14) and (100) include a phosphino group (—BH₂), a group represented by —BR^{E}R^{E} that is obtained by substituting H of a phosphino group (—BH₂) with R^{E},R^{E}, and a group represented by —P(O)R^{F}R^{F}.

Herein, when R^{F} is an alkyl group, the group represented by —BR^{E}R^{E} is an alkylphosphino group, preferably a substituted or—unsubstituted alkylphosphino. The alkyl group as R^{F} is preferably an alkyl group having 1 to 30 carbon atoms below.

When R^{F} is an aryl group, the group represented by —PR^{F}R^{F} is an arylphosphino group, preferably a substituted or unsubstituted arylphosphino. The aryl group as R^{F} is preferably an aromatic hydrocarbon group having 6 to 40 ring carbon atoms below.

When R^{F} is a heteroaryl group, the group represented by —PR^{F}R^{F} is a heteroarylphosphino group, preferably a substituted or unsubstituted heteroarylphosphino. The heteroaryl group as R^{F} is preferably a hetercyclic group having 5 to 40 ring atoms below.

The substituted or unsubstituted acyl group in the formulae (1), (1-1) to (1-4), (1-21), (11), (12), (12A), (12B), (12A-1) to (12A-3), (12B-1) to (12B-6), (14) and (100) is represented by —CO—R^{D}.

Herein, when R^{D} is an alkyl group, the acyl group is an alkylcarbonyl group, preferably a substituted or unsubstituted alkylcarbonyl group having 2 to 30 carbon atoms. The alkyl group as R^{D} is preferably an alkyl group having 1 to 30 carbon atoms below. Examples of the alkylcarbonyl group include an acetyl group, propionyl group, butyryl group, valeryl group, pivaloyl group, palmitoyl group, stearoyl group and oleoyl group.

Moreover, when R^{D} is an aryl group, the acyl group is an arylcarbonyl group (also referred to as an aroyl group), preferably a substituted or unsubstituted arylcarbonyl group having 6 to 40 ring carbon atoms. The aryl group as R^{D} is preferably an aromatic hydrocarbon group having 6 to 40 ring carbon atoms below. Examples of the arylcarbonyl group include a benzoyl group, toluoyl group, salicyloyl group, cinnamoyl group, naphthoyl group and phthaloyl group.

Moreover, when R^{D} is a heteroaryl group, the represented by —CO—R^{D} is a heteroarylcarbonyl group, preferably a substituted or unsubstituted heteroarylcarbonyl group having 5 to 40 ring atoms. The heteroaryl group as R^{D} is preferably a hetercyclic group having 5 to 40 ring atoms below. Examples of the heteroarylcarbonyl group include a furoyl group, pyrrolylcarbonyl group, pyridylcarbonyl group and thienylcarbonyl group.

Note that a formyl group (—CO—H) in which R^{D} is substituted with a hydrogen atom is included in the examples of the acyl group.

Examples of the substituted or unsubstituted boryl group in the formulae (1), (1-1) to (1-4), (1-21), (11), (12), (12A), (12B), (12A-1) to (12A-3), (12B-1) to (12B-6), (14) and (100) include an amino group (—NH₂) and an amino group in which H of an amino group (—NH₂) are substituted with substituents. Examples of the substituted amino group include an alkylamino group substituted with a substituted or unsubstituted alkyl having 1 to 30 carbon atoms, an arylamino group substituted with a substituted or unsubstituted aryl group having 6 to 40 ring carbon atoms, a heteroarylamino group substituted with a substituted or unsubstituted heteroaryl group having 5 to 40 ring atoms, and an acylamino group substituted with a substituted or unsubstituted acyl group having 2 to 30 carbon atoms.

The alkyl group having 1 to 30 carbon atoms in the alkylamino group is preferably an alkyl group having 1 to 30 carbon atoms listed below. When the amino group is substituted with two alkyl groups, the two alkyl groups may be mutually the same or different.

The aryl group having 6 to 40 ring carbon atoms in the arylamino group is preferably an aromatic hydrocarbon group having 6 to 40 ring carbon atoms. The arylamino group is preferably an amino group substituted with a phenyl group. When the amino group is substituted with two aryl groups, the two aryl groups may be mutually the same or different.

The heteroaryl group having 5 to 40 ring atoms in the heteroarylamino group is preferably a heterocyclic group having 5 to 40 ring atoms below. When the amino group is substituted with two heteroaryl groups, the two heteroaryl groups may be mutually the same or different.

The acyl group having 2 to 30 carbon atoms in the acylamino group is preferably selected from the above acyl group.

The substituted amino group may be an amino group substituted with two selected from a hydrogen atom, alkyl group, aryl group, heteroaryl group and acyl group.

The substituted amino group may be an amino group substituted with an alkyl group and an aryl group. Examples of the substituted amino group include an alkylarylamino group, alkylheteroarylamino group, arylheteroarylamino group, alkylacylamino group and arylacylamino group.

Examples of the substituted or unsubstituted silyl group in the formulae (1), (1-1) to (1-4), (1-21), (11), (12), (12A), (12B), (12A-1) to (12A-3), (12B-1) to (12B-6), (14) and (100) include an unsubstituted silyl group, an alkylsilyl group substituted with a substituted or unsubstituted alkyl group having 1 to 30 carbon atoms, an arylsilyl group substituted with a substituted or unsubstituted aryl group having 6 to 40 ring carbon atoms, and a heteroarylsilyl group substituted with a substituted or unsubstituted heteroaryl group having 5 to 40 ring atoms.

The alkylsilyl group is exemplified by a trialkylsilyl group having the above alkyl group having 1 to 30 carbon atoms. Specific examples of the trialkylsilyl group include a trimethylsilyl group, triethylsilyl group, tri-n-butylsilyl group, tri-n-octylsilyl group, triisobutylsilyl group, dimethylethylsilyl group, dimethylisopropylsilyl group, dimethyl-n-propylsilyl group, dimethyl-n-butylsilyl group, dimethyl-t-butylsilyl group, diethylisopropylsilyl group, vinyldimethylsilyl group, propyldimethylsilyl group and triisopropylsilyl group. Three alkyl groups in the trialkylsilyl group may be mutually the same or different.

The arylsilyl group is exemplified by a triarylsilyl group including three of aromatic hydrocarbon groups each having 6 to 40 ring carbon atoms below. The triarylsilyl group preferably has 18 to 30 carbon atoms. Three aryl groups may be mutually the same or different.

The heteroarylsilyl group is exemplified by a triheteroarylsilyl group having three of heterocyclic groups each having 5 to 40 ring atoms below. Three heteroaryl groups may be mutually the same or different.

The substituted silyl group may be a silyl group substituted with at least two groups selected from an alkyl group, aryl group and heteroaryl group.

The substituted silyl group may be a silyl group substituted with an alkyl group and an aryl group. Examples of the substituted silyl group include an alkylarylsilyl group, dialkylarylsilyl group, diarylsilyl group, alkkyldiarylsilyl group and triarlysilyl group. A plurality of aryl groups or a plurality of alkyl groups may be mutually the same or different.

The dialkylarylsilyl group is exemplified by a dialkylarylsilyl group having two of the examples of the alkyl group having 1 to 30 carbon atoms and one of the examples of the aromatic hydrocarbon group having 6 to 40 ring carbon atoms. The dialkylarylsilyl group preferably has 8 to 30 carbon atoms. The two alkyl groups may be mutually the same or different.

The alkyldiarylsilyl group is exemplified by an alkyldiarylsilyl group including one of the alkyl group listed as the examples of the alkyl group having 1 to 30 carbon atoms and two of the aryl group listed as the examples of the aryl group having 6 to 40 ring carbon atoms. The alkyldiarylsilyl group preferably has 13 to 30 carbon atoms. The two aryl groups may be mutually the same or different.

Examples of the above arylsilyl group are a phenyldimethylsilyl group, a diphenylmethylsilyl group, a diphenyl-t-butylsilyl group and a triphenylsilyl group.

The substituted silyl group may be a silyl group substituted with an alkyl group and a heteroaryl group, a silyl group substituted with an aryl group and a heteroaryl group, and a silyl group substituted with an alkyl group, aryl group and heteroaryl group.

The substituted or unsubstituted alkyl group having 1 to 30 carbon atoms in the formulae (1), (1-1) to (1-4), (1-21), (11), (12), (12A), (12B), (12A-1) to (12A-3), (12B-1) to (12B-6), (14) and (100) may be linear, branched or cyclic. Examples of the substituted alkyl group having 1 to 30 carbon atoms include a haloalkyl group. The haloalkyl group is exemplified by a haloalkyl group obtained by substituting the alkyl group having 1 to 30 carbon atoms with one or more halogen atoms. Examples of the substituted or unsubstituted linear or branched alkyl group are a methyl group, ethyl group, propyl group, isopropyl group, n-butyl group, s-butyl group, isobutyl group, t-butyl group, n-pentyl group, n-hexyl group, n-heptyl group, n-octyl group, n-nonyl group, n-decyl group, n-undecyl group, n-dodecyl group, n-tridecyl group, n-tetradecyl group, n-pentadecyl group, n-hexadecyl group, n-heptadecyl group, n-octadecyl group, neopentyl group, 1-methylpentyl group, 2-methylpentyl group, 1-pentylhexyl group, 1-butylpentyl group, 1-heptyloctyl group, 3-methylpentyl group, hydroxymethyl group, 1-hydroxyethyl group, 2-hydroxyethyl group, 2-hydroxyisobutyl group, 1,2-dihydroxyethyl group, 1,3-dihydroxyisopropyl group, 2,3-dihydroxy-t-butyl group, 1,2,3-trihydroxypropyl group, chloromethyl group, 1-chloroethyl group, 2-chloroethyl group, 2-chloroisobutyl group, 1,2-dichloroethyl group, 1,3-dichloroisopropyl group, 2,3-dichloro-t-butyl group, 1,2,3-trichloropropyl group, bromomethyl group, 1-bromoethyl group, 2-bromoethyl group, 2-bromoisobutyl group, 1,2-dibromoethyl group, 1,3-dibromoisopropyl group, 2,3-dibromo-t-butyl group, 1,2,3-tribromopropyl group, iodomethyl group, 1-iodoethyl group, 2-iodoethyl group, 2-iodoisobutyl group, 1,2-diiodoethyl group, 1,3-diiodoisopropyl group, 2,3-diiodo-t-butyl group, 1,2,3-triiodopropyl group, aminomethyl group, 1-aminoethyl group, 2-aminoethyl group, 2-aminoisobutyl group, 1,2-diaminoethyl group, 1,3-diaminoisopropyl group, 2,3-diamino-t-butyl group, 1,2,3-triaminopropyl group, cyanomethyl group, 1-cyanoethyl group, 2-cyanoethyl group, 2-cyanoisobutyl group, 1,2-dicyanoethyl group, 1,3-dicyanoisopropyl group, 2,3-dicyano-t-butyl group, 1,2,3-tricyanopropyl group, nitromethyl group, 1-nitroethyl group, 2-nitroethyl group, 1,2-dinitroethyl group, 2,3-dinitro-t-butyl group, 1,2,3-trinitropropyl group, fluoromethyl group, difluoromethyl group, trifluoromethyl group, fluoroethyl group, 2,2,2-trifluoroethyl group and 1,1,1,3,3,3-hexafluoro-2-propyl group.

The substituted or unsubstituted cyclic alkyl group (cycloalkyl group) is preferably a substituted or unsubstituted cycloalkyl group having 3 to 30 carbon atoms. Examples of the cycloalkyl group include a cyclopropyl group, cyclobutyl group, cyclopentyl group, cyclohexyl group, cyclooctyl group, 4-methylcyclohexyl group, 3,5-tetramethylcyclohexyl group, 1-adamantyl group, 2-adamantyl group, 1-norbornyl group and 2-norbornyl group.

Among the above alkyl group, an alkyl group having 1 to 10 carbon atoms is preferable. An alkyl group having 1 to 8 carbon atoms is more preferable. An alkyl group having 1 to 6 carbon atoms is particularly preferable. Among the above alkyl group, a methyl group, isopropyl group, t-butyl group and cyclohexyl group are preferable.

The substituted or unsubstituted alkenyl group having 1 to 30 carbon atoms in the formulae (1), (1-1) to (1-4), (1-21), (11), (12), (12A), (12B), (12A-1) to (12A-3), (12B-1) to (12B-6), (14) and (100) may be linear, branched or cyclic. Examples of the alkenyl group are a vinyl group, propenyl group, butenyl group, oleyl group, eicosapentaenyl group, docosahexaenyl group, styryl group, 2,2-diphenylvinyl group, 1,2,2-triphenylvinyl group and 2-phenyl-6-propenyl group. Among the above alkenyl group, a vinyl group is preferable.

Examples of the substituted or unsubstituted alkynyl group having 2 to 30 carbon atoms in the formulae (1), (1-1) to (1-4), (1-21), (11), (12), (12A), (12B), (12A-1) to (12A-3), (12B-1) to (12B-6), (14) and (100) include ethynyl, propynyl and 2-phenylethynyl. Among the above alkynyl group, an ethynyl group is preferable.

The aralkyl group having 7 to 40 carbon atoms in the formulae (1), (1-1) to (1-4), (1-21), (11), (12), (12A), (12B), (12A-1) to (12A-3), (12B-1) to (12B-6), (14) and (100) is represented by —R^{E}—R^{F}. R^{E} is exemplified by an alkylene group provided as a divalent group derived from the alkyl group having 1 to 30 carbon atoms described above. R^{F} is exemplified by the examples of the aromatic hydrocarbon group having 6 to 40 ring carbon atoms. In the aralkyl group, an aryl group moiety has 6 to 40 carbon atoms, preferably 6 to 20 carbon atoms, more preferably 6 to 12 carbon atoms. In the aralkyl group, an alkyl group moiety has 1 to 30 carbon atoms, preferably 1 to 20 carbon atoms, more preferably 1 to 10 carbon atoms, further preferably 1 to 6 carbon atoms. Examples of the aralkyl group are a benzyl group, 2-phenylpropane-2-yl group, 1-phenylethyl group, 2-phenylethyl group, 1-phenylisopropyl group, 2-phenylisopropyl group, phenyl-t-butyl group, α-naphthylmethyl group, 1-α-naphthylethyl group, 2-α-naphthylethyl group, 1-α-naphthylisopropyl group, 2-α-naphthylisopropyl group, P-naphthylmethyl group, 1-β-naphthylethyl group, 2-β-naphthylethyl group, 1-β-naphthylisopropyl group, 2-β-naphthylisopropyl group, 1-pyrorylmethyl group, 2-(1-pyroryl)ethyl group, p-methylbenzyl group, m-methylbenzyl group, o-methylbenzyl group, p-chlorobenzyl group, m-chlorobenzyl group, o-chlorobenzyl group, p-bromobenzyl group, m-bromobenzyl group, o-bromobenzyl group, p-iodobenzyl group, m-iodobenzyl group, o-iodobenzyl group, p-hydroxybenzyl group, m-hydroxybenzyl group, o-hydroxybenzyl group, p-aminobenzyl group, m-aminobenzyl group, o-aminobenzyl group, p-nitrobenzyl group, m-nitrobenzyl group, o-nitrobenzyl group, p-cyanobenzyl group, m-cyanobenzyl group, o-cyanobenzyl group, 1-hydroxy-2-phenylisopropyl group and 1-chloro-2-phenylisopropyl group.

In the formulae (1), (1-1) to (1-4), (1-21), (11), (12), (12A), (12B), (12A-1) to (12A-3), (12B-1) to (12B-6), (14) and (100), the substituted or unsubstituted alkoxy group having 1 to 30 carbon atoms, substituted or unsubstituted aryloxy group having 6 to 40 ring carbon atoms, and substituted or unsubstituted heteroaryloxy group having 5 to 40 ring carbon atom are represented by —OR^{A}.

Herein, when R^{A} is an alkyl group, the group represented by —OR^{A} is an alkoxy group, preferably a substituted or unsubstituted alkoxy group having 1 to 30 carbon atoms. The alkyl group as R^{A} is preferably an alkyl group having 1 to 30 carbon atoms above. Examples of the alkoxy group include a methoxy group, ethoxy group, propoxy group, butoxy group, pentyloxy group and hexyloxy group. Among the alkoxy group, an alkoxy group having 1 to 10 carbon atoms is preferable and an alkoxy group having 1 to 8 carbon atoms is more preferable. An alkoxy group having 1 to 4 carbon atoms is particularly preferable.

Here, examples of the substituted or unsubstituted alkoxy group include a haloalkoxy group that is obtained by substituting an alkyl group (R^{A}) with one or more halogen atoms described above.

Moreover, when R^{A} is an aryl group, the group represented by —OR^{A} is an aryloxy group, preferably a substituted or unsubstituted aryloxy group having 6 to 40 ring carbon atoms. The aryl group as R^{A} is preferably an aromatic hydrocarbon group having 6 to 40 ring carbon atoms below. The aryloxy group is exemplified by a phenoxy group.

Here, examples of the substituted or unsubstituted aryloxy group include a haloaryloxy group that is obtained by substituting an aryl group (R^{A}) with one or more halogen atoms described above.

Moreover, when R^{A} is a heteroaryl group, the group represented by —OR^{A} is a heteroaryloxy group, preferably a substituted or unsubstituted heteroaryloxy group having 5 to 40 ring atoms. The heteroaryl group as R^{A} is preferably a hetercyclic group having 5 to 40 ring atoms below.

In the formulae (1), (1-1) to (1-4), (1-21), (11), (12), (12A), (12B), (12A-1) to (12A-3), (12B-1) to (12B-6), (14) and (100), the substituted or unsubstituted alkylthio group having 1 to 30 carbon atoms, substituted or unsubstituted arylthio group having 6 to 40 ring carbon atoms, and substituted or unsubstituted heteroarylthio group having 5 to 40 ring carbon atom are represented by —SR^{C}.

Herein, when R^{C} is an alkyl group, the group represented by —SR^{C} is an alkylthio group, preferably a substituted or unsubstituted alkylthio group having 1 to 30 carbon atoms. The alkyl group as R^{C} is preferably an alkyl group having 1 to 30 carbon atoms above.

Moreover, when R^{C} is an aryl group, the group represented by —SR^{C} is an arylthio group, preferably a substituted or unsubstituted arylthio group having 6 to 40 ring carbon atoms. The aryl group as R^{C} is preferably an aromatic hydrocarbon group having 6 to 40 ring carbon atoms below.

Moreover, when R^{C} is a heteroaryl group, the group represented by —SR^{C} is a heteroarylthio group, preferably a substituted or unsubstituted heteroarylthio group having 5 to 40 ring atoms. The heteroaryl group as R^{C} is preferably a hetercyclic group having 5 to 40 ring atoms below.

In the formulae (1), (1-1) to (1-4), (1-21), (11), (12), (12A), (12B), (12A-1) to (12A-3), (12B-1) to (12B-6), (14) and (100), the substituted or unsubstituted heteroarylcarbonyl group having 6 to 40 ring carbon atoms, substituted or unsubstituted alkoxycarbonyl group having 2 to 30 carbon atoms, and substituted or unsubstituted aryloxycarbonyl group having 6 to 40 ring carbon atom are represented by —COOR^{B}.

Herein, when R^{B} is an alkyl group, the group represented by —COOR^{B} is an alkoxycarbonyl group, preferably a substituted or unsubstituted alkoxycarbonyl group having 2 to 30 carbon atoms. The alkyl group as R^{B} is preferably an alkyl group having 1 to 30 carbon atoms above.

Moreover, when R^{B} is an aryl group, the group represented by —COOR^{B} is an aryloxycarbonyl group, preferably a substituted or unsubstituted aryloxycarbonyl group having 7 to 40 carbon atoms. The aryl group as R^{B} is preferably an aromatic hydrocarbon group having 6 to 40 ring carbon atoms below.

Moreover, when R^{B} is a heteroaryl group, the group represented by —COOR^{B} is a heteroarylcarbonyl group, preferably a substituted or unsubstituted heteroarylcarbonyl group having 5 to 40 ring atoms. The heteroaryl group as R^{B} is preferably a hetercyclic group having 5 to 40 ring atoms below.

Examples of the aromatic hydrocarbon group having 6 to 40 ring carbon atoms in the formulae (1), (1A) to (1D), (1-1) to (1-4), (1-21), (11), (12), (12A), (12B), (12A-1) to (12A-3), (12B-1) to (12B-6), (14) and (100) include a non-fused aromatic hydrocarbon group and a fused aromatic hydrocarbon group. Specific examples of the aromatic hydrocarbon group include a phenyl group, naphthyl group, anthryl group, phenanthryl group, biphenyl group, terphenyl group, quaterphenyl group, fluoranthenyl group, pyrenyl group, triphenylenyl group, phenanthrenyl group, fluorenyl group, 9,9-dimethylfluorenyl group, spirofluorenyl group, benzo[c]phenanthrenyl group, benzo[a]triphenylenyl group, naphtho[1,2-c]phenanthrenyl group, naphtho[1,2-a]triphenylenyl group, dibenzo[a,c]triphenylenyl group and benzo[b]fluoranthenyl group. Among the above aromatic hydrocarbon group, an aromatic hydrocarbon group having 6 to 30 ring carbon atoms is more preferable. An aromatic hydrocarbon group having 6 to 20 ring carbon atoms is further preferable. An aromatic hydrocarbon group having 6 to 12 ring carbon atoms is particularly preferable.

Examples of the heterocyclic group having 5 to 40 ring atoms in the formulae (1), (1A) to (1D), (1-1) to (1-4), (1-21), (11), (12), (12A), (12B), (12A-1) to (12A-3), (12B-1) to (12B-6), (14) and (100) include a non-fused heterocyclic group and a fused heterocyclic group. Specific examples of the heterocyclic group include a pyroryl group, pyrazinyl group, pyridinyl group, indolyl group, isoindolyl group, furyl group, benzofuranyl group, isobenzofuranyl group, dibenzofuranyl group, dibenzothiophenyl group, quinolyl group, isoquinolyl group, quinoxalinyl group, carbazolyl group, phenanthrydinyl group, acridinyl group, phenanthrolinyl group, thienyl group, and group derived from a pyridine ring, pyrazine ring, pyrimidine ring, pyridazine ring, triazine ring, indole ring, quinoline ring, acridine ring, pyrrolidine ring, dioxane ring, piperidine ring, morpholine ring, piperazine ring, carbazole ring, furan ring, thiophene ring, oxazole ring, oxadiazole ring, benzoxazole ring, thiazole ring, thiadiazole ring, benzothiazole ring, triazole ring, imidazole ring, benzimidazole ring, pyrane ring, dibenzofuran ring, benzo[c]dibenzofuran ring and silafluorene ring. Among the above heterocyclic group, a heterocyclic group having 5 to 40 ring atoms is more preferable. A heterocyclic group having 5 to 20 ring atoms is further preferable. A heterocyclic group having 5 to 12 ring atoms is particularly preferable.

Examples of the polyvalent linear, branched or cyclic aliphatic hydrocarbon group when L¹, L¹¹, L¹² and L¹²¹ are the linking groups in the formulae (11), (14) and (1-1) to (1-4) include: a polyvalent group derived from the polyvalent linear, branched or cyclic alkyl group having 1 to 30 carbon atoms; a polyvalent group derived from the polyvalent linear, branched or cyclic alkenyl group having 1 to 30 carbon atoms; and a polyvalent group derived from the polyvalent linear, branched or cyclic alkynyl group having 1 to 30 carbon atoms, among which a divalent or trivalent group is preferable and a divalent group is more preferable. The divalent group may be substituted with the above-described substituents. Specific examples of the divalent group include a methylene group, ethylene group, acethylenylene and vinylidene group.

Examples of the polyvalent aromatic hydrocarbon group having 6 to 40 ring carbon atoms when L¹, L¹¹, L¹² and L¹²¹ are the linking groups in the formulae (11), (14) and (1-1) to (1-4) include a polyvalent group derived from the aromatic hydrocarbon group having 6 to 40 ring carbon atoms, among which a divalent or trivalent group is preferable and a divalent group is more preferable. Specifically, a divalent group derived from a phenyl group, biphenyl group, naphthyl group and 9,9-dimethylfluorenyl group is preferable. The divalent group may be substituted with the above-described substituents.

Examples of the polyvalent heterocyclic group having 5 to 40 ring atoms when L¹, L¹¹, L¹² and L¹²¹ are the linking groups in the formulae (11), (14) and (1-1) to (1-4) include a polyvalent group derived from the heterocyclic group having 5 to 40 ring atoms, among which a divalent or trivalent group is preferable and a divalent group is more preferable. Specifically, a divalent group derived from a pyridyl group, pyrimidyl group, dibenzofuranyl group, silafluorenyl group and carbazolyl group is preferable. The divalent group may be substituted with the above-described substituents.

In the formula (12), the rest of X¹¹ to X¹⁸ except for the carbon atom to be bonded to L¹ are preferably CR^{Y}, in which R^{Y} is preferably a hydrogen atom or alkyl group, particularly preferably a hydrogen atom.

In the invention, “carbon atoms forming a ring (ring carbon atoms)” mean carbon atoms forming a saturated ring, an unsaturated ring, or an aromatic ring. “Atoms forming a ring (ring atoms)” mean carbon atoms and hetero atoms forming a hetero ring including a saturated ring, unsaturated ring, or aromatic ring.

In the invention, a hydrogen atom includes isotope having different numbers of neutrons, specifically, protium, deuterium and tritium.

Examples of the substituent meant by “substituted or unsubstituted” are the above-described aryl group, heteroaryl group, alkyl group (linear or branched alkyl group, cycloalkyl group and haloalkyl group), alkoxy group, aryloxy group, aralkyl group, haloalkoxy group, alkylsilyl group, dialkylarylsilyl group, alkyldiarylsilyl group, triarylsilyl group, halogen atom, cyano group, hydroxyl group, nitro group and carboxy group. In addition, the alkenyl group and alkynyl group are also usable.

Among the above substituents, an aryl group, heteroaryl group, alkyl group, halogen atom, alkylsilyl group, arylsilyl group and cyano group are preferable. More preferable substituents are one listed as the preferable substituents described for each substituent.

“Unsubstituted” in “substituted or unsubstituted” means that a group is not substituted by the above-described substituents but bonded with a hydrogen atom.

Herein, “a to b carbon atoms” in the description of “substituted or unsubstituted XX group having a to b carbon atoms” represent carbon atoms of an unsubstituted XX group and does not include carbon atoms of a substituted XX group.

The same description as the above applies to “substituted or unsubstituted” in the following compound or a partial structure thereof.

Specific examples of the compound represented by the formula (1) are shown below, but the invention is not limited to thereto.

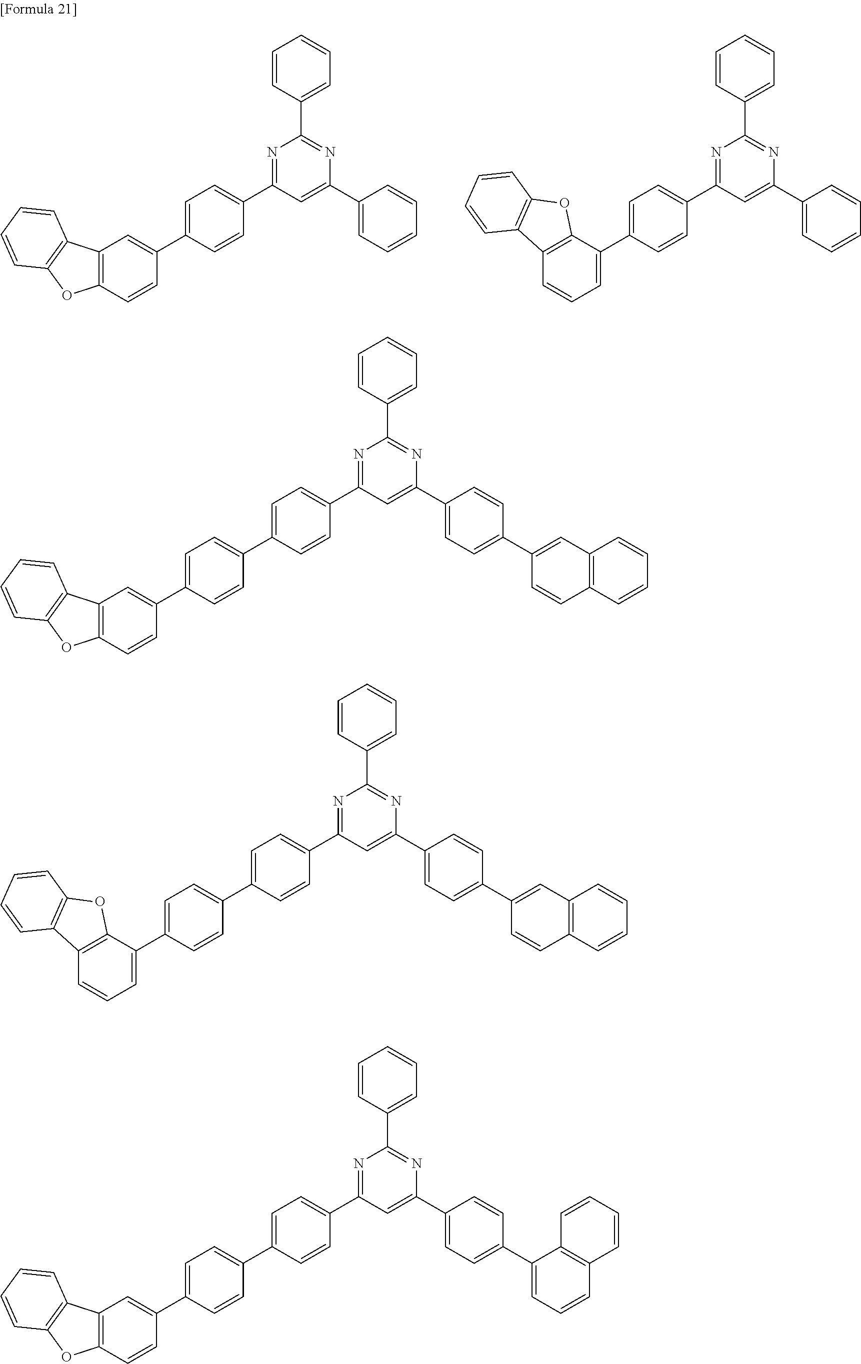 [SMILES: c1ccc(-c2cc(-c3ccc(-c4ccc5oc6ccccc6c5c4)cc3)nc(-c3ccccc3)n2)cc1 ; c1ccc(-c2cc(-c3ccc(-c4cccc5c4oc4ccccc45)cc3)nc(-c3ccccc3)n2)cc1 ; c1ccc(-c2nc(-c3ccc(-c4ccc(-c5ccc6oc7ccccc7c6c5)cc4)cc3)cc(-c3ccc(-c4ccc5ccccc5c4)cc3)n2)cc1 ; c1ccc(-c2nc(-c3ccc(-c4ccc(-c5cccc6c5oc5ccccc56)cc4)cc3)cc(-c3ccc(-c4ccc5ccccc5c4)cc3)n2)cc1 ; c1ccc(-c2nc(-c3ccc(-c4ccc(-c5ccc6oc7ccccc7c6c5)cc4)cc3)cc(-c3ccc(-c4cccc5ccccc45)cc3)n2)cc1]

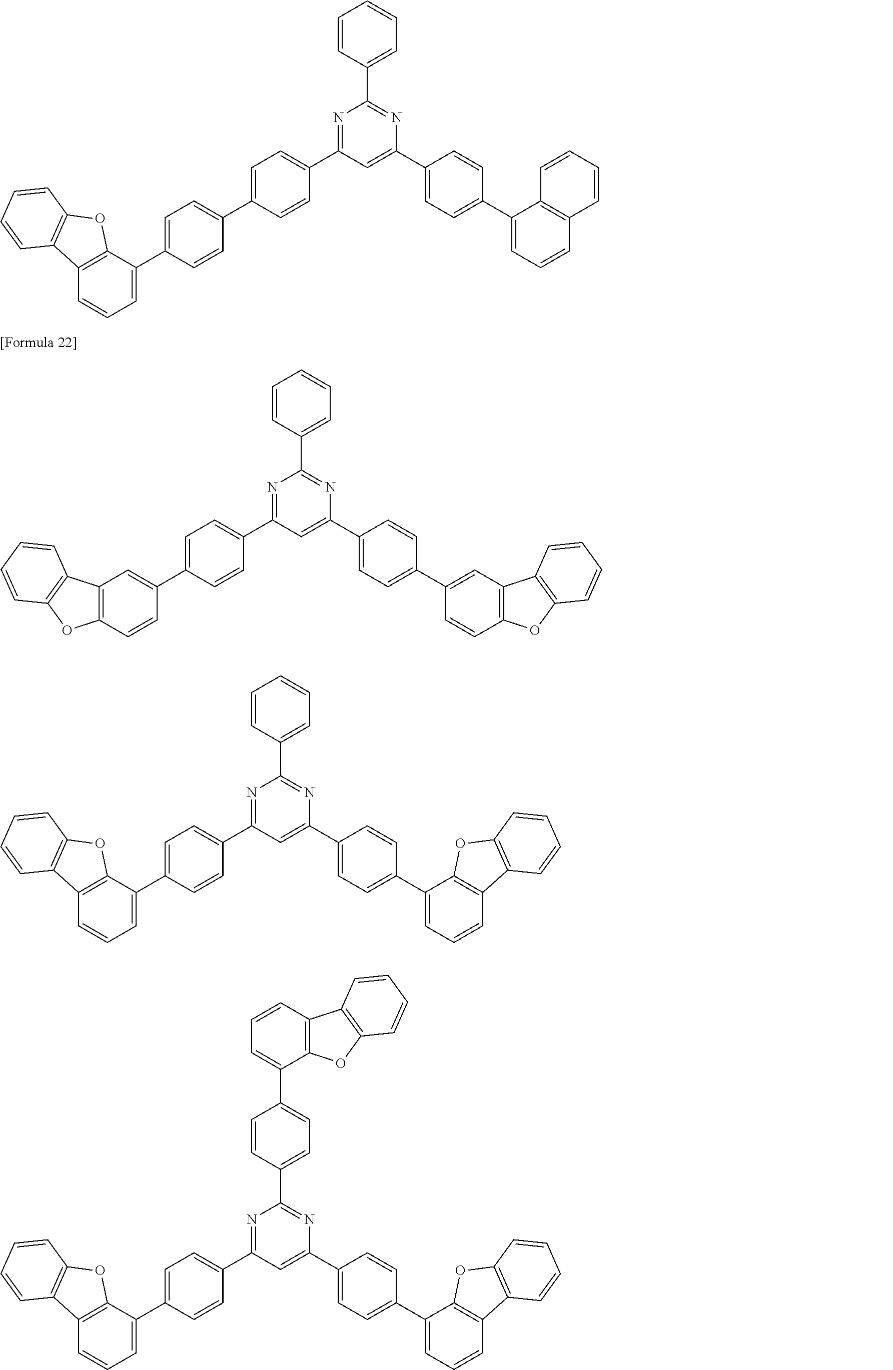 [SMILES: c1ccc(-c2nc(-c3ccc(-c4ccc(-c5cccc6c5oc5ccccc56)cc4)cc3)cc(-c3ccc(-c4cccc5ccccc45)cc3)n2)cc1 ; c1ccc(-c2nc(-c3ccc(-c4ccc5oc6ccccc6c5c4)cc3)cc(-c3ccc(-c4ccc5oc6ccccc6c5c4)cc3)n2)cc1 ; c1ccc(-c2nc(-c3ccc(-c4cccc5c4oc4ccccc45)cc3)cc(-c3ccc(-c4cccc5c4oc4ccccc45)cc3)n2)cc1 ; c1ccc2c(c1)oc1c(-c3ccc(-c4cc(-c5ccc(-c6cccc7c6oc6ccccc67)cc5)nc(-c5ccc(-c6cccc7c6oc6ccccc67)cc5)n4)cc3)cccc12]

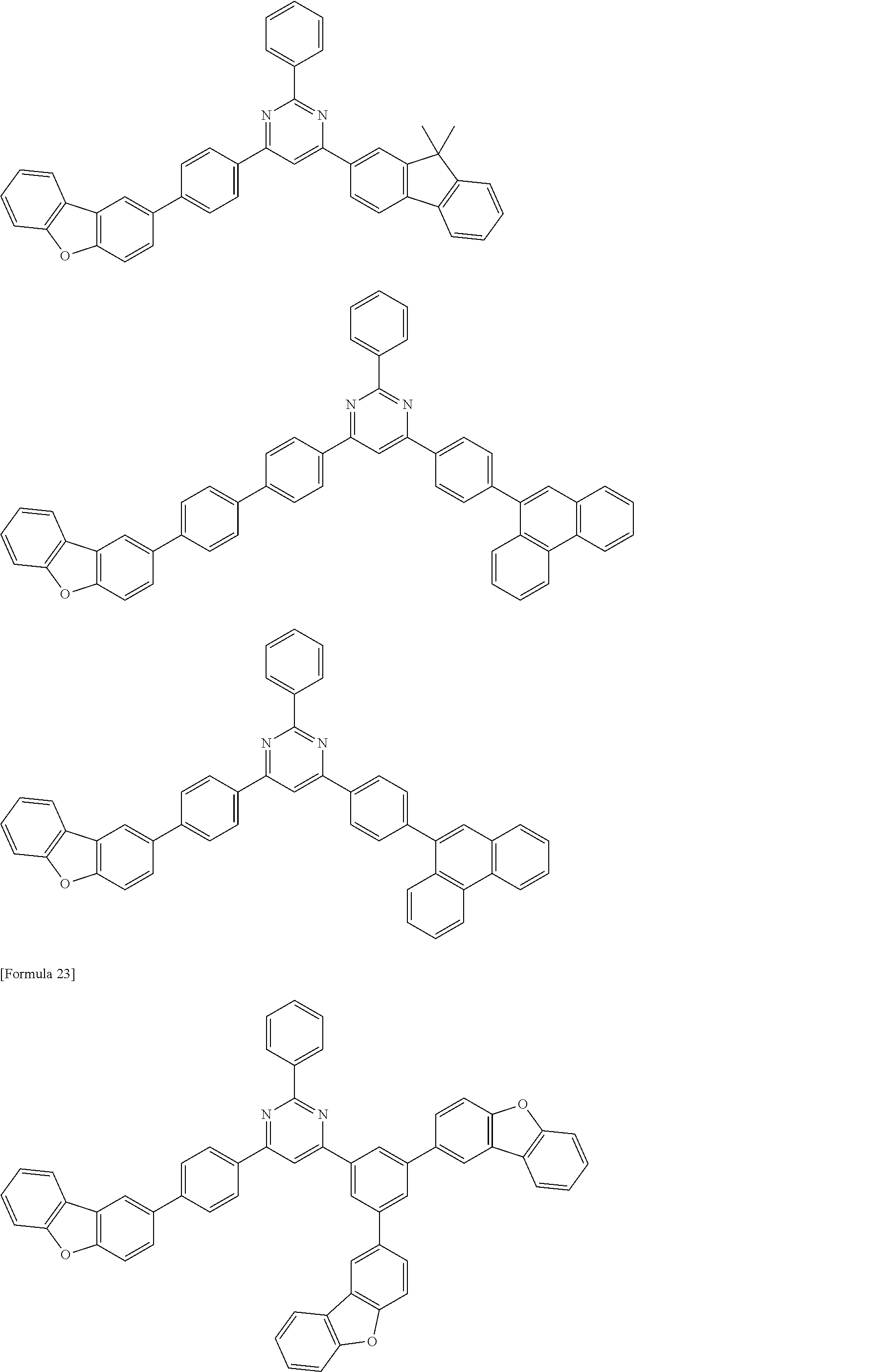 [SMILES: CC1(C)c2ccccc2-c2ccc(-c3cc(-c4ccc(-c5ccc6oc7ccccc7c6c5)cc4)nc(-c4ccccc4)n3)cc21 ; c1ccc(-c2nc(-c3ccc(-c4ccc(-c5ccc6oc7ccccc7c6c5)cc4)cc3)cc(-c3ccc(-c4cc5ccccc5c5ccccc45)cc3)n2)cc1 ; c1ccc(-c2nc(-c3ccc(-c4ccc5oc6ccccc6c5c4)cc3)cc(-c3ccc(-c4cc5ccccc5c5ccccc45)cc3)n2)cc1 ; c1ccc(-c2nc(-c3ccc(-c4ccc5oc6ccccc6c5c4)cc3)cc(-c3cc(-c4ccc5oc6ccccc6c5c4)cc(-c4ccc5oc6ccccc6c5c4)c3)n2)cc1]

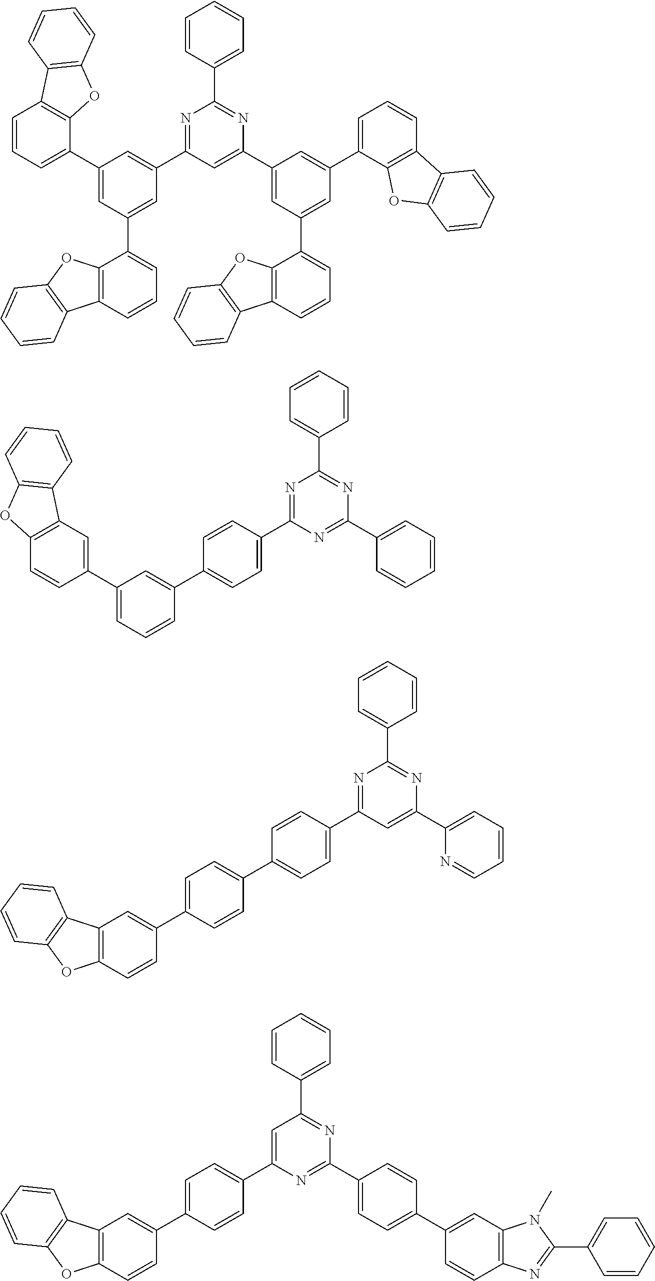 [SMILES: c1ccc(-c2nc(-c3cc(-c4cccc5c4oc4ccccc45)cc(-c4cccc5c4oc4ccccc45)c3)cc(-c3cc(-c4cccc5c4oc4ccccc45)cc(-c4cccc5c4oc4ccccc45)c3)n2)cc1 ; c1ccc(-c2nc(-c3ccccc3)nc(-c3ccc(-c4cccc(-c5ccc6oc7ccccc7c6c5)c4)cc3)n2)cc1 ; c1ccc(-c2nc(-c3ccc(-c4ccc(-c5ccc6oc7ccccc7c6c5)cc4)cc3)cc(-c3ccccn3)n2)cc1 ; Cn1c(-c2ccccc2)nc2ccc(-c3ccc(-c4nc(-c5ccccc5)cc(-c5ccc(-c6ccc7oc8ccccc8c7c6)cc5)n4)cc3)cc21]

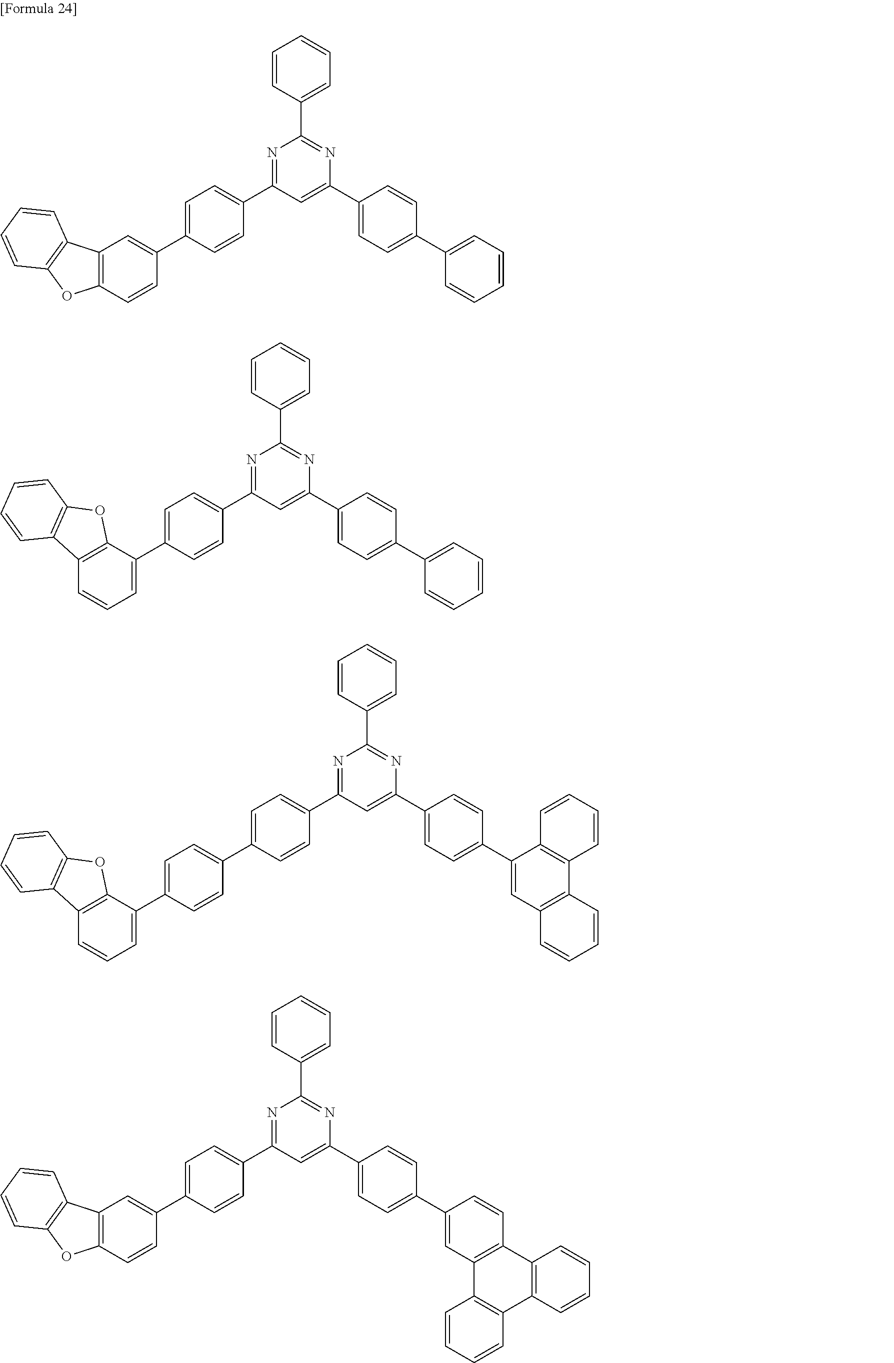 [SMILES: c1ccc(-c2ccc(-c3cc(-c4ccc(-c5ccc6oc7ccccc7c6c5)cc4)nc(-c4ccccc4)n3)cc2)cc1 ; c1ccc(-c2ccc(-c3cc(-c4ccc(-c5cccc6c5oc5ccccc56)cc4)nc(-c4ccccc4)n3)cc2)cc1 ; c1ccc(-c2nc(-c3ccc(-c4ccc(-c5cccc6c5oc5ccccc56)cc4)cc3)cc(-c3ccc(-c4cc5ccccc5c5ccccc45)cc3)n2)cc1 ; c1ccc(-c2nc(-c3ccc(-c4ccc5oc6ccccc6c5c4)cc3)cc(-c3ccc(-c4ccc5c6ccccc6c6ccccc6c5c4)cc3)n2)cc1]

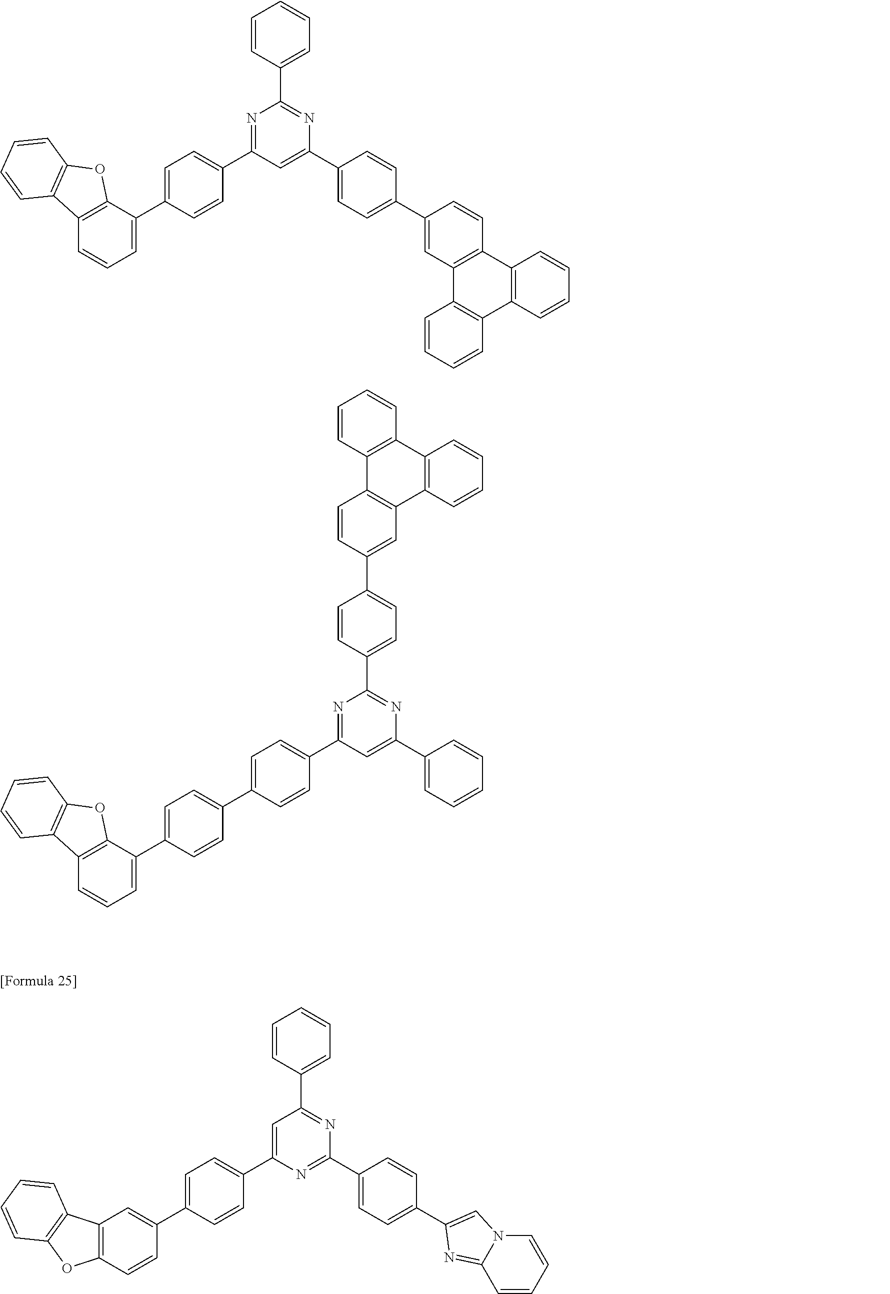 [SMILES: c1ccc(-c2nc(-c3ccc(-c4ccc5c6ccccc6c6ccccc6c5c4)cc3)cc(-c3ccc(-c4cccc5c4oc4ccccc45)cc3)n2)cc1 ; c1ccc(-c2cc(-c3ccc(-c4ccc(-c5cccc6c5oc5ccccc56)cc4)cc3)nc(-c3ccc(-c4ccc5c6ccccc6c6ccccc6c5c4)cc3)n2)cc1 ; c1ccc(-c2cc(-c3ccc(-c4ccc5oc6ccccc6c5c4)cc3)nc(-c3ccc(-c4cn5ccccc5n4)cc3)n2)cc1]

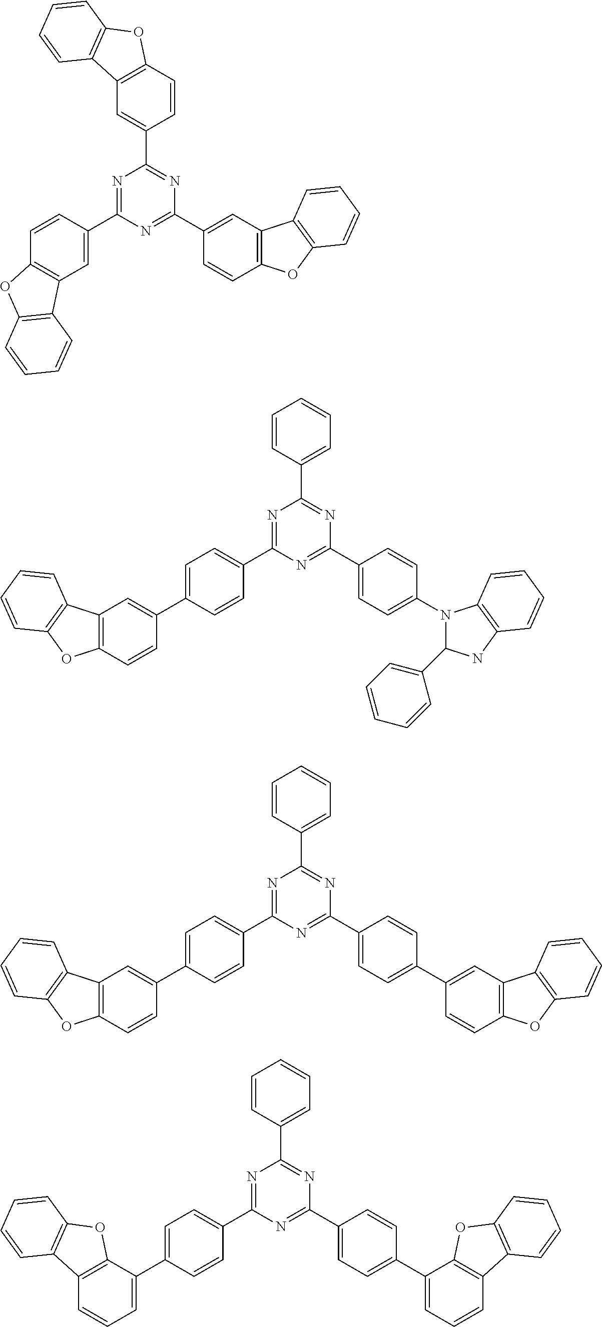 [SMILES: c1ccc2c(c1)oc1ccc(-c3nc(-c4ccc5oc6ccccc6c5c4)nc(-c4ccc5oc6ccccc6c5c4)n3)cc12 ; c1ccc(-c2nc(-c3ccc(-c4ccc5oc6ccccc6c5c4)cc3)nc(-c3ccc(N4c5ccccc5NC4c4ccccc4)cc3)n2)cc1 ; c1ccc(-c2nc(-c3ccc(-c4ccc5oc6ccccc6c5c4)cc3)nc(-c3ccc(-c4ccc5oc6ccccc6c5c4)cc3)n2)cc1 ; c1ccc(-c2nc(-c3ccc(-c4cccc5c4oc4ccccc45)cc3)nc(-c3ccc(-c4cccc5c4oc4ccccc45)cc3)n2)cc1]

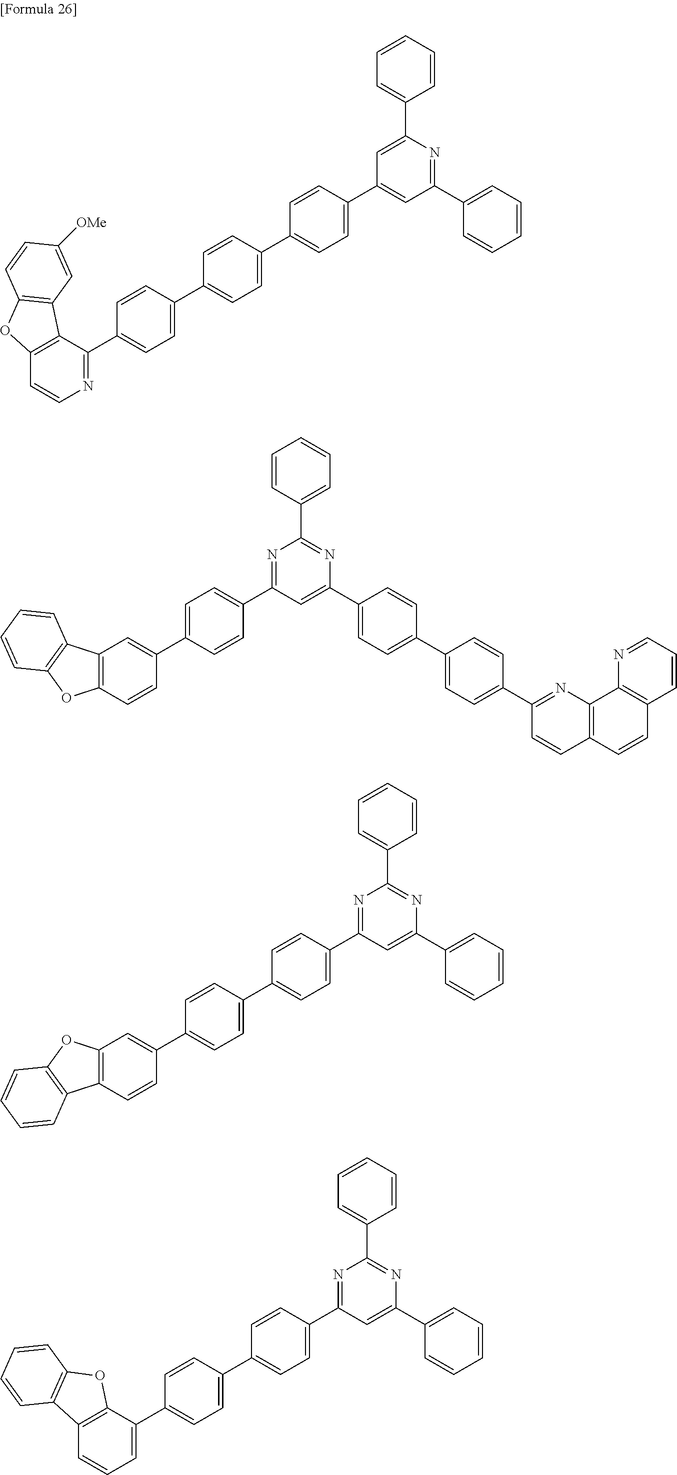 [SMILES: COc1ccc2oc3ccnc(-c4ccc(-c5ccc(-c6ccc(-c7cc(-c8ccccc8)nc(-c8ccccc8)c7)cc6)cc5)cc4)c3c2c1 ; c1ccc(-c2nc(-c3ccc(-c4ccc(-c5ccc6ccc7cccnc7c6n5)cc4)cc3)cc(-c3ccc(-c4ccc5oc6ccccc6c5c4)cc3)n2)cc1 ; c1ccc(-c2cc(-c3ccc(-c4ccc(-c5ccc6c(c5)oc5ccccc56)cc4)cc3)nc(-c3ccccc3)n2)cc1 ; c1ccc(-c2cc(-c3ccc(-c4ccc(-c5cccc6c5oc5ccccc56)cc4)cc3)nc(-c3ccccc3)n2)cc1]

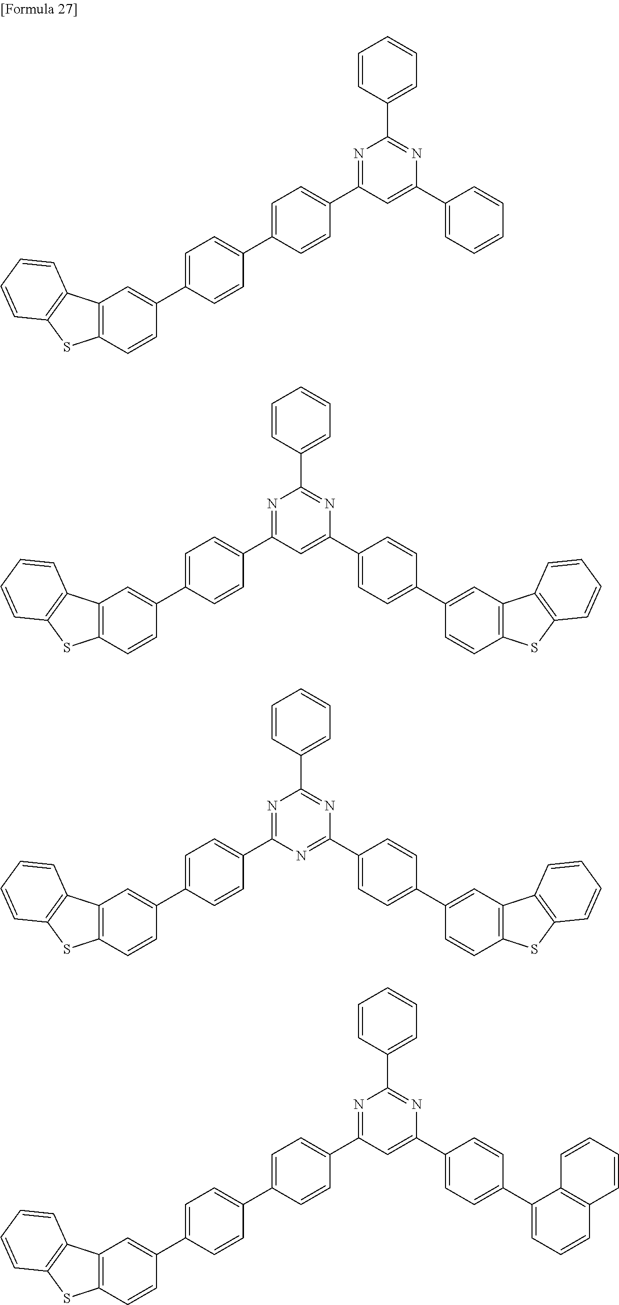 [SMILES: c1ccc(-c2cc(-c3ccc(-c4ccc(-c5ccc6sc7ccccc7c6c5)cc4)cc3)nc(-c3ccccc3)n2)cc1 ; c1ccc(-c2nc(-c3ccc(-c4ccc5sc6ccccc6c5c4)cc3)cc(-c3ccc(-c4ccc5sc6ccccc6c5c4)cc3)n2)cc1 ; c1ccc(-c2nc(-c3ccc(-c4ccc5sc6ccccc6c5c4)cc3)nc(-c3ccc(-c4ccc5sc6ccccc6c5c4)cc3)n2)cc1 ; c1ccc(-c2nc(-c3ccc(-c4ccc(-c5ccc6sc7ccccc7c6c5)cc4)cc3)cc(-c3ccc(-c4cccc5ccccc45)cc3)n2)cc1]

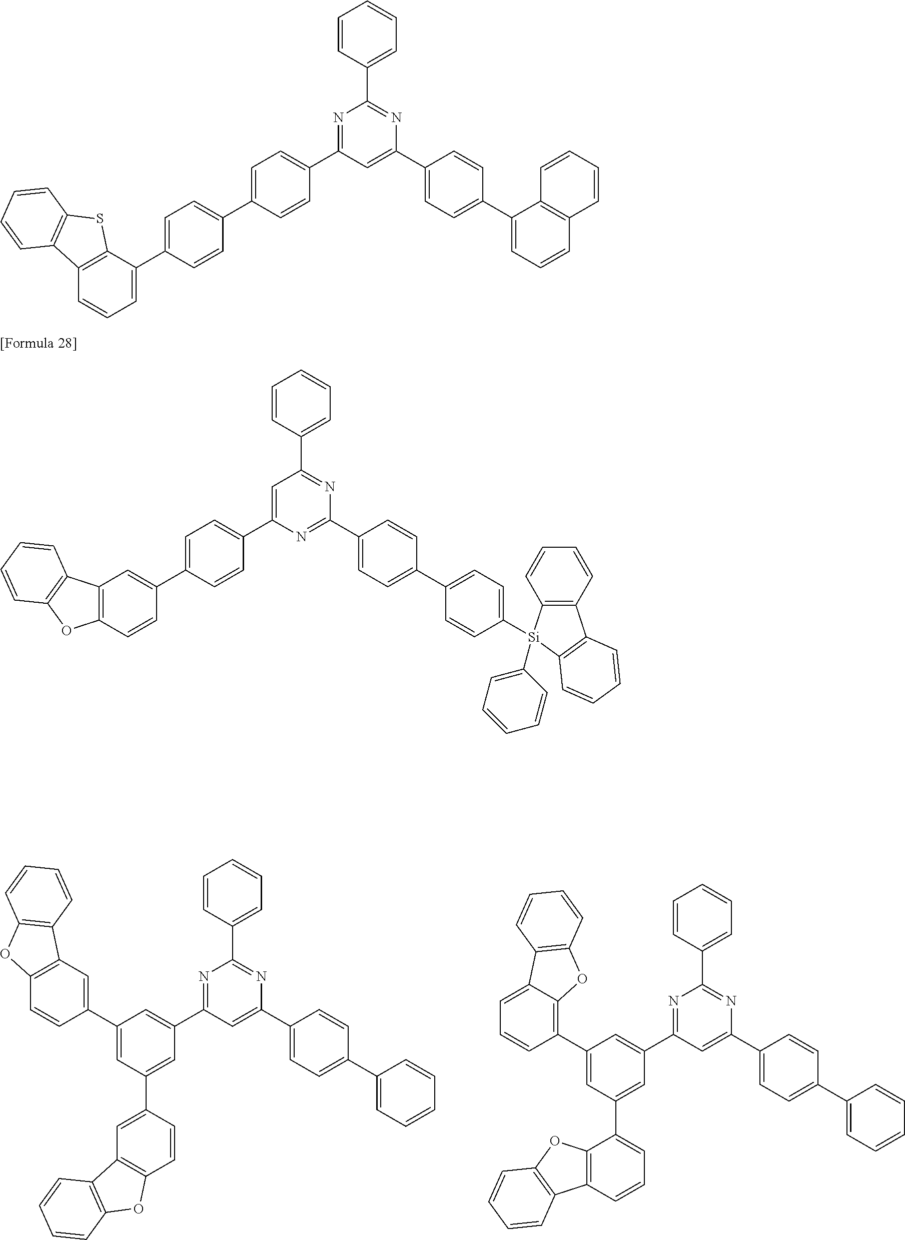 [SMILES: c1ccc(-c2nc(-c3ccc(-c4ccc(-c5cccc6c5sc5ccccc56)cc4)cc3)cc(-c3ccc(-c4cccc5ccccc45)cc3)n2)cc1 ; c1ccc(-c2cc(-c3ccc(-c4ccc5oc6ccccc6c5c4)cc3)nc(-c3ccc(-c4ccc([Si]5(c6ccccc6)c6ccccc6-c6ccccc65)cc4)cc3)n2)cc1 ; c1ccc(-c2ccc(-c3cc(-c4cc(-c5ccc6oc7ccccc7c6c5)cc(-c5ccc6oc7ccccc7c6c5)c4)nc(-c4ccccc4)n3)cc2)cc1 ; c1ccc(-c2ccc(-c3cc(-c4cc(-c5cccc6c5oc5ccccc56)cc(-c5cccc6c5oc5ccccc56)c4)nc(-c4ccccc4)n3)cc2)cc1]

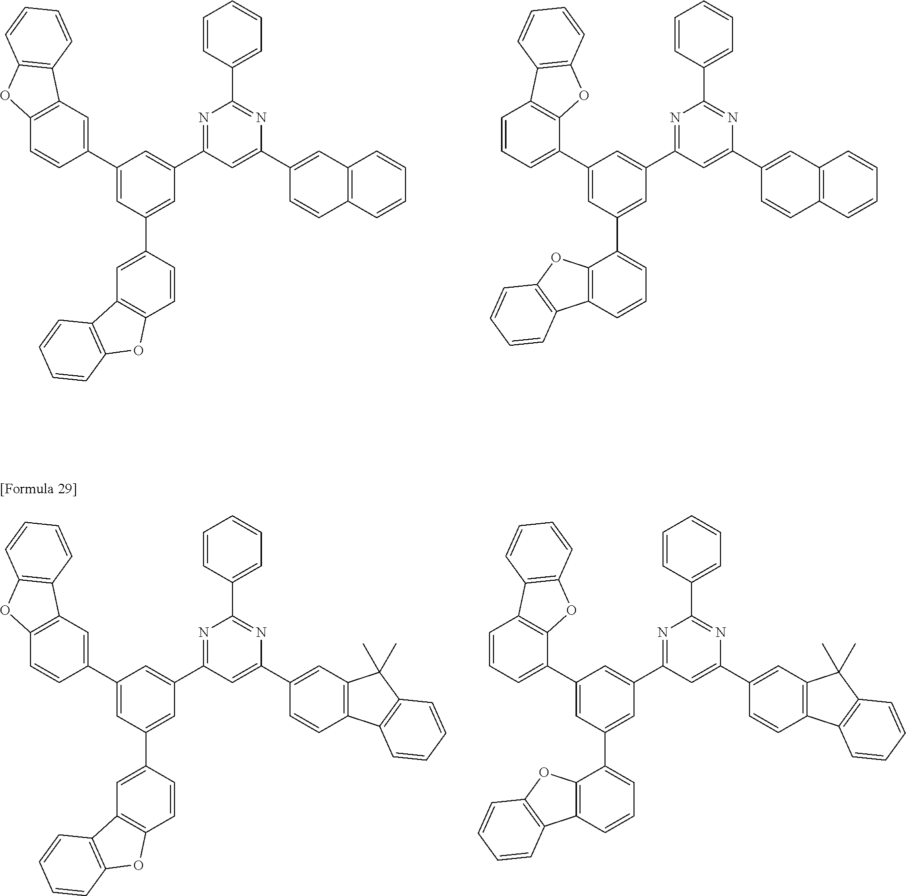 [SMILES: c1ccc(-c2nc(-c3cc(-c4ccc5oc6ccccc6c5c4)cc(-c4ccc5oc6ccccc6c5c4)c3)cc(-c3ccc4ccccc4c3)n2)cc1 ; c1ccc(-c2nc(-c3cc(-c4cccc5c4oc4ccccc45)cc(-c4cccc5c4oc4ccccc45)c3)cc(-c3ccc4ccccc4c3)n2)cc1 ; CC1(C)c2ccccc2-c2ccc(-c3cc(-c4cc(-c5ccc6oc7ccccc7c6c5)cc(-c5ccc6oc7ccccc7c6c5)c4)nc(-c4ccccc4)n3)cc21 ; CC1(C)c2ccccc2-c2ccc(-c3cc(-c4cc(-c5cccc6c5oc5ccccc56)cc(-c5cccc6c5oc5ccccc56)c4)nc(-c4ccccc4)n3)cc21]

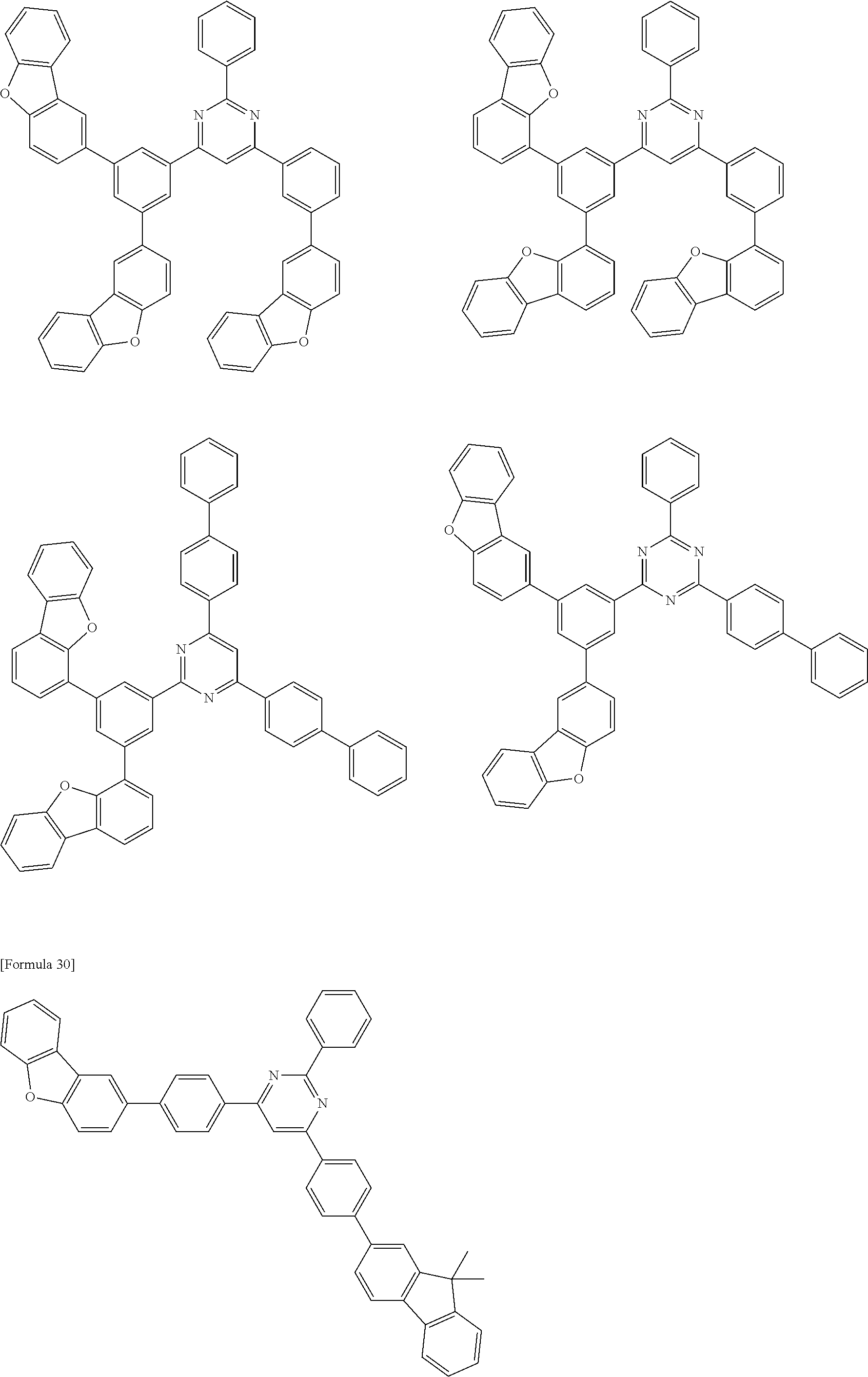

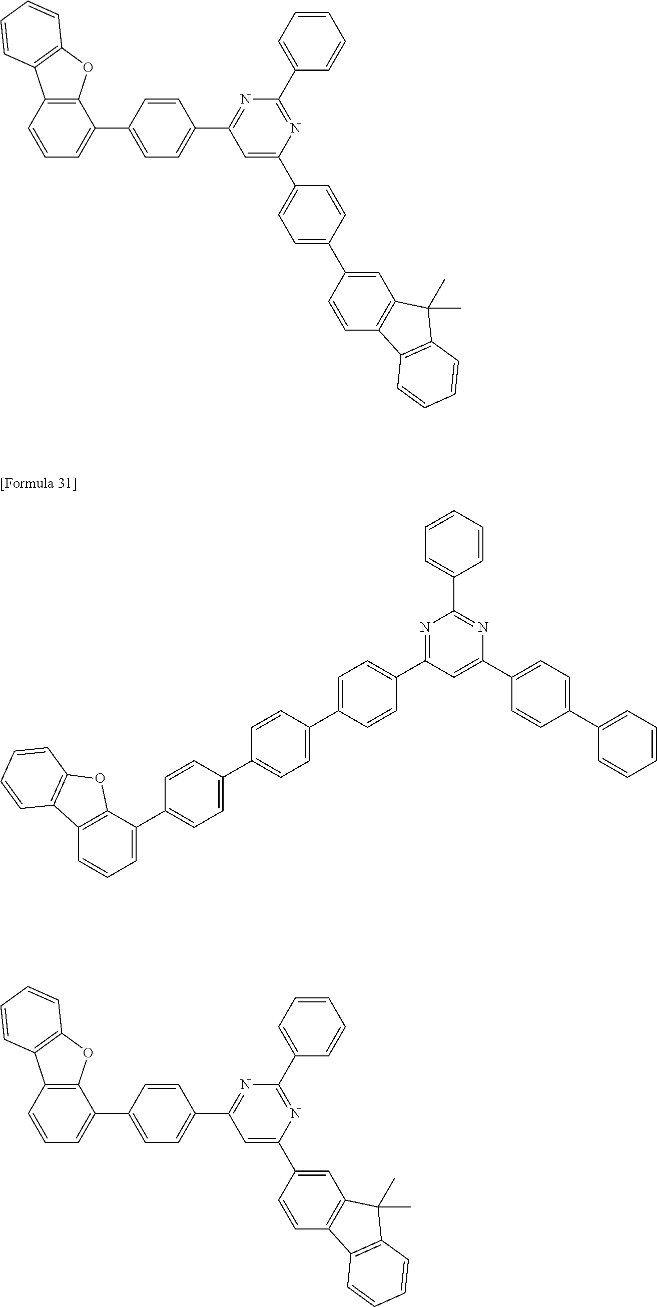 [SMILES: CC1(C)c2ccccc2-c2ccc(-c3ccc(-c4cc(-c5ccc(-c6cccc7c6oc6ccccc67)cc5)nc(-c5ccccc5)n4)cc3)cc21 ; c1ccc(-c2ccc(-c3cc(-c4ccc(-c5ccc(-c6ccc(-c7cccc8c7oc7ccccc78)cc6)cc5)cc4)nc(-c4ccccc4)n3)cc2)cc1 ; CC1(C)c2ccccc2-c2ccc(-c3cc(-c4ccc(-c5cccc6c5oc5ccccc56)cc4)nc(-c4ccccc4)n3)cc21]

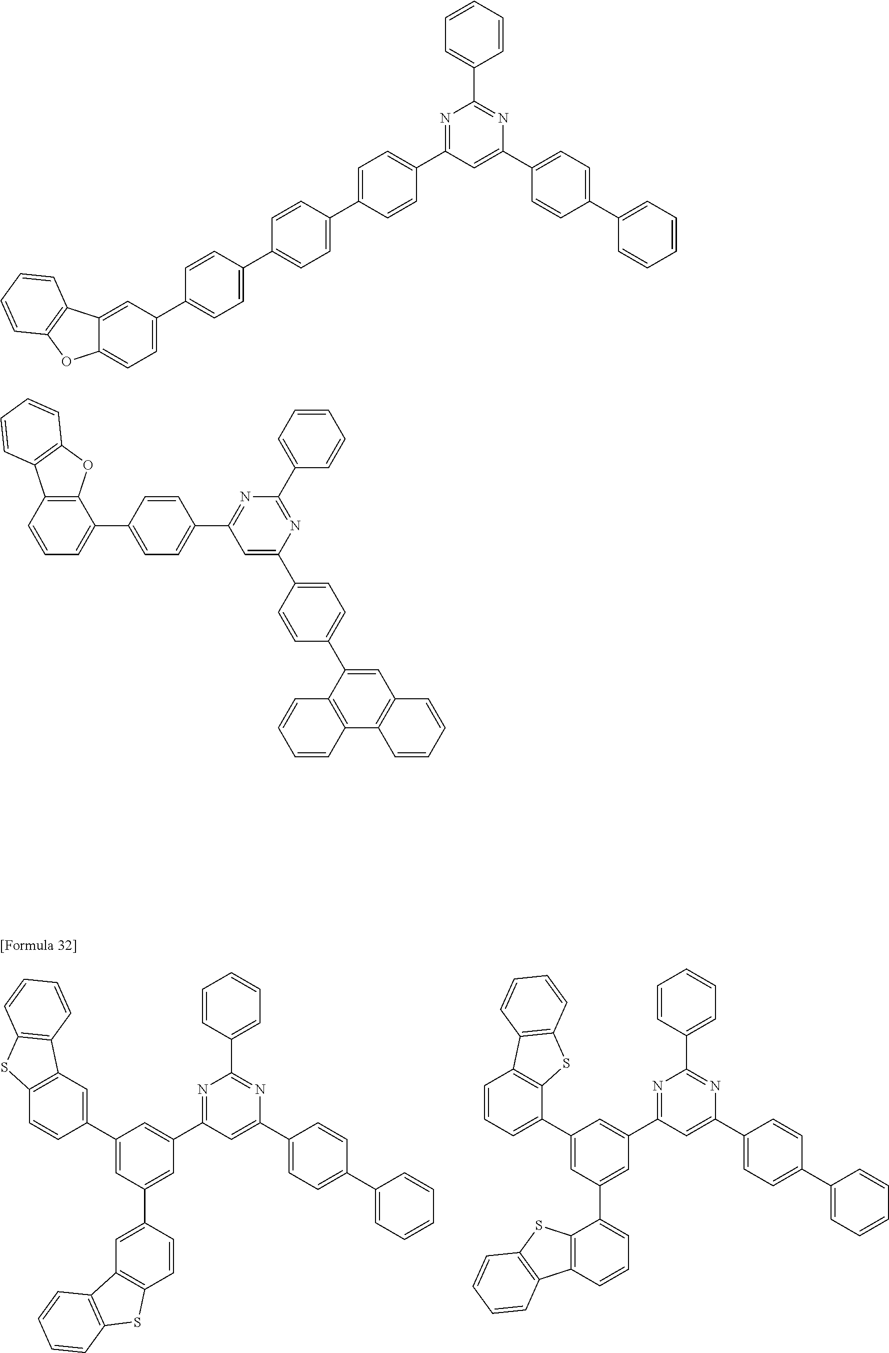 [SMILES: c1ccc(-c2ccc(-c3cc(-c4ccc(-c5ccc(-c6ccc(-c7ccc8oc9ccccc9c8c7)cc6)cc5)cc4)nc(-c4ccccc4)n3)cc2)cc1 ; c1ccc(-c2nc(-c3ccc(-c4cc5ccccc5c5ccccc45)cc3)cc(-c3ccc(-c4cccc5c4oc4ccccc45)cc3)n2)cc1 ; c1ccc(-c2ccc(-c3cc(-c4cc(-c5ccc6sc7ccccc7c6c5)cc(-c5ccc6sc7ccccc7c6c5)c4)nc(-c4ccccc4)n3)cc2)cc1 ; c1ccc(-c2ccc(-c3cc(-c4cc(-c5cccc6c5sc5ccccc56)cc(-c5cccc6c5sc5ccccc56)c4)nc(-c4ccccc4)n3)cc2)cc1]

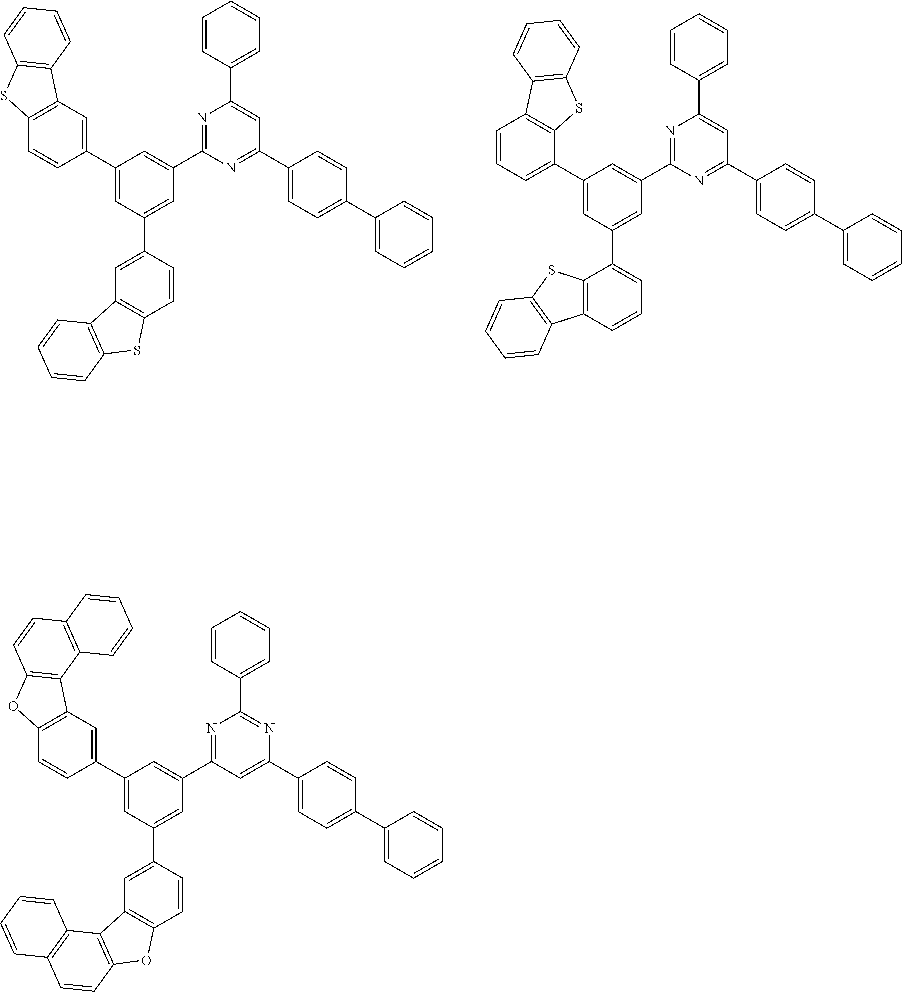 [SMILES: c1ccc(-c2ccc(-c3cc(-c4ccccc4)nc(-c4cc(-c5ccc6sc7ccccc7c6c5)cc(-c5ccc6sc7ccccc7c6c5)c4)n3)cc2)cc1 ; c1ccc(-c2ccc(-c3cc(-c4ccccc4)nc(-c4cc(-c5cccc6c5sc5ccccc56)cc(-c5cccc6c5sc5ccccc56)c4)n3)cc2)cc1 ; c1ccc(-c2ccc(-c3cc(-c4cc(-c5ccc6oc7ccc8ccccc8c7c6c5)cc(-c5ccc6oc7ccc8ccccc8c7c6c5)c4)nc(-c4ccccc4)n3)cc2)cc1]

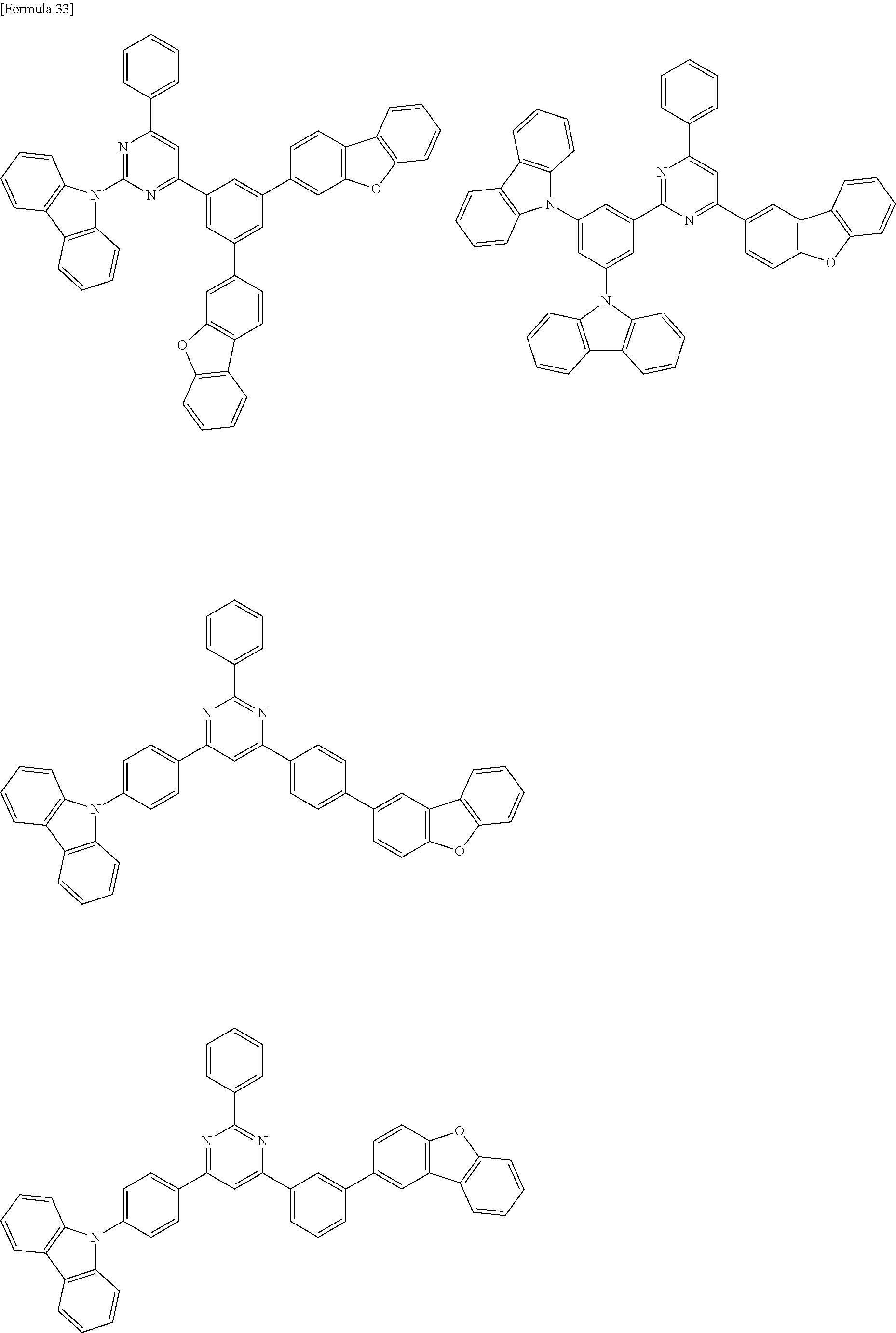 [SMILES: c1ccc(-c2cc(-c3cc(-c4ccc5c(c4)oc4ccccc45)cc(-c4ccc5c(c4)oc4ccccc45)c3)nc(-n3c4ccccc4c4ccccc43)n2)cc1 ; c1ccc(-c2cc(-c3ccc4oc5ccccc5c4c3)nc(-c3cc(-n4c5ccccc5c5ccccc54)cc(-n4c5ccccc5c5ccccc54)c3)n2)cc1 ; c1ccc(-c2nc(-c3ccc(-c4ccc5oc6ccccc6c5c4)cc3)cc(-c3ccc(-n4c5ccccc5c5ccccc54)cc3)n2)cc1 ; c1ccc(-c2nc(-c3ccc(-n4c5ccccc5c5ccccc54)cc3)cc(-c3cccc(-c4ccc5oc6ccccc6c5c4)c3)n2)cc1]

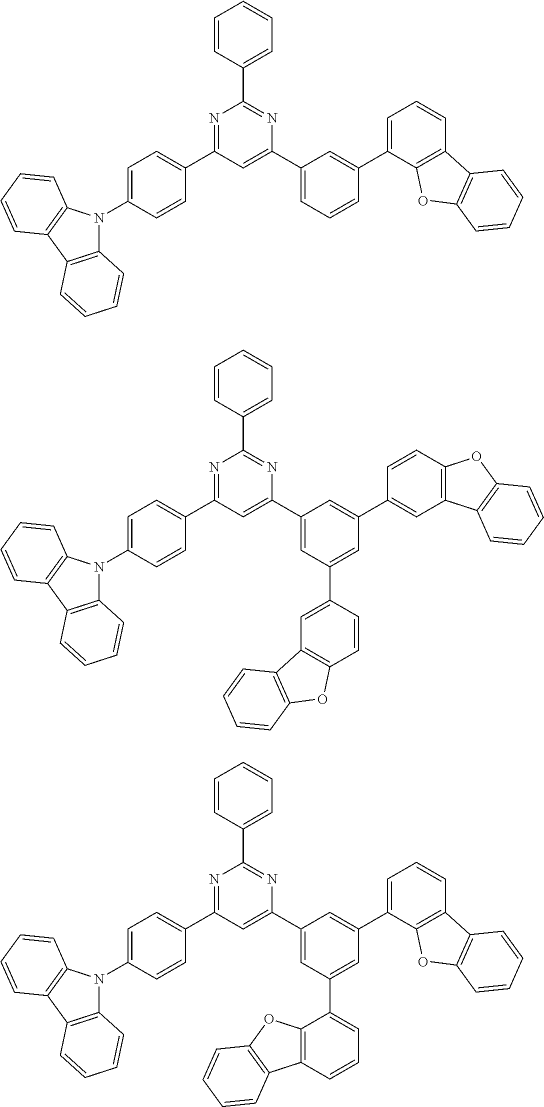

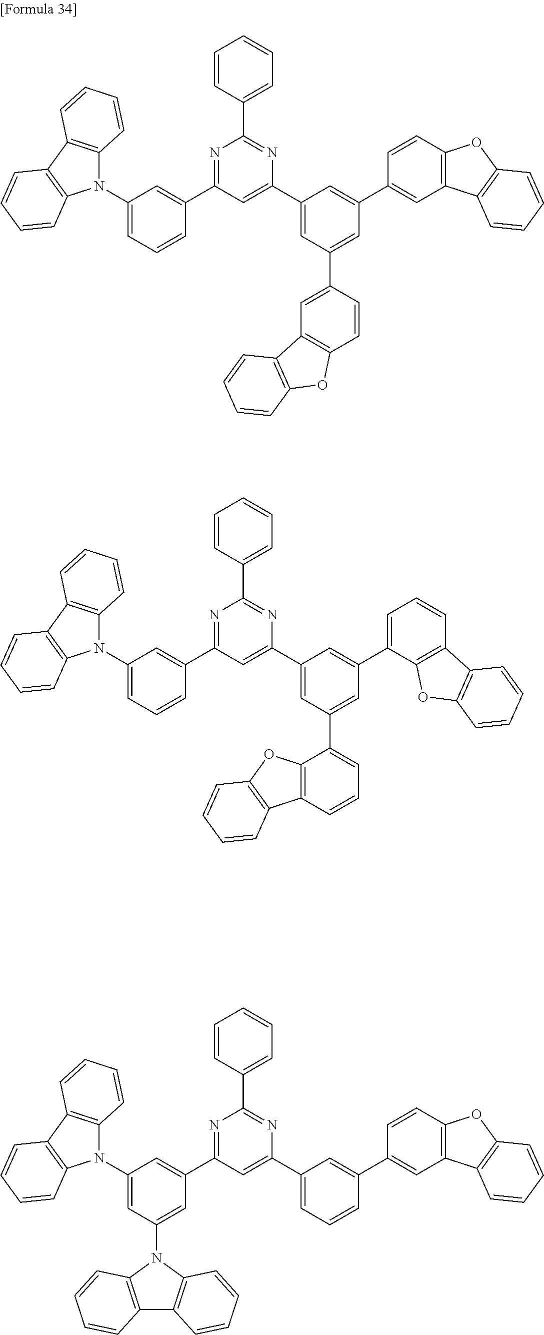 [SMILES: c1ccc(-c2nc(-c3cc(-c4ccc5oc6ccccc6c5c4)cc(-c4ccc5oc6ccccc6c5c4)c3)cc(-c3cccc(-n4c5ccccc5c5ccccc54)c3)n2)cc1 ; c1ccc(-c2nc(-c3cc(-c4cccc5c4oc4ccccc45)cc(-c4cccc5c4oc4ccccc45)c3)cc(-c3cccc(-n4c5ccccc5c5ccccc54)c3)n2)cc1 ; c1ccc(-c2nc(-c3cccc(-c4ccc5oc6ccccc6c5c4)c3)cc(-c3cc(-n4c5ccccc5c5ccccc54)cc(-n4c5ccccc5c5ccccc54)c3)n2)cc1]

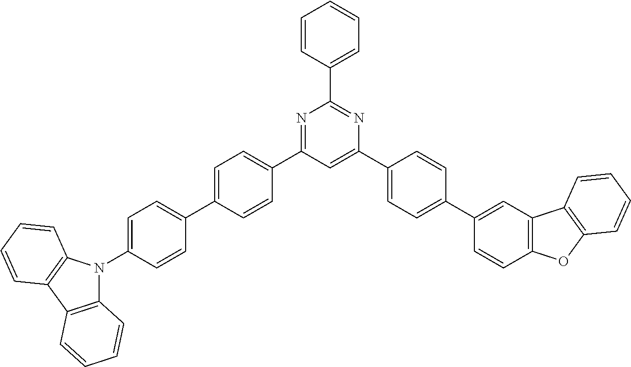 [SMILES: c1ccc(-c2nc(-c3ccc(-c4ccc(-n5c6ccccc6c6ccccc65)cc4)cc3)cc(-c3ccc(-c4ccc5oc6ccccc6c5c4)cc3)n2)cc1]

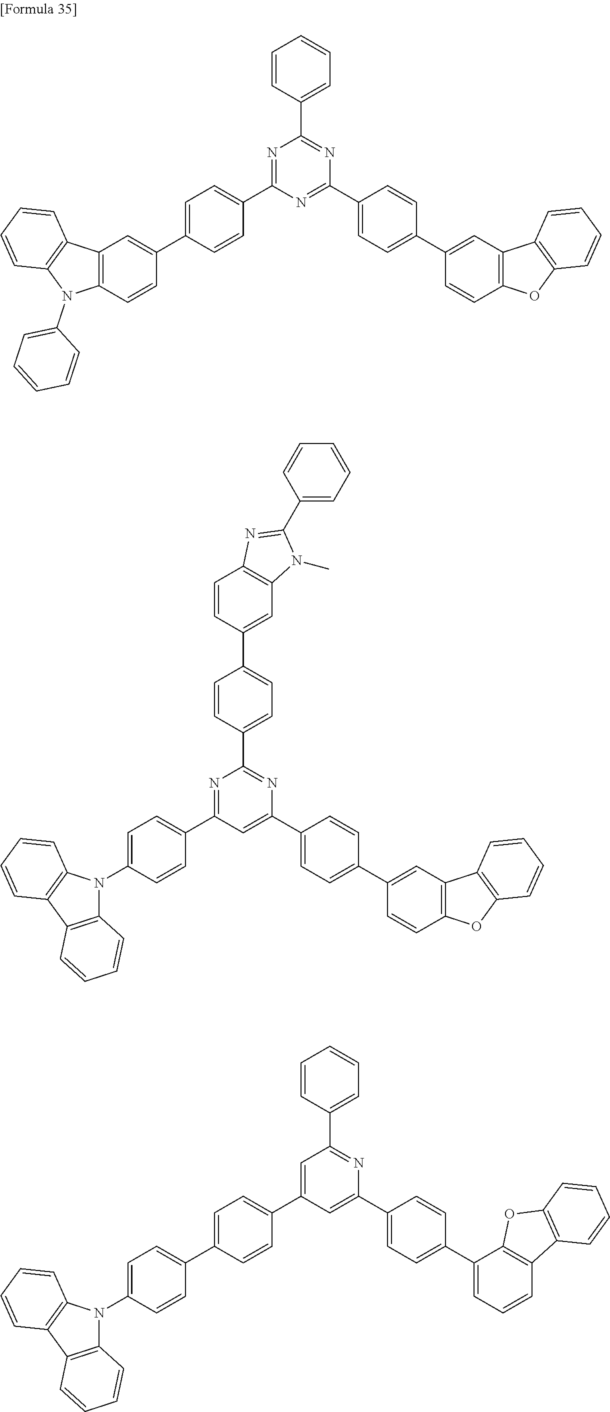 [SMILES: c1ccc(-c2nc(-c3ccc(-c4ccc5oc6ccccc6c5c4)cc3)nc(-c3ccc(-c4ccc5c(c4)c4ccccc4n5-c4ccccc4)cc3)n2)cc1 ; Cn1c(-c2ccccc2)nc2ccc(-c3ccc(-c4nc(-c5ccc(-c6ccc7oc8ccccc8c7c6)cc5)cc(-c5ccc(-n6c7ccccc7c7ccccc76)cc5)n4)cc3)cc21 ; c1ccc(-c2cc(-c3ccc(-c4ccc(-n5c6ccccc6c6ccccc65)cc4)cc3)cc(-c3ccc(-c4cccc5c4oc4ccccc45)cc3)n2)cc1]

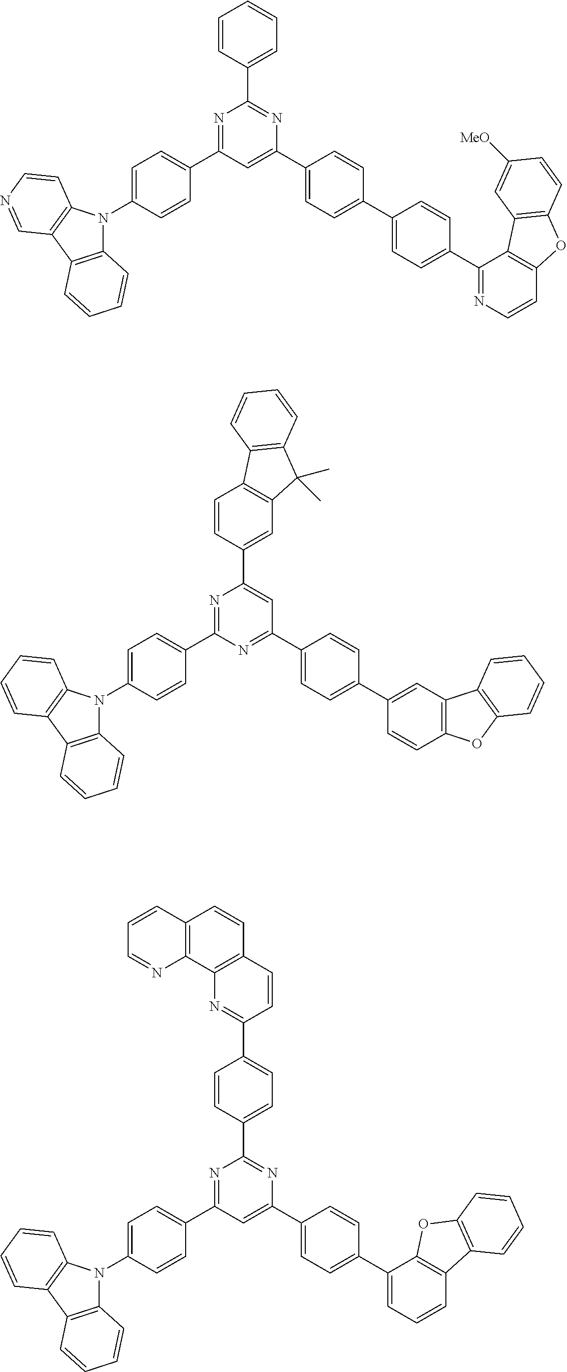 [SMILES: COc1ccc2oc3ccnc(-c4ccc(-c5ccc(-c6cc(-c7ccc(-n8c9ccccc9c9cnccc98)cc7)nc(-c7ccccc7)n6)cc5)cc4)c3c2c1 ; CC1(C)c2ccccc2-c2ccc(-c3cc(-c4ccc(-c5ccc6oc7ccccc7c6c5)cc4)nc(-c4ccc(-n5c6ccccc6c6ccccc65)cc4)n3)cc21 ; c1cnc2c(c1)ccc1ccc(-c3ccc(-c4nc(-c5ccc(-c6cccc7c6oc6ccccc67)cc5)cc(-c5ccc(-n6c7ccccc7c7ccccc76)cc5)n4)cc3)nc12]

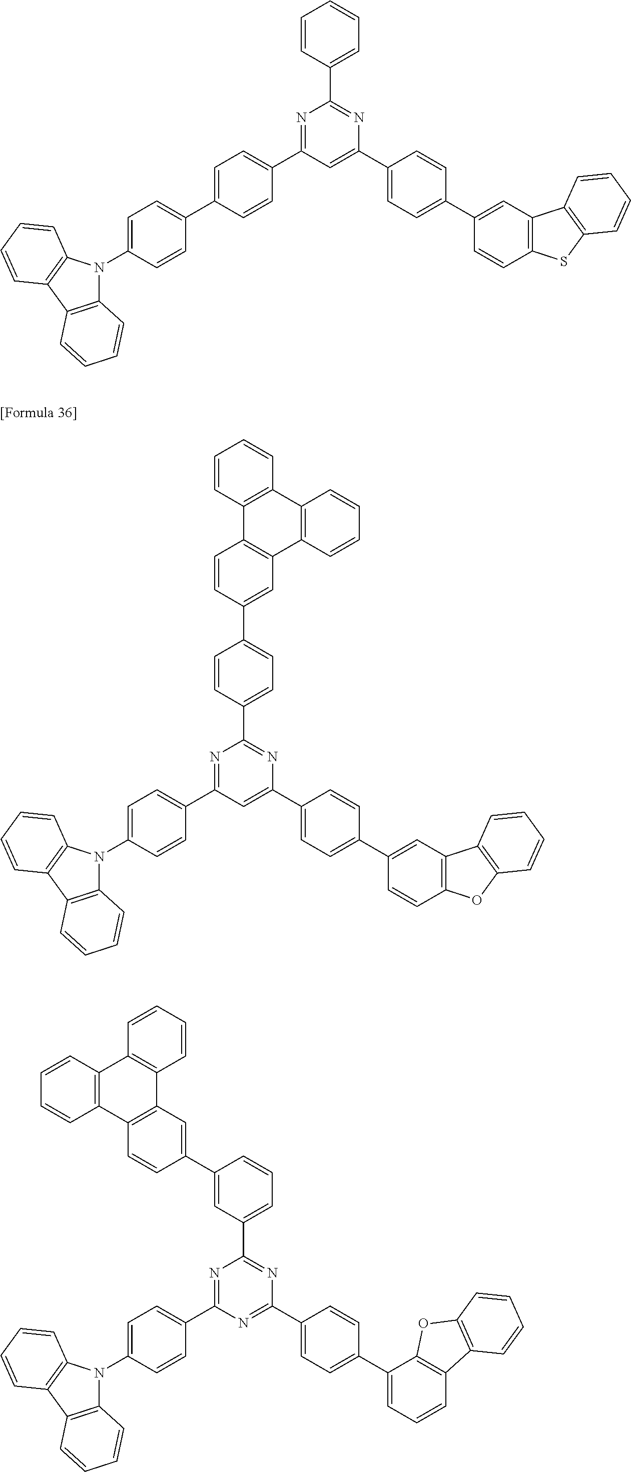 [SMILES: c1ccc(-c2nc(-c3ccc(-c4ccc(-n5c6ccccc6c6ccccc65)cc4)cc3)cc(-c3ccc(-c4ccc5sc6ccccc6c5c4)cc3)n2)cc1 ; c1ccc2c(c1)oc1ccc(-c3ccc(-c4cc(-c5ccc(-n6c7ccccc7c7ccccc76)cc5)nc(-c5ccc(-c6ccc7c8ccccc8c8ccccc8c7c6)cc5)n4)cc3)cc12 ; c1cc(-c2ccc3c4ccccc4c4ccccc4c3c2)cc(-c2nc(-c3ccc(-c4cccc5c4oc4ccccc45)cc3)nc(-c3ccc(-n4c5ccccc5c5ccccc54)cc3)n2)c1]

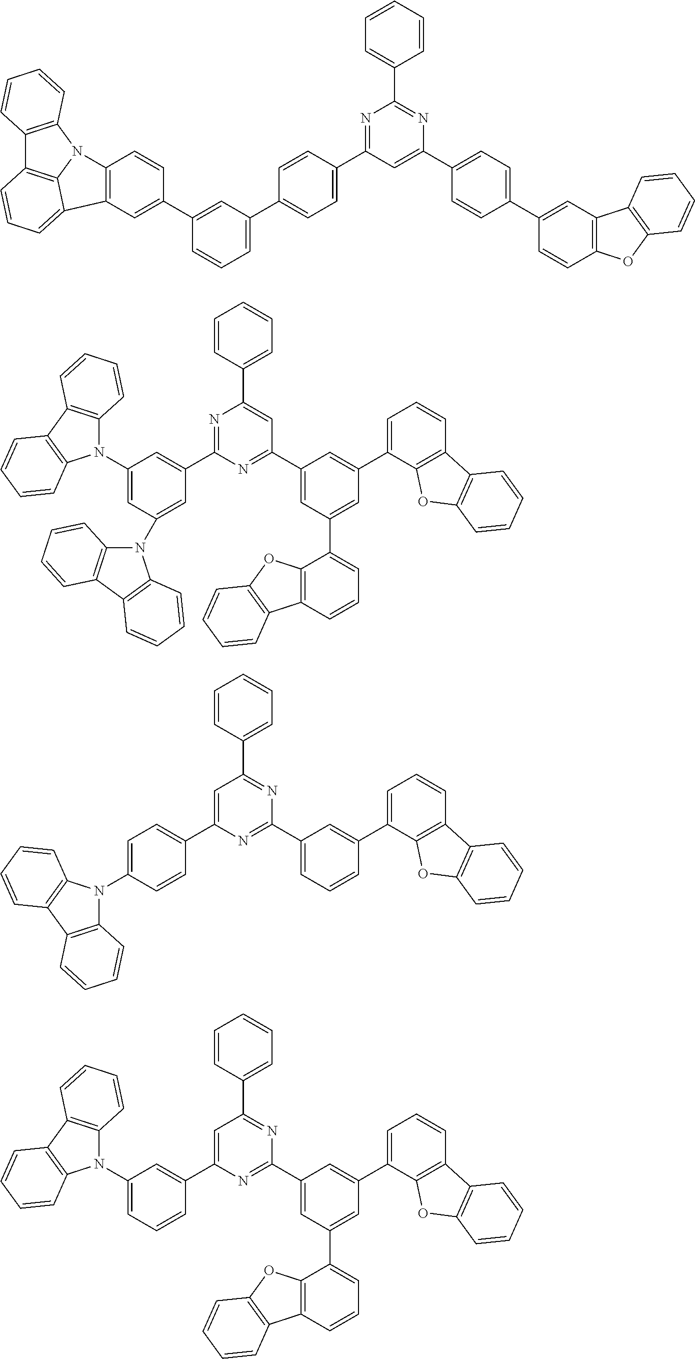

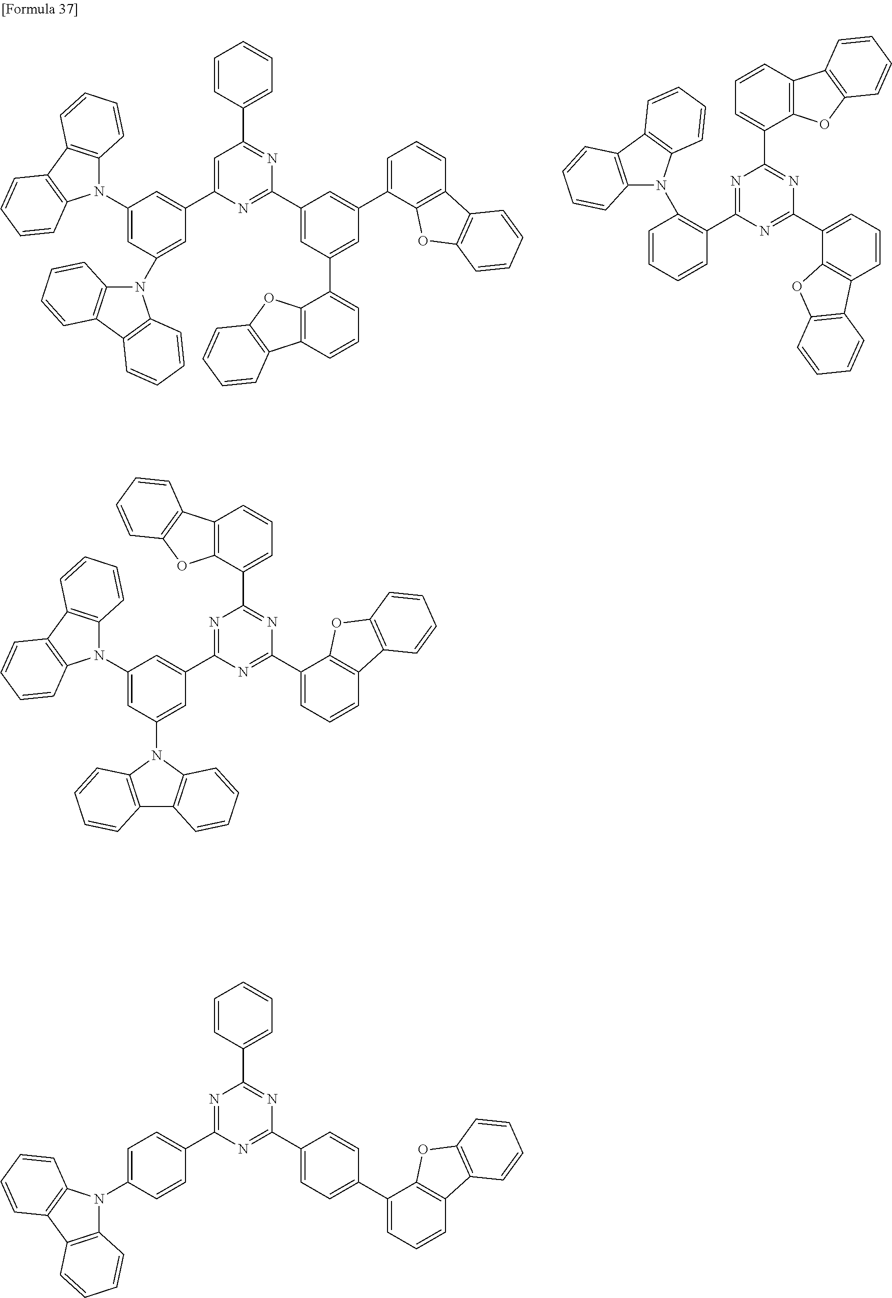 [SMILES: c1ccc(-c2cc(-c3cc(-n4c5ccccc5c5ccccc54)cc(-n4c5ccccc5c5ccccc54)c3)nc(-c3cc(-c4cccc5c4oc4ccccc45)cc(-c4cccc5c4oc4ccccc45)c3)n2)cc1 ; c1ccc(-n2c3ccccc3c3ccccc32)c(-c2nc(-c3cccc4c3oc3ccccc34)nc(-c3cccc4c3oc3ccccc34)n2)c1 ; c1ccc2c(c1)oc1c(-c3nc(-c4cc(-n5c6ccccc6c6ccccc65)cc(-n5c6ccccc6c6ccccc65)c4)nc(-c4cccc5c4oc4ccccc45)n3)cccc12 ; c1ccc(-c2nc(-c3ccc(-c4cccc5c4oc4ccccc45)cc3)nc(-c3ccc(-n4c5ccccc5c5ccccc54)cc3)n2)cc1]

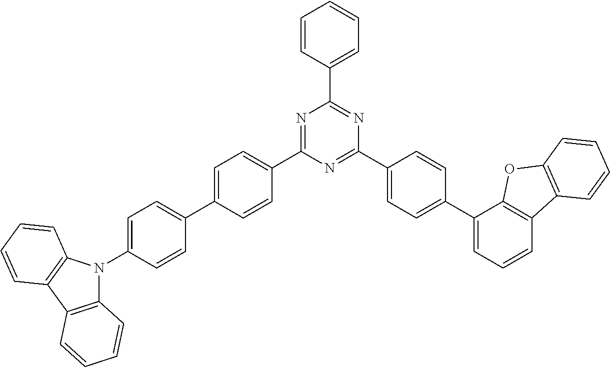 [SMILES: c1ccc(-c2nc(-c3ccc(-c4ccc(-n5c6ccccc6c6ccccc65)cc4)cc3)nc(-c3ccc(-c4cccc5c4oc4ccccc45)cc3)n2)cc1]

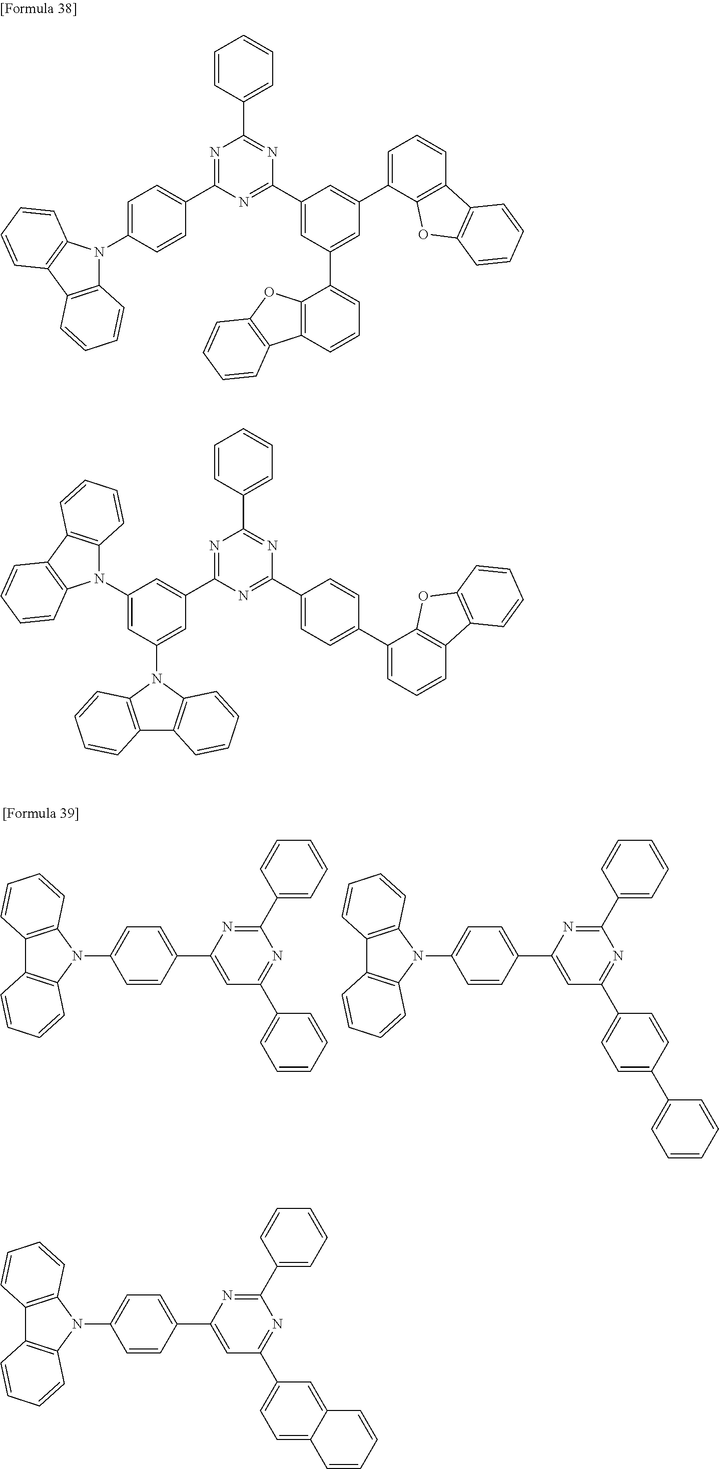

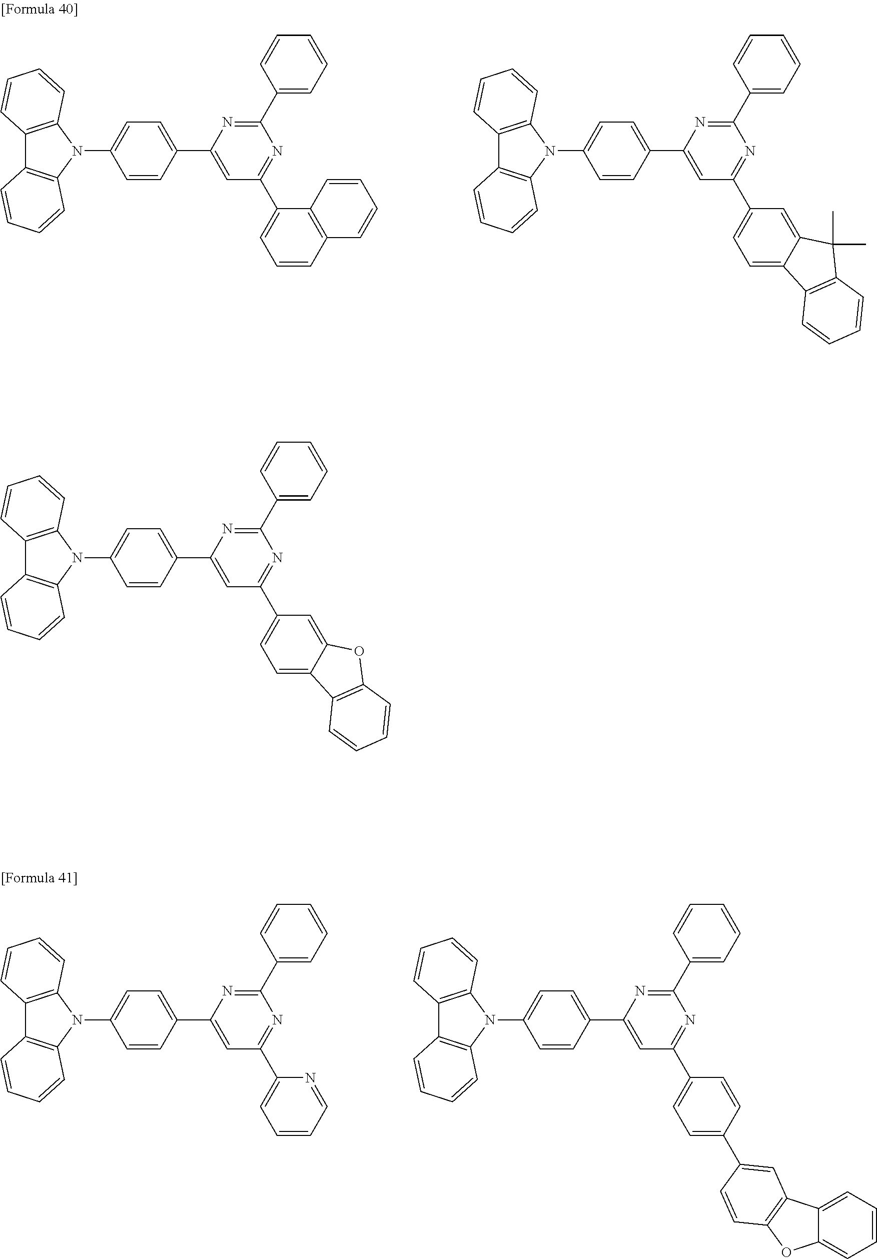 [SMILES: c1ccc(-c2nc(-c3ccc(-n4c5ccccc5c5ccccc54)cc3)cc(-c3cccc4ccccc34)n2)cc1 ; CC1(C)c2ccccc2-c2ccc(-c3cc(-c4ccc(-n5c6ccccc6c6ccccc65)cc4)nc(-c4ccccc4)n3)cc21 ; c1ccc(-c2nc(-c3ccc(-n4c5ccccc5c5ccccc54)cc3)cc(-c3ccc4c(c3)oc3ccccc34)n2)cc1 ; c1ccc(-c2nc(-c3ccc(-n4c5ccccc5c5ccccc54)cc3)cc(-c3ccccn3)n2)cc1 ; c1ccc(-c2nc(-c3ccc(-c4ccc5oc6ccccc6c5c4)cc3)cc(-c3ccc(-n4c5ccccc5c5ccccc54)cc3)n2)cc1]

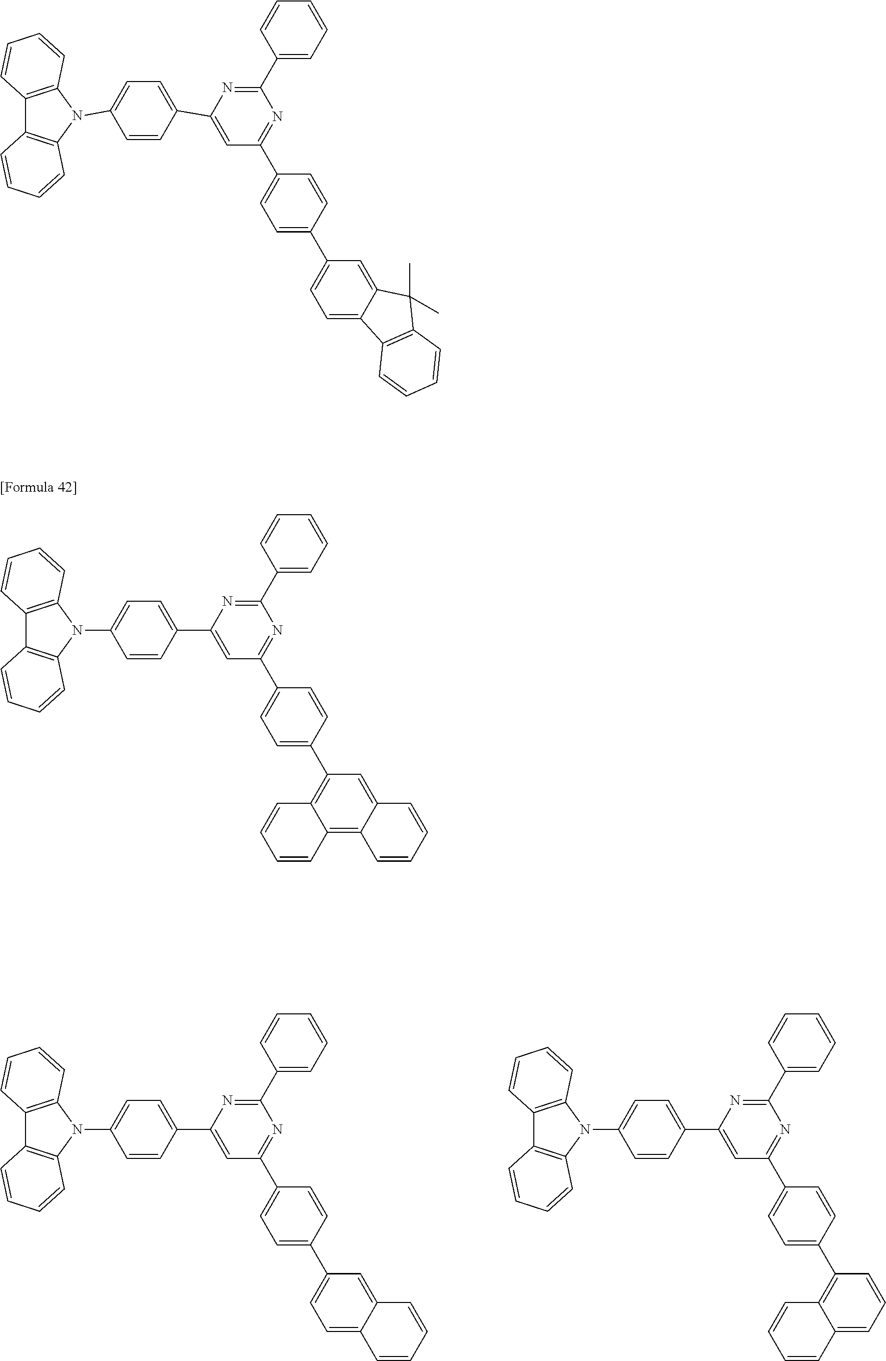 [SMILES: CC1(C)c2ccccc2-c2ccc(-c3ccc(-c4cc(-c5ccc(-n6c7ccccc7c7ccccc76)cc5)nc(-c5ccccc5)n4)cc3)cc21 ; c1ccc(-c2nc(-c3ccc(-c4cc5ccccc5c5ccccc45)cc3)cc(-c3ccc(-n4c5ccccc5c5ccccc54)cc3)n2)cc1 ; c1ccc(-c2nc(-c3ccc(-c4ccc5ccccc5c4)cc3)cc(-c3ccc(-n4c5ccccc5c5ccccc54)cc3)n2)cc1 ; c1ccc(-c2nc(-c3ccc(-c4cccc5ccccc45)cc3)cc(-c3ccc(-n4c5ccccc5c5ccccc54)cc3)n2)cc1]

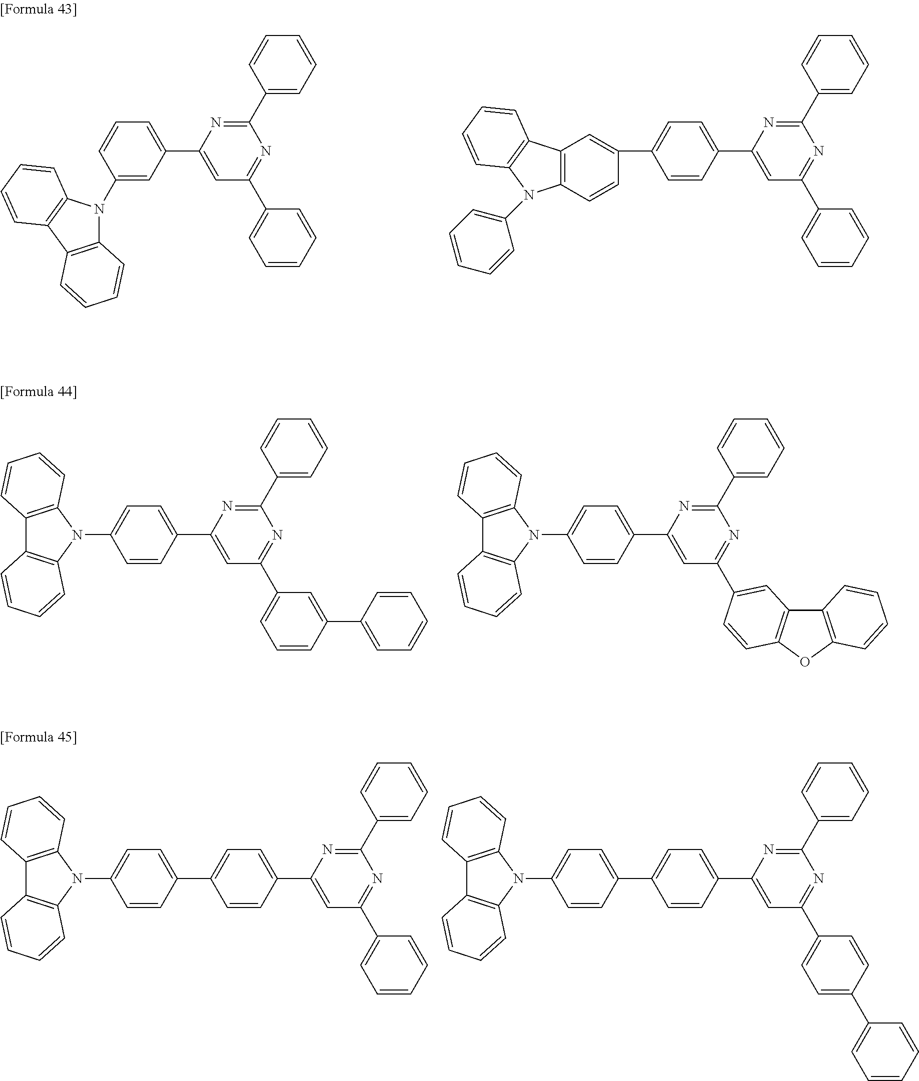

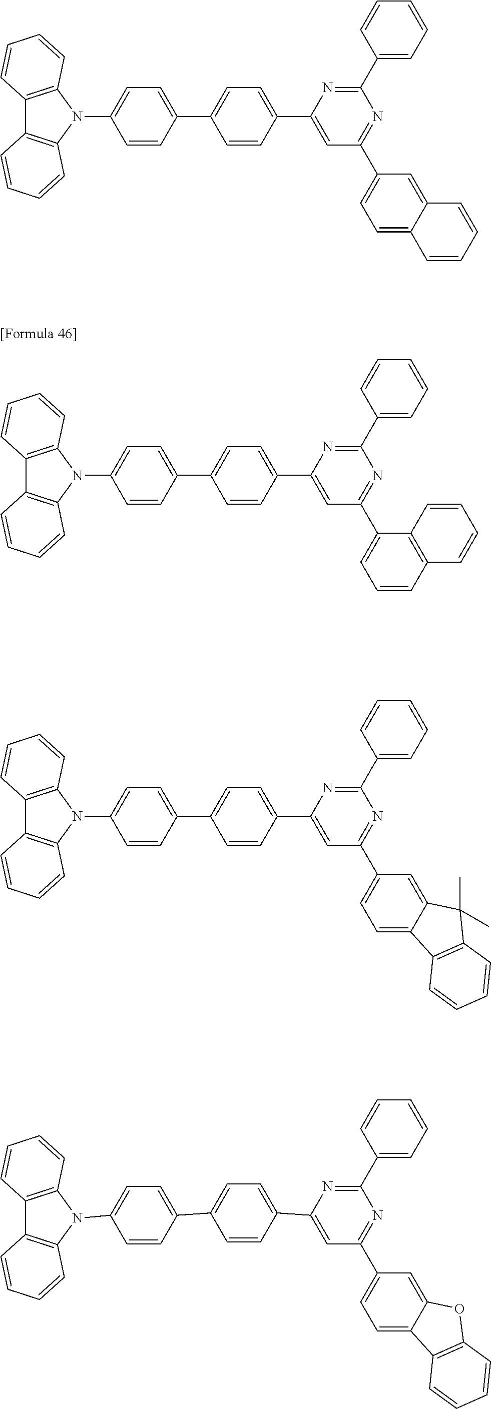 [SMILES: c1ccc(-c2nc(-c3ccc(-c4ccc(-n5c6ccccc6c6ccccc65)cc4)cc3)cc(-c3ccc4ccccc4c3)n2)cc1 ; c1ccc(-c2nc(-c3ccc(-c4ccc(-n5c6ccccc6c6ccccc65)cc4)cc3)cc(-c3cccc4ccccc34)n2)cc1 ; CC1(C)c2ccccc2-c2ccc(-c3cc(-c4ccc(-c5ccc(-n6c7ccccc7c7ccccc76)cc5)cc4)nc(-c4ccccc4)n3)cc21 ; c1ccc(-c2nc(-c3ccc(-c4ccc(-n5c6ccccc6c6ccccc65)cc4)cc3)cc(-c3ccc4c(c3)oc3ccccc34)n2)cc1]

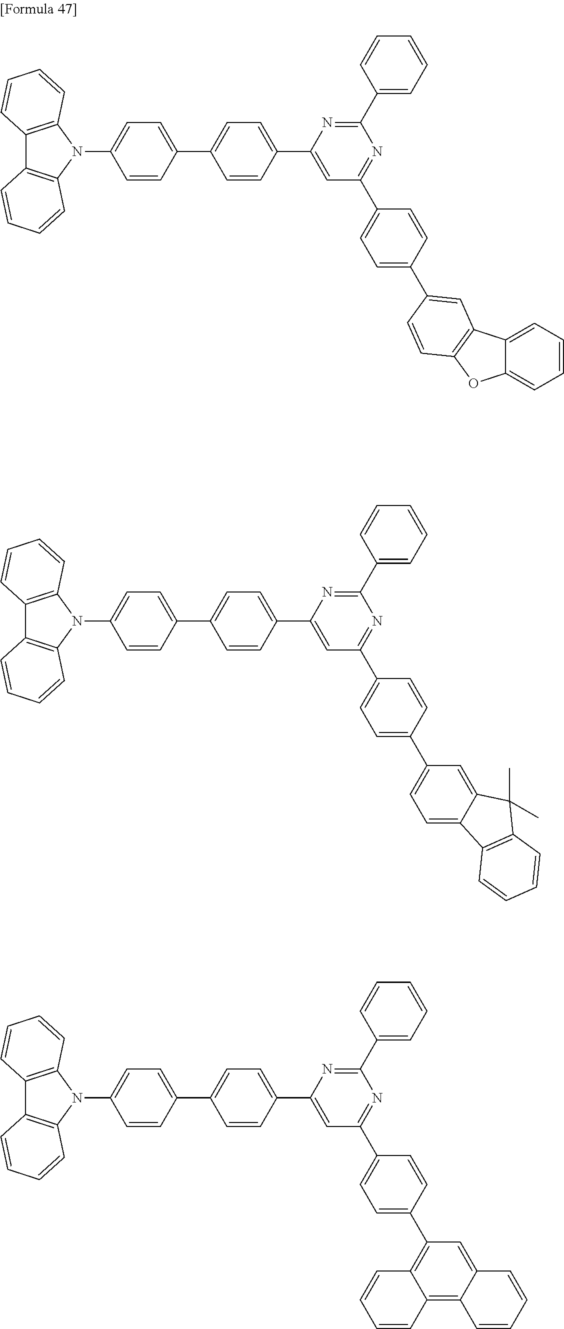 [SMILES: c1ccc(-c2nc(-c3ccc(-c4ccc(-n5c6ccccc6c6ccccc65)cc4)cc3)cc(-c3ccc(-c4ccc5oc6ccccc6c5c4)cc3)n2)cc1 ; CC1(C)c2ccccc2-c2ccc(-c3ccc(-c4cc(-c5ccc(-c6ccc(-n7c8ccccc8c8ccccc87)cc6)cc5)nc(-c5ccccc5)n4)cc3)cc21 ; c1ccc(-c2nc(-c3ccc(-c4ccc(-n5c6ccccc6c6ccccc65)cc4)cc3)cc(-c3ccc(-c4cc5ccccc5c5ccccc45)cc3)n2)cc1]

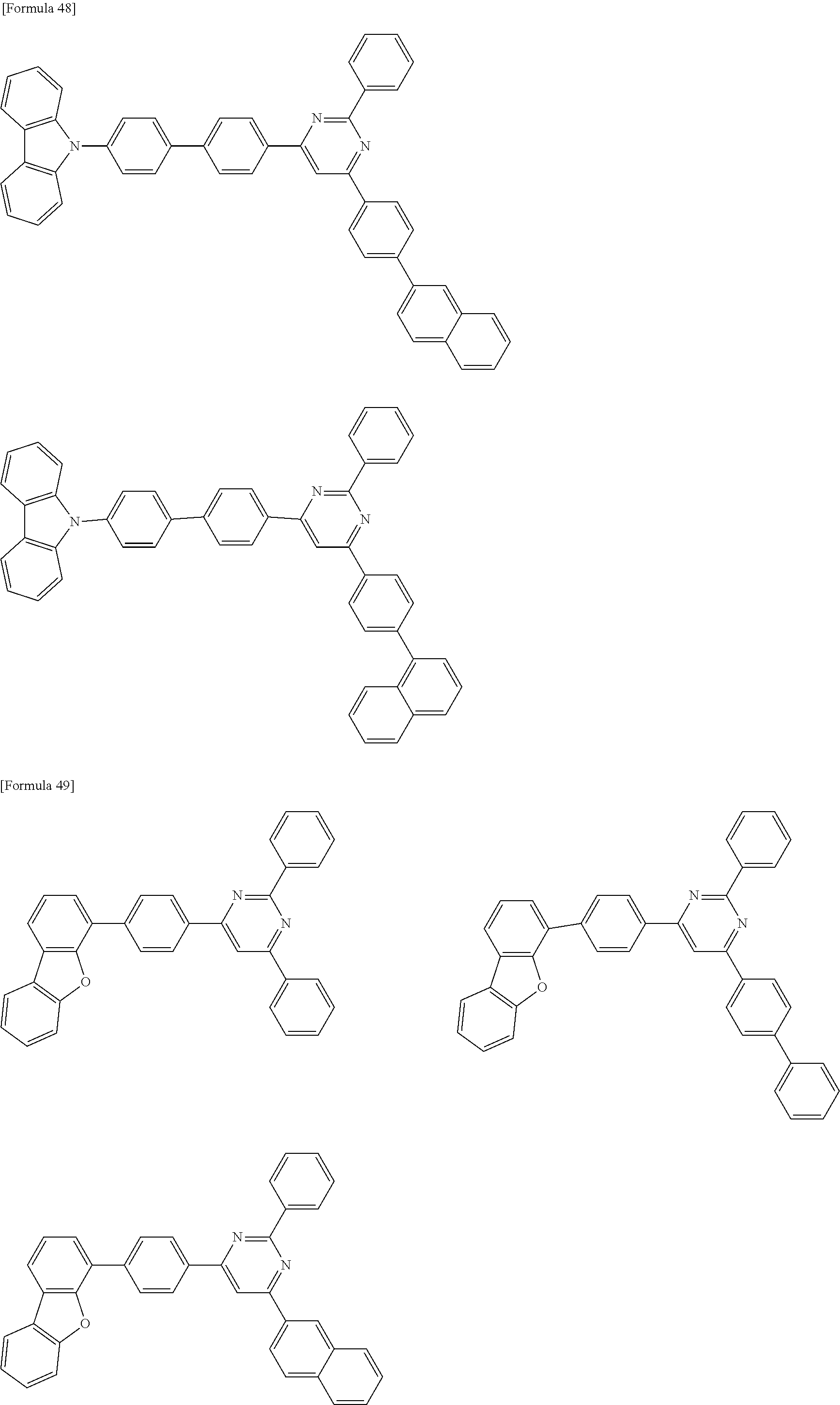 [SMILES: c1ccc(-c2nc(-c3ccc(-c4ccc(-n5c6ccccc6c6ccccc65)cc4)cc3)cc(-c3ccc(-c4ccc5ccccc5c4)cc3)n2)cc1 ; c1ccc(-c2nc(-c3ccc(-c4ccc(-n5c6ccccc6c6ccccc65)cc4)cc3)cc(-c3ccc(-c4cccc5ccccc45)cc3)n2)cc1 ; c1ccc(-c2cc(-c3ccc(-c4cccc5c4oc4ccccc45)cc3)nc(-c3ccccc3)n2)cc1 ; c1ccc(-c2ccc(-c3cc(-c4ccc(-c5cccc6c5oc5ccccc56)cc4)nc(-c4ccccc4)n3)cc2)cc1 ; c1ccc(-c2nc(-c3ccc(-c4cccc5c4oc4ccccc45)cc3)cc(-c3ccc4ccccc4c3)n2)cc1]

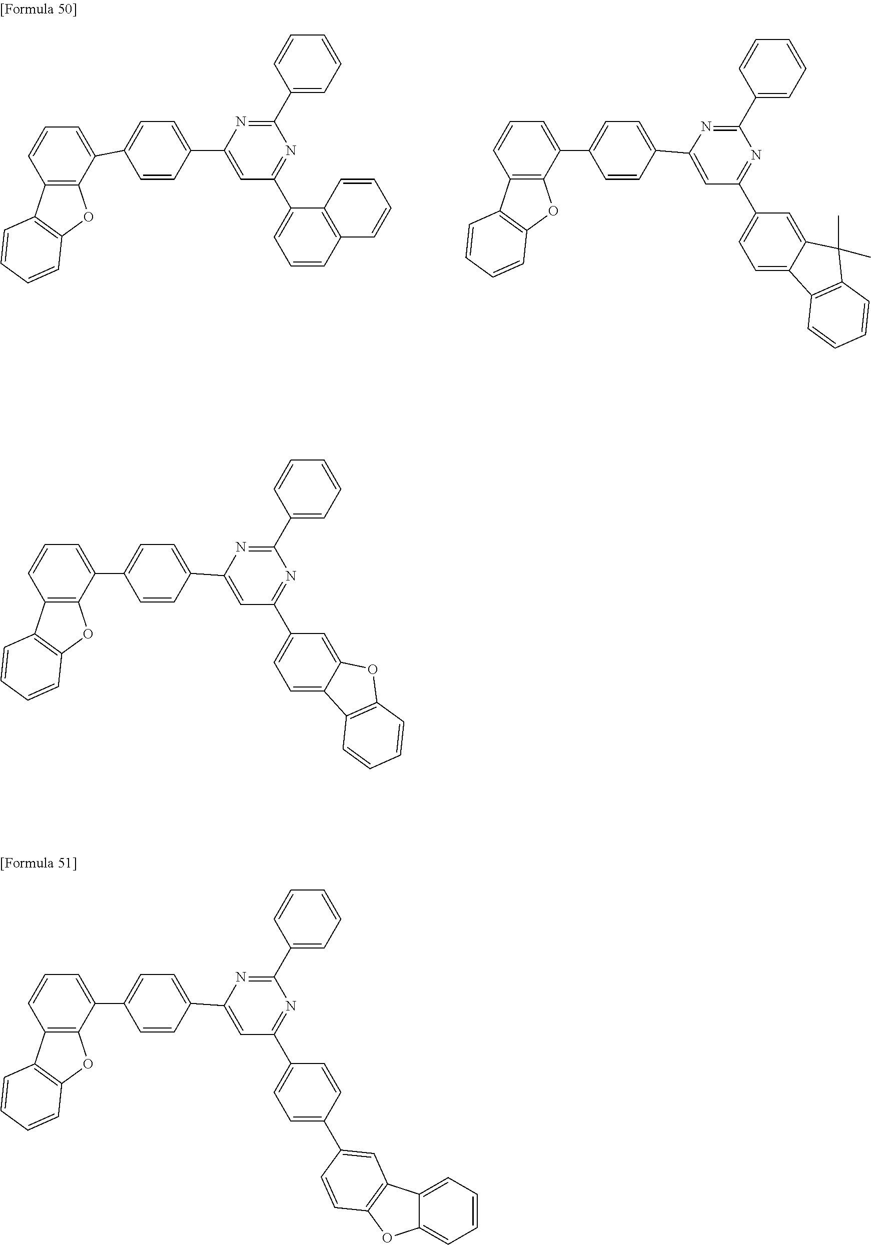 [SMILES: c1ccc(-c2nc(-c3ccc(-c4cccc5c4oc4ccccc45)cc3)cc(-c3cccc4ccccc34)n2)cc1 ; CC1(C)c2ccccc2-c2ccc(-c3cc(-c4ccc(-c5cccc6c5oc5ccccc56)cc4)nc(-c4ccccc4)n3)cc21 ; c1ccc(-c2nc(-c3ccc(-c4cccc5c4oc4ccccc45)cc3)cc(-c3ccc4c(c3)oc3ccccc34)n2)cc1 ; c1ccc(-c2nc(-c3ccc(-c4ccc5oc6ccccc6c5c4)cc3)cc(-c3ccc(-c4cccc5c4oc4ccccc45)cc3)n2)cc1]

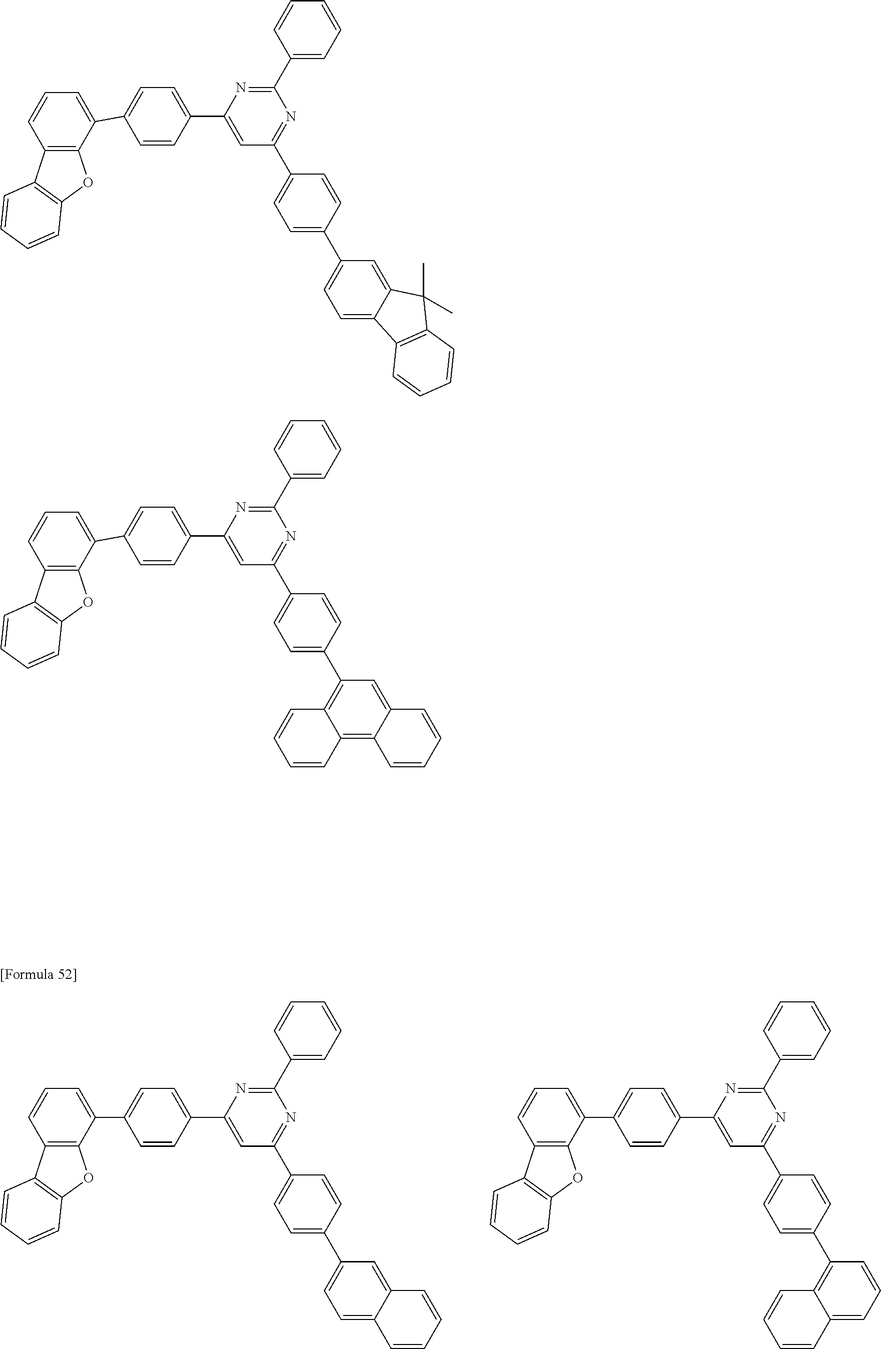 [SMILES: CC1(C)c2ccccc2-c2ccc(-c3ccc(-c4cc(-c5ccc(-c6cccc7c6oc6ccccc67)cc5)nc(-c5ccccc5)n4)cc3)cc21 ; c1ccc(-c2nc(-c3ccc(-c4cc5ccccc5c5ccccc45)cc3)cc(-c3ccc(-c4cccc5c4oc4ccccc45)cc3)n2)cc1 ; c1ccc(-c2nc(-c3ccc(-c4ccc5ccccc5c4)cc3)cc(-c3ccc(-c4cccc5c4oc4ccccc45)cc3)n2)cc1 ; c1ccc(-c2nc(-c3ccc(-c4cccc5ccccc45)cc3)cc(-c3ccc(-c4cccc5c4oc4ccccc45)cc3)n2)cc1]

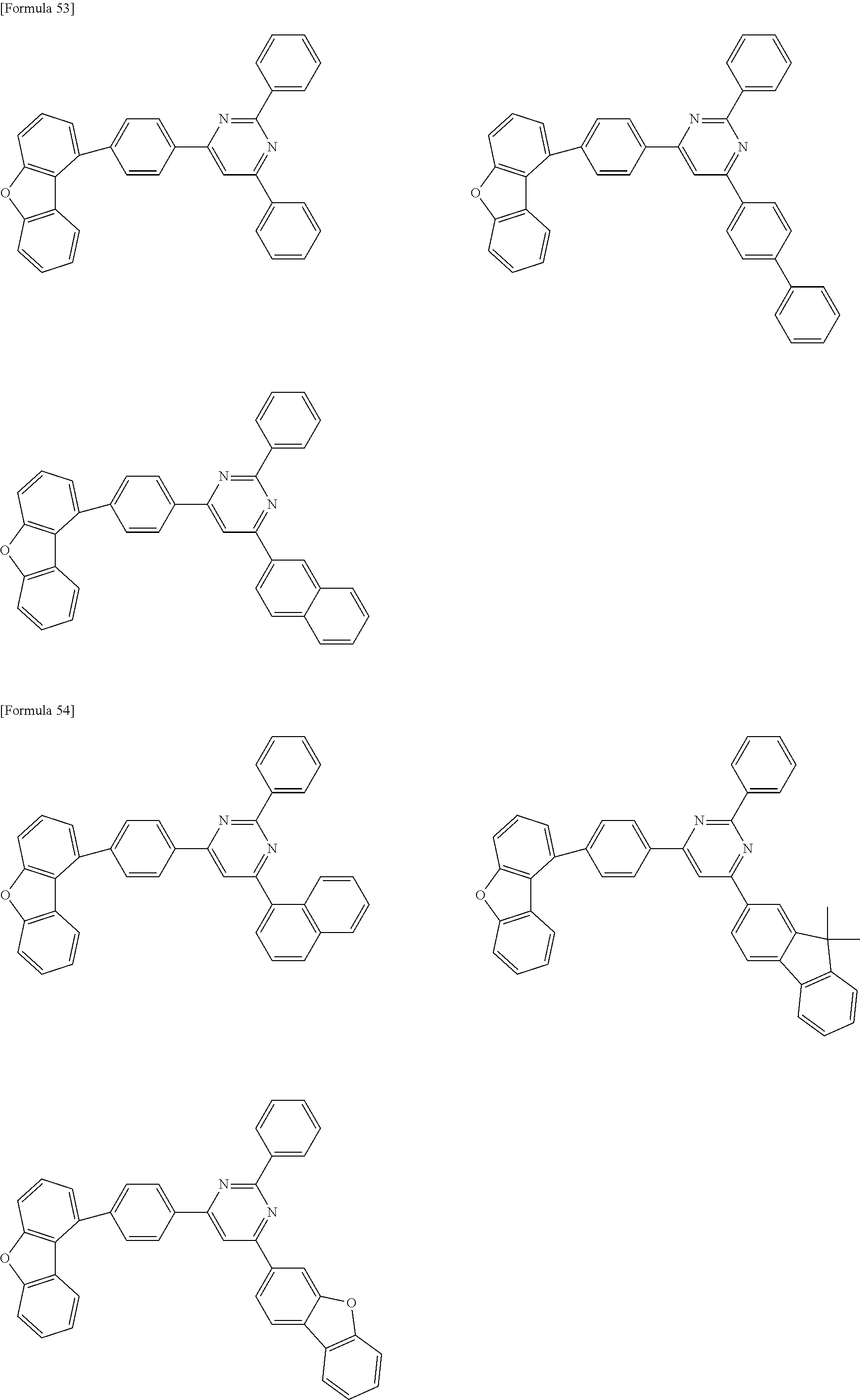 [SMILES: c1ccc(-c2cc(-c3ccc(-c4cccc5oc6ccccc6c45)cc3)nc(-c3ccccc3)n2)cc1 ; c1ccc(-c2ccc(-c3cc(-c4ccc(-c5cccc6oc7ccccc7c56)cc4)nc(-c4ccccc4)n3)cc2)cc1 ; c1ccc(-c2nc(-c3ccc(-c4cccc5oc6ccccc6c45)cc3)cc(-c3ccc4ccccc4c3)n2)cc1 ; c1ccc(-c2nc(-c3ccc(-c4cccc5oc6ccccc6c45)cc3)cc(-c3cccc4ccccc34)n2)cc1 ; CC1(C)c2ccccc2-c2ccc(-c3cc(-c4ccc(-c5cccc6oc7ccccc7c56)cc4)nc(-c4ccccc4)n3)cc21 ; c1ccc(-c2nc(-c3ccc(-c4cccc5oc6ccccc6c45)cc3)cc(-c3ccc4c(c3)oc3ccccc34)n2)cc1]

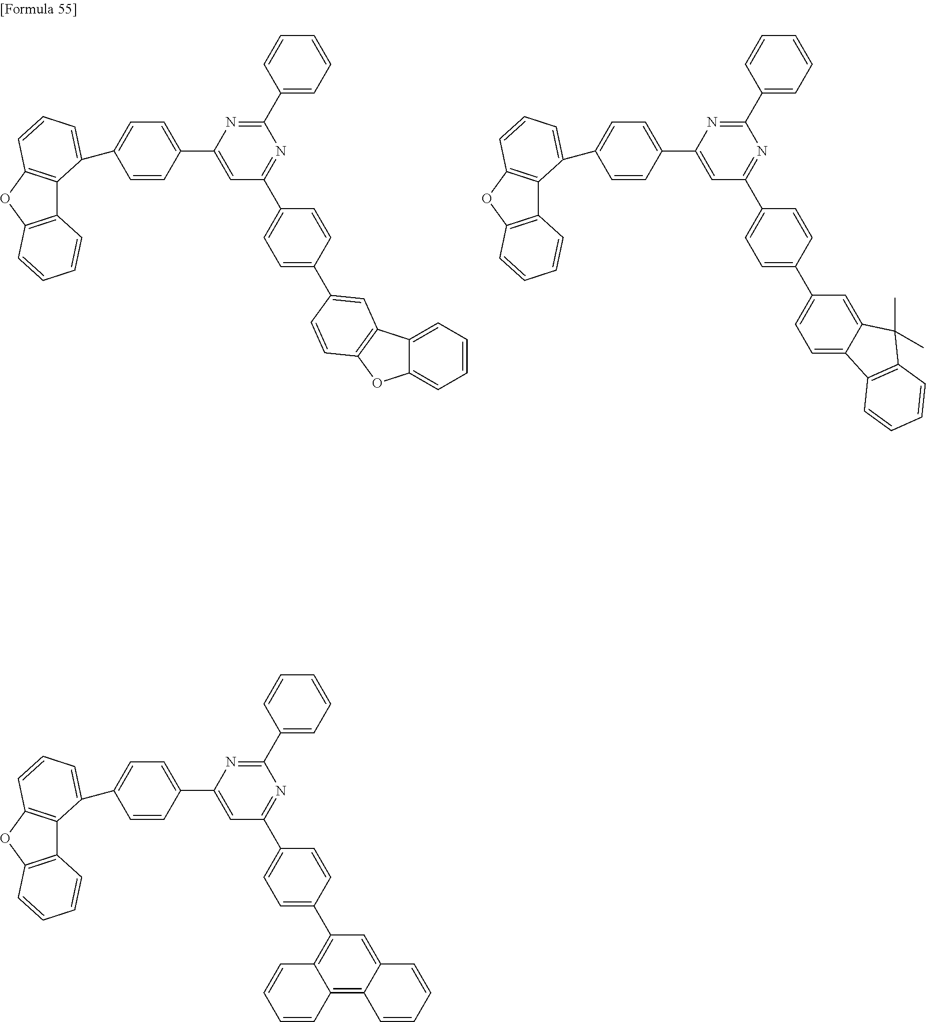 [SMILES: c1ccc(-c2nc(-c3ccc(-c4ccc5oc6ccccc6c5c4)cc3)cc(-c3ccc(-c4cccc5oc6ccccc6c45)cc3)n2)cc1 ; CC1(C)c2ccccc2-c2ccc(-c3ccc(-c4cc(-c5ccc(-c6cccc7oc8ccccc8c67)cc5)nc(-c5ccccc5)n4)cc3)cc21 ; c1ccc(-c2nc(-c3ccc(-c4cc5ccccc5c5ccccc45)cc3)cc(-c3ccc(-c4cccc5oc6ccccc6c45)cc3)n2)cc1]

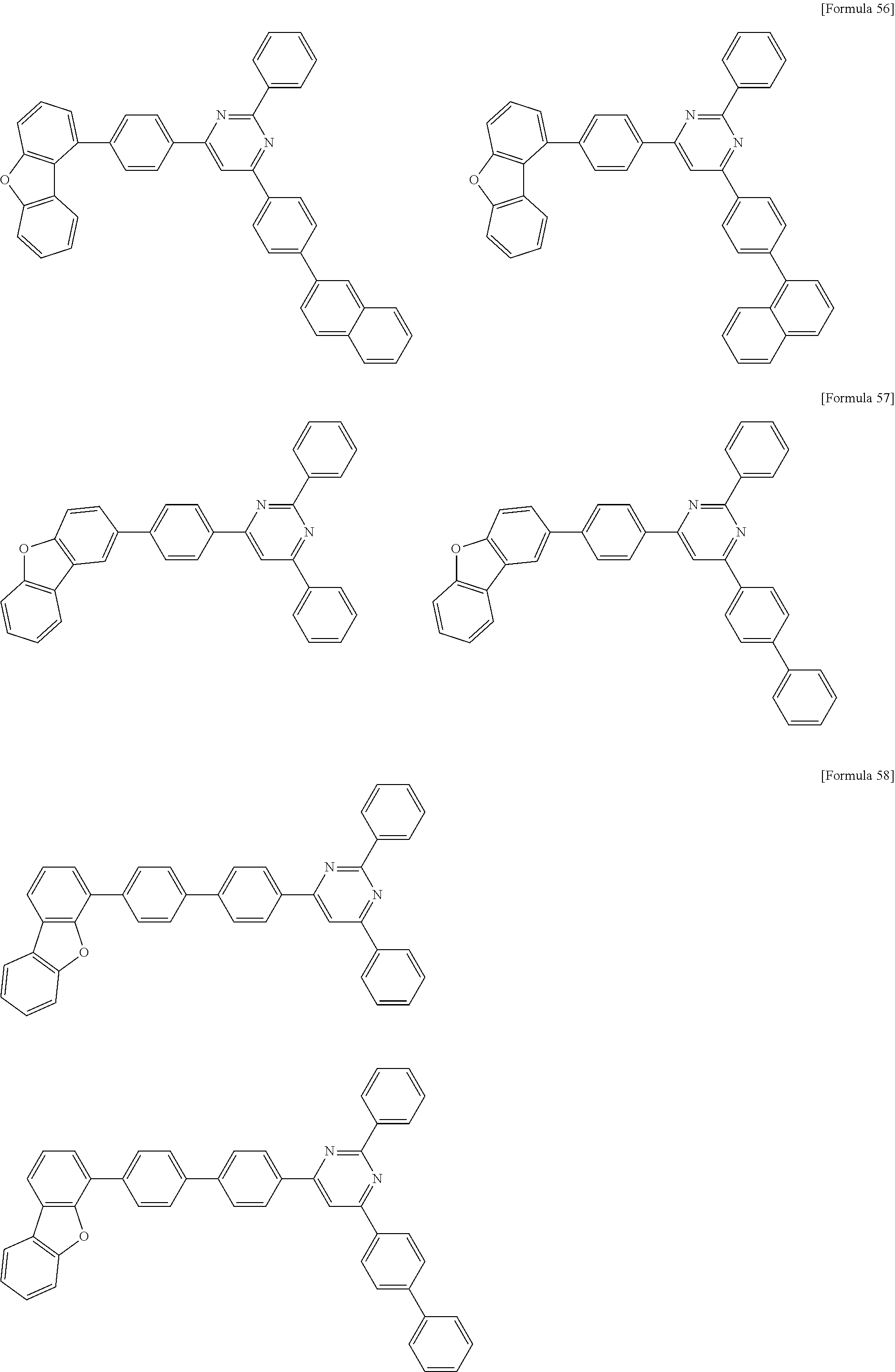 [SMILES: c1ccc(-c2nc(-c3ccc(-c4ccc5ccccc5c4)cc3)cc(-c3ccc(-c4cccc5oc6ccccc6c45)cc3)n2)cc1 ; c1ccc(-c2nc(-c3ccc(-c4cccc5ccccc45)cc3)cc(-c3ccc(-c4cccc5oc6ccccc6c45)cc3)n2)cc1 ; c1ccc(-c2cc(-c3ccc(-c4ccc5oc6ccccc6c5c4)cc3)nc(-c3ccccc3)n2)cc1 ; c1ccc(-c2ccc(-c3cc(-c4ccc(-c5ccc6oc7ccccc7c6c5)cc4)nc(-c4ccccc4)n3)cc2)cc1 ; c1ccc(-c2cc(-c3ccc(-c4ccc(-c5cccc6c5oc5ccccc56)cc4)cc3)nc(-c3ccccc3)n2)cc1 ; c1ccc(-c2ccc(-c3cc(-c4ccc(-c5ccc(-c6cccc7c6oc6ccccc67)cc5)cc4)nc(-c4ccccc4)n3)cc2)cc1]

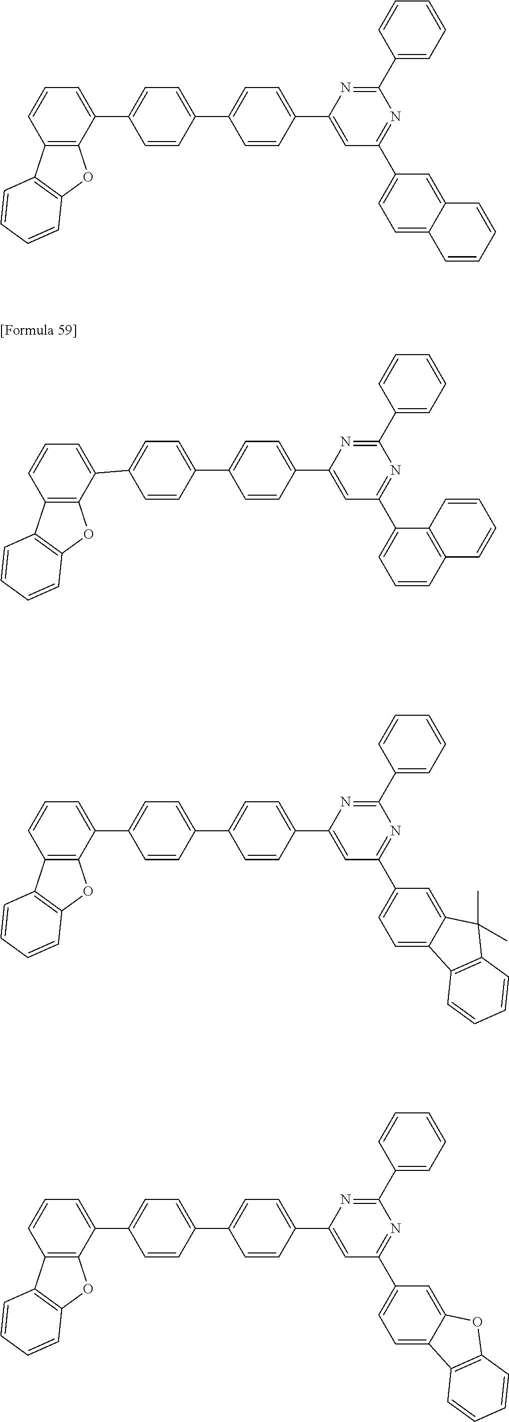 [SMILES: c1ccc(-c2nc(-c3ccc(-c4ccc(-c5cccc6c5oc5ccccc56)cc4)cc3)cc(-c3ccc4ccccc4c3)n2)cc1 ; c1ccc(-c2nc(-c3ccc(-c4ccc(-c5cccc6c5oc5ccccc56)cc4)cc3)cc(-c3cccc4ccccc34)n2)cc1 ; CC1(C)c2ccccc2-c2ccc(-c3cc(-c4ccc(-c5ccc(-c6cccc7c6oc6ccccc67)cc5)cc4)nc(-c4ccccc4)n3)cc21 ; c1ccc(-c2nc(-c3ccc(-c4ccc(-c5cccc6c5oc5ccccc56)cc4)cc3)cc(-c3ccc4c(c3)oc3ccccc34)n2)cc1]

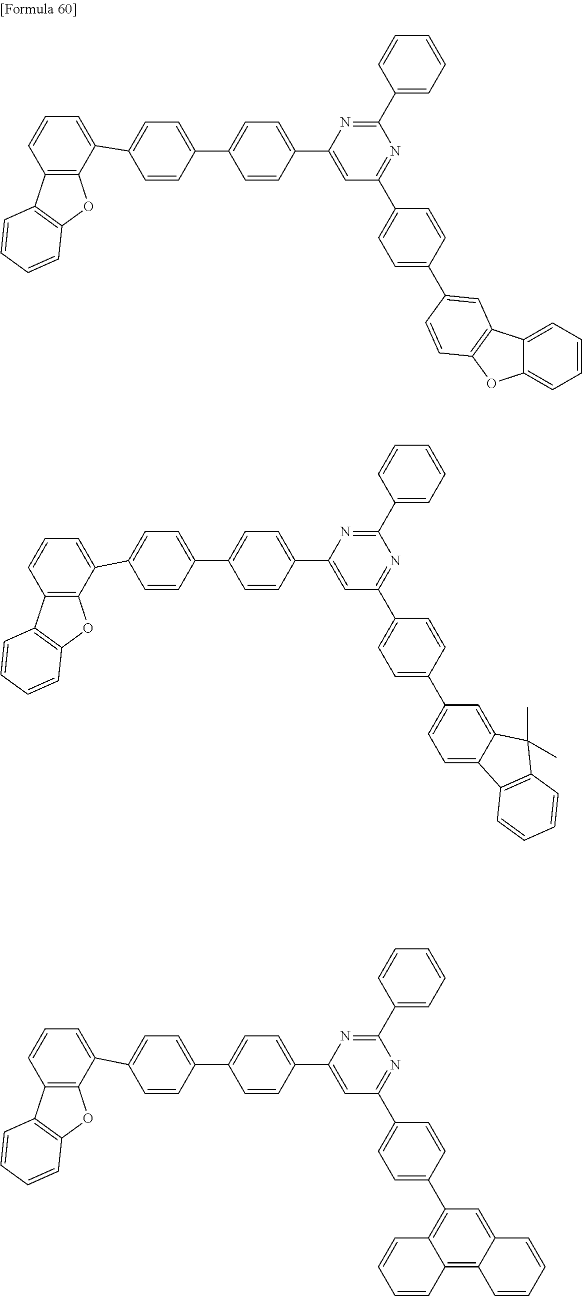 [SMILES: c1ccc(-c2nc(-c3ccc(-c4ccc(-c5cccc6c5oc5ccccc56)cc4)cc3)cc(-c3ccc(-c4ccc5oc6ccccc6c5c4)cc3)n2)cc1 ; CC1(C)c2ccccc2-c2ccc(-c3ccc(-c4cc(-c5ccc(-c6ccc(-c7cccc8c7oc7ccccc78)cc6)cc5)nc(-c5ccccc5)n4)cc3)cc21 ; c1ccc(-c2nc(-c3ccc(-c4ccc(-c5cccc6c5oc5ccccc56)cc4)cc3)cc(-c3ccc(-c4cc5ccccc5c5ccccc45)cc3)n2)cc1]

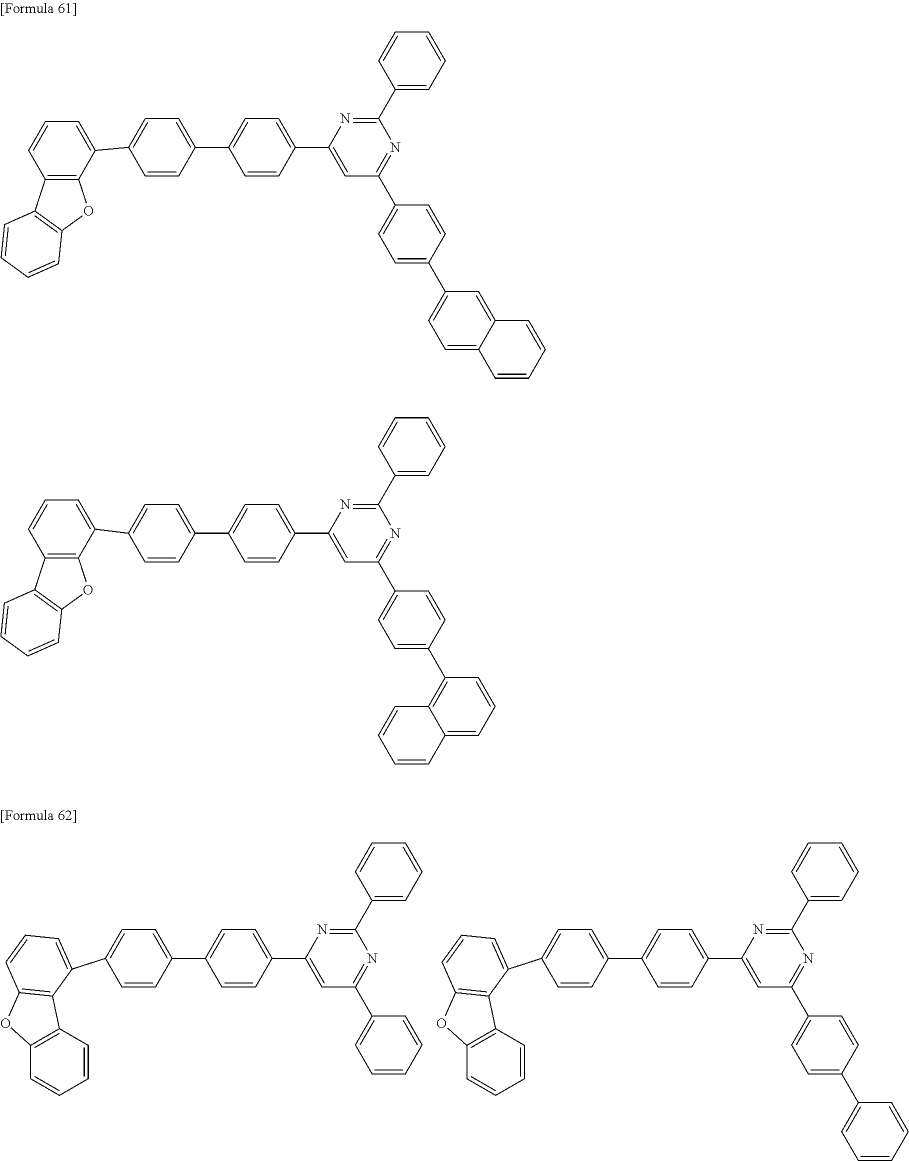

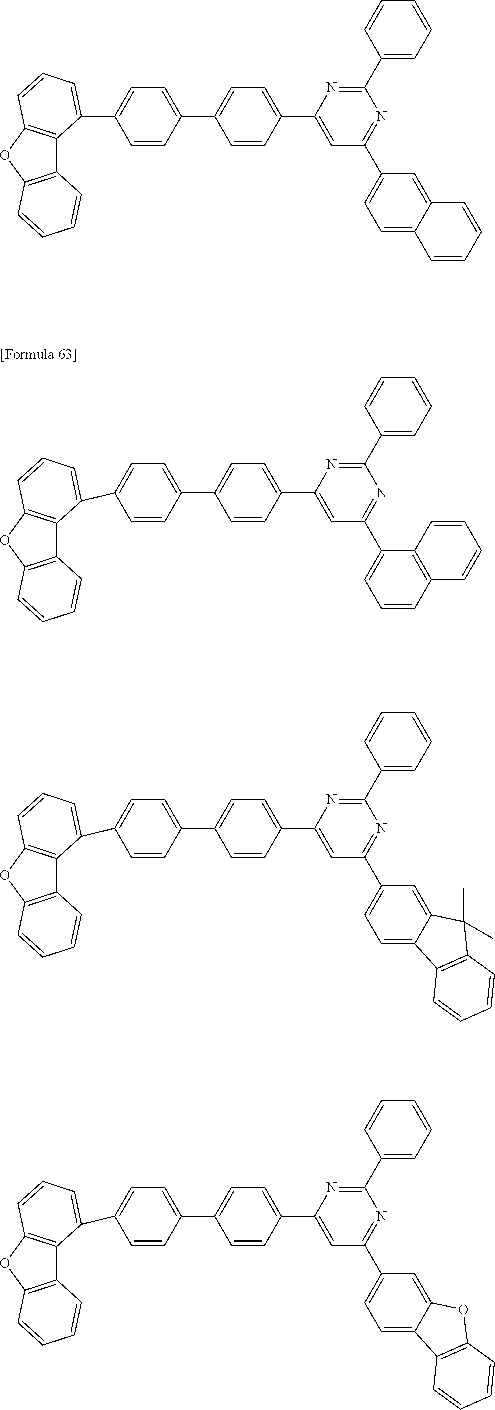 [SMILES: c1ccc(-c2nc(-c3ccc(-c4ccc(-c5cccc6oc7ccccc7c56)cc4)cc3)cc(-c3ccc4ccccc4c3)n2)cc1 ; c1ccc(-c2nc(-c3ccc(-c4ccc(-c5cccc6oc7ccccc7c56)cc4)cc3)cc(-c3cccc4ccccc34)n2)cc1 ; CC1(C)c2ccccc2-c2ccc(-c3cc(-c4ccc(-c5ccc(-c6cccc7oc8ccccc8c67)cc5)cc4)nc(-c4ccccc4)n3)cc21 ; c1ccc(-c2nc(-c3ccc(-c4ccc(-c5cccc6oc7ccccc7c56)cc4)cc3)cc(-c3ccc4c(c3)oc3ccccc34)n2)cc1]

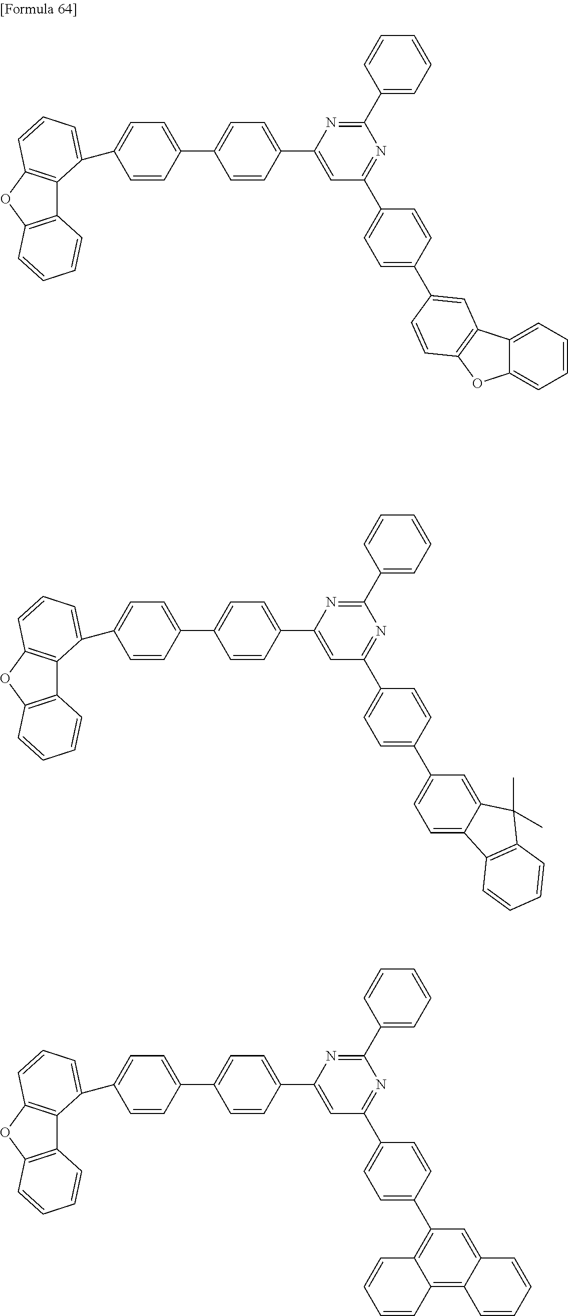 [SMILES: c1ccc(-c2nc(-c3ccc(-c4ccc(-c5cccc6oc7ccccc7c56)cc4)cc3)cc(-c3ccc(-c4ccc5oc6ccccc6c5c4)cc3)n2)cc1 ; CC1(C)c2ccccc2-c2ccc(-c3ccc(-c4cc(-c5ccc(-c6ccc(-c7cccc8oc9ccccc9c78)cc6)cc5)nc(-c5ccccc5)n4)cc3)cc21 ; c1ccc(-c2nc(-c3ccc(-c4ccc(-c5cccc6oc7ccccc7c56)cc4)cc3)cc(-c3ccc(-c4cc5ccccc5c5ccccc45)cc3)n2)cc1]

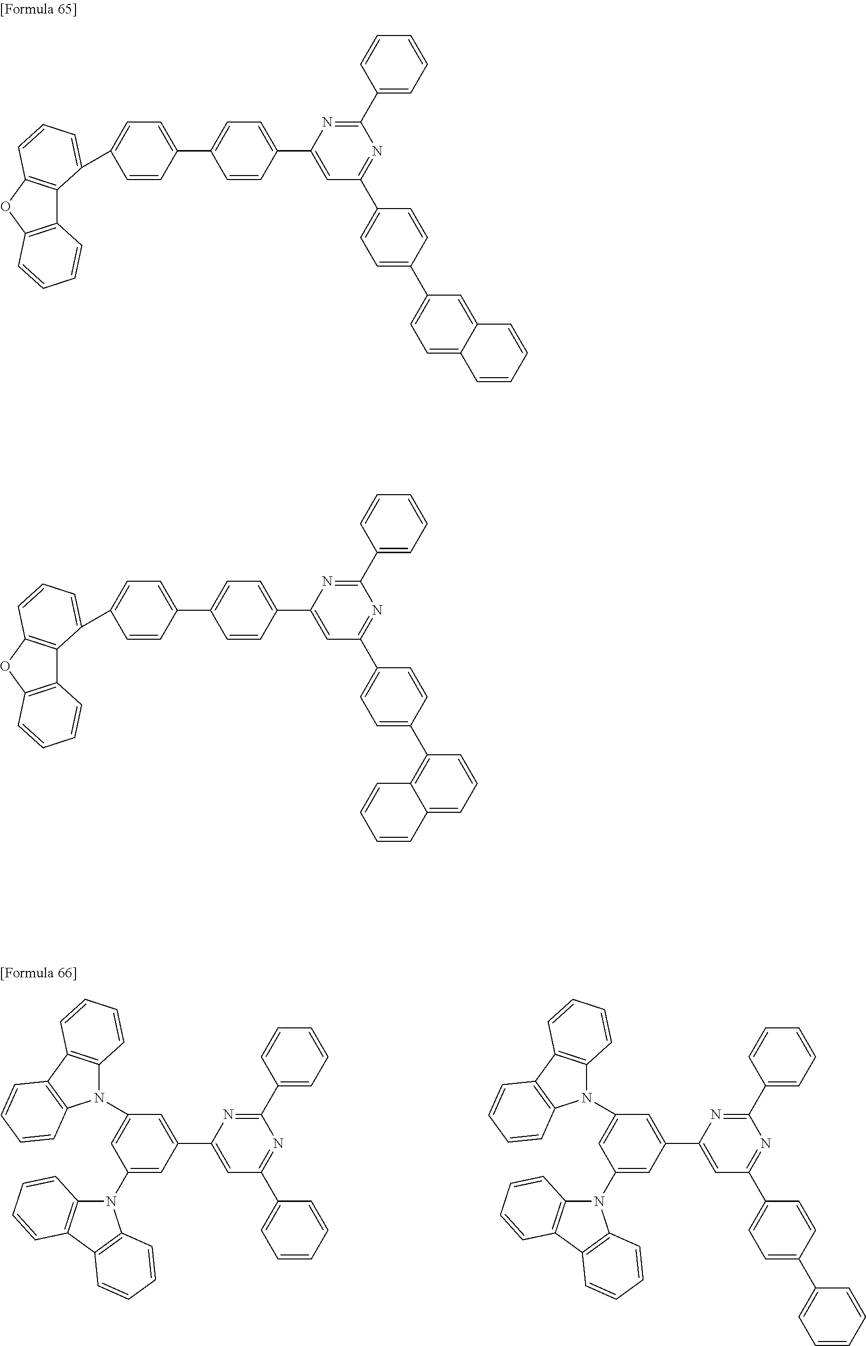 [SMILES: c1ccc(-c2nc(-c3ccc(-c4ccc(-c5cccc6oc7ccccc7c56)cc4)cc3)cc(-c3ccc(-c4ccc5ccccc5c4)cc3)n2)cc1 ; c1ccc(-c2nc(-c3ccc(-c4ccc(-c5cccc6oc7ccccc7c56)cc4)cc3)cc(-c3ccc(-c4cccc5ccccc45)cc3)n2)cc1 ; c1ccc(-c2cc(-c3cc(-n4c5ccccc5c5ccccc54)cc(-n4c5ccccc5c5ccccc54)c3)nc(-c3ccccc3)n2)cc1 ; c1ccc(-c2ccc(-c3cc(-c4cc(-n5c6ccccc6c6ccccc65)cc(-n5c6ccccc6c6ccccc65)c4)nc(-c4ccccc4)n3)cc2)cc1]

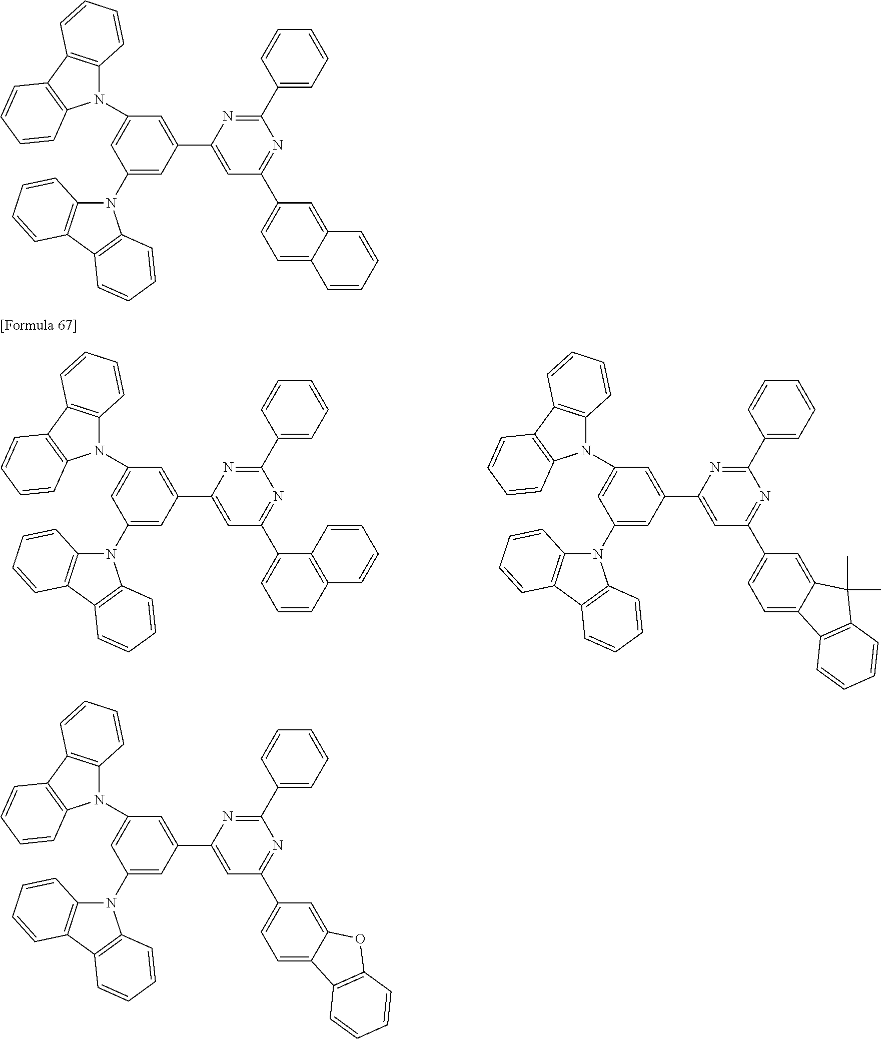 [SMILES: c1ccc(-c2nc(-c3cc(-n4c5ccccc5c5ccccc54)cc(-n4c5ccccc5c5ccccc54)c3)cc(-c3ccc4ccccc4c3)n2)cc1 ; c1ccc(-c2nc(-c3cc(-n4c5ccccc5c5ccccc54)cc(-n4c5ccccc5c5ccccc54)c3)cc(-c3cccc4ccccc34)n2)cc1 ; CC1(C)c2ccccc2-c2ccc(-c3cc(-c4cc(-n5c6ccccc6c6ccccc65)cc(-n5c6ccccc6c6ccccc65)c4)nc(-c4ccccc4)n3)cc21 ; c1ccc(-c2nc(-c3cc(-n4c5ccccc5c5ccccc54)cc(-n4c5ccccc5c5ccccc54)c3)cc(-c3ccc4c(c3)oc3ccccc34)n2)cc1]

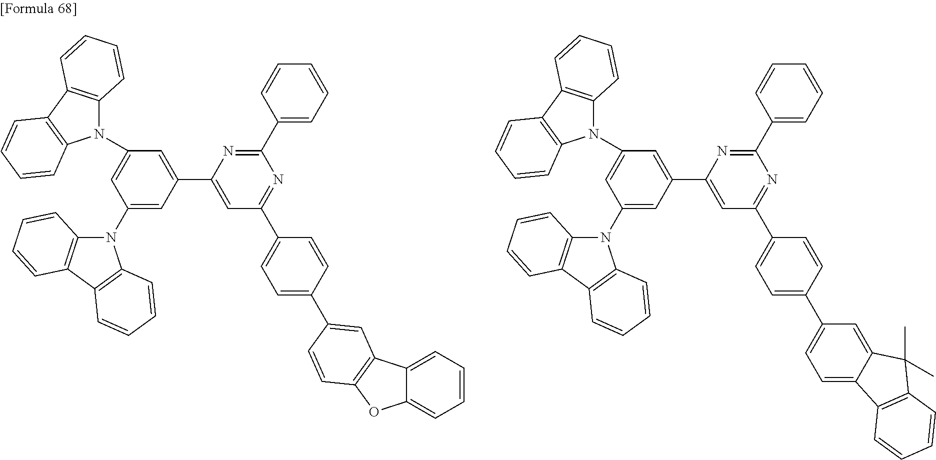 [SMILES: c1ccc(-c2nc(-c3ccc(-c4ccc5oc6ccccc6c5c4)cc3)cc(-c3cc(-n4c5ccccc5c5ccccc54)cc(-n4c5ccccc5c5ccccc54)c3)n2)cc1 ; CC1(C)c2ccccc2-c2ccc(-c3ccc(-c4cc(-c5cc(-n6c7ccccc7c7ccccc76)cc(-n6c7ccccc7c7ccccc76)c5)nc(-c5ccccc5)n4)cc3)cc21]

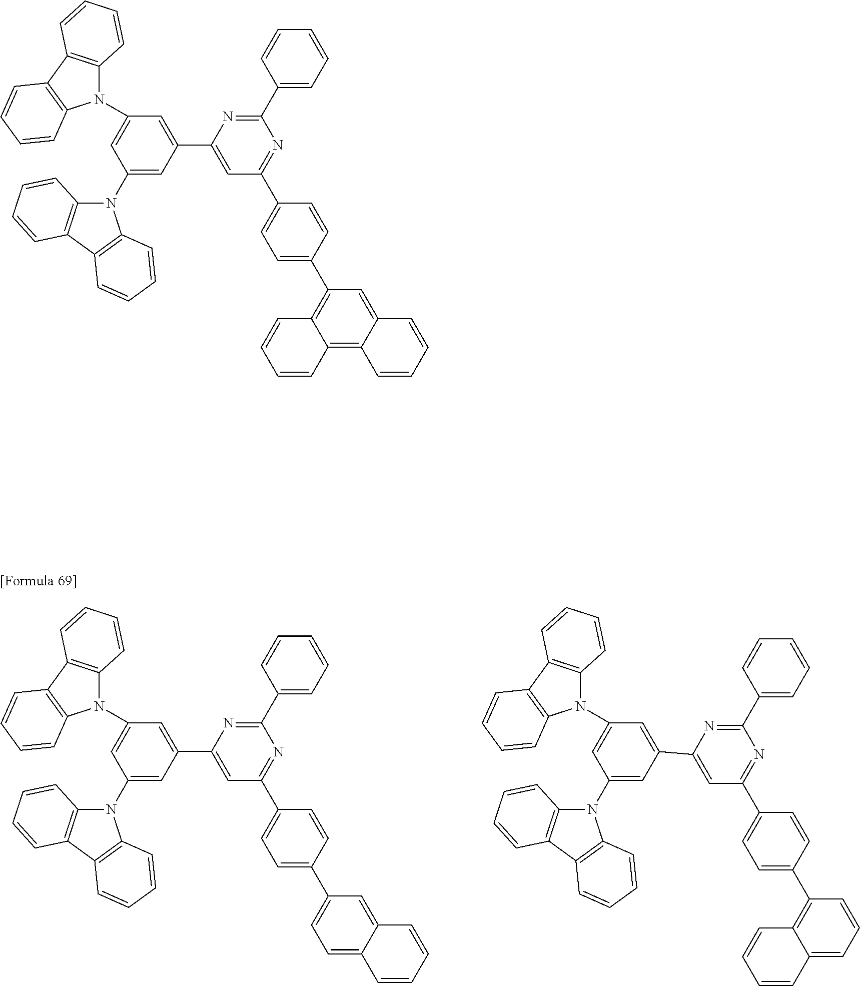 [SMILES: c1ccc(-c2nc(-c3ccc(-c4cc5ccccc5c5ccccc45)cc3)cc(-c3cc(-n4c5ccccc5c5ccccc54)cc(-n4c5ccccc5c5ccccc54)c3)n2)cc1 ; c1ccc(-c2nc(-c3ccc(-c4ccc5ccccc5c4)cc3)cc(-c3cc(-n4c5ccccc5c5ccccc54)cc(-n4c5ccccc5c5ccccc54)c3)n2)cc1 ; c1ccc(-c2nc(-c3ccc(-c4cccc5ccccc45)cc3)cc(-c3cc(-n4c5ccccc5c5ccccc54)cc(-n4c5ccccc5c5ccccc54)c3)n2)cc1]

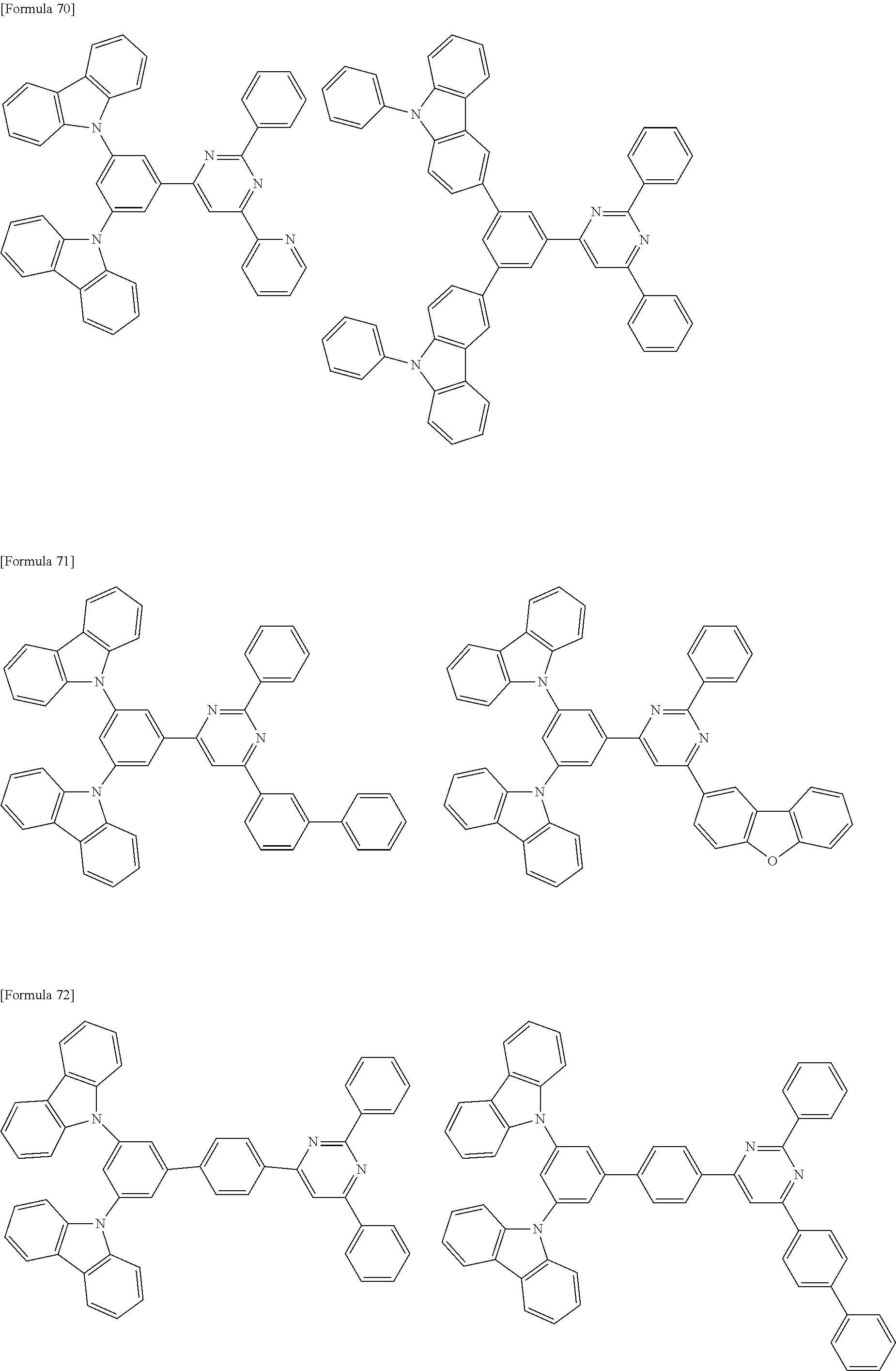

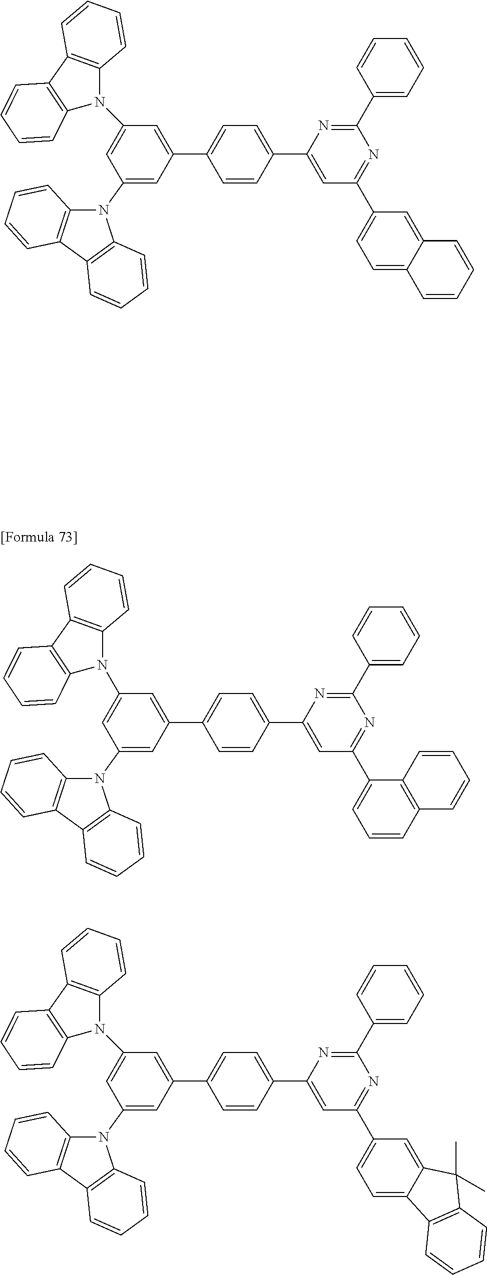 [SMILES: c1ccc(-c2nc(-c3ccc(-c4cc(-n5c6ccccc6c6ccccc65)cc(-n5c6ccccc6c6ccccc65)c4)cc3)cc(-c3ccc4ccccc4c3)n2)cc1 ; c1ccc(-c2nc(-c3ccc(-c4cc(-n5c6ccccc6c6ccccc65)cc(-n5c6ccccc6c6ccccc65)c4)cc3)cc(-c3cccc4ccccc34)n2)cc1 ; CC1(C)c2ccccc2-c2ccc(-c3cc(-c4ccc(-c5cc(-n6c7ccccc7c7ccccc76)cc(-n6c7ccccc7c7ccccc76)c5)cc4)nc(-c4ccccc4)n3)cc21]

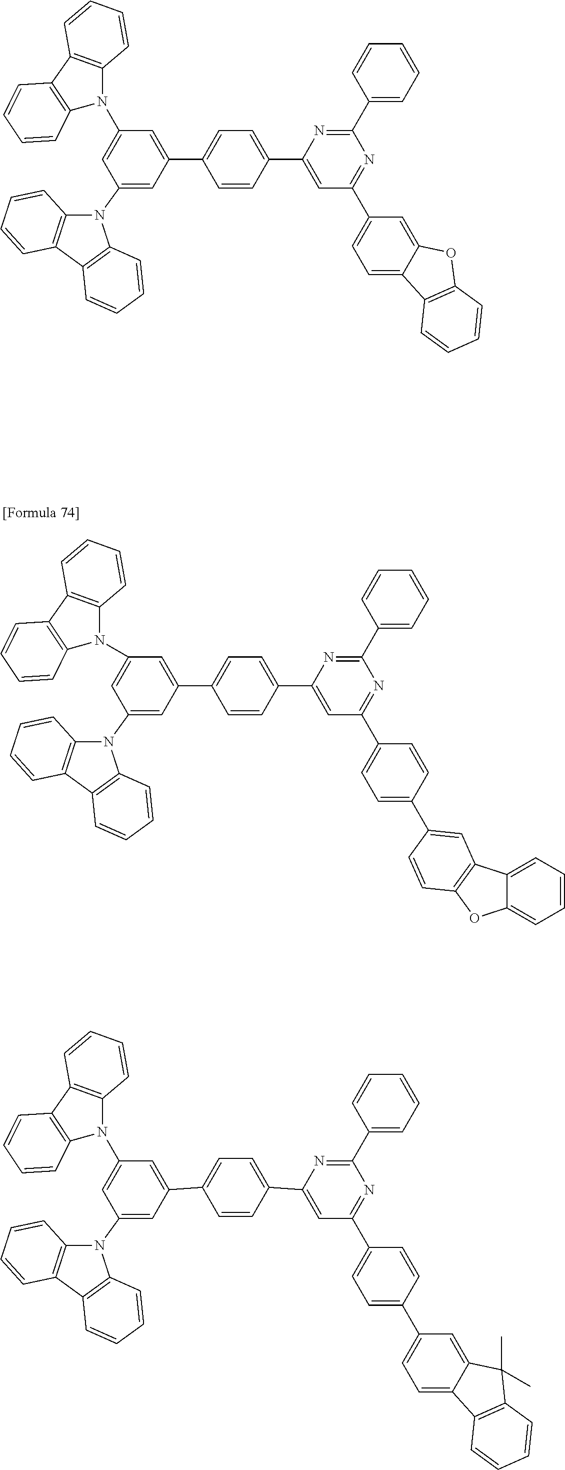 [SMILES: c1ccc(-c2nc(-c3ccc(-c4cc(-n5c6ccccc6c6ccccc65)cc(-n5c6ccccc6c6ccccc65)c4)cc3)cc(-c3ccc4c(c3)oc3ccccc34)n2)cc1 ; c1ccc(-c2nc(-c3ccc(-c4cc(-n5c6ccccc6c6ccccc65)cc(-n5c6ccccc6c6ccccc65)c4)cc3)cc(-c3ccc(-c4ccc5oc6ccccc6c5c4)cc3)n2)cc1 ; CC1(C)c2ccccc2-c2ccc(-c3ccc(-c4cc(-c5ccc(-c6cc(-n7c8ccccc8c8ccccc87)cc(-n7c8ccccc8c8ccccc87)c6)cc5)nc(-c5ccccc5)n4)cc3)cc21]

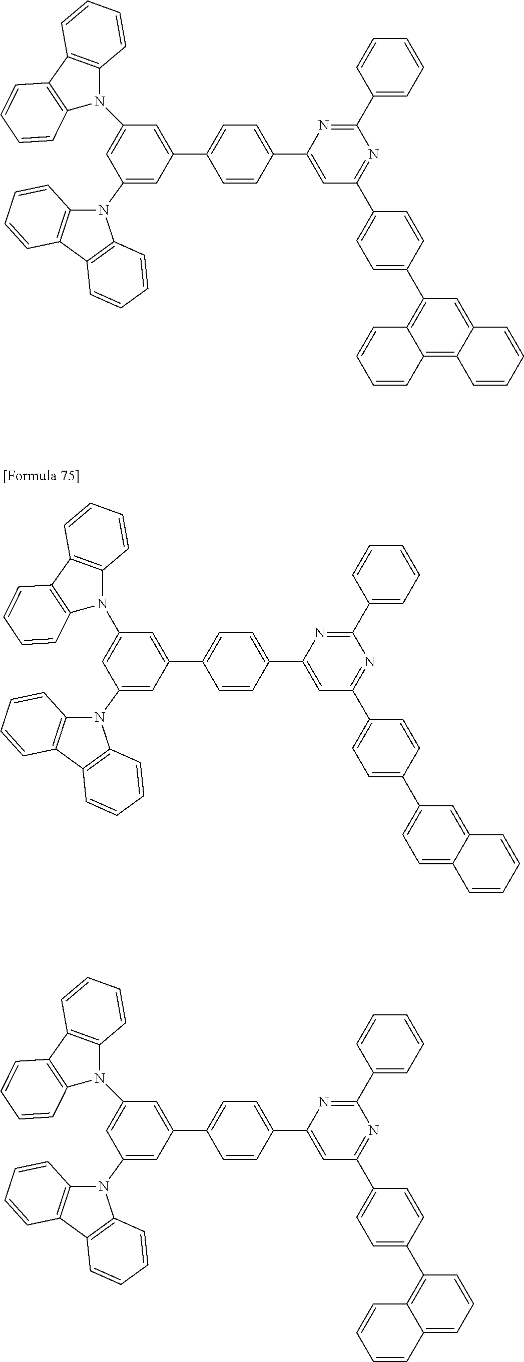 [SMILES: c1ccc(-c2nc(-c3ccc(-c4cc(-n5c6ccccc6c6ccccc65)cc(-n5c6ccccc6c6ccccc65)c4)cc3)cc(-c3ccc(-c4cc5ccccc5c5ccccc45)cc3)n2)cc1 ; c1ccc(-c2nc(-c3ccc(-c4cc(-n5c6ccccc6c6ccccc65)cc(-n5c6ccccc6c6ccccc65)c4)cc3)cc(-c3ccc(-c4ccc5ccccc5c4)cc3)n2)cc1 ; c1ccc(-c2nc(-c3ccc(-c4cc(-n5c6ccccc6c6ccccc65)cc(-n5c6ccccc6c6ccccc65)c4)cc3)cc(-c3ccc(-c4cccc5ccccc45)cc3)n2)cc1]

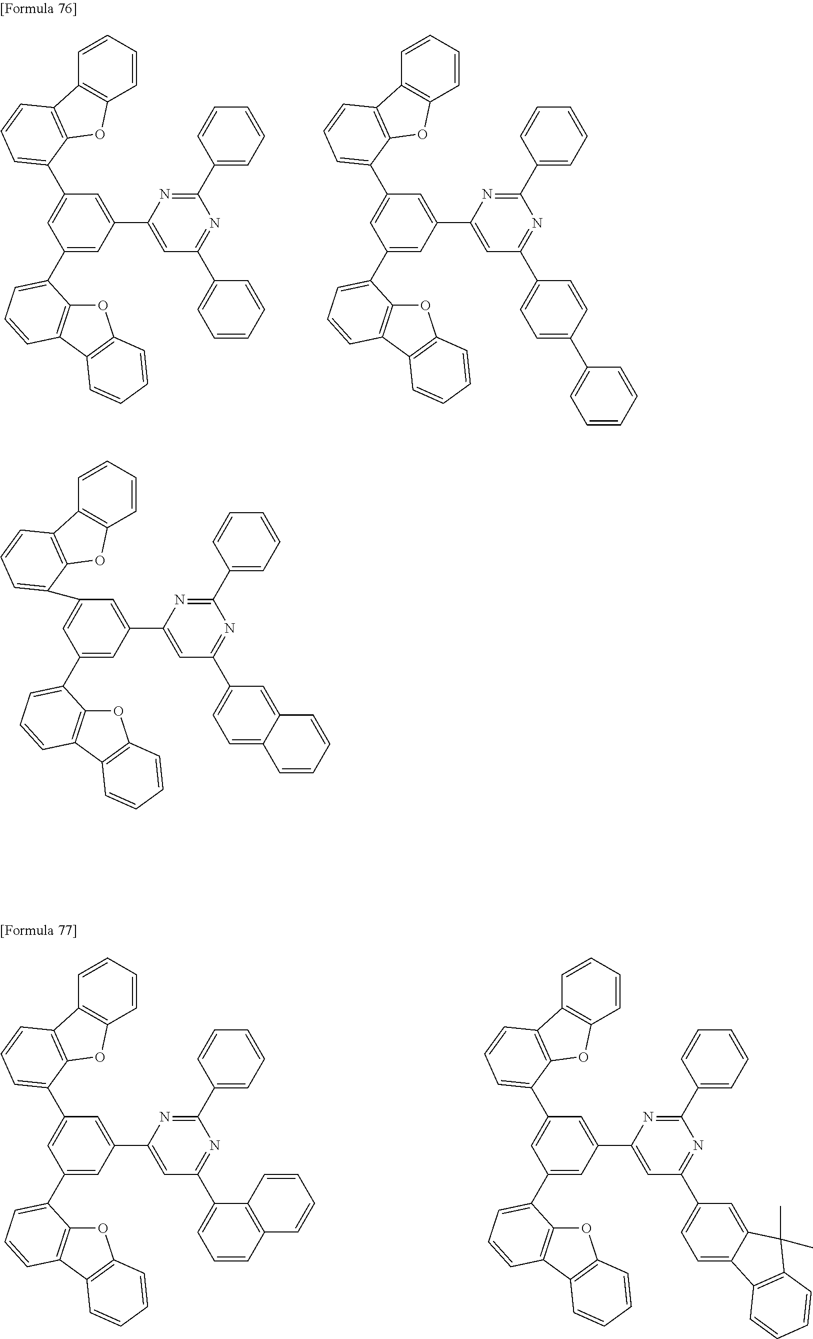 [SMILES: c1ccc(-c2cc(-c3cc(-c4cccc5c4oc4ccccc45)cc(-c4cccc5c4oc4ccccc45)c3)nc(-c3ccccc3)n2)cc1 ; c1ccc(-c2ccc(-c3cc(-c4cc(-c5cccc6c5oc5ccccc56)cc(-c5cccc6c5oc5ccccc56)c4)nc(-c4ccccc4)n3)cc2)cc1 ; c1ccc(-c2nc(-c3cc(-c4cccc5c4oc4ccccc45)cc(-c4cccc5c4oc4ccccc45)c3)cc(-c3ccc4ccccc4c3)n2)cc1 ; c1ccc(-c2nc(-c3cc(-c4cccc5c4oc4ccccc45)cc(-c4cccc5c4oc4ccccc45)c3)cc(-c3cccc4ccccc34)n2)cc1 ; CC1(C)c2ccccc2-c2ccc(-c3cc(-c4cc(-c5cccc6c5oc5ccccc56)cc(-c5cccc6c5oc5ccccc56)c4)nc(-c4ccccc4)n3)cc21]

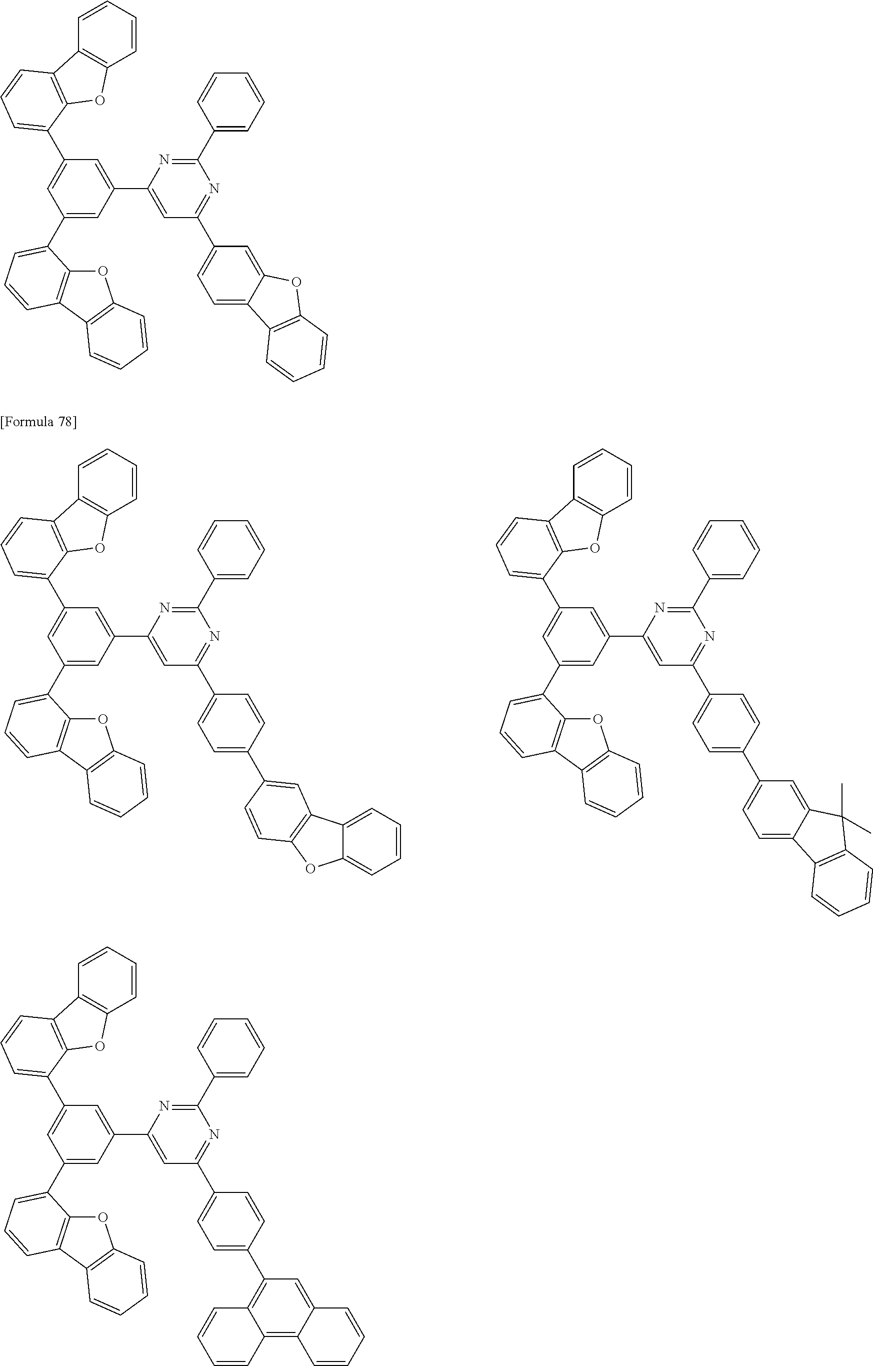 [SMILES: c1ccc(-c2nc(-c3cc(-c4cccc5c4oc4ccccc45)cc(-c4cccc5c4oc4ccccc45)c3)cc(-c3ccc4c(c3)oc3ccccc34)n2)cc1 ; c1ccc(-c2nc(-c3ccc(-c4ccc5oc6ccccc6c5c4)cc3)cc(-c3cc(-c4cccc5c4oc4ccccc45)cc(-c4cccc5c4oc4ccccc45)c3)n2)cc1 ; CC1(C)c2ccccc2-c2ccc(-c3ccc(-c4cc(-c5cc(-c6cccc7c6oc6ccccc67)cc(-c6cccc7c6oc6ccccc67)c5)nc(-c5ccccc5)n4)cc3)cc21 ; c1ccc(-c2nc(-c3ccc(-c4cc5ccccc5c5ccccc45)cc3)cc(-c3cc(-c4cccc5c4oc4ccccc45)cc(-c4cccc5c4oc4ccccc45)c3)n2)cc1]

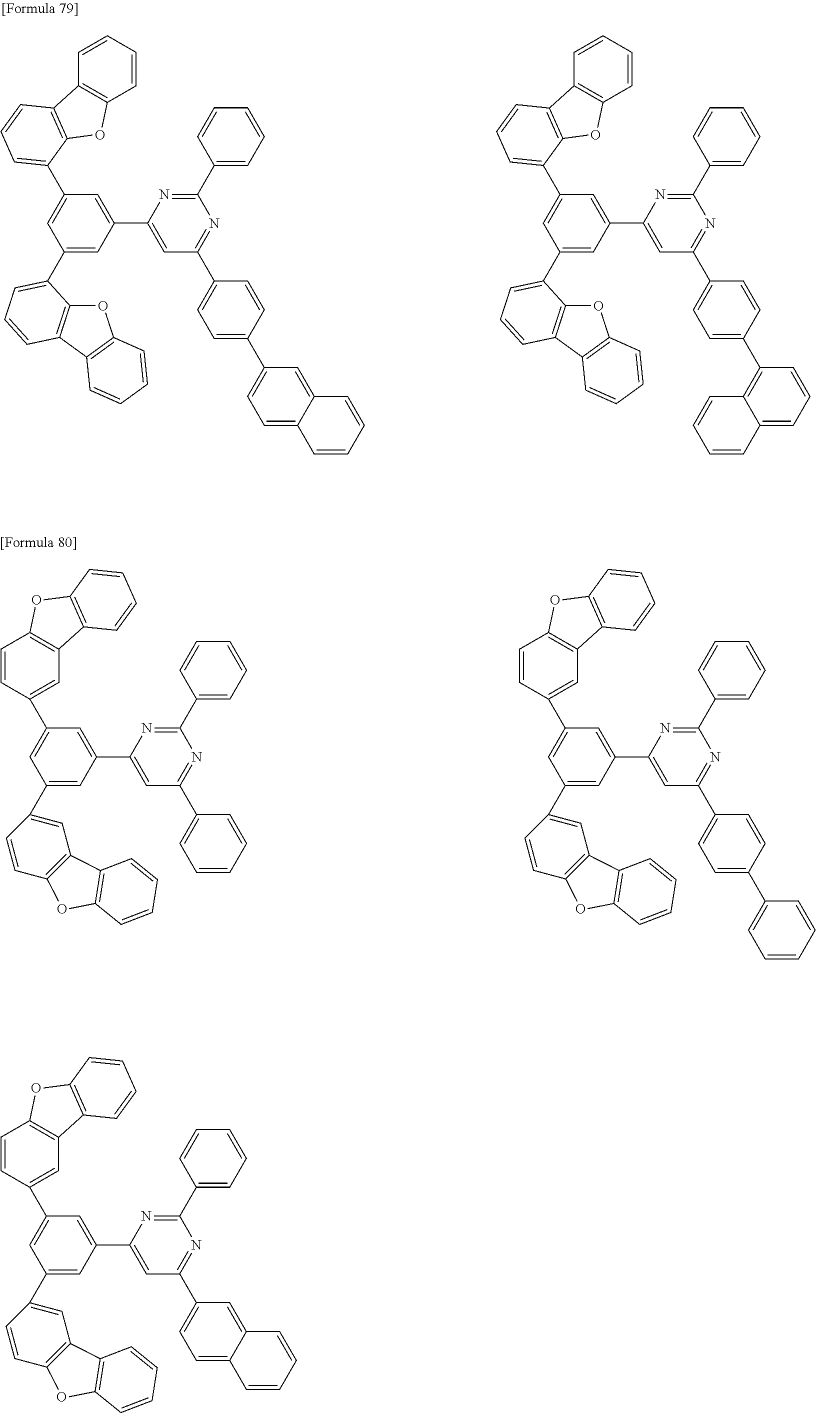 [SMILES: c1ccc(-c2nc(-c3ccc(-c4ccc5ccccc5c4)cc3)cc(-c3cc(-c4cccc5c4oc4ccccc45)cc(-c4cccc5c4oc4ccccc45)c3)n2)cc1 ; c1ccc(-c2nc(-c3ccc(-c4cccc5ccccc45)cc3)cc(-c3cc(-c4cccc5c4oc4ccccc45)cc(-c4cccc5c4oc4ccccc45)c3)n2)cc1 ; c1ccc(-c2cc(-c3cc(-c4ccc5oc6ccccc6c5c4)cc(-c4ccc5oc6ccccc6c5c4)c3)nc(-c3ccccc3)n2)cc1 ; c1ccc(-c2ccc(-c3cc(-c4cc(-c5ccc6oc7ccccc7c6c5)cc(-c5ccc6oc7ccccc7c6c5)c4)nc(-c4ccccc4)n3)cc2)cc1 ; c1ccc(-c2nc(-c3cc(-c4ccc5oc6ccccc6c5c4)cc(-c4ccc5oc6ccccc6c5c4)c3)cc(-c3ccc4ccccc4c3)n2)cc1]

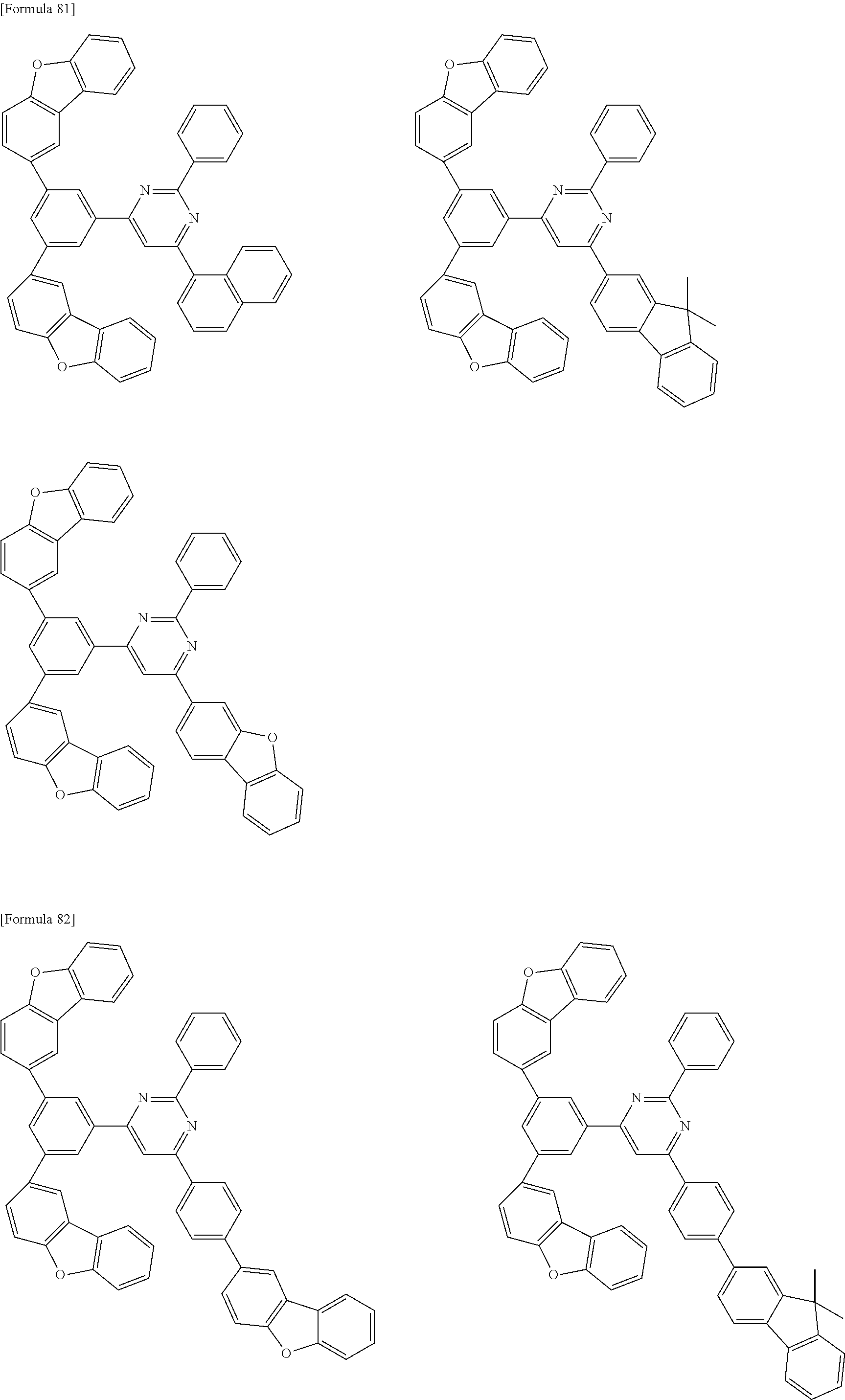

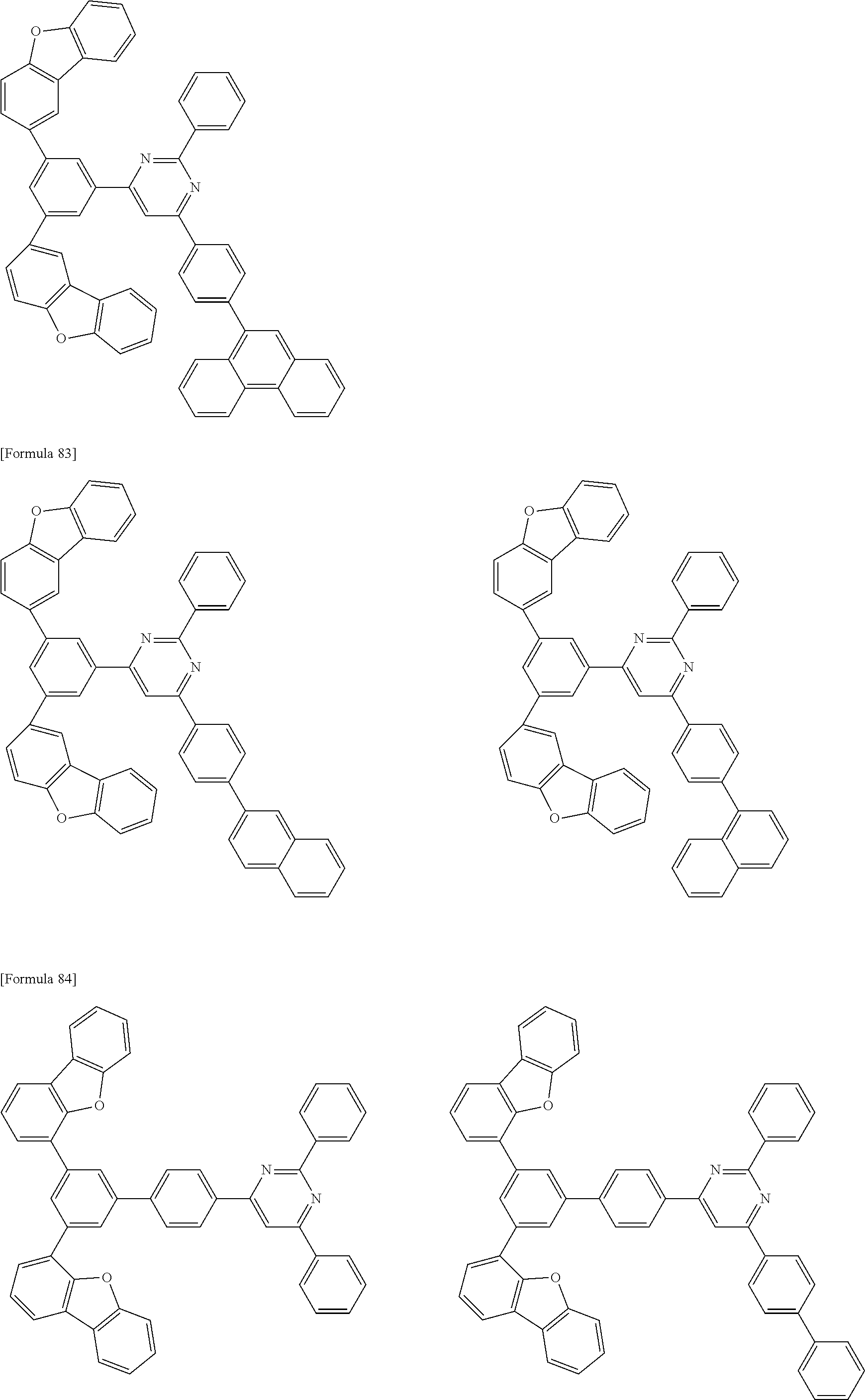

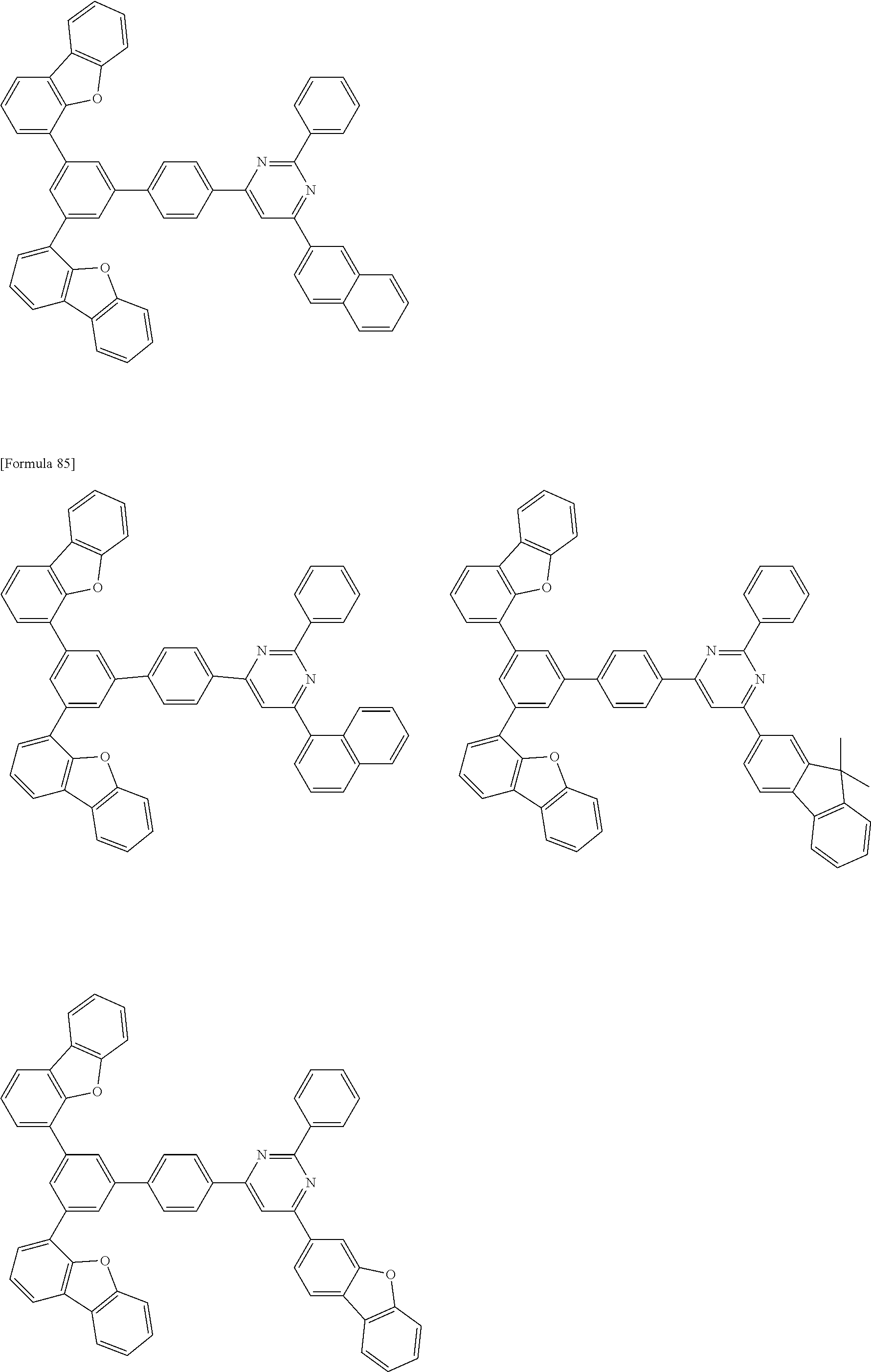 [SMILES: c1ccc(-c2nc(-c3ccc(-c4cc(-c5cccc6c5oc5ccccc56)cc(-c5cccc6c5oc5ccccc56)c4)cc3)cc(-c3ccc4ccccc4c3)n2)cc1 ; c1ccc(-c2nc(-c3ccc(-c4cc(-c5cccc6c5oc5ccccc56)cc(-c5cccc6c5oc5ccccc56)c4)cc3)cc(-c3cccc4ccccc34)n2)cc1 ; CC1(C)c2ccccc2-c2ccc(-c3cc(-c4ccc(-c5cc(-c6cccc7c6oc6ccccc67)cc(-c6cccc7c6oc6ccccc67)c5)cc4)nc(-c4ccccc4)n3)cc21 ; c1ccc(-c2nc(-c3ccc(-c4cc(-c5cccc6c5oc5ccccc56)cc(-c5cccc6c5oc5ccccc56)c4)cc3)cc(-c3ccc4c(c3)oc3ccccc34)n2)cc1]

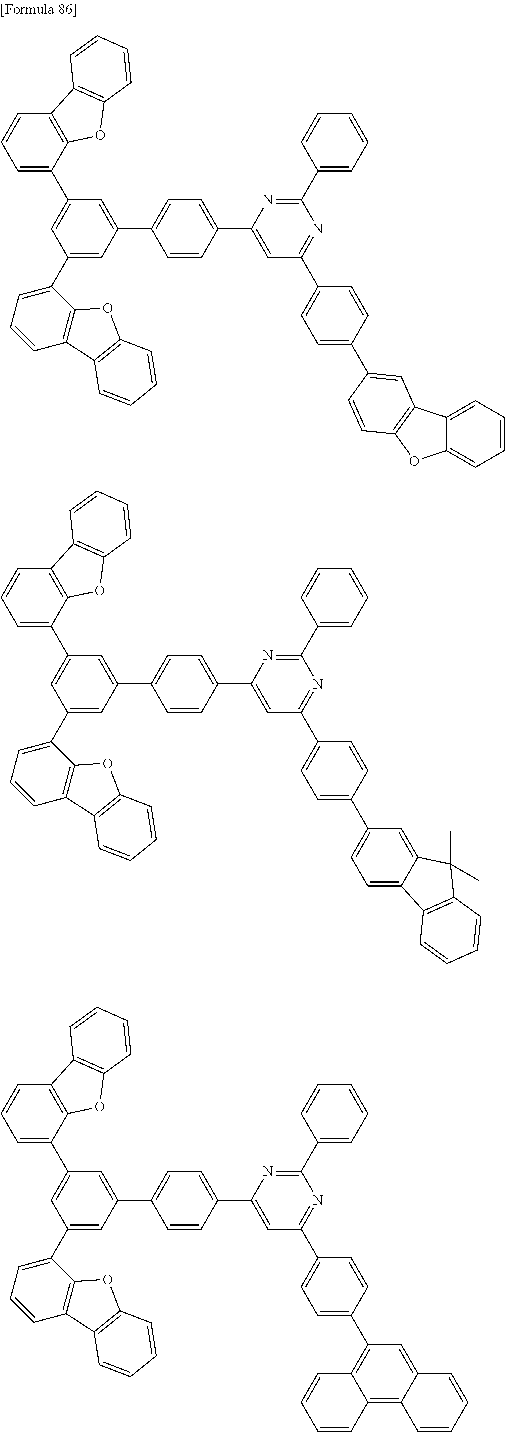 [SMILES: c1ccc(-c2nc(-c3ccc(-c4cc(-c5cccc6c5oc5ccccc56)cc(-c5cccc6c5oc5ccccc56)c4)cc3)cc(-c3ccc(-c4ccc5oc6ccccc6c5c4)cc3)n2)cc1 ; CC1(C)c2ccccc2-c2ccc(-c3ccc(-c4cc(-c5ccc(-c6cc(-c7cccc8c7oc7ccccc78)cc(-c7cccc8c7oc7ccccc78)c6)cc5)nc(-c5ccccc5)n4)cc3)cc21 ; c1ccc(-c2nc(-c3ccc(-c4cc(-c5cccc6c5oc5ccccc56)cc(-c5cccc6c5oc5ccccc56)c4)cc3)cc(-c3ccc(-c4cc5ccccc5c5ccccc45)cc3)n2)cc1]

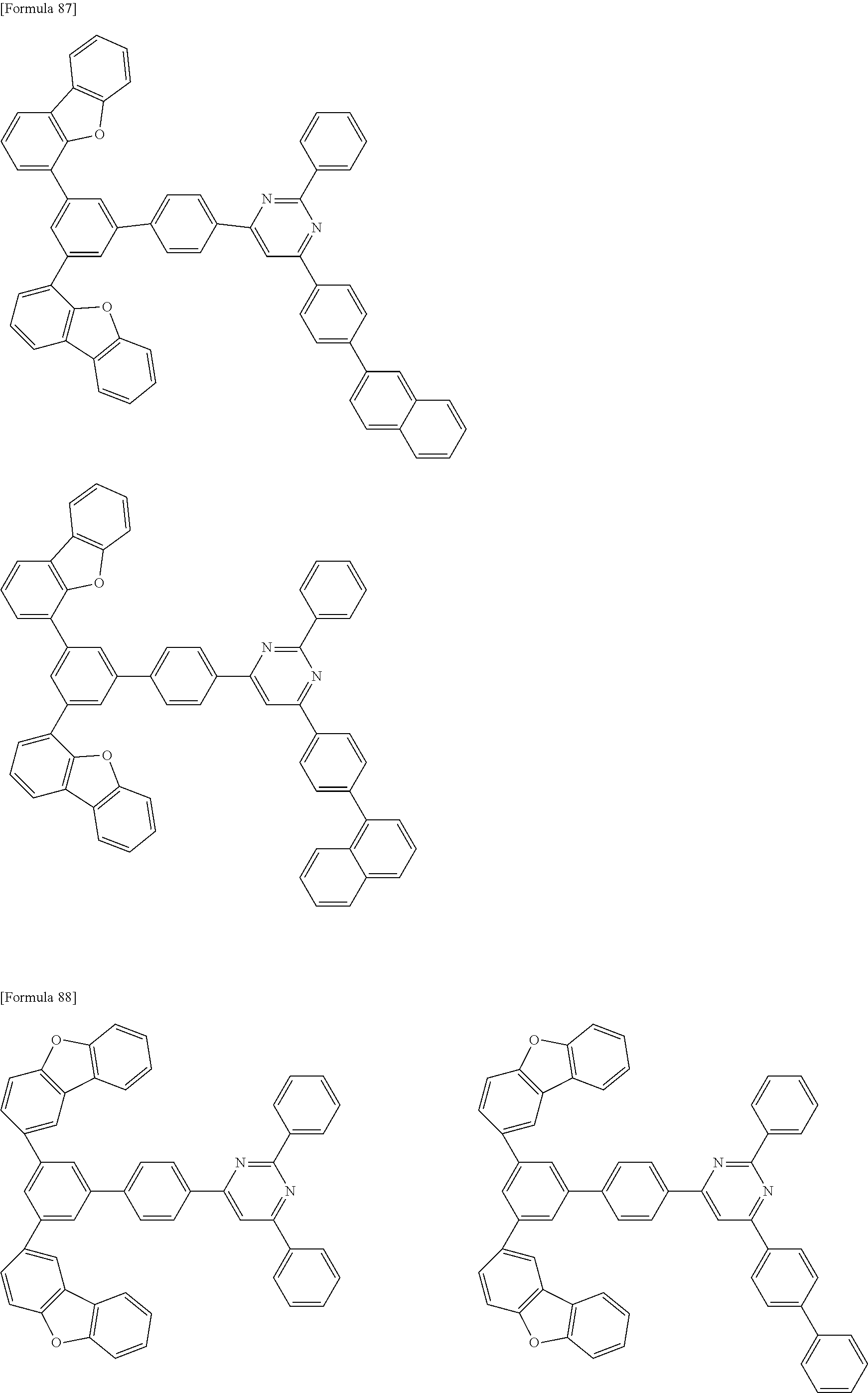 [SMILES: c1ccc(-c2nc(-c3ccc(-c4cc(-c5cccc6c5oc5ccccc56)cc(-c5cccc6c5oc5ccccc56)c4)cc3)cc(-c3ccc(-c4ccc5ccccc5c4)cc3)n2)cc1 ; c1ccc(-c2nc(-c3ccc(-c4cc(-c5cccc6c5oc5ccccc56)cc(-c5cccc6c5oc5ccccc56)c4)cc3)cc(-c3ccc(-c4cccc5ccccc45)cc3)n2)cc1 ; c1ccc(-c2cc(-c3ccc(-c4cc(-c5ccc6oc7ccccc7c6c5)cc(-c5ccc6oc7ccccc7c6c5)c4)cc3)nc(-c3ccccc3)n2)cc1 ; c1ccc(-c2ccc(-c3cc(-c4ccc(-c5cc(-c6ccc7oc8ccccc8c7c6)cc(-c6ccc7oc8ccccc8c7c6)c5)cc4)nc(-c4ccccc4)n3)cc2)cc1]

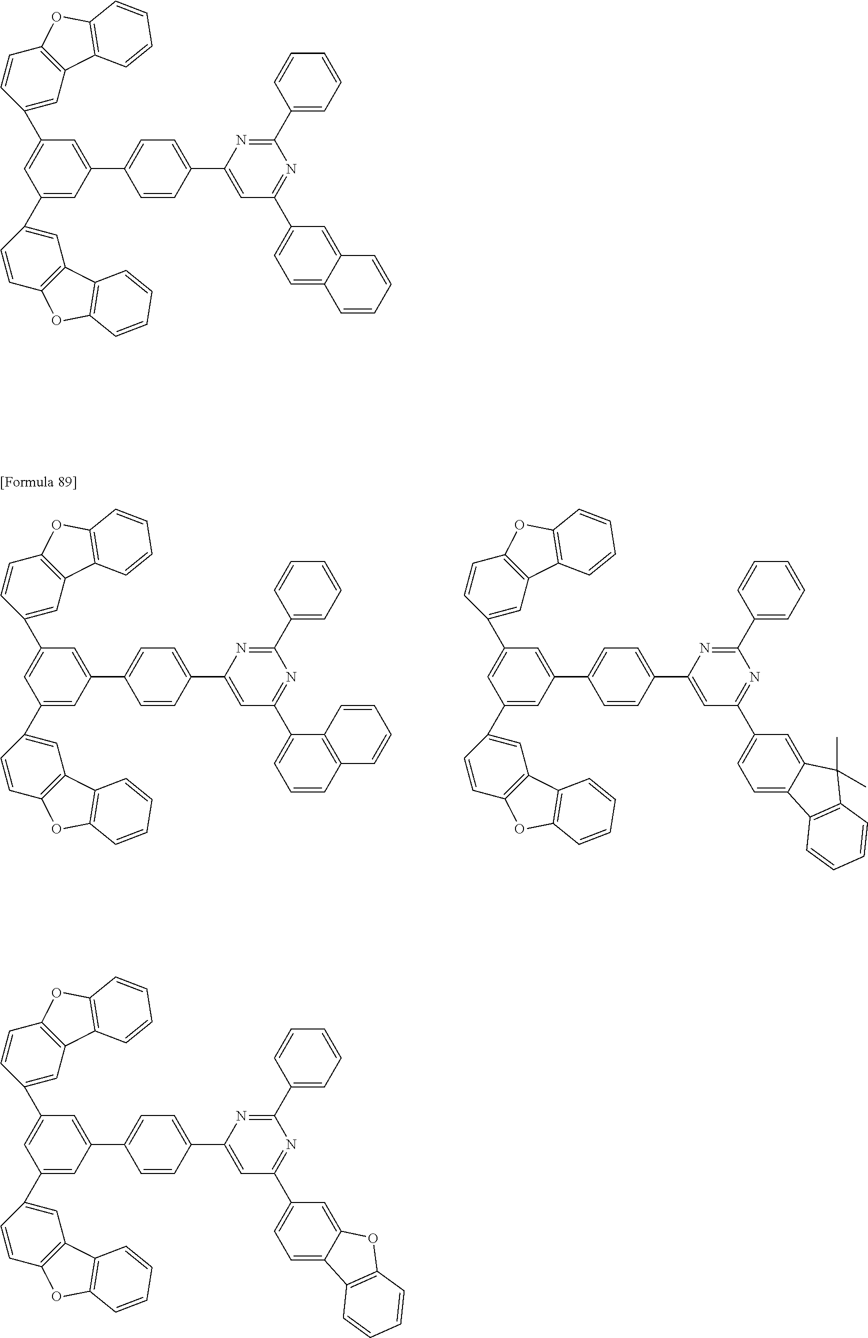 [SMILES: c1ccc(-c2nc(-c3ccc(-c4cc(-c5ccc6oc7ccccc7c6c5)cc(-c5ccc6oc7ccccc7c6c5)c4)cc3)cc(-c3ccc4ccccc4c3)n2)cc1 ; c1ccc(-c2nc(-c3ccc(-c4cc(-c5ccc6oc7ccccc7c6c5)cc(-c5ccc6oc7ccccc7c6c5)c4)cc3)cc(-c3cccc4ccccc34)n2)cc1 ; CC1(C)c2ccccc2-c2ccc(-c3cc(-c4ccc(-c5cc(-c6ccc7oc8ccccc8c7c6)cc(-c6ccc7oc8ccccc8c7c6)c5)cc4)nc(-c4ccccc4)n3)cc21 ; c1ccc(-c2nc(-c3ccc(-c4cc(-c5ccc6oc7ccccc7c6c5)cc(-c5ccc6oc7ccccc7c6c5)c4)cc3)cc(-c3ccc4c(c3)oc3ccccc34)n2)cc1]

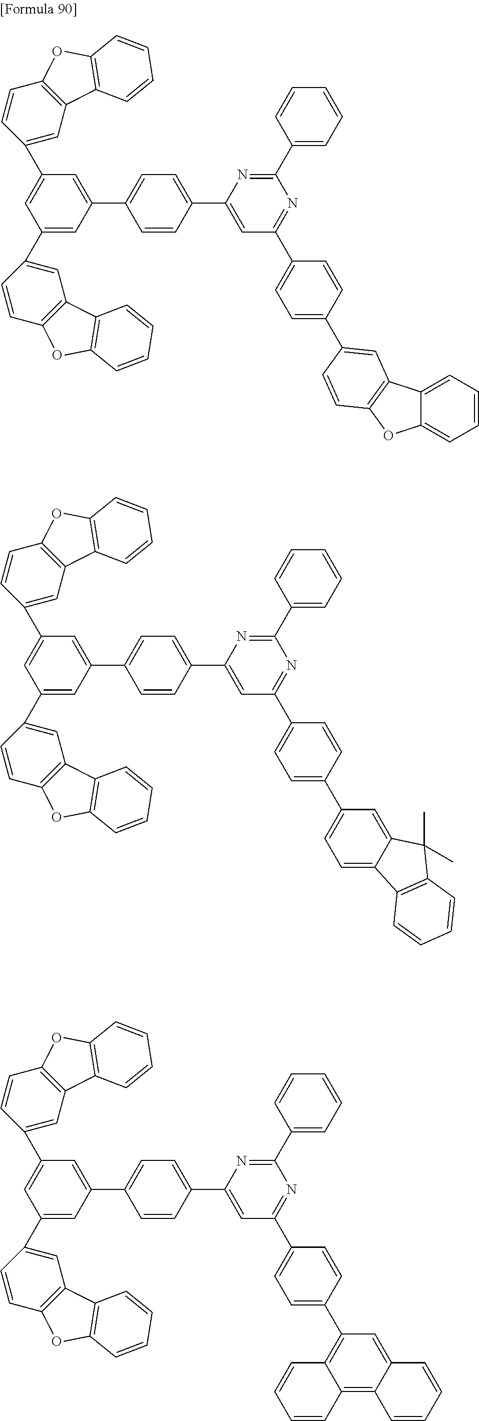 [SMILES: c1ccc(-c2nc(-c3ccc(-c4cc(-c5ccc6oc7ccccc7c6c5)cc(-c5ccc6oc7ccccc7c6c5)c4)cc3)cc(-c3ccc(-c4ccc5oc6ccccc6c5c4)cc3)n2)cc1 ; CC1(C)c2ccccc2-c2ccc(-c3ccc(-c4cc(-c5ccc(-c6cc(-c7ccc8oc9ccccc9c8c7)cc(-c7ccc8oc9ccccc9c8c7)c6)cc5)nc(-c5ccccc5)n4)cc3)cc21 ; c1ccc(-c2nc(-c3ccc(-c4cc(-c5ccc6oc7ccccc7c6c5)cc(-c5ccc6oc7ccccc7c6c5)c4)cc3)cc(-c3ccc(-c4cc5ccccc5c5ccccc45)cc3)n2)cc1]

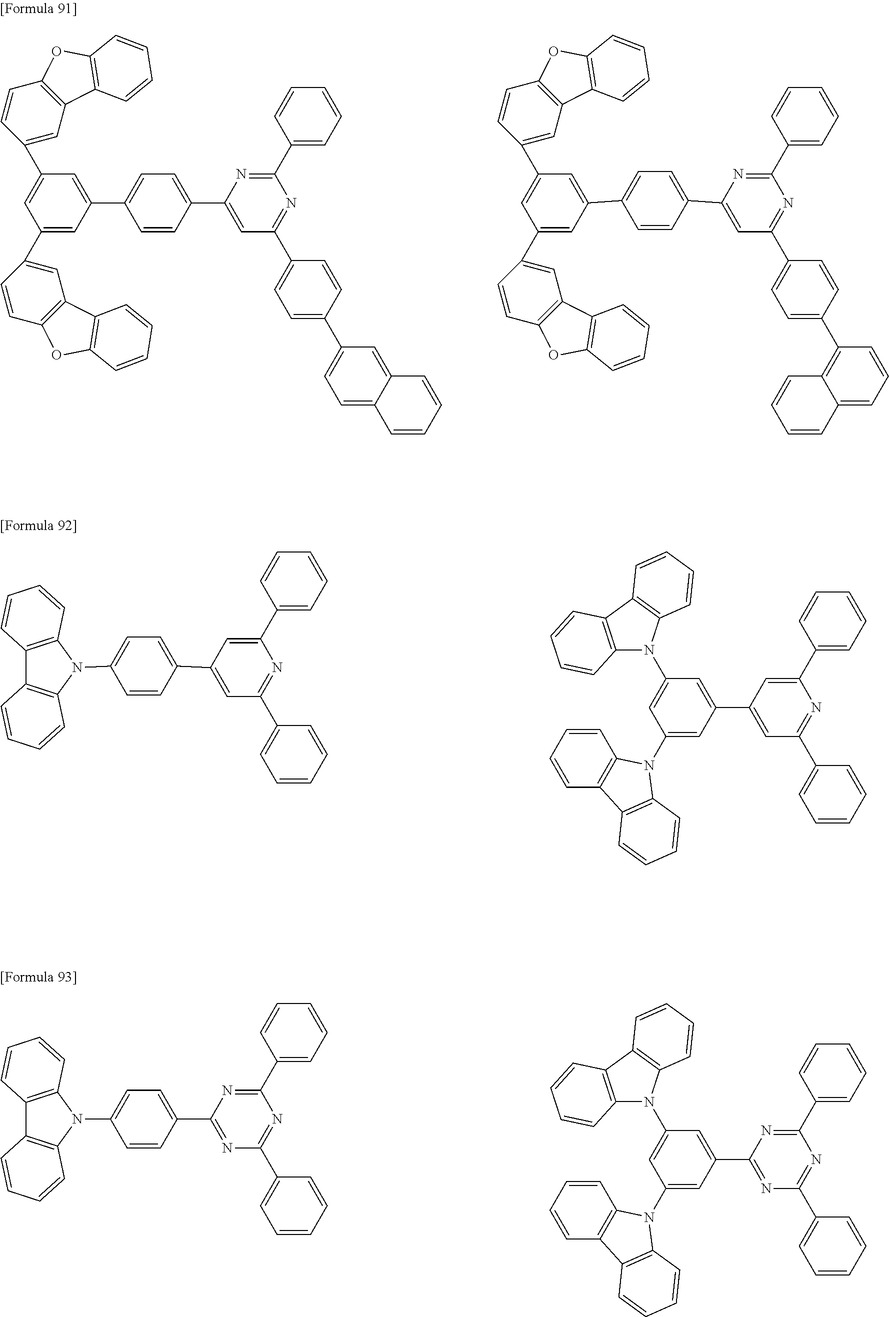

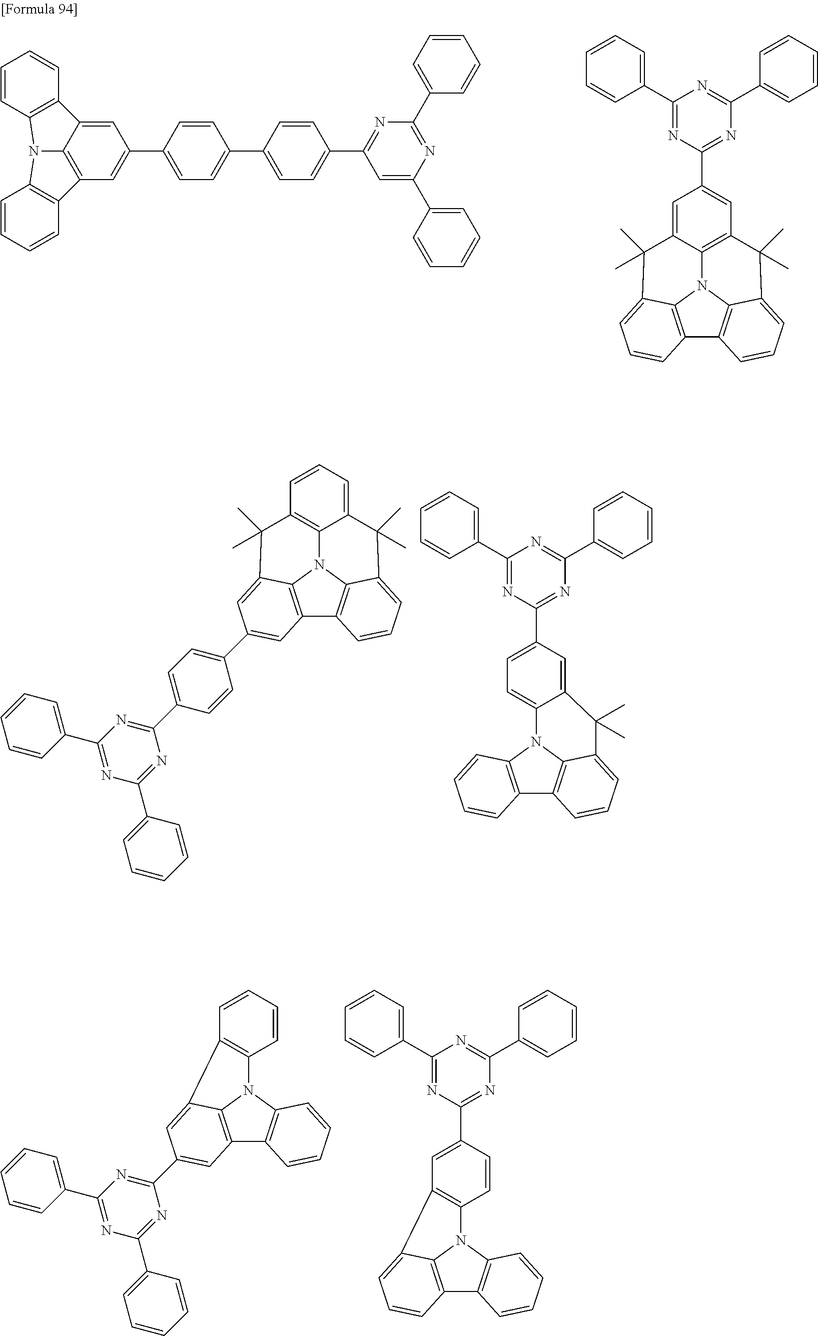

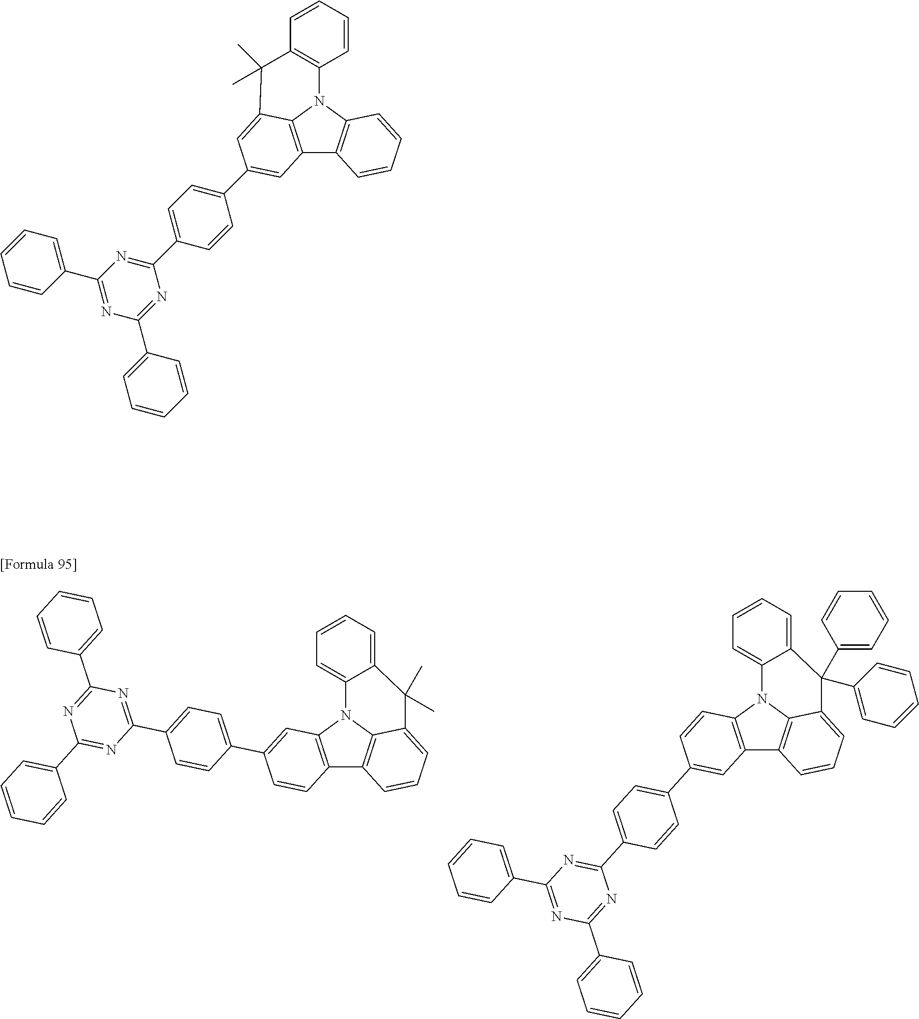 [SMILES: CC1(C)c2ccccc2-n2c3ccccc3c3cc(-c4ccc(-c5nc(-c6ccccc6)nc(-c6ccccc6)n5)cc4)cc1c32 ; CC1(C)c2ccccc2-n2c3cc(-c4ccc(-c5nc(-c6ccccc6)nc(-c6ccccc6)n5)cc4)ccc3c3cccc1c32 ; c1ccc(-c2nc(-c3ccccc3)nc(-c3ccc(-c4ccc5c(c4)c4cccc6c4n5-c4ccccc4C6(c4ccccc4)c4ccccc4)cc3)n2)cc1]

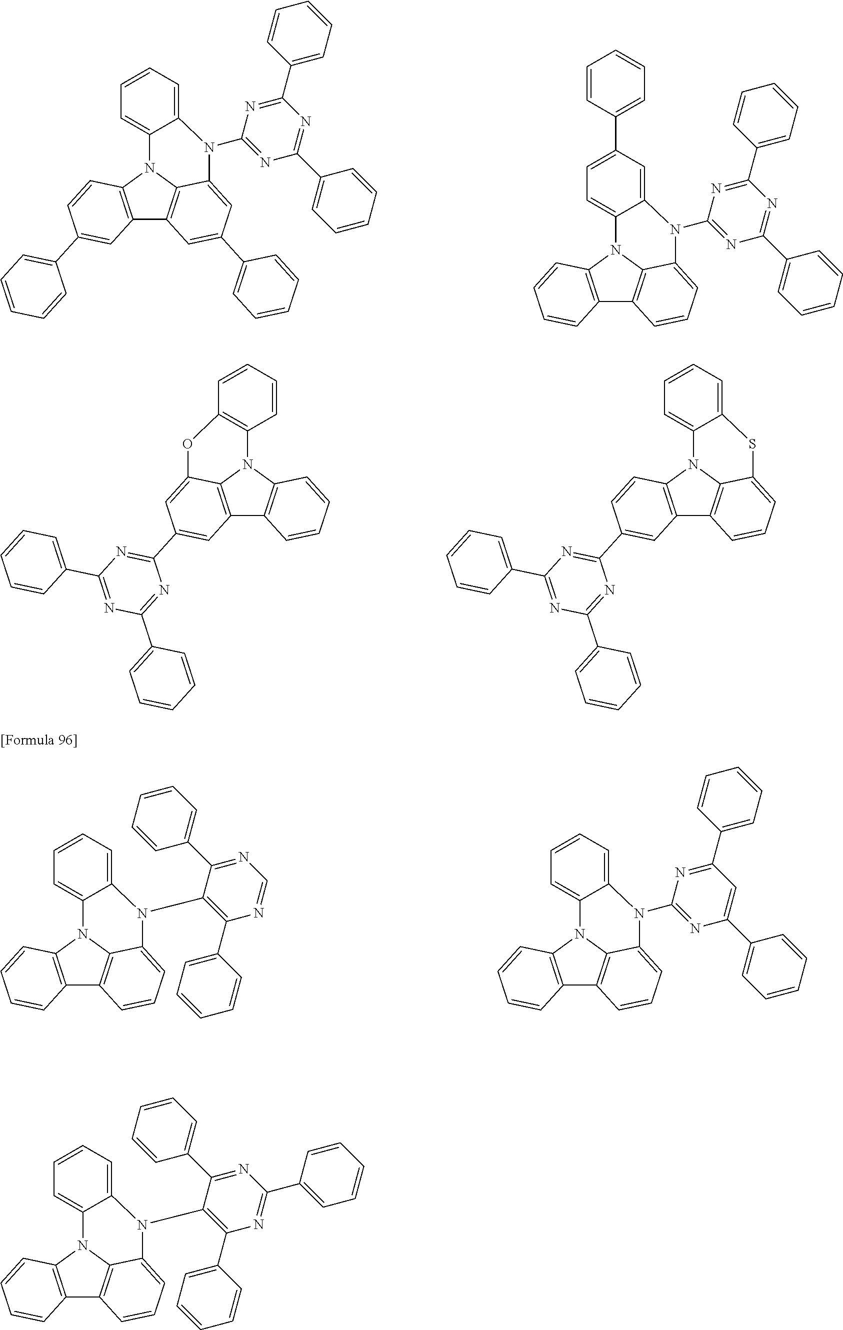

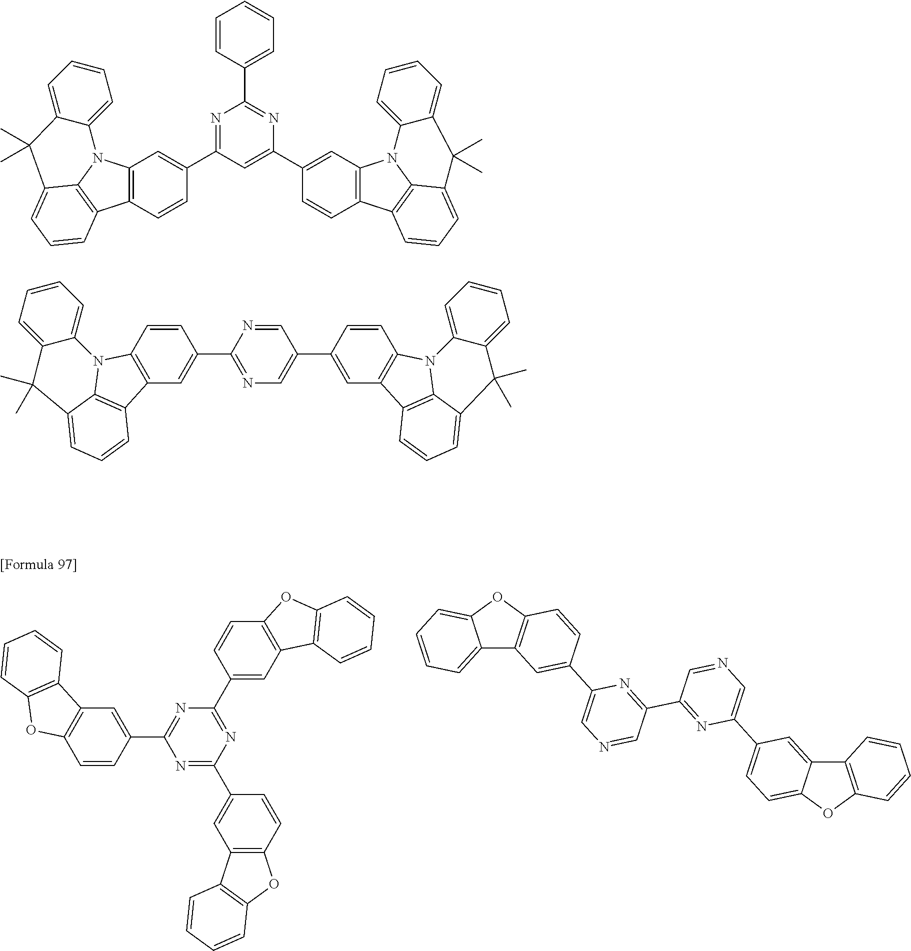

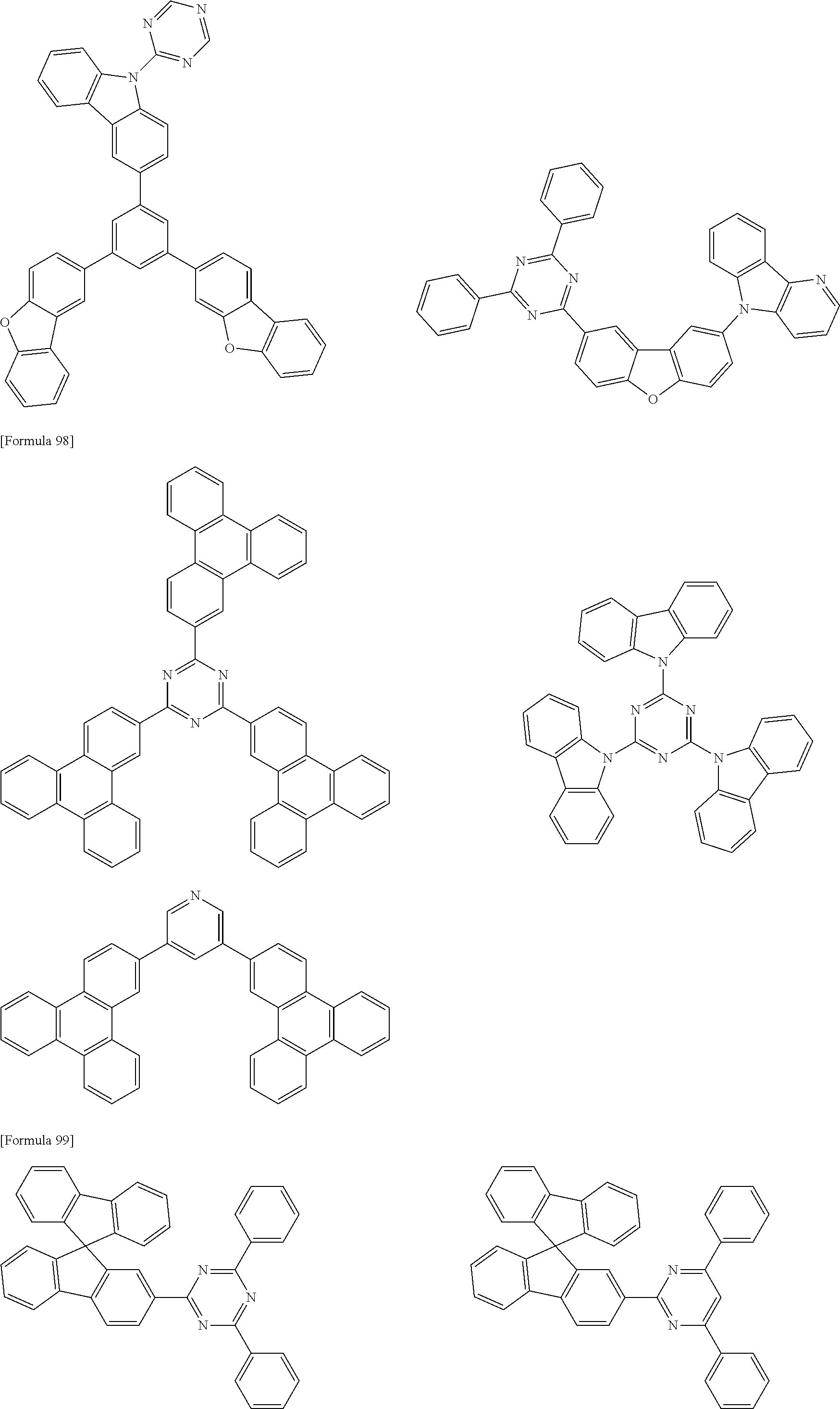

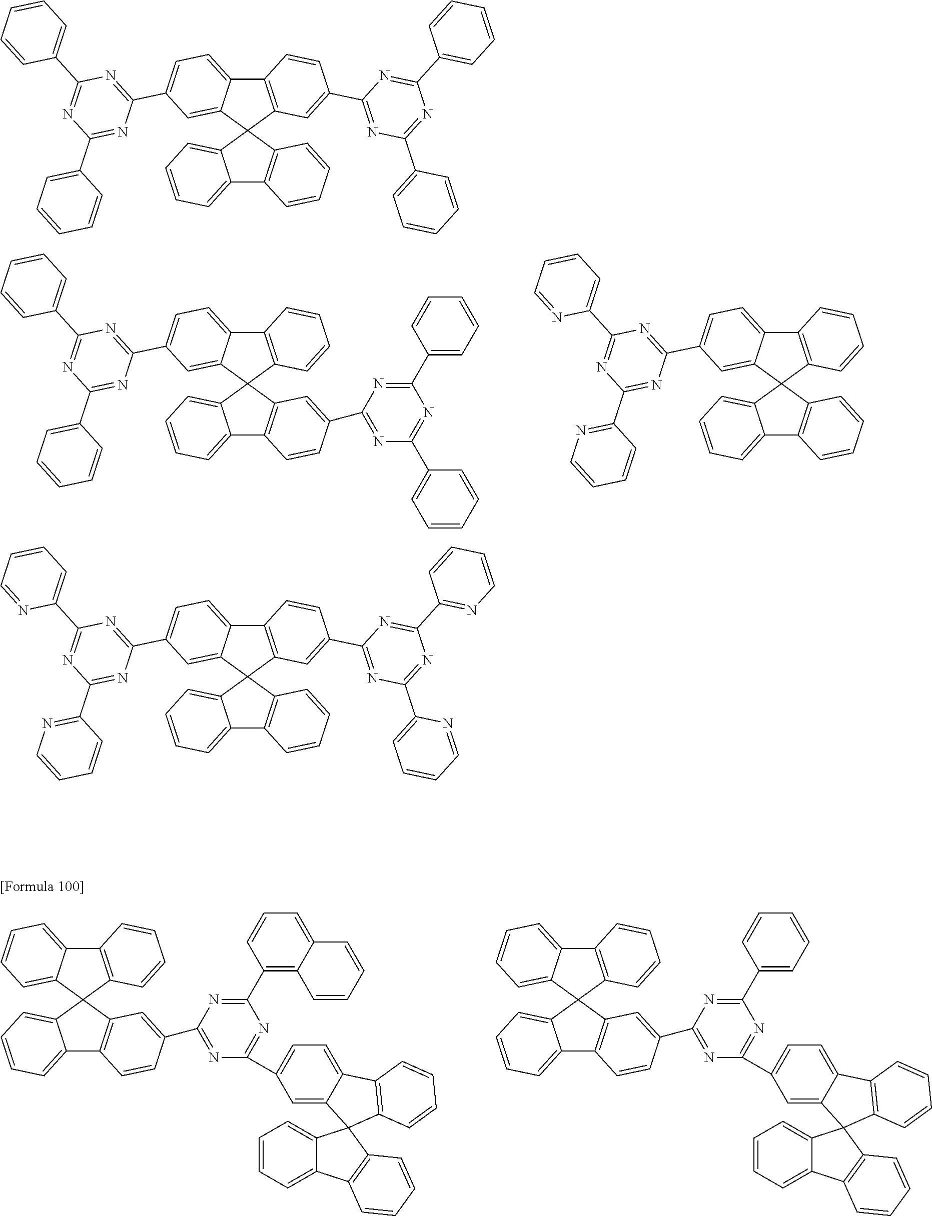

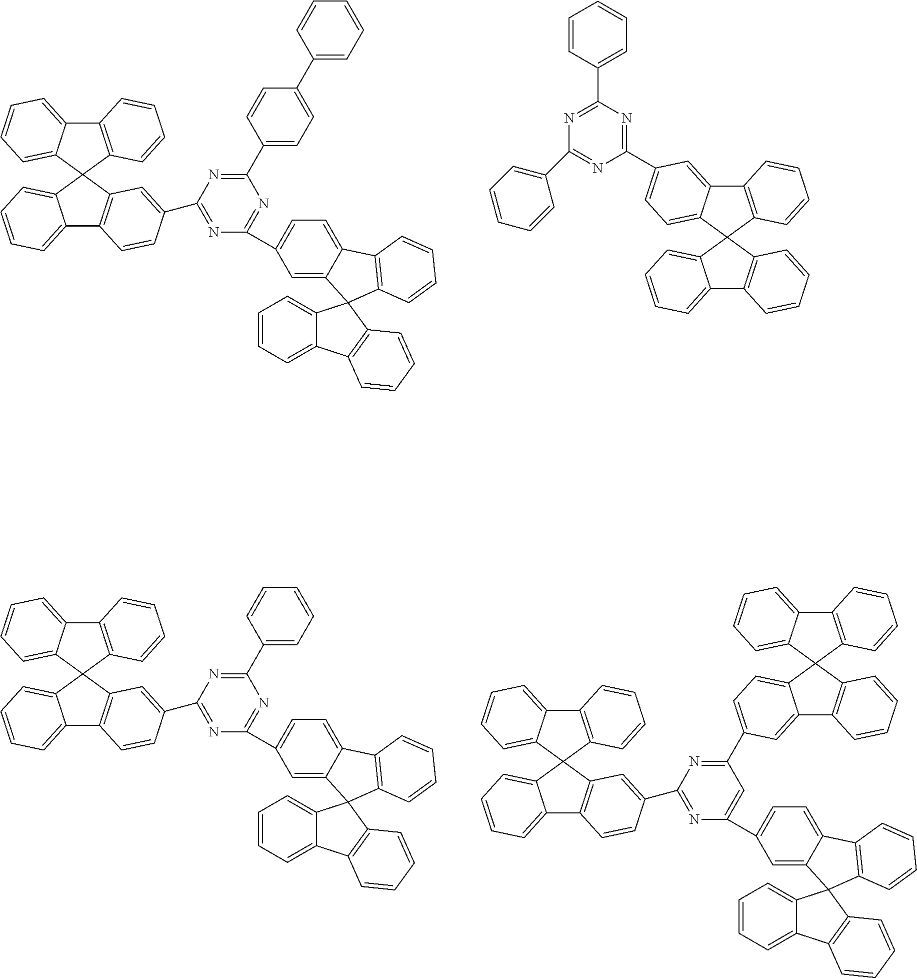 [SMILES: c1ccc(-c2ccc(-c3nc(-c4ccc5c(c4)C4(c6ccccc6-c6ccccc64)c4ccccc4-5)nc(-c4ccc5c(c4)C4(c6ccccc6-c6ccccc64)c4ccccc4-5)n3)cc2)cc1 ; c1ccc(-c2nc(-c3ccccc3)nc(-c3ccc4c(c3)-c3ccccc3C43c4ccccc4-c4ccccc43)n2)cc1 ; c1ccc(-c2nc(-c3ccc4c(c3)C3(c5ccccc5-c5ccccc53)c3ccccc3-4)nc(-c3ccc4c(c3)C3(c5ccccc5-c5ccccc53)c3ccccc3-4)n2)cc1 ; c1ccc2c(c1)-c1ccccc1C21c2ccccc2-c2cc(-c3cc(-c4ccc5c(c4)C4(c6ccccc6-c6ccccc64)c4ccccc4-5)nc(-c4ccc5c(c4)C4(c6ccccc6-c6ccccc64)c4ccccc4-5)n3)ccc21]

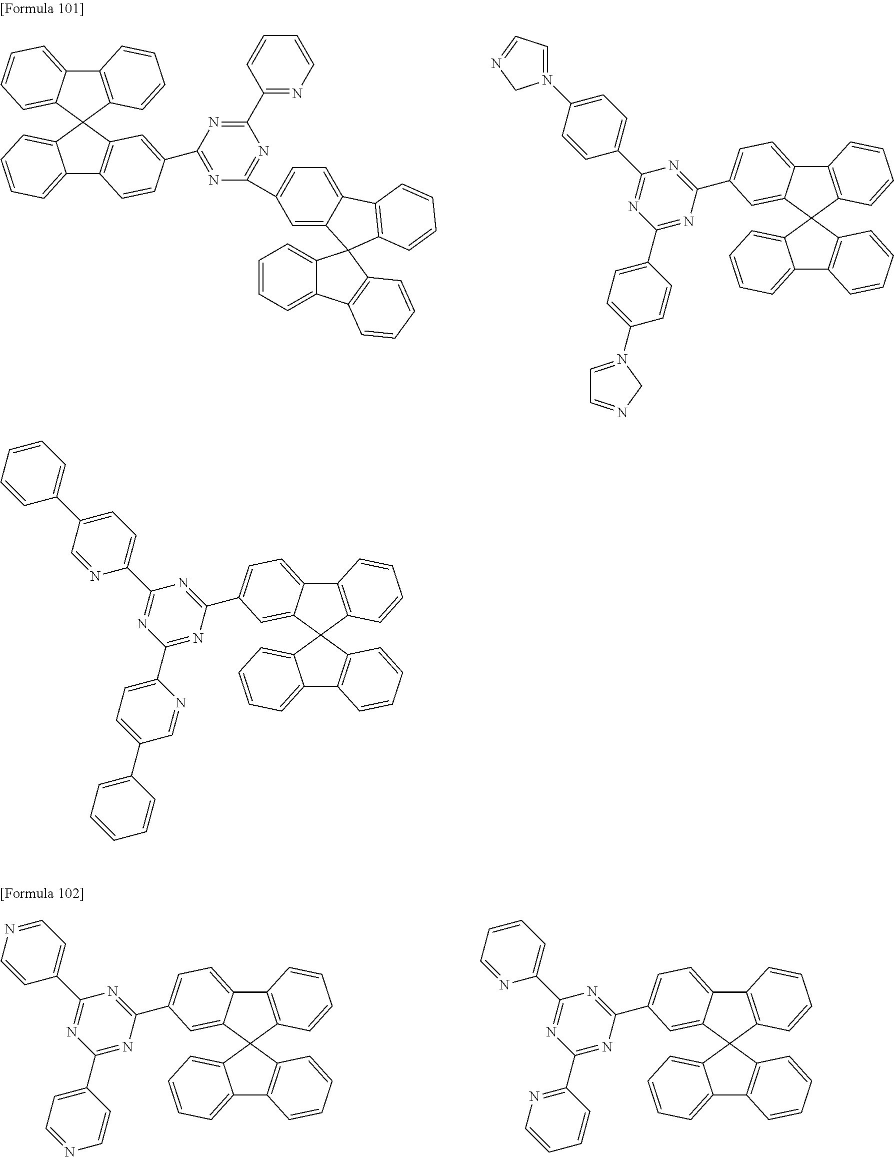

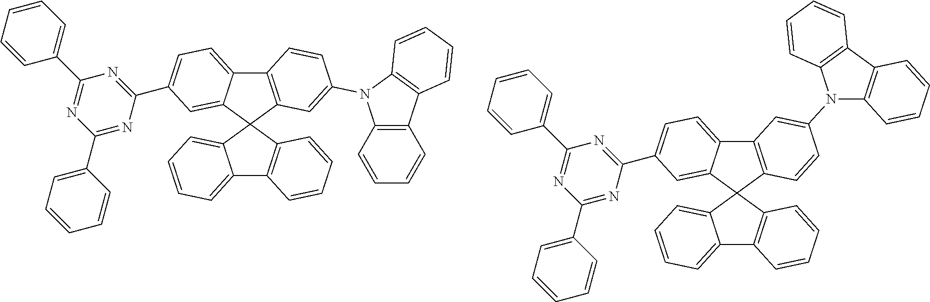 [SMILES: c1ccc(-c2nc(-c3ccccc3)nc(-c3ccc4c(c3)C3(c5ccccc5-c5ccccc53)c3cc(-n5c6ccccc6c6ccccc65)ccc3-4)n2)cc1 ; c1ccc(-c2nc(-c3ccccc3)nc(-c3ccc4c(c3)C3(c5ccccc5-c5ccccc53)c3ccc(-n5c6ccccc6c6ccccc65)cc3-4)n2)cc1]

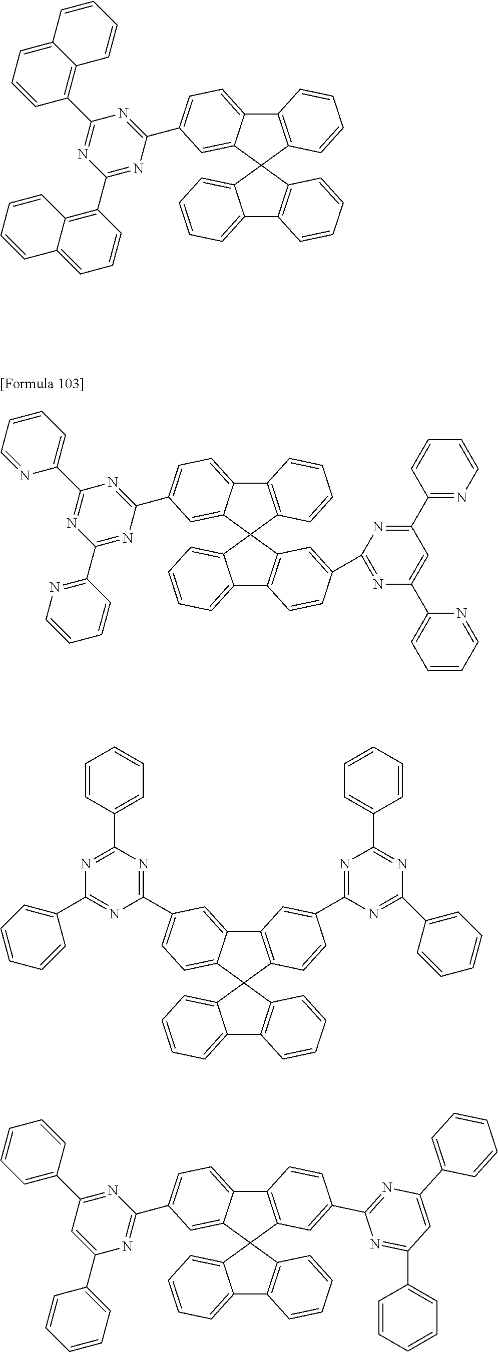 [SMILES: c1ccc2c(c1)-c1ccccc1C21c2ccccc2-c2ccc(-c3nc(-c4cccc5ccccc45)nc(-c4cccc5ccccc45)n3)cc21 ; c1ccc(-c2cc(-c3ccccn3)nc(-c3ccc4c(c3)C3(c5ccccc5-4)c4ccccc4-c4ccc(-c5nc(-c6ccccn6)nc(-c6ccccn6)n5)cc43)n2)nc1 ; c1ccc(-c2nc(-c3ccccc3)nc(-c3ccc4c(c3)-c3cc(-c5nc(-c6ccccc6)nc(-c6ccccc6)n5)ccc3C43c4ccccc4-c4ccccc43)n2)cc1 ; c1ccc(-c2cc(-c3ccccc3)nc(-c3ccc4c(c3)C3(c5ccccc5-c5ccccc53)c3cc(-c5nc(-c6ccccc6)cc(-c6ccccc6)n5)ccc3-4)n2)cc1]

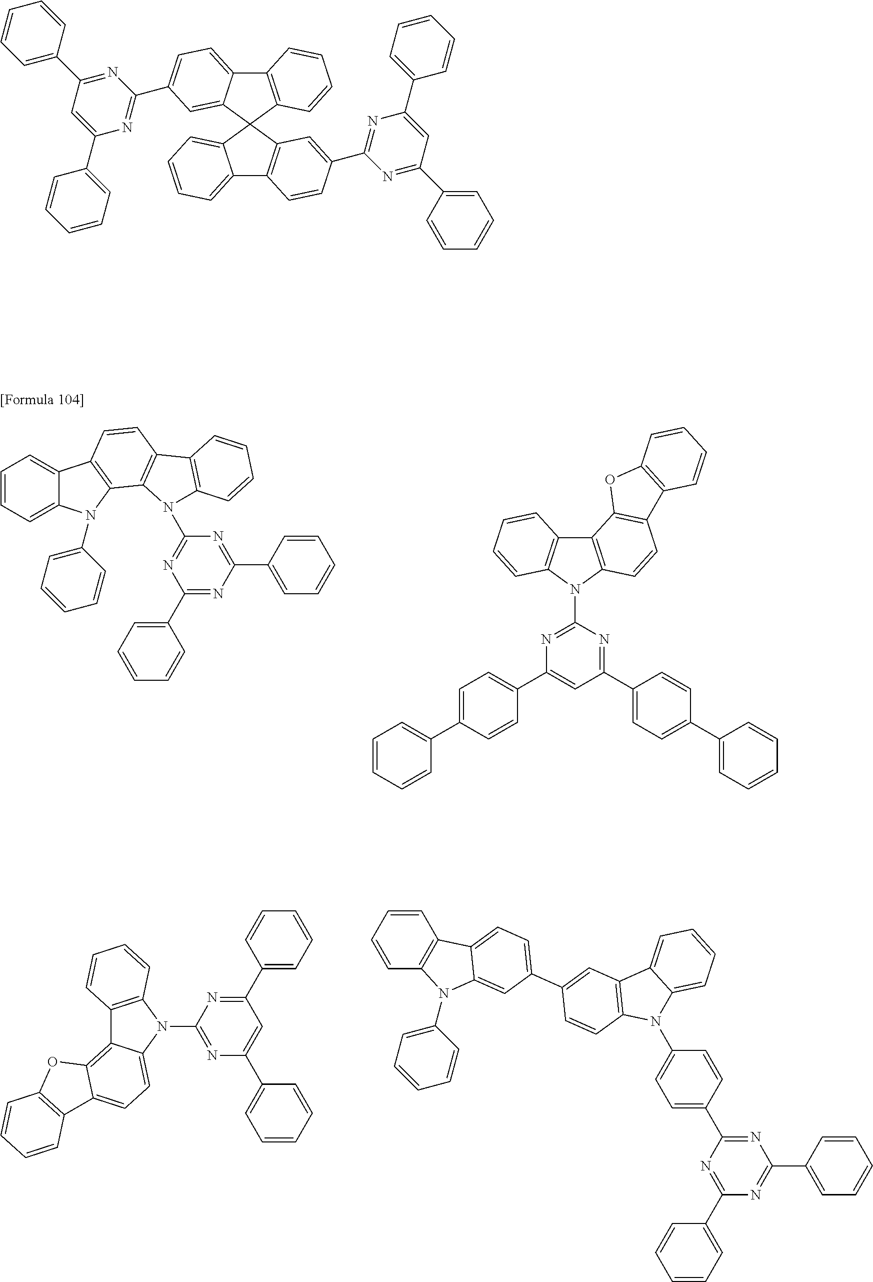 [SMILES: c1ccc(-c2cc(-c3ccccc3)nc(-c3ccc4c(c3)C3(c5ccccc5-4)c4ccccc4-c4ccc(-c5nc(-c6ccccc6)cc(-c6ccccc6)n5)cc43)n2)cc1 ; c1ccc(-c2nc(-c3ccccc3)nc(-n3c4ccccc4c4ccc5c6ccccc6n(-c6ccccc6)c5c43)n2)cc1 ; c1ccc(-c2ccc(-c3cc(-c4ccc(-c5ccccc5)cc4)nc(-n4c5ccccc5c5c6oc7ccccc7c6ccc54)n3)cc2)cc1 ; c1ccc(-c2cc(-c3ccccc3)nc(-n3c4ccccc4c4c5oc6ccccc6c5ccc43)n2)cc1 ; c1ccc(-c2nc(-c3ccccc3)nc(-c3ccc(-n4c5ccccc5c5cc(-c6ccc7c8ccccc8n(-c8ccccc8)c7c6)ccc54)cc3)n2)cc1]

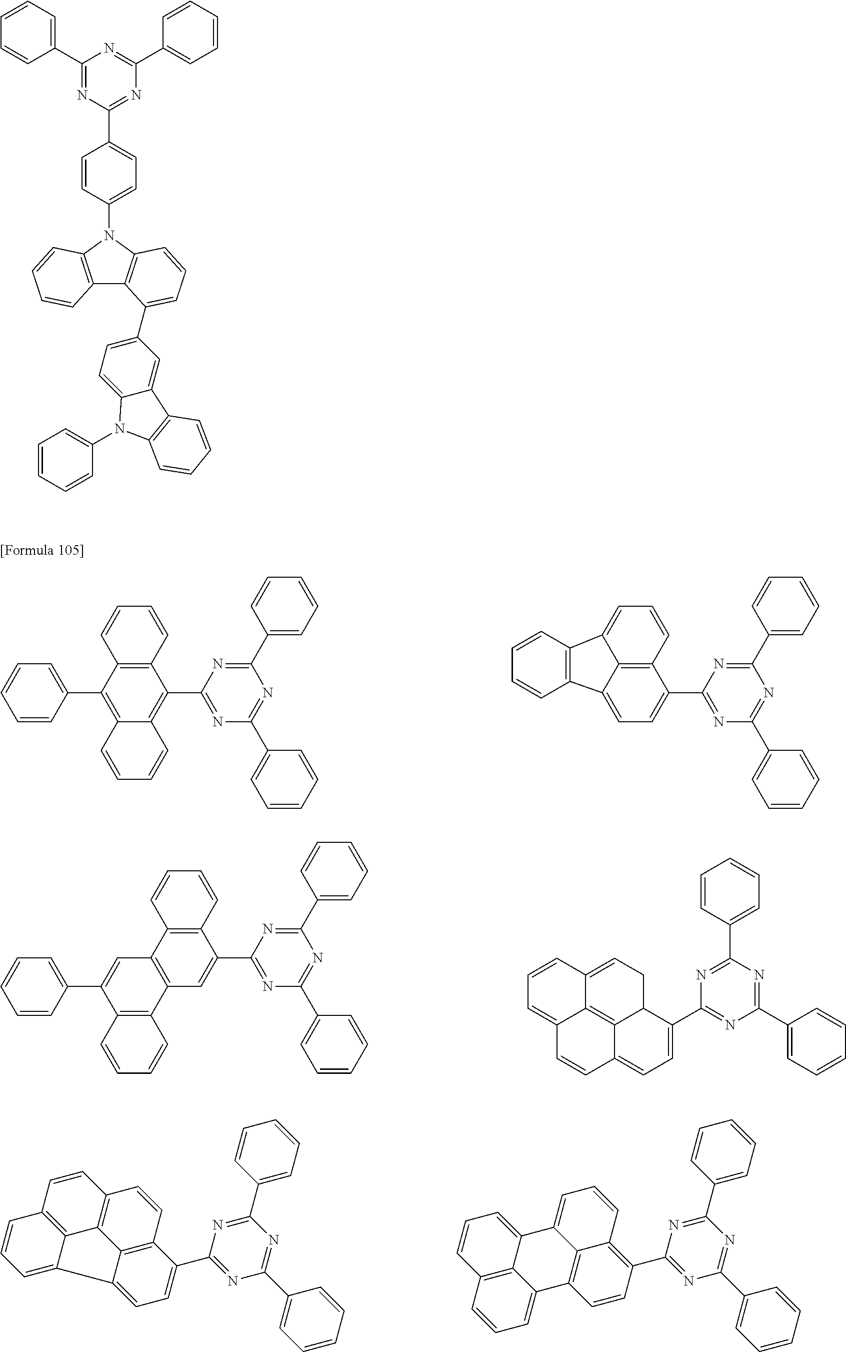

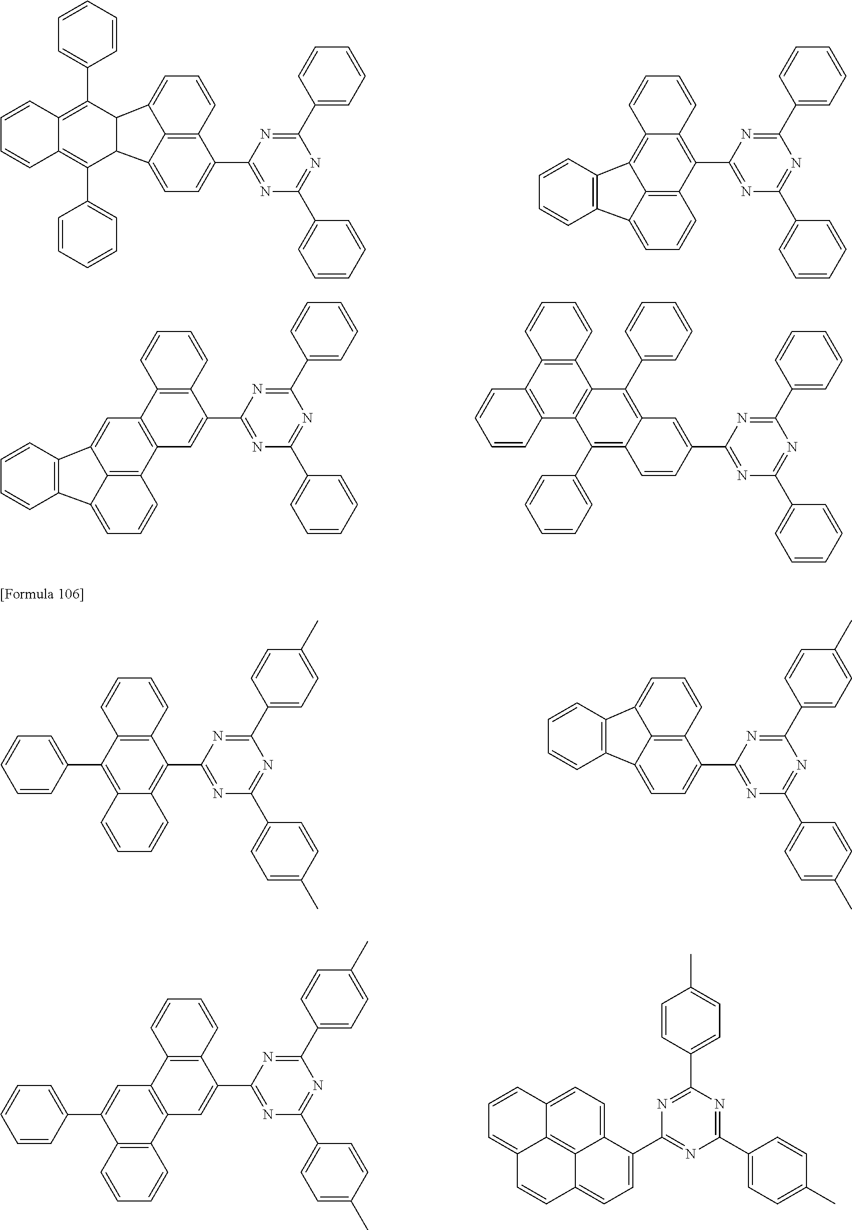

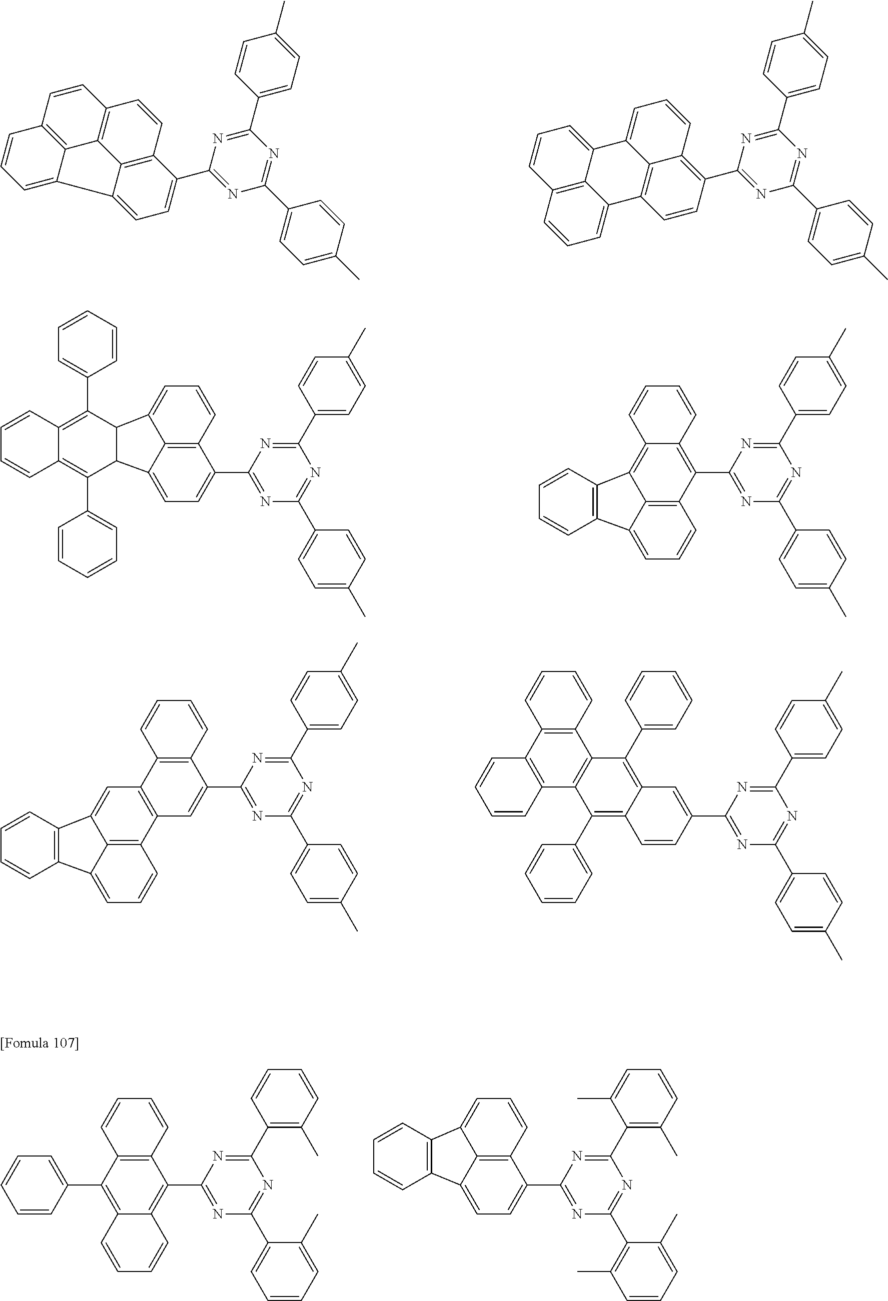

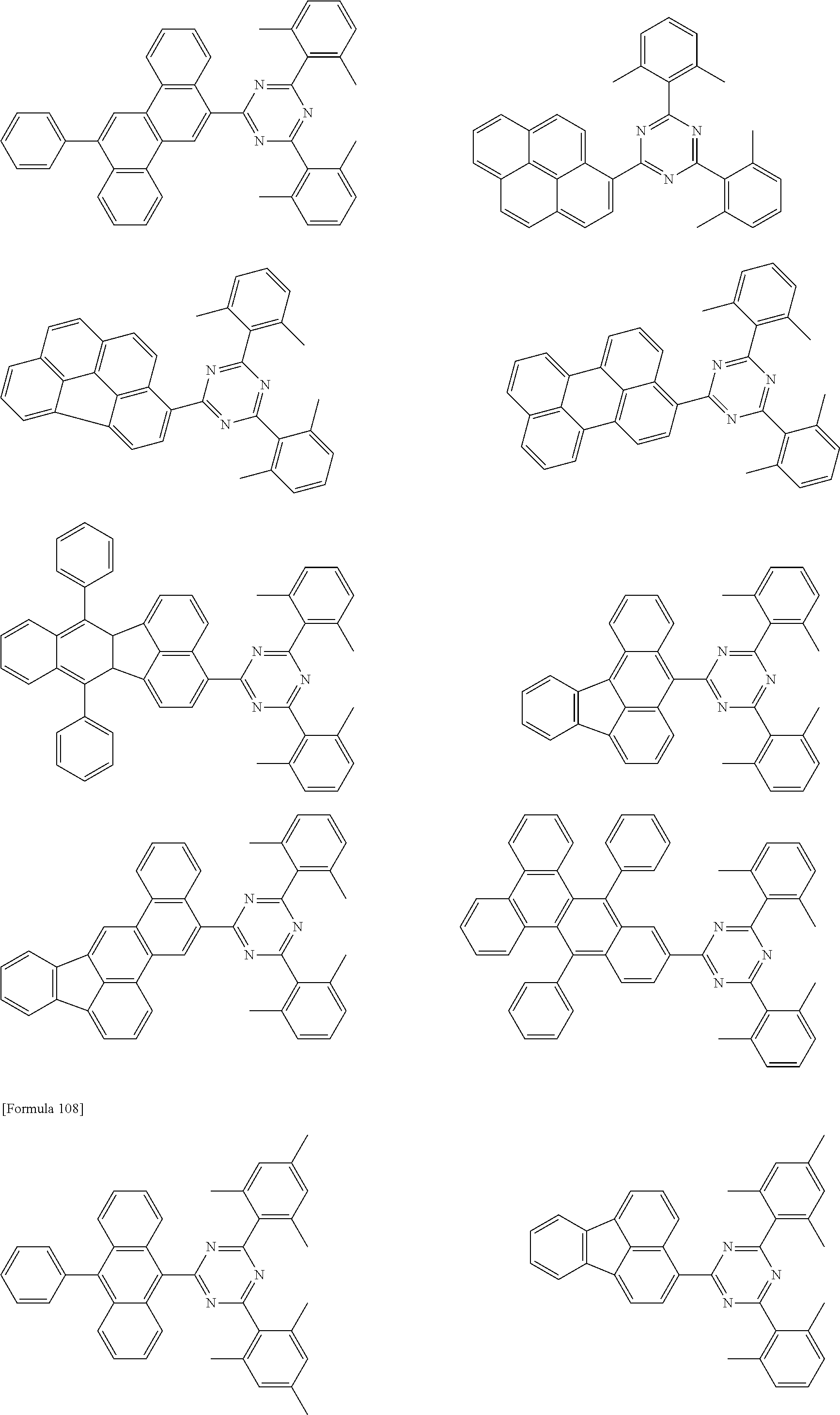

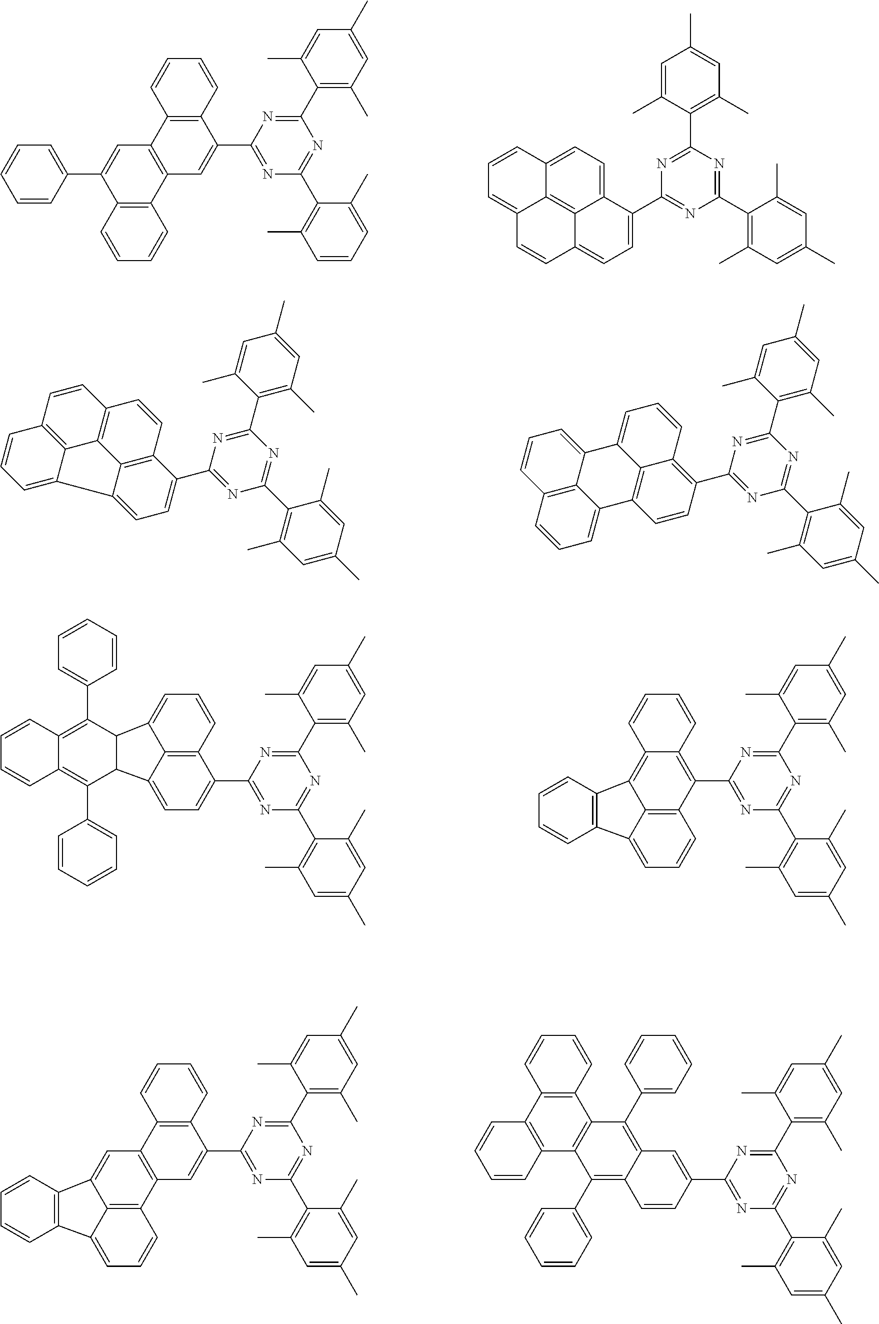

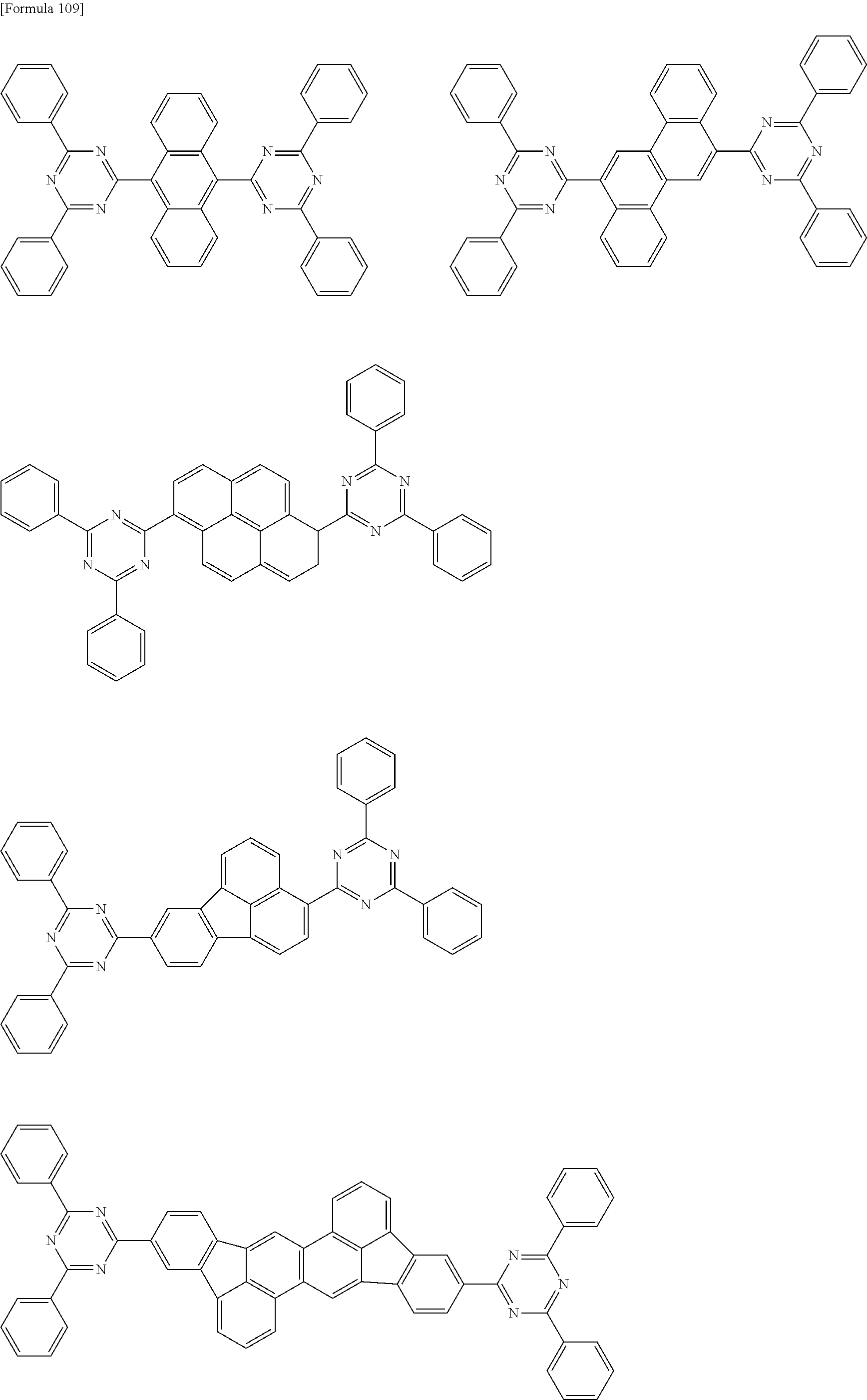 [SMILES: c1ccc(-c2nc(-c3ccccc3)nc(-c3c4ccccc4c(-c4nc(-c5ccccc5)nc(-c5ccccc5)n4)c4ccccc34)n2)cc1 ; c1ccc(-c2nc(-c3ccccc3)nc(-c3cc4c5ccccc5c(-c5nc(-c6ccccc6)nc(-c6ccccc6)n5)cc4c4ccccc34)n2)cc1 ; C1=c2ccc3c(-c4nc(-c5ccccc5)nc(-c5ccccc5)n4)ccc4ccc(c2c43)C(c2nc(-c3ccccc3)nc(-c3ccccc3)n2)C1 ; c1ccc(-c2nc(-c3ccccc3)nc(-c3ccc4c(c3)-c3cccc5c(-c6nc(-c7ccccc7)nc(-c7ccccc7)n6)ccc-4c35)n2)cc1 ; c1ccc(-c2nc(-c3ccccc3)nc(-c3ccc4c(c3)c3cccc5c6cc7c8ccc(-c9nc(-c%10ccccc%10)nc(-c%10ccccc%10)n9)cc8c8cccc(c6cc4c35)c87)n2)cc1]

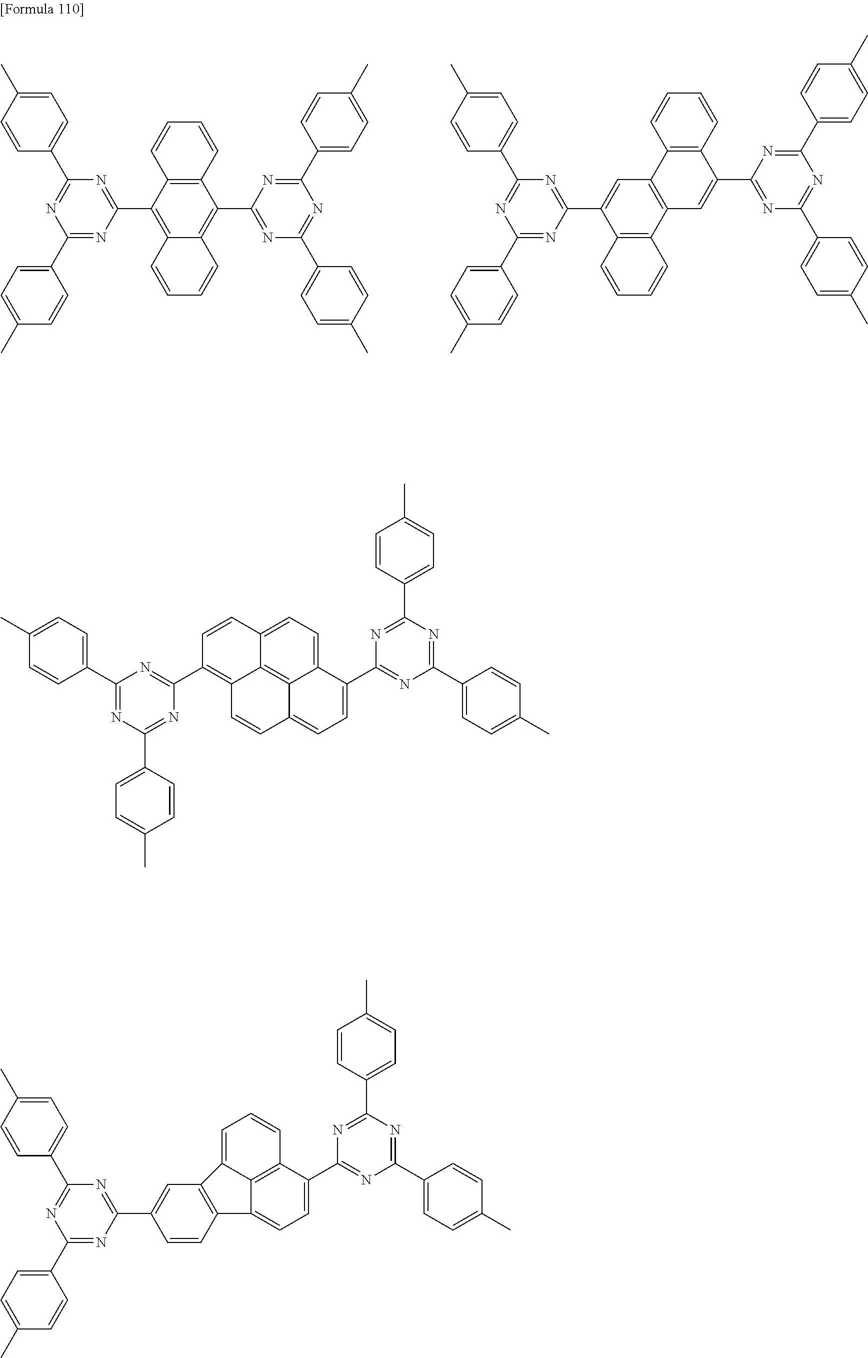

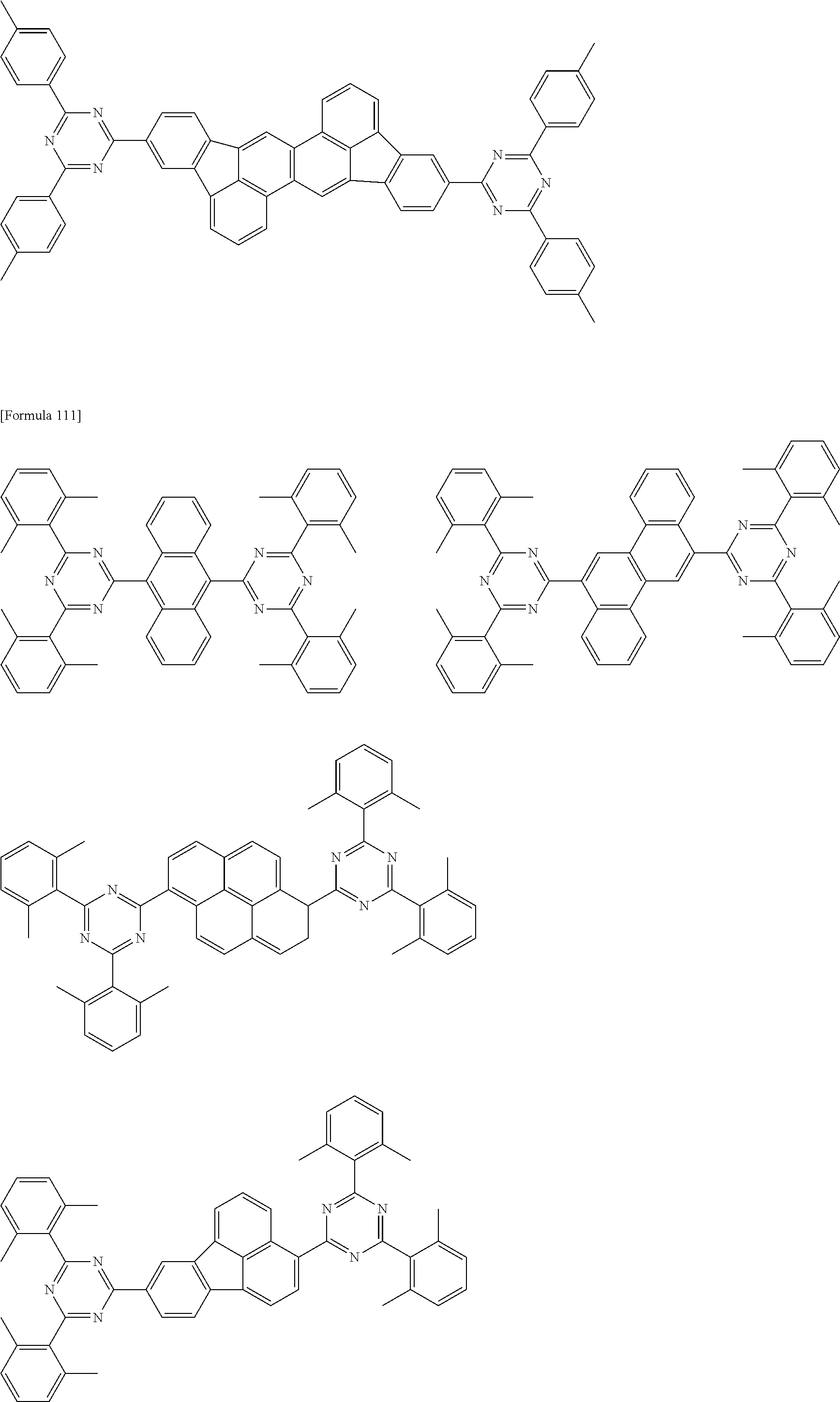

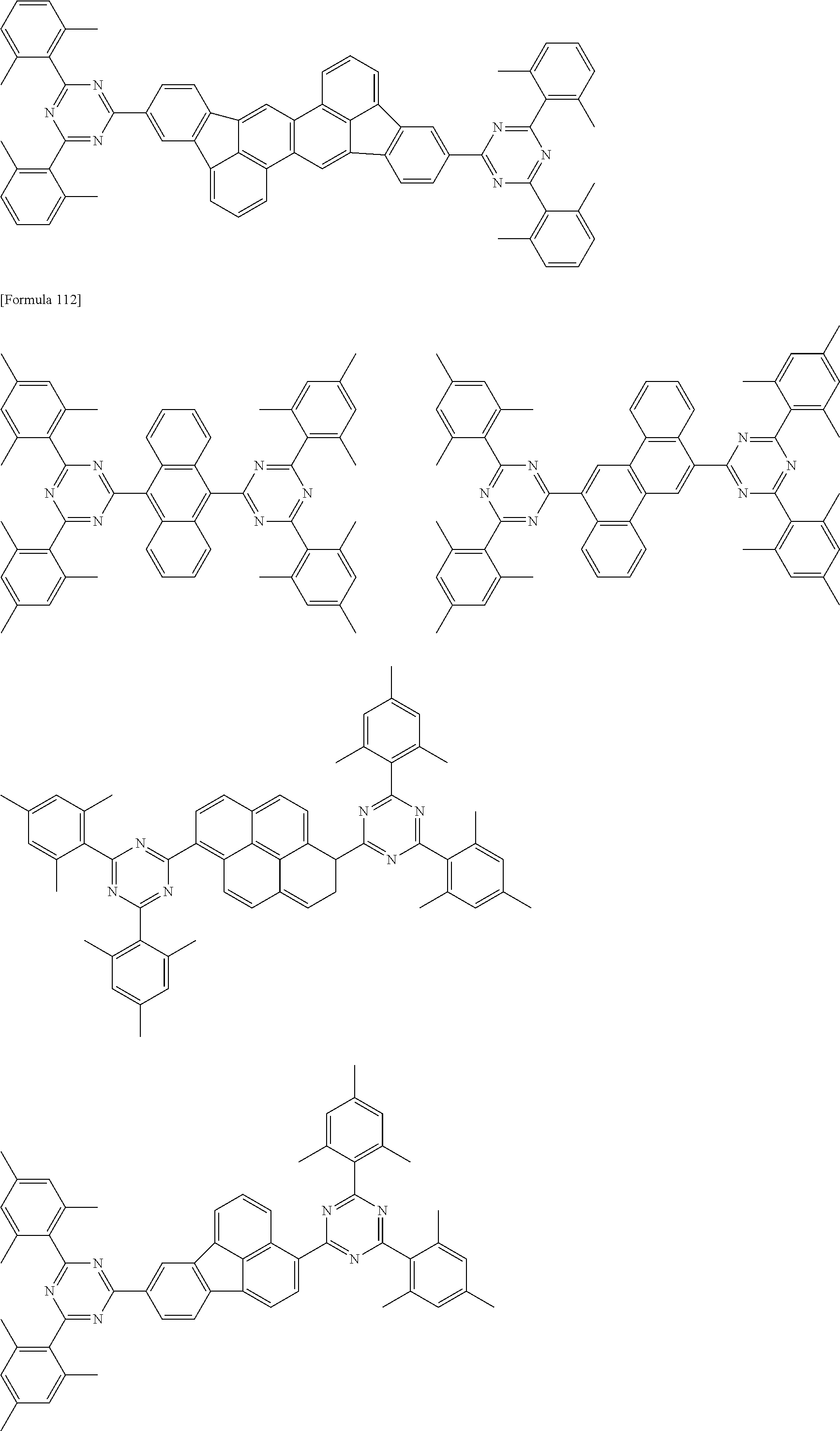

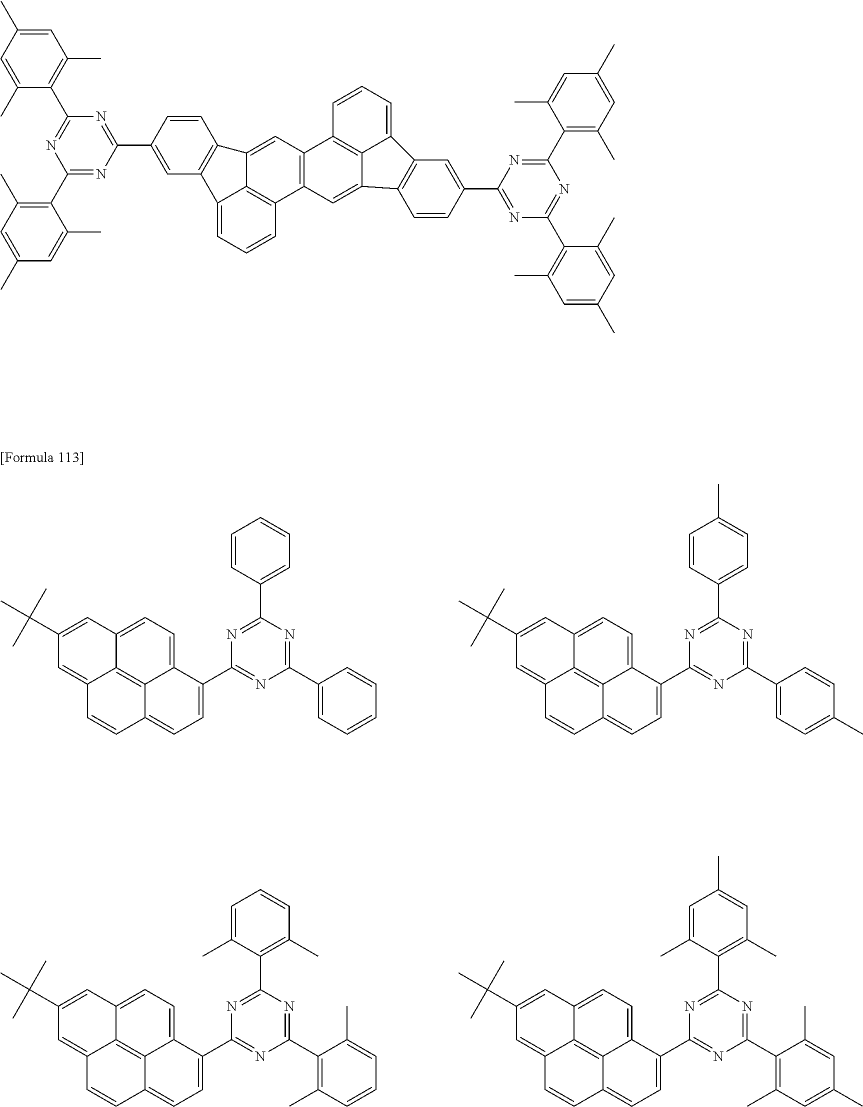

In the organic EL device 1 in the exemplary embodiment, the first electron transporting layer is preferably provided near the emitting layer, more preferably adjacent to the emitting layer. The first electron transporting layer prevents triplet excitons generated in the emitting layer from being diffused to an electron transporting zone and traps the triplet excitons within the emitting layer, thereby increasing a density of the triplet excitons. With this function, the first electron transporting layer has a function of efficiently causing TTF (Triplet-Triplet Fusion) phenomenon in which singlet excitons are generated by collision and fusion of the triplet excitons.

The first electron transporting layer also serves for efficiently injecting the electrons to the emitting layer. When the electron injecting performance to the emitting layer are deteriorated, the density of the triplet excitons is decreased since the electron-hole recombination in the emitting layer is decreased. When the density of the triplet excitons is decreased, the frequency of collision of the triplet excitons is reduced, whereby the TTF phenomenon does not occur efficiently.

Accordingly, an organic EL device with a higher efficient is obtainable with the first electron transporting layer containing the compound represented by the formula (1) adjacent to the emitting layer.

### Second Electron Transporting Layer

The second electron transporting layer of the organic EL device 1 of the first exemplary embodiment contains a compound represented by a formula (2) below.

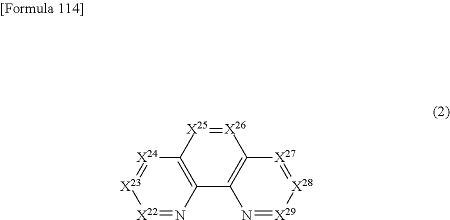

In the formula (2), X²² to X²⁹ each independently represent a nitrogen atom, CR²¹, or a carbon atom to be bonded to a group represented by a formula (21) below.

At least one of X²² to X²⁹ is a carbon atom to be bonded to a group represented by a formula (21) below. When a plurality of ones of X²² to X²⁹ are carbon atoms to be bonded to the group represented by the formula (21) below, a plurality of the groups represented by the formula (21) are the same or different.

R²¹ represents the same as R of the formula (1).

R²¹ of adjacent CR²¹ in X²² to X²⁹ are bonded to each other to form a cyclic structure or are not bonded.

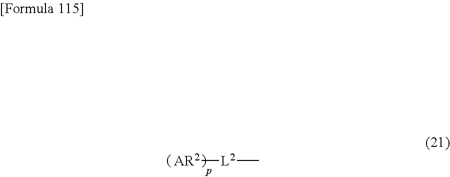

In the formula (21), p is an integer of 1 to 5.

Ar² is a substituted or unsubstituted aromatic hydrocarbon group having 6 to 40 ring carbon atoms or a substituted or unsubstituted heterocyclic group having 5 to 40 ring atoms. When p is from 2 to 5, a plurality of Ar² are mutually the same or different.

L² is a single bond or a linking group. The linking group in L² is a substituted or unsubstituted polyvalent linear, branched or cyclic aliphatic hydrocarbon group having 1 to 30 carbon atoms, a substituted or unsubstituted polyvalent aromatic hydrocarbon group having 6 to 40 ring carbon atoms, a substituted or unsubstituted polyvalent heterocyclic group having 5 to 40 ring atoms, or a polyvalent multiple linking group provided by bonding two or three groups selected from the aromatic hydrocarbon group and the heterocyclic group.

In the multiple linking group, the aromatic hydrocarbon group and the heterocyclic group forming the multiple linking group are mutually the same or different. The aromatic hydrocarbon group and the heterocyclic group which are adjacent to each other may be mutually bonded to form a ring.

The heterocyclic group having 5 to 40 ring atoms in Ar² of the formula (21) includes a substituted or unsubstituted group derived from the compound represented by the formula (2). However, the compound represented by the formula (2) contains 6 or less of Ar² that is a substituted or unsubstituted group derived from the compound represented by the formula (2).

Moreover, adjacent Ar² and L² are bonded to each other to form a cyclic structure or are not bonded.

L² bonded to any one of carbon atoms in X²² to X²⁹ of the formula (2) and a carbon atom or R²¹ of CR²¹ in one of X²² to X²⁹ adjacent to the carbon atom bonded to L² are further bonded to each other to form a ring, or are not bonded.

X²² or X²⁹ in the formula (2) is preferably a carbon atom to be bonded to the group represented by the formula (21).

Alternatively, X²² and X²⁹ in the formula (2) are preferably carbon atoms to be bonded to the group represented by the formula (21).

In the formula (21), Ar² is preferably a substituted or unsubstituted fused aromatic hydrocarbon group having 8 to 20 ring carbon atoms.

Examples of the fused aromatic hydrocarbon group having 8 to 20 ring carbon atoms include groups derived from naphthalene, anthracene, acephenanthrylene, aceanthrylene, benzanthracene, triphenylene, pyrene, chrysene, naphthacene, fluorene, phenanthrene, fluoranthene and benzofluoranthene.

In the formula (21), it is preferable that L² is a linking group and Ar² is a substituted or unsubstituted fused aromatic hydrocarbon group having 8 to 20 ring carbon atoms.

Examples of the fused aromatic hydrocarbon group having 8 to 20 ring carbon atoms include groups derived from naphthalene, anthracene, acephenanthrylene, aceanthrylene, benzanthracene, triphenylene, pyrene, chrysene, naphthacene, fluorene, phenanthrene, fluoranthene and benzofluoranthene.

In the formula (21), Ar² is also preferably a substituted or unsubstituted heterocyclic group having 5 to 40 ring atoms.

In the formula (21), Ar² is also preferably a substituted or unsubstituted group derived from the compound represented by the formula (2). At this time, the compound represented by the formula (2) preferably contains 6 or less of Ar² that is a substituted or unsubstituted group derived from the compound represented by the formula (2), more preferably contains 4 or less of Ar². Further preferably, the compound of the formula (2) contains 2 or less of Ar². Particularly preferably, the compound of the formula (2) contains a single Ar².

In the formula (21), Ar² is preferably represented by a formula (22) below.

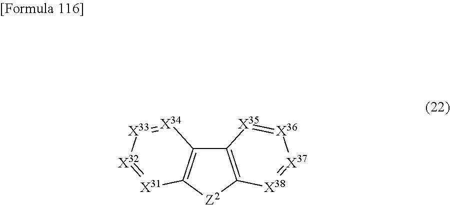

In the formula (22), X³¹ to X³⁸ each independently represent a nitrogen atom or CR²³. Z² is an oxygen atom, sulfur atom, NR²⁴, CR²⁵R²⁶, or SiR²⁷R²⁸.

R²³ to R²⁸ represent the same as R of the formula (1).

However, one of R²³ to R²⁸ is a single bond to be bonded with L² of the formula (21).

When the formula (22) includes a plurality of R²³, the plurality of R²³ are mutually the same or different.

R²³ in adjacent CR²³ may be bonded together to form a saturated or unsaturated ring. When Z² is one of NR²⁴, CR²⁵R²⁶ and SiR²⁷R²⁸ and at least one of X²¹ and X²⁸ is CR²³, one of R²⁴ to R²⁸ may be bonded to R²³ to form a ring, or alternatively, Z² may be directly bonded to R²³ to form a ring.

The rest of R²³ to R²⁸ except for a single bond to be bonded to L² may be further bonded to L² of the formula (21) to form a saturated or unsaturated ring.

The formula (21) is also preferably represented by a formula (23) below.

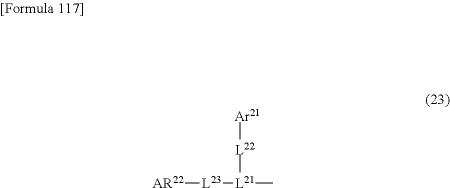

In the formula (23), L²¹ is a linking group. The linking group in L²¹ is a substituted or unsubstituted trivalent linear, branched or cyclic aliphatic hydrocarbon group having 1 to 30 carbon atoms, a substituted or unsubstituted trivalent aromatic hydrocarbon group having 6 to 40 ring carbon atoms, or a substituted or unsubstituted trivalent heterocyclic group having 5 to 40 ring atoms.

L²² and L²³ are each independently a single bond or a linking group. The linking group in L²² and L²³ is a substituted or unsubstituted divalent linear, branched or cyclic aliphatic hydrocarbon group having 1 to 30 carbon atoms, a substituted or unsubstituted divalent aromatic hydrocarbon group having 6 to 40 ring carbon atoms, or a substituted or unsubstituted divalent heterocyclic group having 5 to 40 ring atoms.

Ar²¹ and Ar²² represent the same as Ar² of the formula (2). The heterocyclic group having 5 to 30 ring atoms in Ar²¹ includes a substituted or unsubstituted group derived from the compound represented by the formula (2).

In the group represented by the formula (23), Ar²¹ is preferably a substituted or unsubstituted group derived from the compound represented by the formula (2) and is preferably represented by a formula (23-1) below.

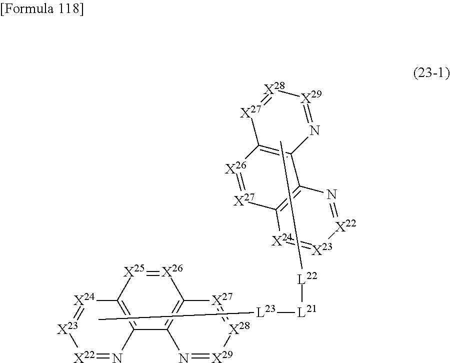

In the formula (23-1), X²² to X²⁹ represent the same as X²² to X²⁹ of the formula (2). L²¹, L²² and L²³ represent the same as L²¹, L²² and L²³ of the formula (23). A plurality of each of X²² to X²⁹ are mutually the same or different.

Note that L²² and L²³ are respectively bonded to any ones of X²² to X²⁹.

The rest of X²² to X²⁹ in the formula (2) except for a carbon atom to be bonded to the group represented by the formula (21) are preferably CR²¹. Specifically, the compound represented by the formula (2) is preferably a 1,10-phenanthroline derivative.

In the formula (23-1), L²¹ is preferably a substituted or unsubstituted trivalent aromatic hydrocarbon group having 6 to 40 ring carbon atoms, particularly preferably a trivalent benzene ring. L²² is preferably a single bond or a substituted or unsubstituted divalent aromatic hydrocarbon group having 6 to 40 ring carbon atoms, more preferably a single bond or a phenylene group.

The compound represented by the formula (2) is preferably a compound represented by a formula (2-1), more preferably a compound represented by a formula (2-2A) or (2-2B). Furthermore, the compound represented by the formula (2-2A) is more preferably represented by a formula (2-3A1) or (2-3A2). The compound represented by the formula (2-2B) is more preferably represented by a formula (2-3B).

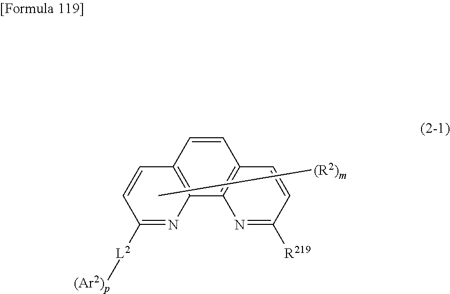

In the formula (2-1), R² and R²¹⁹ represent the same as R of the formula (1). m is an integer of 1 to 6. Ar², L² and p respectively represent the same as Ar², L² and p in the formula (21). Note that R² is bonded to any one(s) of carbon atoms at positions 3 to 8 of a phenanthroline ring.

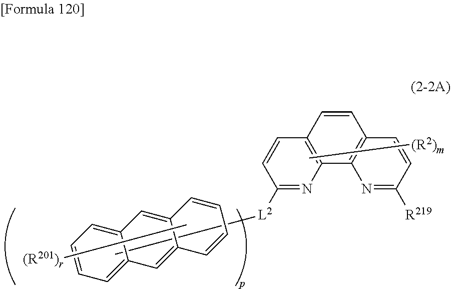

In the formula (2-2A), L² and p respectively represent the same as L² and p in the formula (21).

m is an integer of 1 to 6 and r is an integer of 1 to 9.

R², R²⁰¹ and R²¹⁹ represent the same as R²¹ of the formula (2). A plurality of each of R² and R²⁰¹ are mutually the same or different.

Note that R² is bonded to any one(s) of carbon atoms at positions 3 to 8 of the phenanthroline ring. L² and R²⁰¹ are bonded to any ones of carbon atoms at positions 1 to 10 of the anthracene ring.

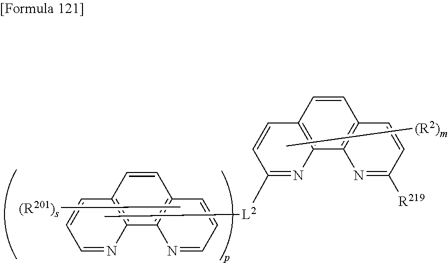

In the formula (2-2B), L² and p respectively represent the same as L² and p in the formula (21).

m is an integer of 1 to 6 and s is an integer of 1 to 7.

R², R²⁰¹ and R²¹⁹ represent the same as R²¹ of the formula (2). A plurality of each of R² and R²⁰¹ are mutually the same or different.

Note that R² is bonded to any one(s) of carbon atoms at positions 3 to 8 of the phenanthroline ring. L² and R²⁰¹ are bonded to any ones of carbon atoms at positions 3 to 8 of the phenanthroline ring.

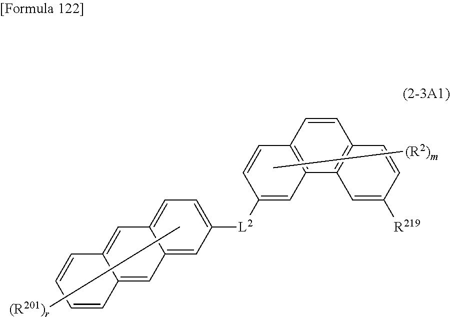

In the formula (2-3A1), L² represents the same as L² of the formula (21).

m is an integer of 1 to 6 and r is an integer of 1 to 9.

R², R²⁰¹ and R²¹⁹ represent the same as R²¹ of the formula (2). A plurality of each of R² and R²⁰¹ are mutually the same or different.

Note that R² is bonded to any one(s) of carbon atoms at positions 3 to 8 of the phenanthroline ring. R²⁰¹ is bonded to any one(s) of carbon atoms at positions 1, 3 and 10 of the anthracene ring.

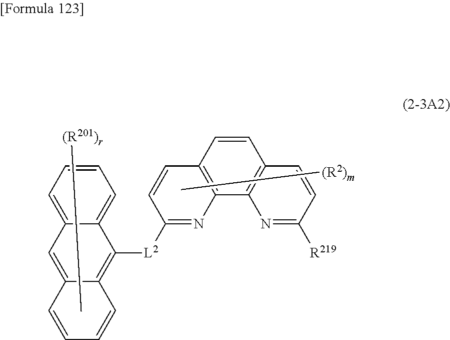

In the formula (2-3A2), L² represents the same as L² of the formula (21).

m is an integer of 1 to 6 and r is an integer of 1 to 9.

R², R²⁰¹ and R²¹⁹ represent the same as R²¹ of the formula (2). A plurality of each of R² and R²⁰¹ are mutually the same or different.

Note that R² is bonded to any one(s) of carbon atoms at positions 3 to 8 of the phenanthroline ring. R²⁰¹ is bonded to any one(s) of carbon atoms at positions 1 to 8 and 10 of the anthracene ring.

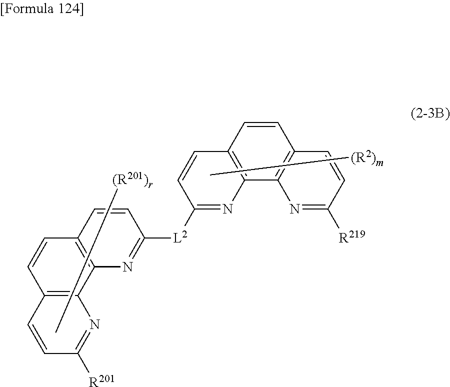

In the formula (2-3B), L², R², R²⁰¹, R²¹⁹ and m respectively represent the same as L², R², R²⁰¹, R²¹⁹ and m of the formula (2-2B).

Note that R² is bonded to any one(s) of carbon atoms at positions 3 to 8 of the phenanthroline ring.

The compound represented by the formula (2-1) is also preferably a compound represented by one of formulae (2-2C) to (2-2G) below.

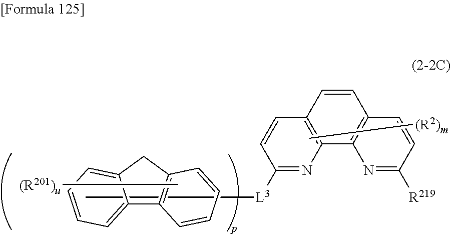

In the formula (2-2C), L³ is not a single bond but a linking group. The linking group in L³ is a substituted or unsubstituted polyvalent linear, branched or cyclic aliphatic hydrocarbon group having 1 to 30 carbon atoms, a substituted or unsubstituted polyvalent amino group, a substituted or unsubstituted polyvalent aromatic hydrocarbon group having 6 to 40 ring carbon atoms, a substituted or unsubstituted polyvalent heterocyclic group having 5 to 40 ring atoms, or a polyvalent multiple linking group provided by bonding two or three groups selected from the aromatic hydrocarbon group and the heterocyclic group.

In the multiple linking group, the aromatic hydrocarbon group and the heterocyclic group forming the multiple linking group are mutually the same or different. The aromatic hydrocarbon group and the heterocyclic group which are adjacent to each other may be mutually bonded to form a ring.

p represents the same as p of the formula (21).

m is an integer of 1 to 6 and u is an integer of 1 to 9.

R², R²⁰¹ and R²¹⁹ represent the same as R²¹ of the formula (2). A plurality of each of R² and R²⁰¹ are mutually the same or different.

Note that R² is bonded to any one(s) of carbon atoms at positions 3 to 8 of the phenanthroline ring. R²⁰¹ and L³ are bonded to any ones of carbon atoms at positions 1 to 9 of the fluorene ring. When u is 2 or more, two R²⁰¹ may be bonded to the position 9 of the fluorene ring.

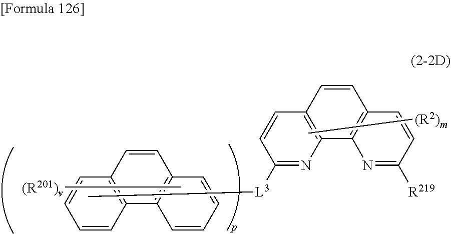

In the formula (2-2D), L³ represents the same as L³ of the formula (2-2C). p represents the same as p of the formula (21).

m is an integer of 1 to 6 and v is an integer of 1 to 9.

R², R²⁰¹ and R²¹⁹ represent the same as R²¹ of the formula (2). A plurality of each of R² and R²⁰¹ are mutually the same or different.

Note that R² is bonded to any one(s) of carbon atoms at positions 3 to 8 of the phenanthroline ring. R²⁰¹ and L² are bonded to any ones of carbon atoms at positions 1 to 10 of the phenanthrene ring.

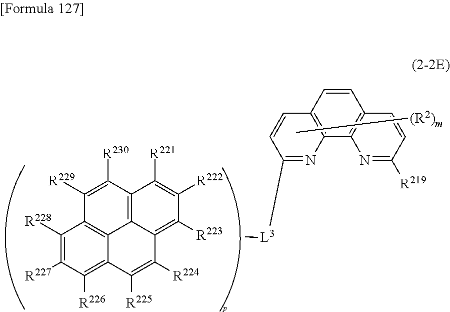

In the formula (2-2E), L³ represents the same as L³ of the formula (2-2C). p represents the same as p of the formula (21). m is an integer of 1 to 6.

R², R²¹⁹ and R²²¹ to R²³⁰ represent the same as R²¹ of the formula (2). However, one of R²²¹ to R²³⁰ is a single bond to be bonded to L³. A plurality of R² are mutually the same or different.

Note that R² is bonded to any one(s) of carbon atoms at positions 3 to 8 of the phenanthroline ring.

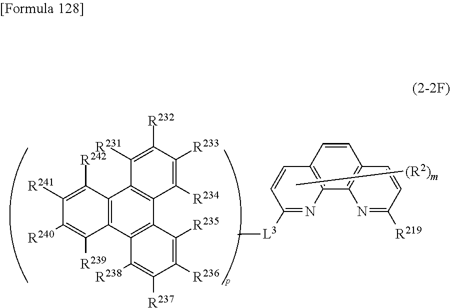

In the formula (2-2F), L³ represents the same as L³ of the formula (2-2C). p represents the same as p of the formula (21).

m is an integer of 1 to 6.

R², R²¹⁹ and R²³¹ to R²⁴² represent the same as R²¹ of the formula (2). However, one of R²³¹ to R²⁴² is a single bond to be bonded to L³. A plurality of R² are mutually the same or different.

Note that R² is bonded to any one(s) of carbon atoms at positions 3 to 8 of the phenanthroline ring.

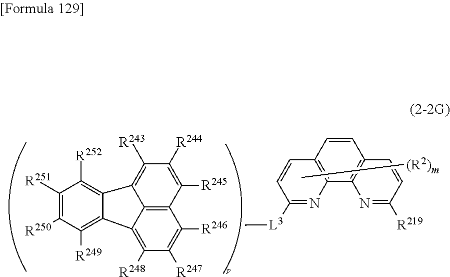

In the formula (2-2G), L³ represents the same as L³ of the formula (2-2C). p represents the same as p of the formula (21).

m is an integer of 1 to 6.

R², R²¹⁹ and R²⁴³ to R²⁵² represent the same as R²¹ of the formula (2). However, one of R²³¹ to R²⁴² is a single bond to be bonded to L². A plurality of R² are mutually the same or different.

Note that R² is bonded to any one(s) of carbon atoms at positions 3 to 8 of the phenanthroline ring.

The compound represented by the formula (2) is preferably a compound represented by a formula (3-1), more preferably a compound represented by a formula (3-2A) or (3-2B). Furthermore, the compound represented by the formula (3-2A) is more preferably represented by a formula (3-3A1) or (3-3A2). The compound represented by the formula (3-2B) is more preferably represented by a formula (3-3B).

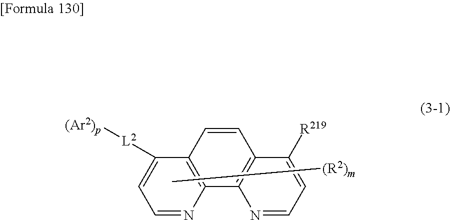

In the formula (3-1), R² and R²¹⁹ represent the same as R of the formula (1). m is an integer of 1 to 6. Ar², L² and p respectively represent the same as Ar², L² and p in the formula (21). Note that R² is bonded to any one(s) of carbon atoms at positions 3 to 8 of the phenanthroline ring.

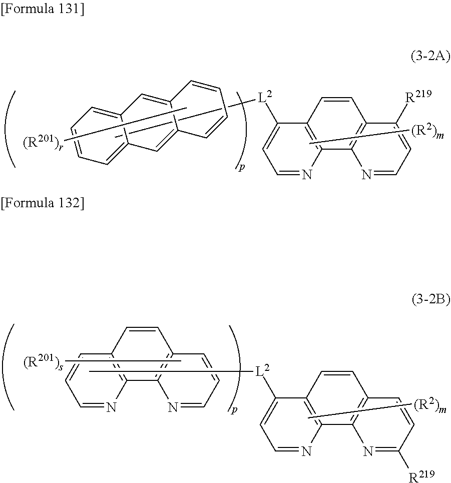

In the formulae (3-2A) and (3-2B), L², R², R²¹⁹, m and p respectively represent the same as L², R², R²¹⁹, m and p of the formula (3-1).

R²⁰¹ represents the same as R². r is an integer of 1 to 9. s is an integer of 1 to 7.

Note that R² is bonded to any one(s) of carbon atoms at positions 3 to 8 of the phenanthroline ring. L² and R²⁰¹ are bonded to any ones of carbon atoms at positions 3 to 8 of the phenanthroline ring.

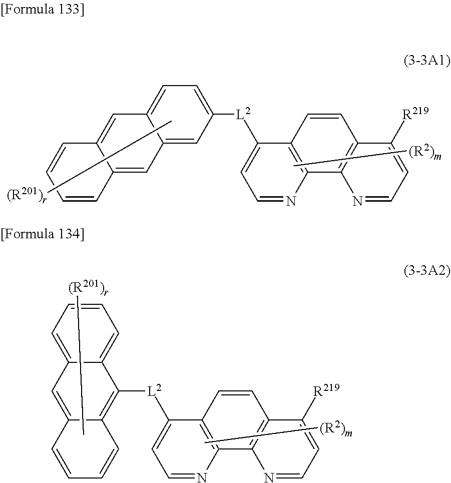

In the formulae (3-3A1) and (3-3A2), L², R², R²⁰¹, R²¹⁹, m and r respectively represent the same as L², R², R²⁰¹, R²¹⁹, m and r of the formula (3-2A).

Note that R² is bonded to any one(s) of carbon atoms at positions 3 to 8. R²⁰¹ is bonded to any one(s) of carbon atoms at positions 1 to 8 and 10 of the anthracene ring.

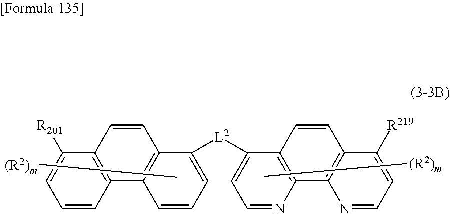

In the formula (3-3B), L², R², R²⁰¹, R²¹⁹ and m respectively represent the same as L², R², R²⁰¹, R²¹⁹ and m of the formula (3-2B).

Note that R² is bonded to any one(s) of carbon atoms at positions 3 to 8.

Specific examples of the substituents described in the formulae (2), (21), (22), (23), (23-1), (2-1), (2-2A) to (2-2G), (2-3A1), (2-3A2), (2-3B), (3-1), (3-2A), (3-2B), (3-3A1), (3-3A2) and (3-3B) are the same as the examples of the substituents described in the formulae (1), (1A) to (1D), (1-1) to (1-4), (1-21), (11), (12), (12A), (12B), (12A-1) to (12A-3), (12B-1) to (12B-6), (14) and (100).

In the formulae (2), (21), (22), (23), (23-1), (2-1), (2-2A) to (2-2G), (2-3A1), (2-3A2), (2-3B), (3-1), (3-2A), (3-2B), (3-3A1), (3-3A2) and (3-3B), “carbon atoms for forming a ring (ring carbon atoms)” and “substituted or unsubstituted” mean the same as the description in the formulae (1), (1A) to (1D), (1-1) to (1-4), (1-21), (11), (12), (12A), (12B), (12A-1) to (12A-3), (12B-1) to (12B-6), (14) and (100).

Specific examples of the compound represented by the formula (2) are shown below, but the invention is not limited to thereto.

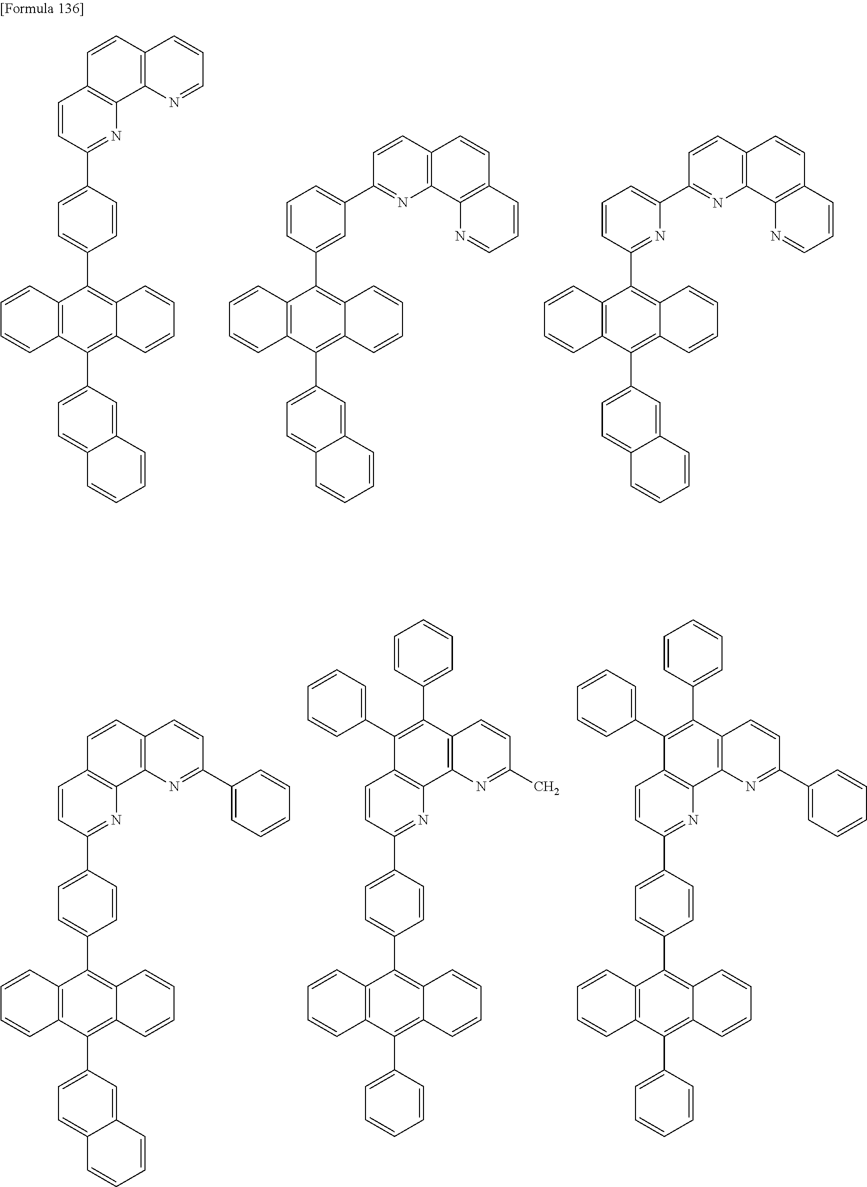 [SMILES: c1ccc2cc(-c3c4ccccc4c(-c4ccc(-c5ccc6ccc7cccnc7c6n5)cc4)c4ccccc34)ccc2c1 ; c1cc(-c2ccc3ccc4cccnc4c3n2)cc(-c2c3ccccc3c(-c3ccc4ccccc4c3)c3ccccc23)c1 ; c1cc(-c2ccc3ccc4cccnc4c3n2)nc(-c2c3ccccc3c(-c3ccc4ccccc4c3)c3ccccc23)c1 ; c1ccc(-c2ccc3ccc4ccc(-c5ccc(-c6c7ccccc7c(-c7ccc8ccccc8c7)c7ccccc67)cc5)nc4c3n2)cc1 ; Cc1ccc2c(-c3ccccc3)c(-c3ccccc3)c3ccc(-c4ccc(-c5c6ccccc6c(-c6ccccc6)c6ccccc56)cc4)nc3c2n1 ; c1ccc(-c2ccc3c(-c4ccccc4)c(-c4ccccc4)c4ccc(-c5ccc(-c6c7ccccc7c(-c7ccccc7)c7ccccc67)cc5)nc4c3n2)cc1]

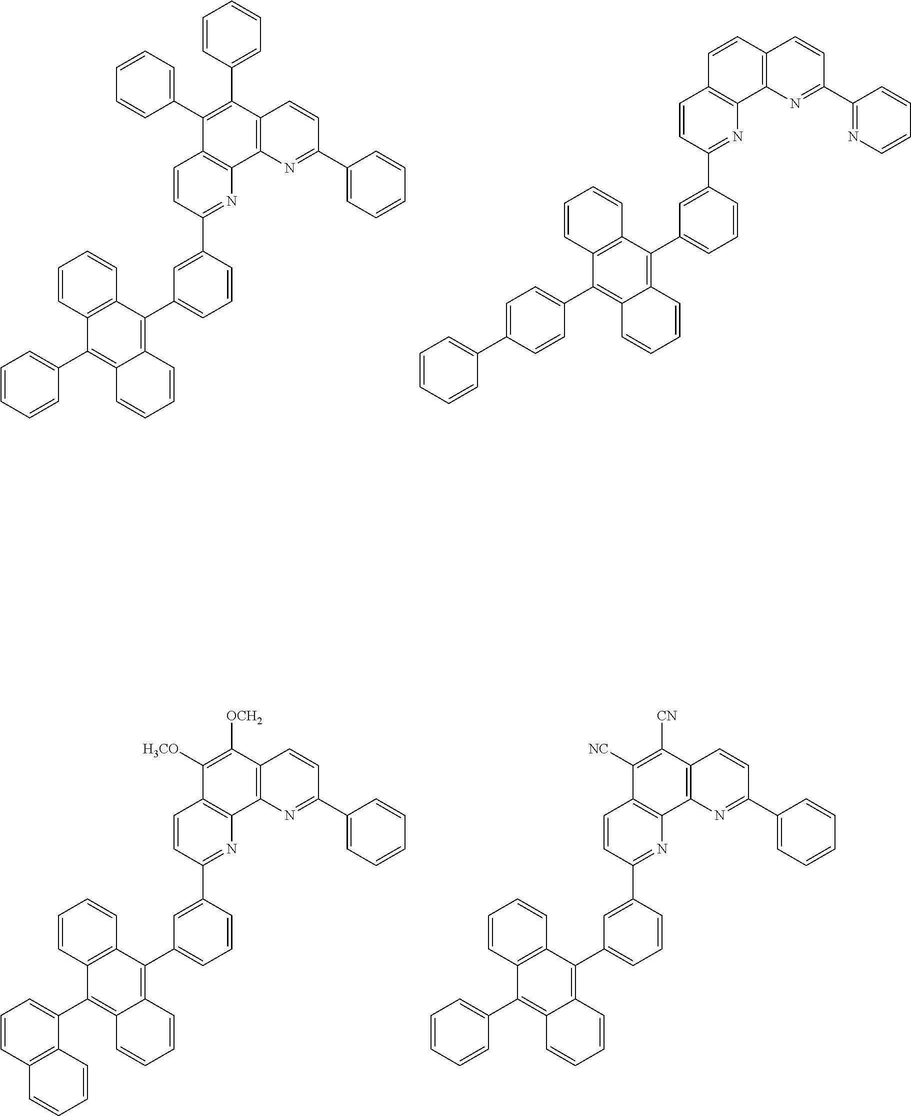

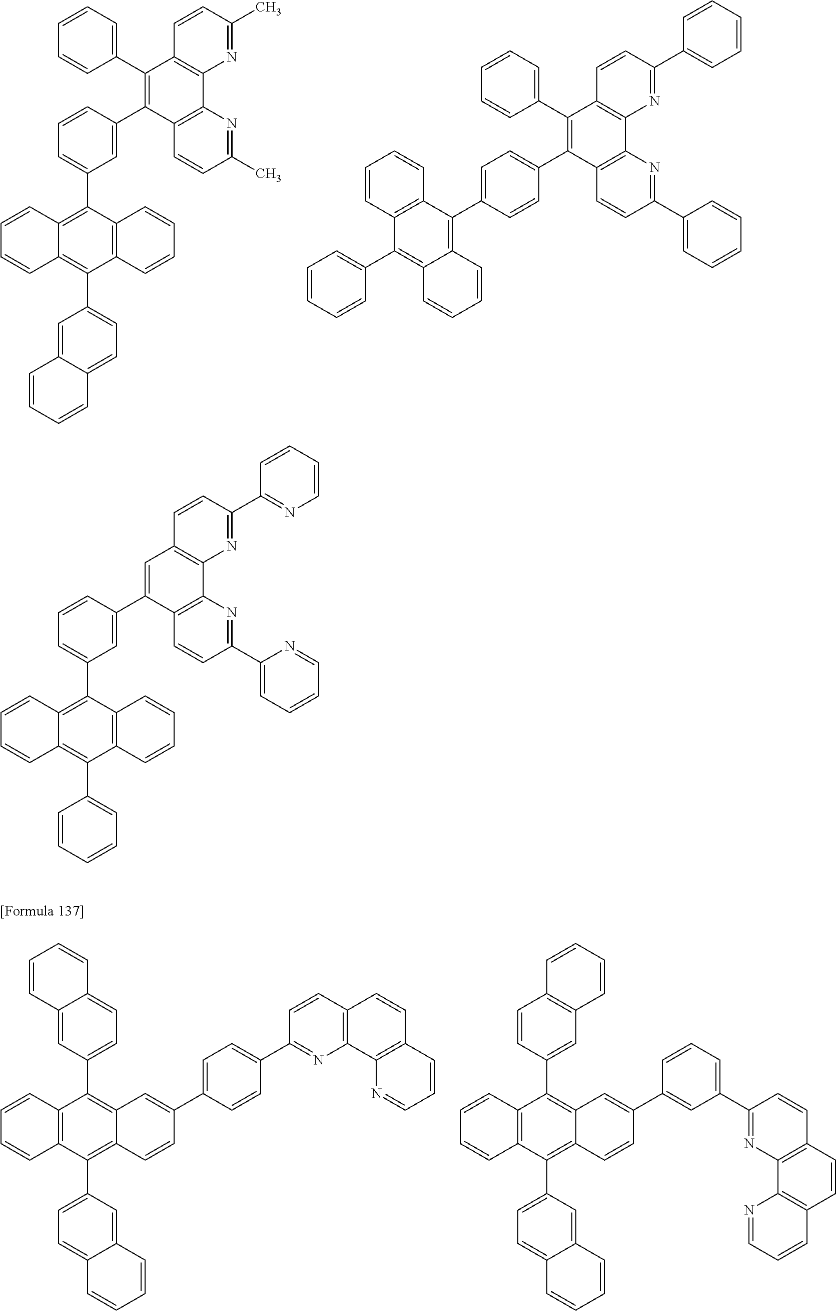

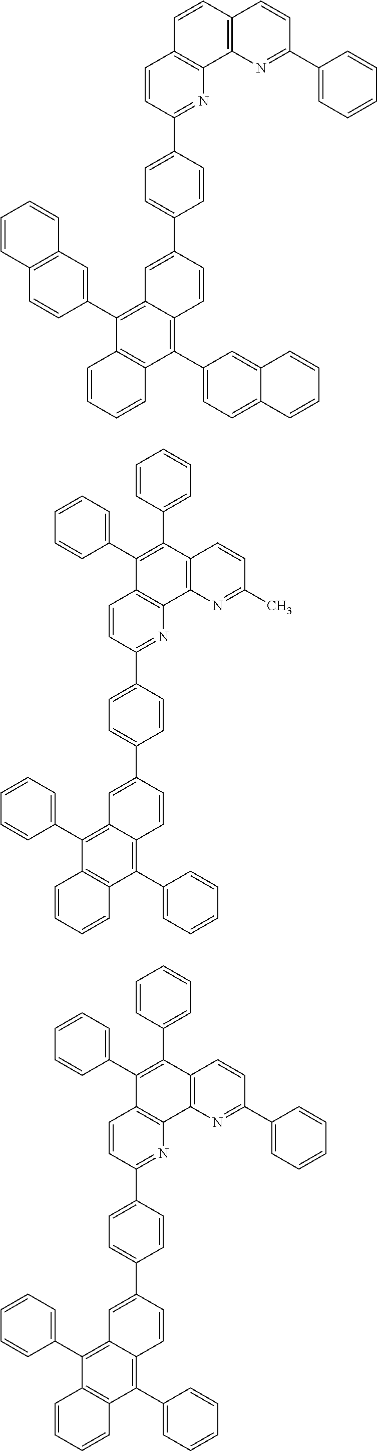

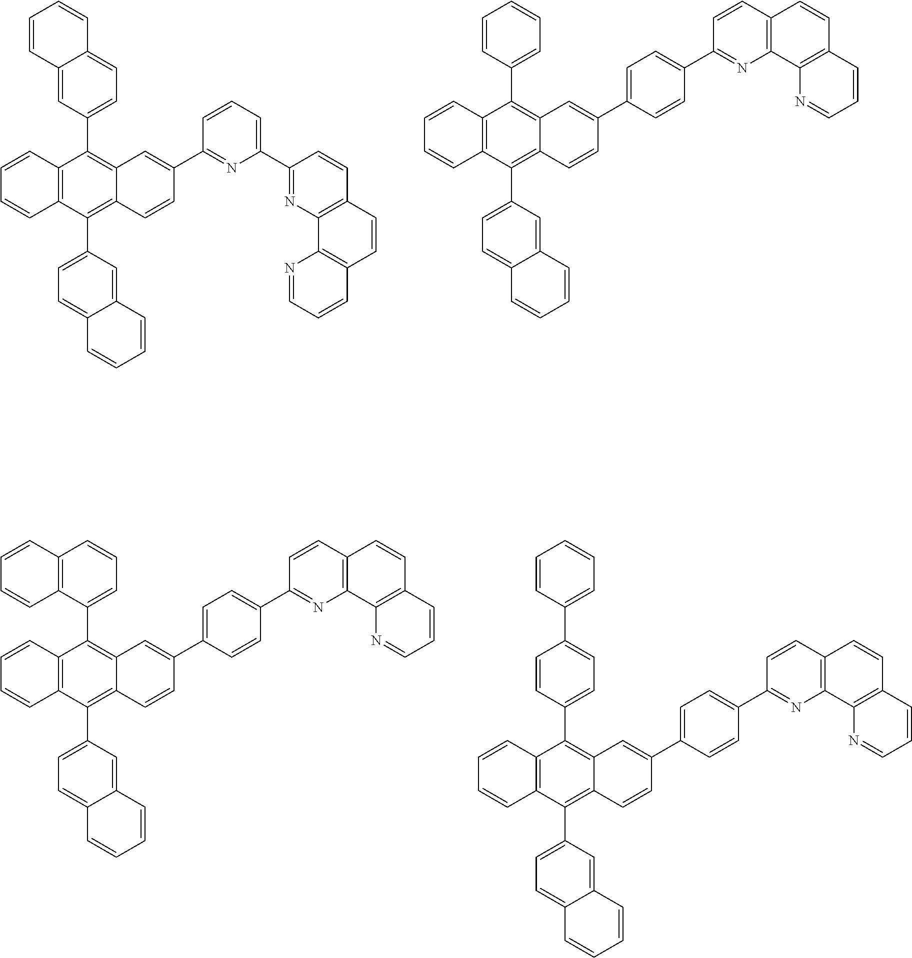 [SMILES: c1cc(-c2ccc3c(-c4ccc5ccccc5c4)c4ccccc4c(-c4ccc5ccccc5c4)c3c2)nc(-c2ccc3ccc4cccnc4c3n2)c1 ; c1ccc(-c2c3ccccc3c(-c3ccc4ccccc4c3)c3ccc(-c4ccc(-c5ccc6ccc7cccnc7c6n5)cc4)cc23)cc1 ; c1ccc2cc(-c3c4ccccc4c(-c4cccc5ccccc45)c4cc(-c5ccc(-c6ccc7ccc8cccnc8c7n6)cc5)ccc34)ccc2c1 ; c1ccc(-c2ccc(-c3c4ccccc4c(-c4ccc5ccccc5c4)c4ccc(-c5ccc(-c6ccc7ccc8cccnc8c7n6)cc5)cc34)cc2)cc1]

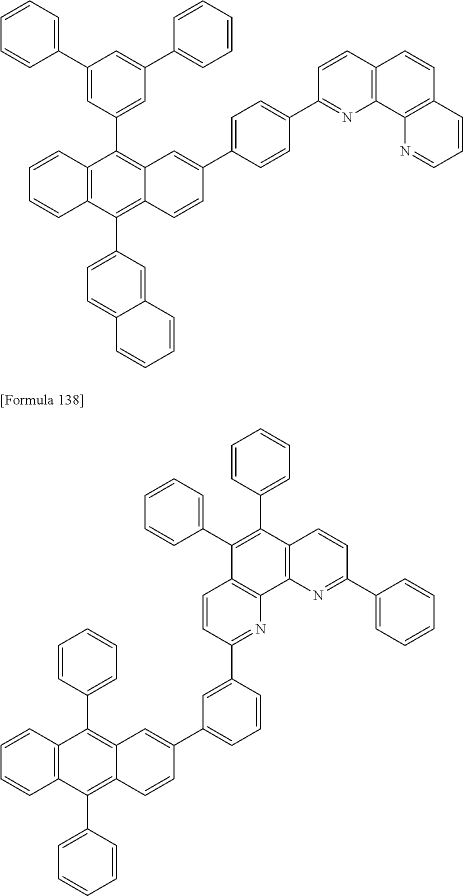 [SMILES: c1ccc(-c2cc(-c3ccccc3)cc(-c3c4ccccc4c(-c4ccc5ccccc5c4)c4ccc(-c5ccc(-c6ccc7ccc8cccnc8c7n6)cc5)cc34)c2)cc1 ; c1ccc(-c2ccc3c(-c4ccccc4)c(-c4ccccc4)c4ccc(-c5cccc(-c6ccc7c(-c8ccccc8)c8ccccc8c(-c8ccccc8)c7c6)c5)nc4c3n2)cc1]

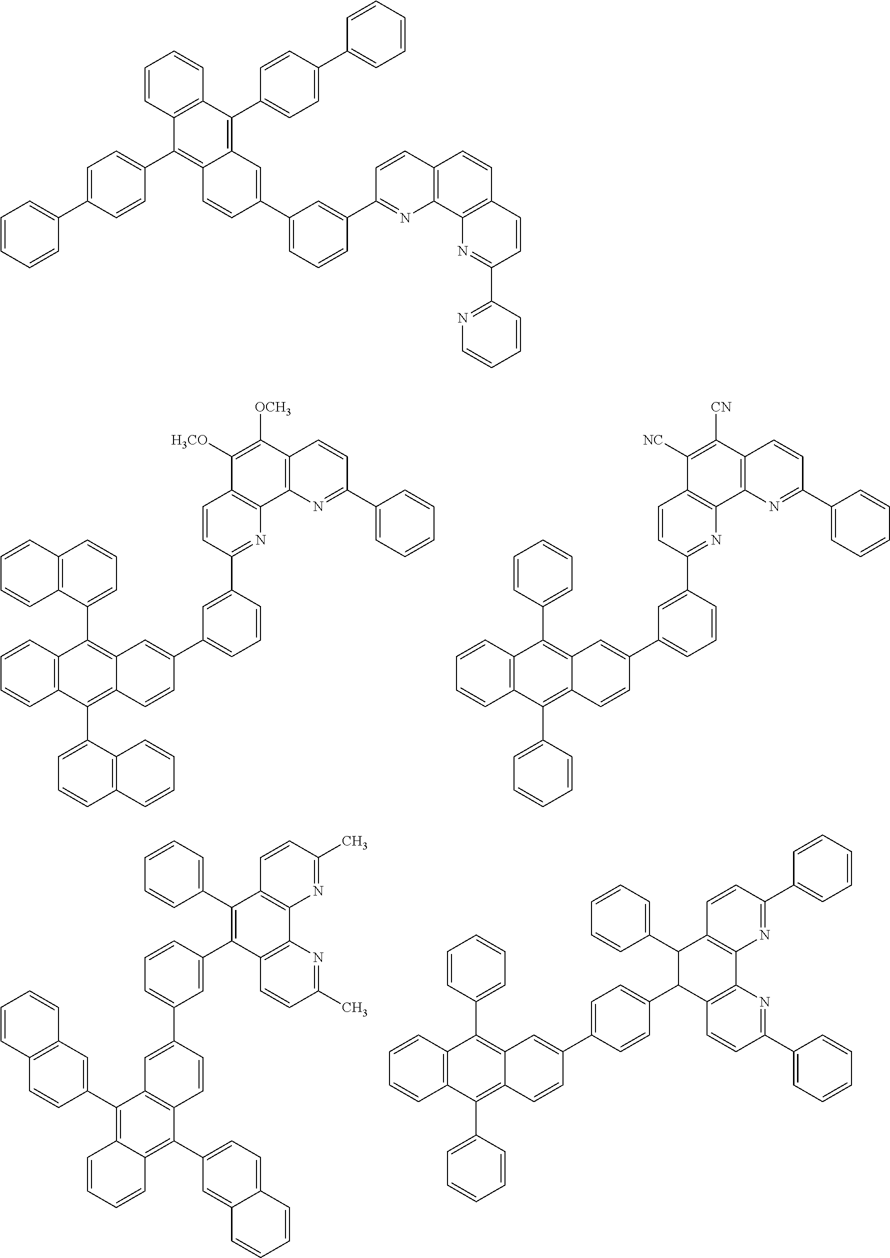

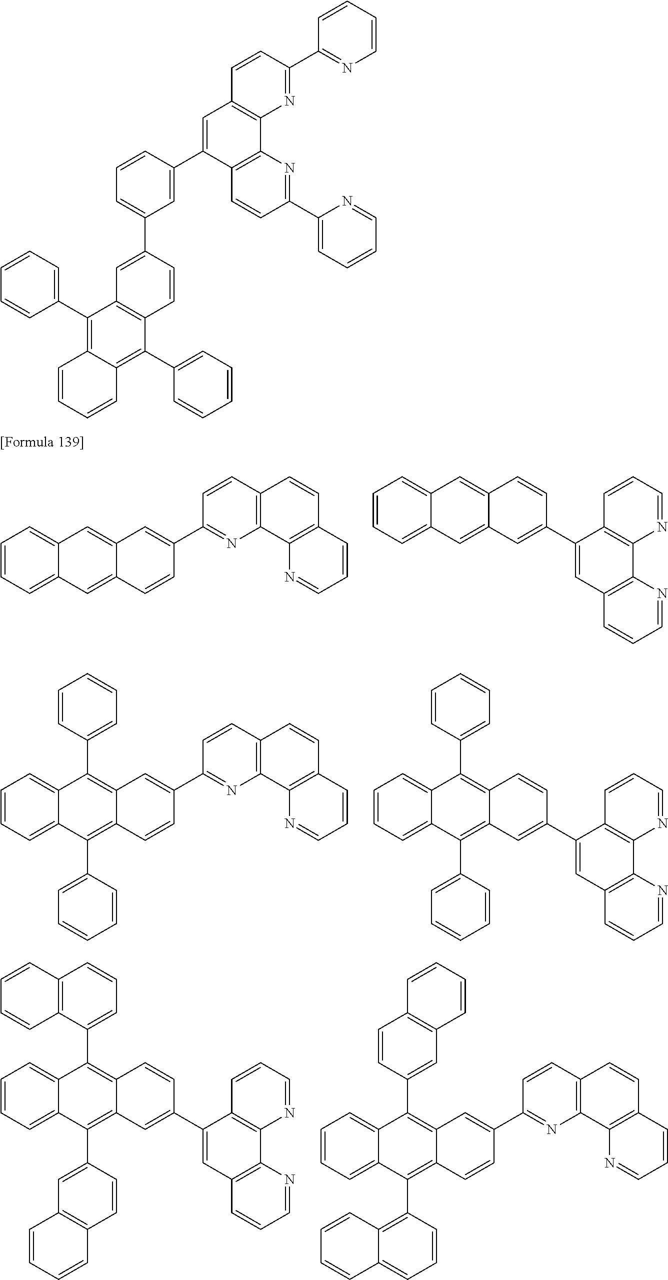

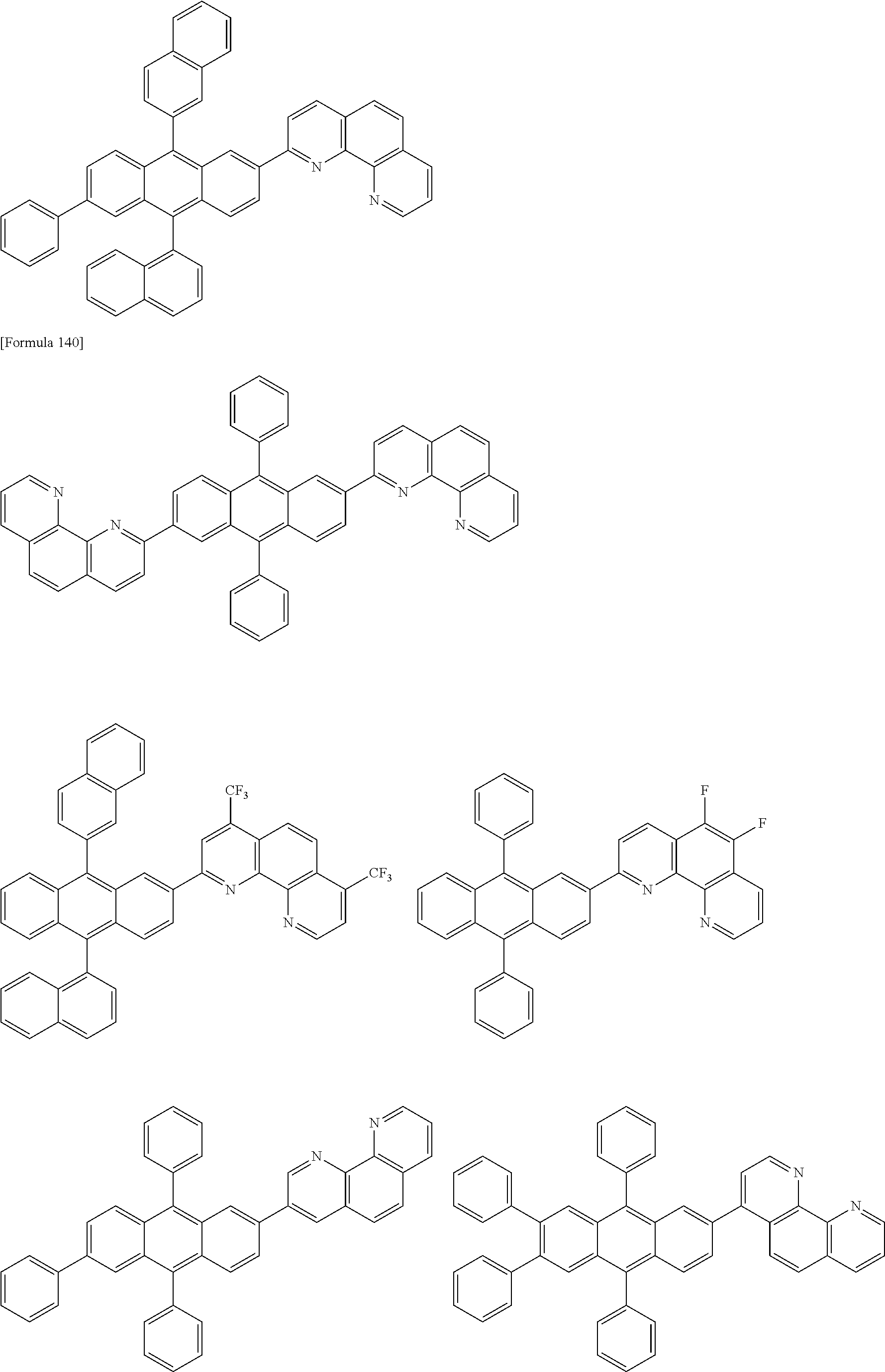

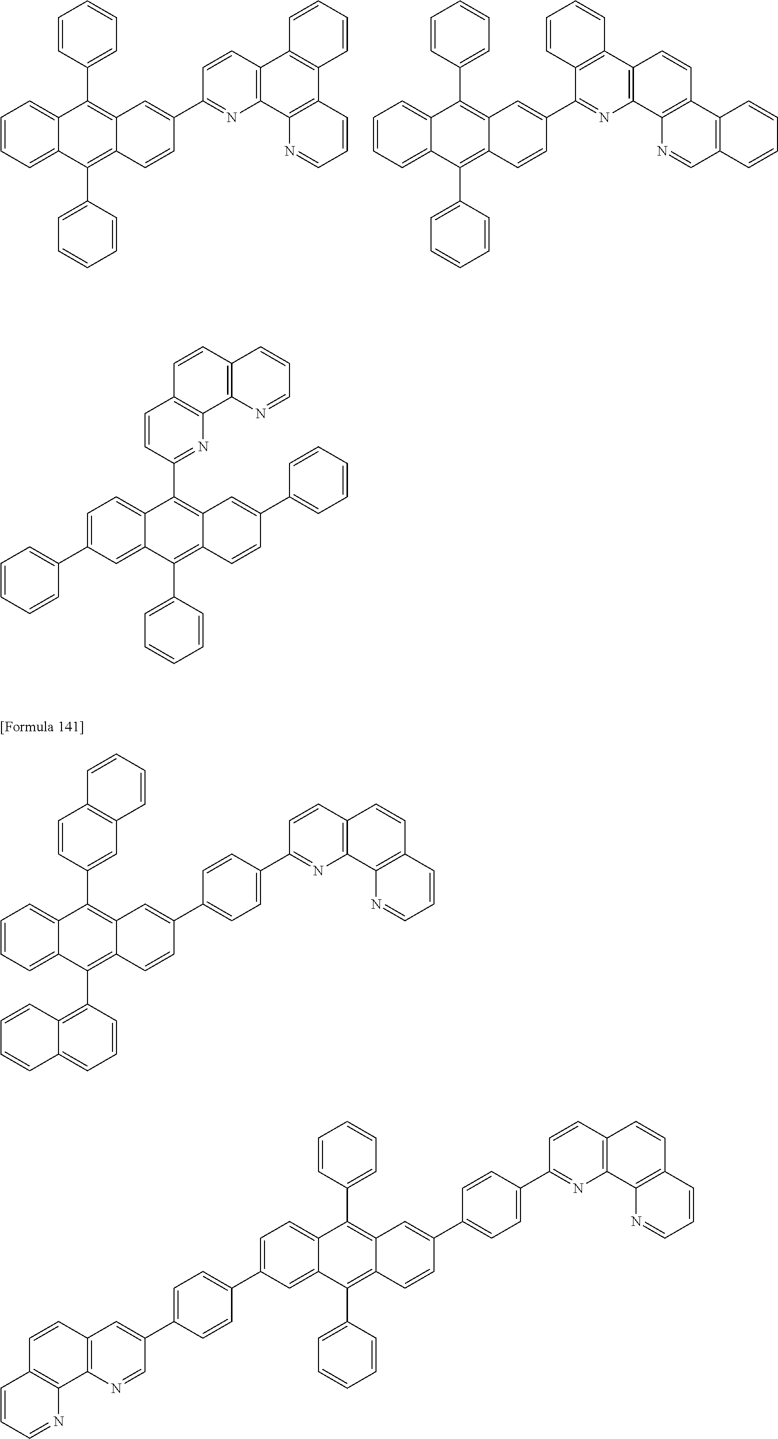

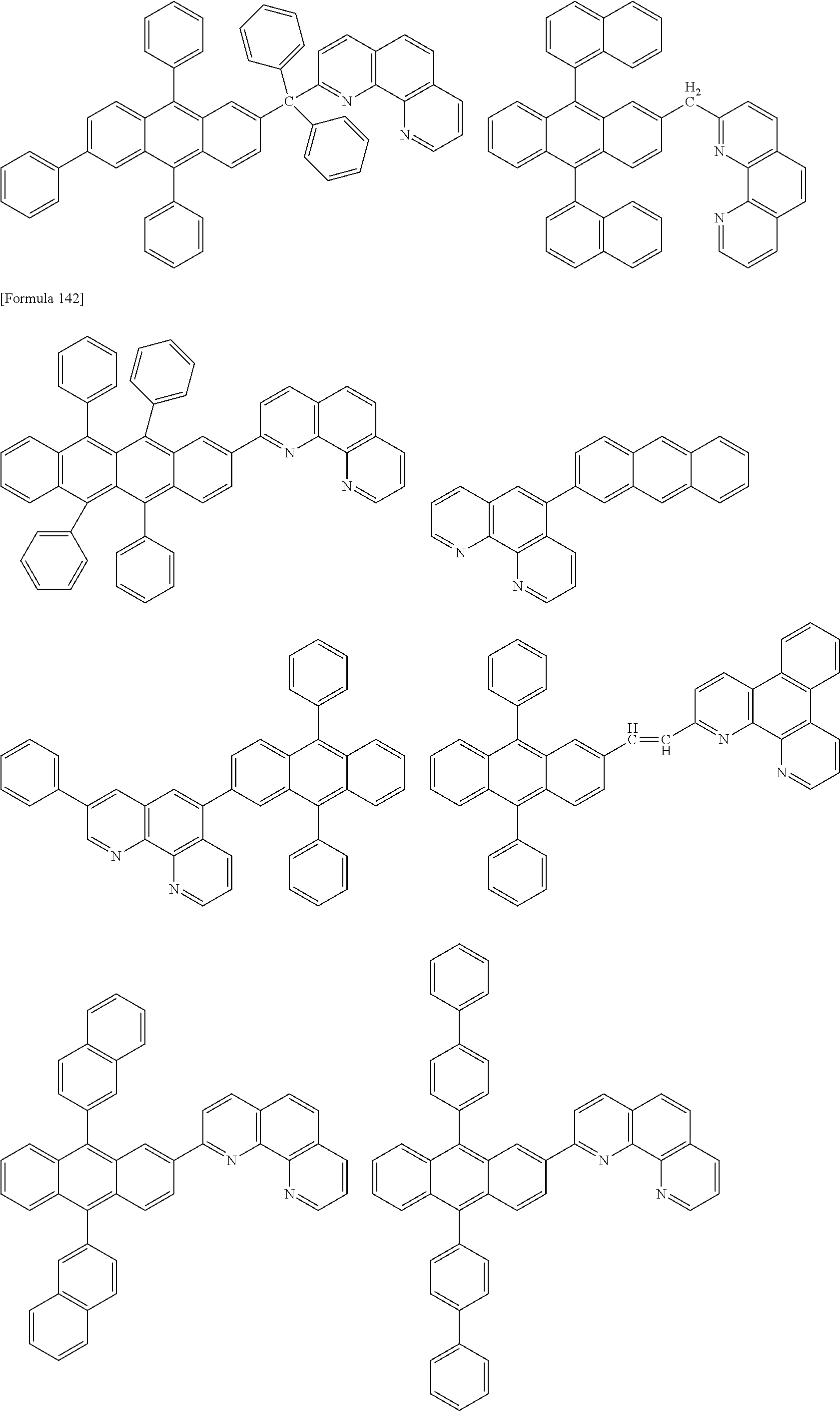

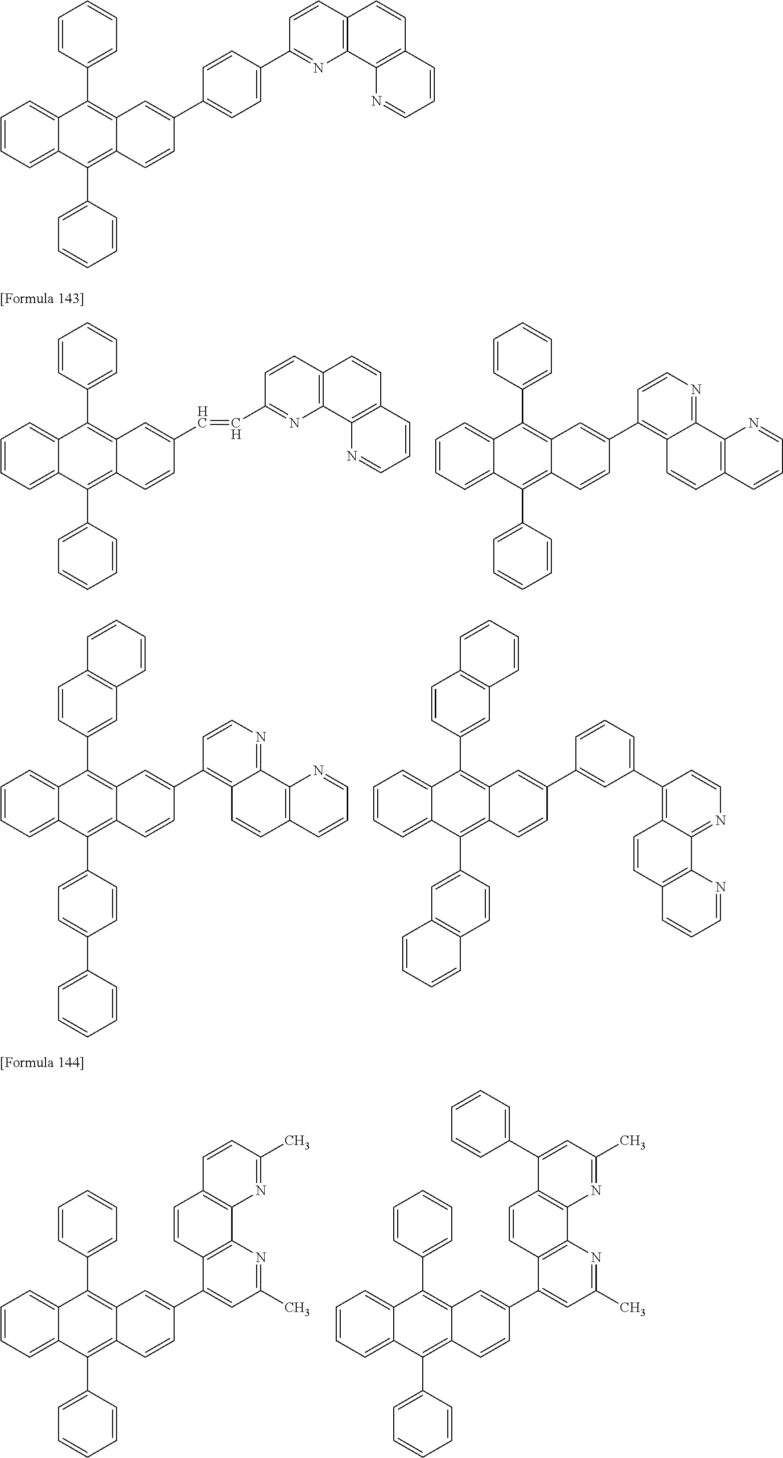

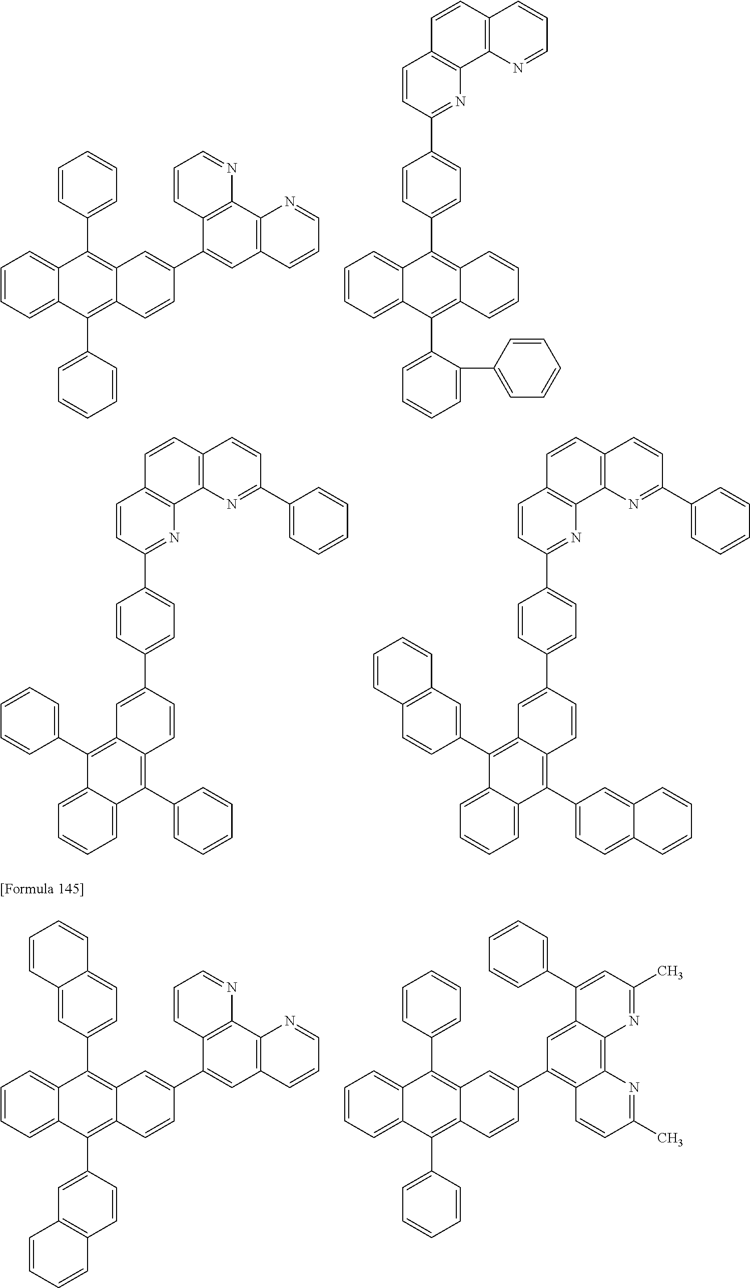

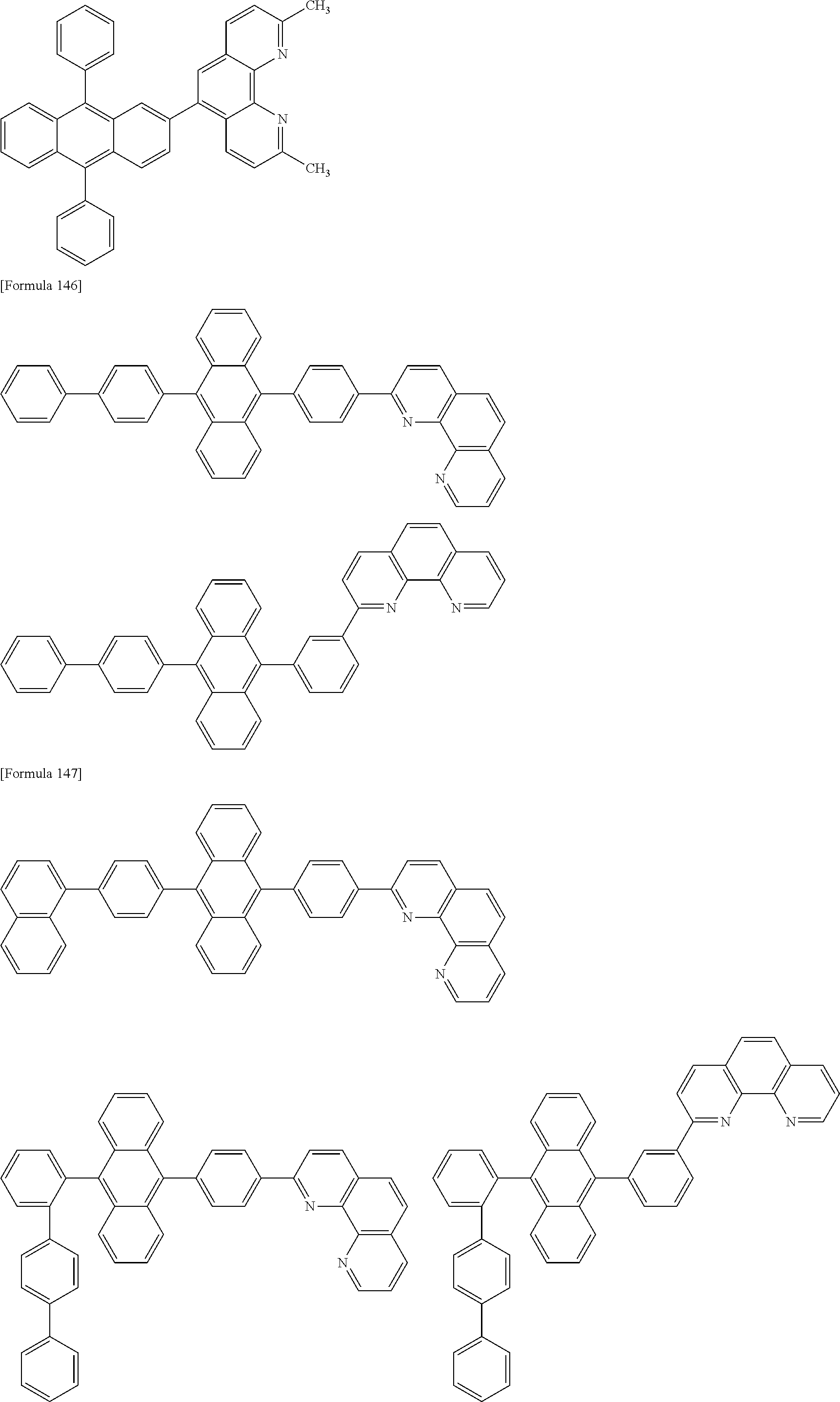

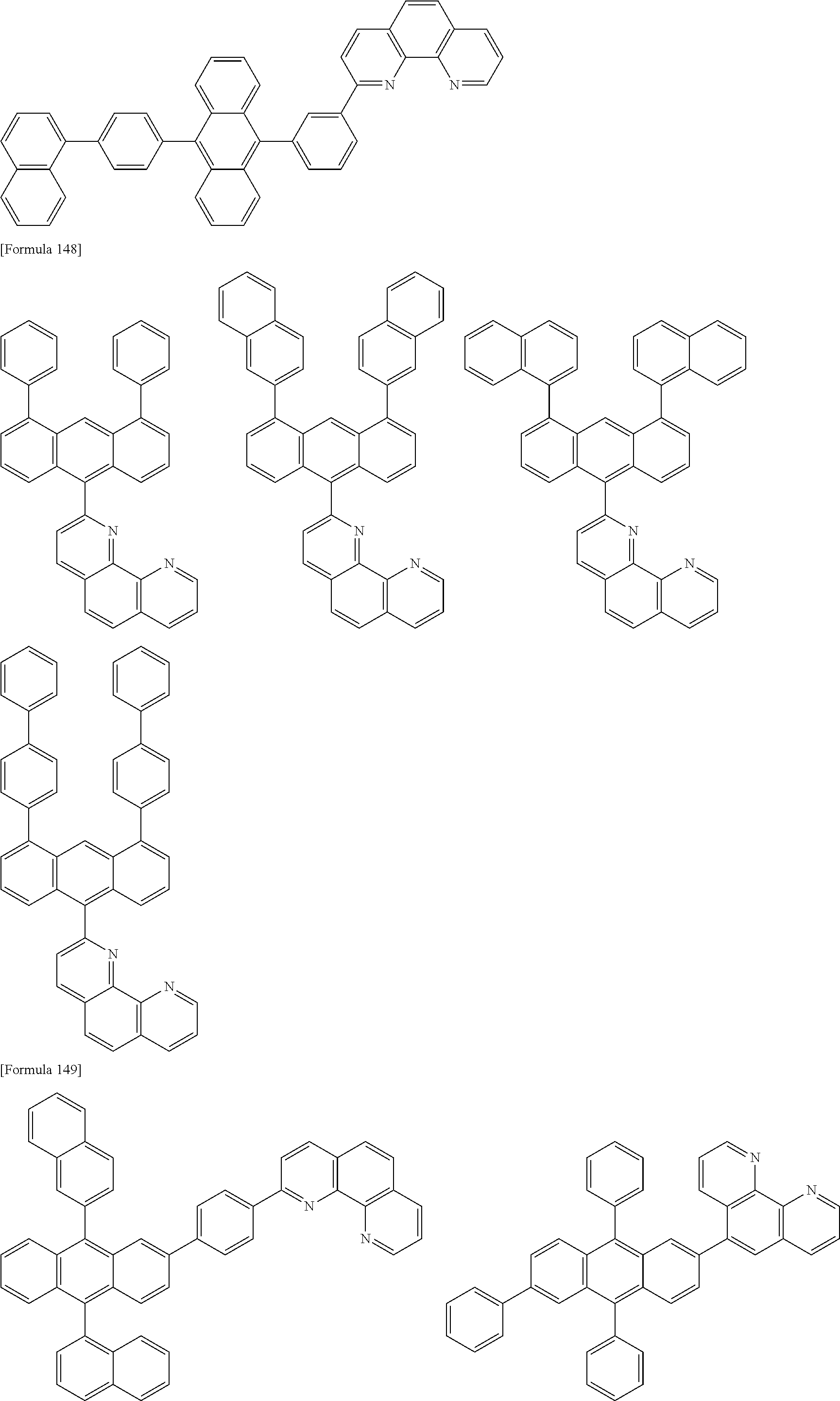

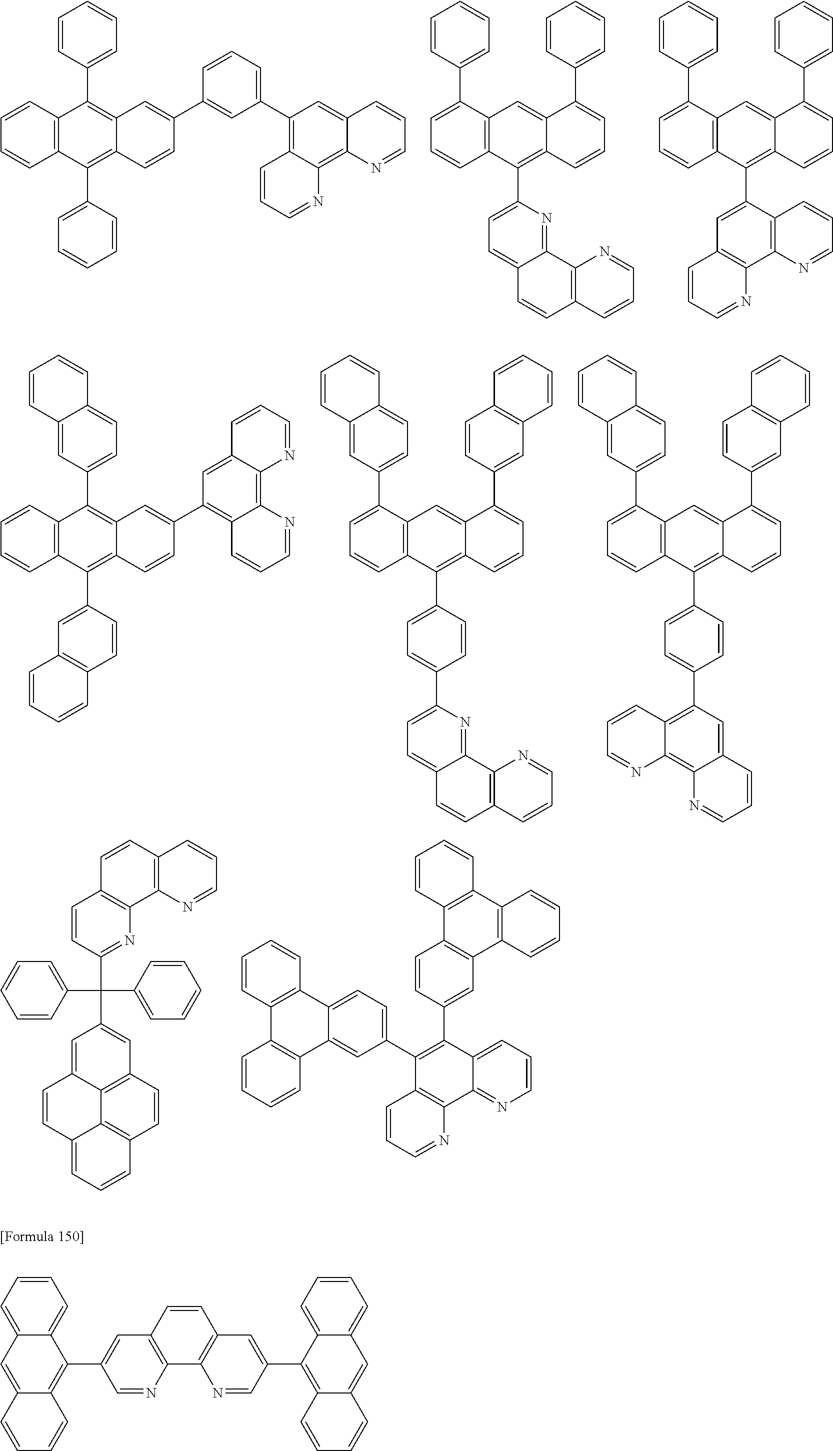

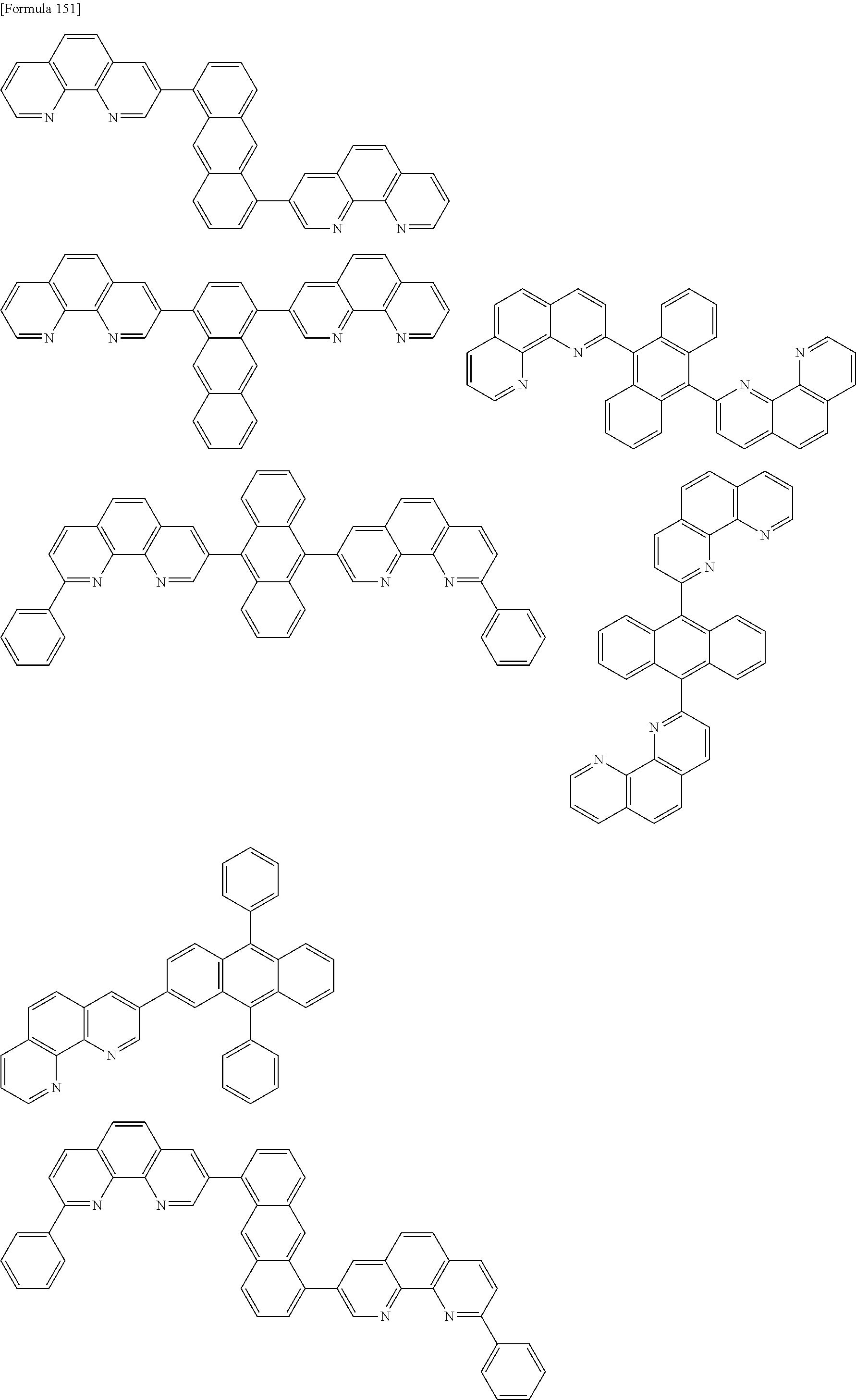

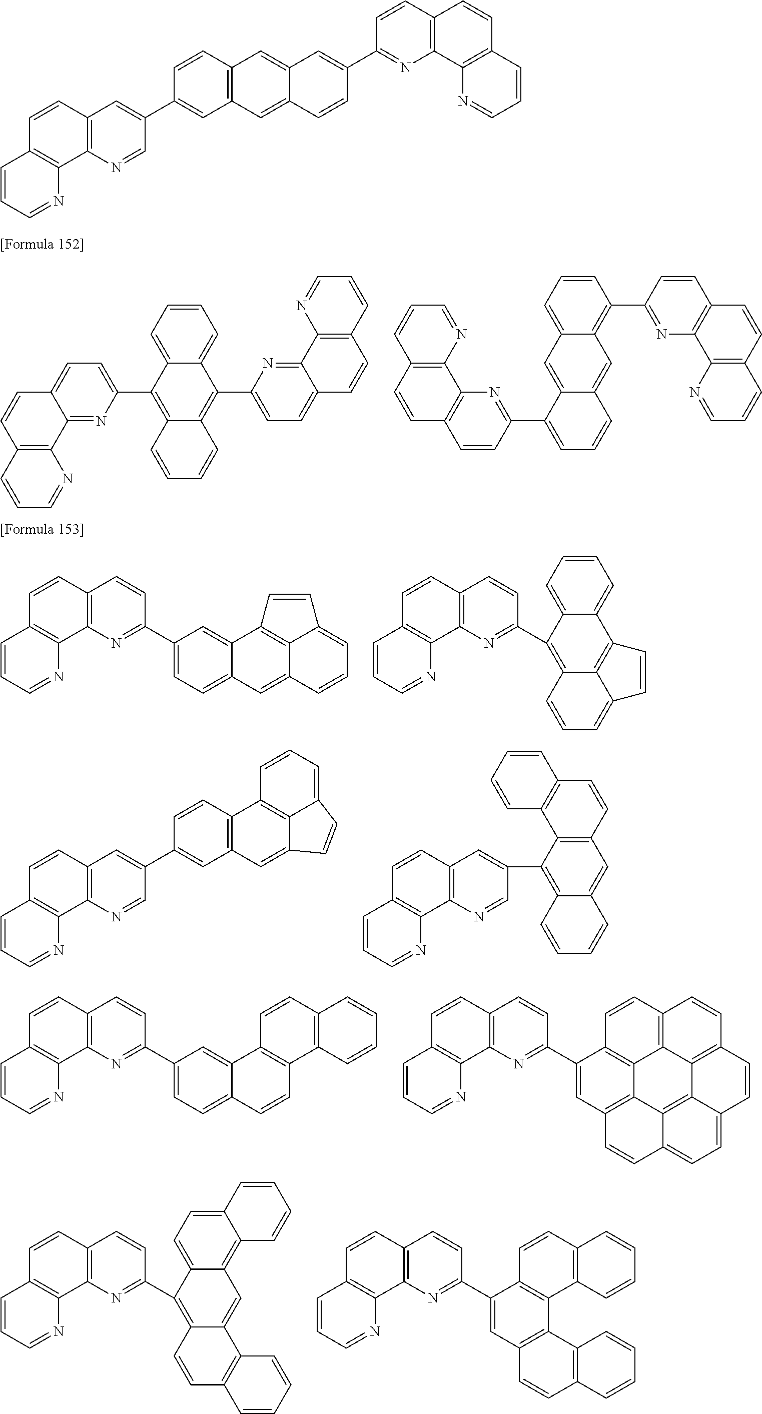

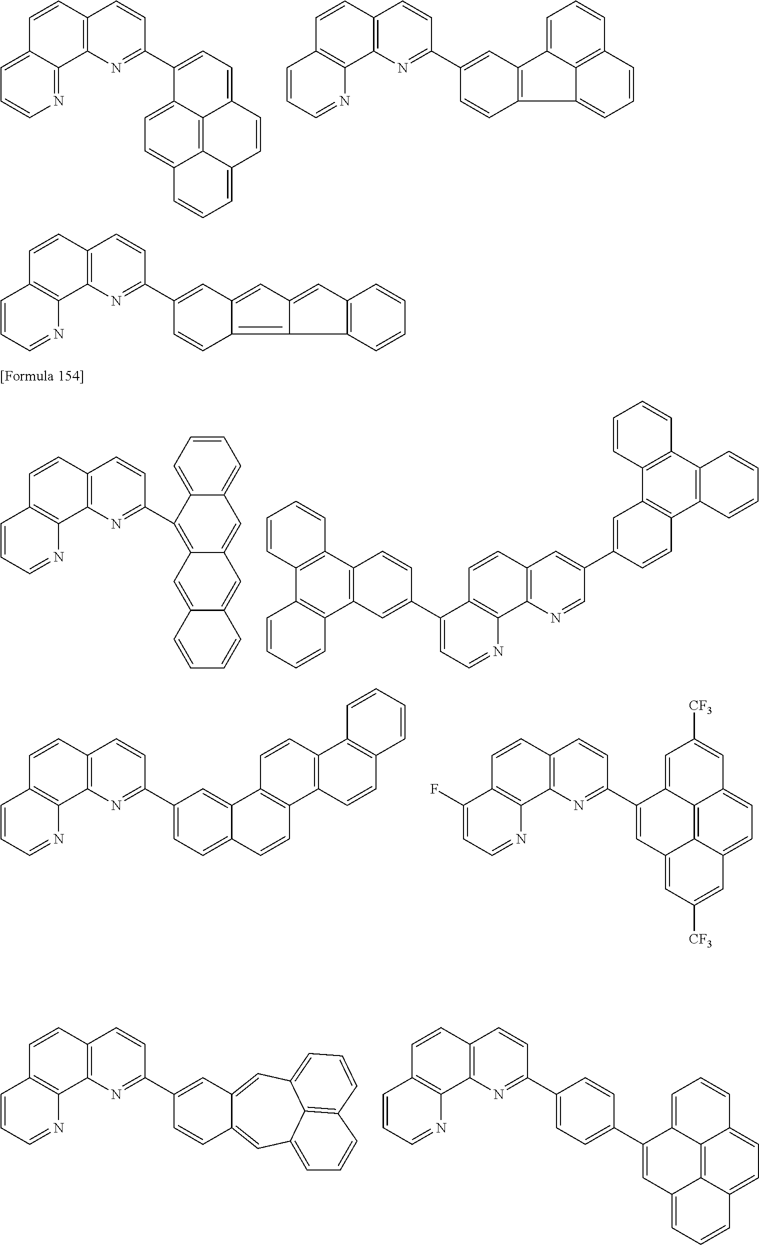

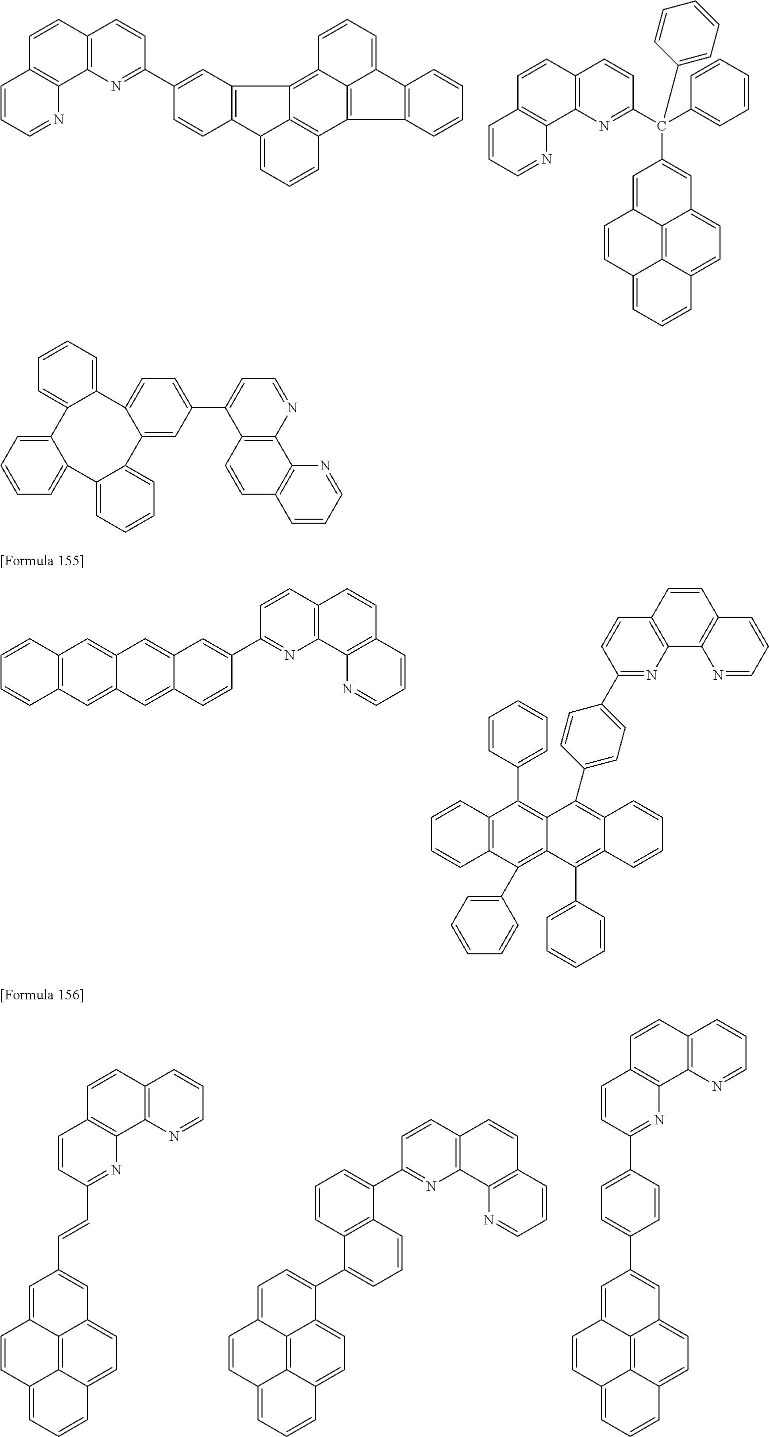

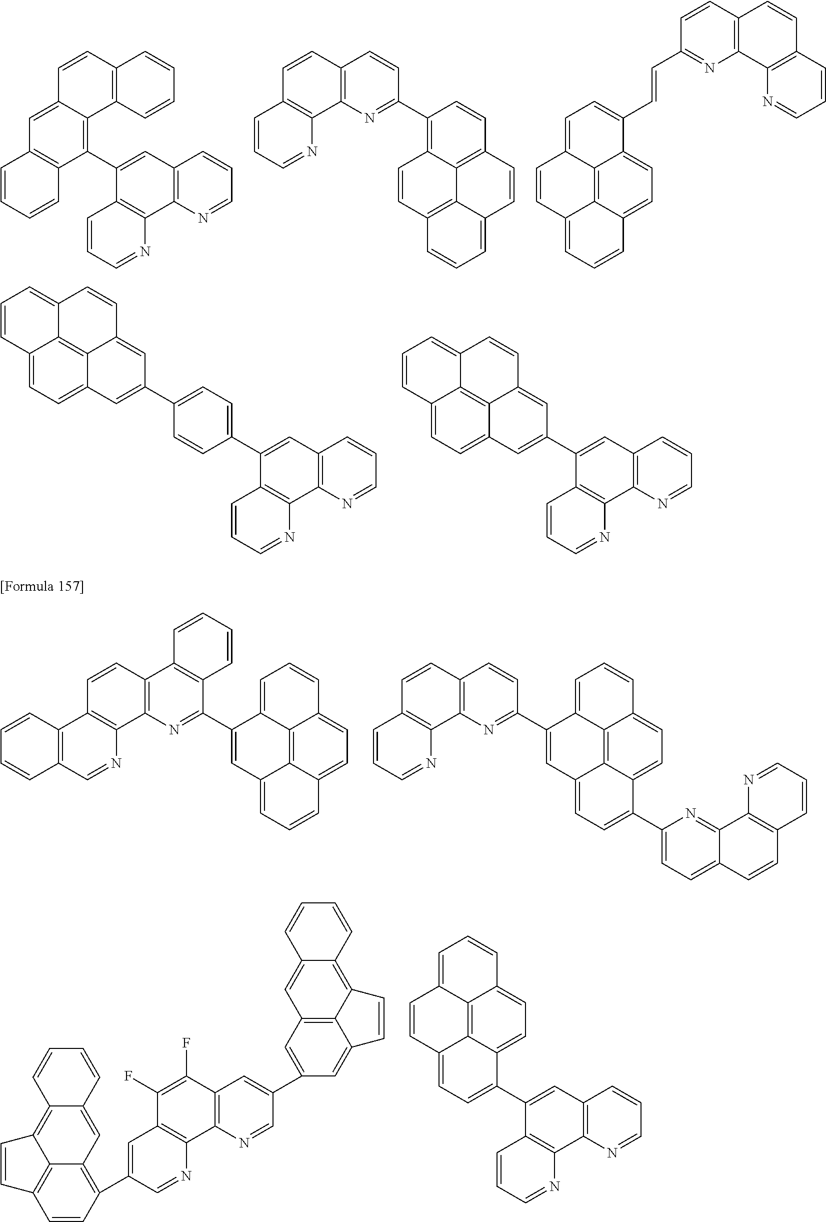

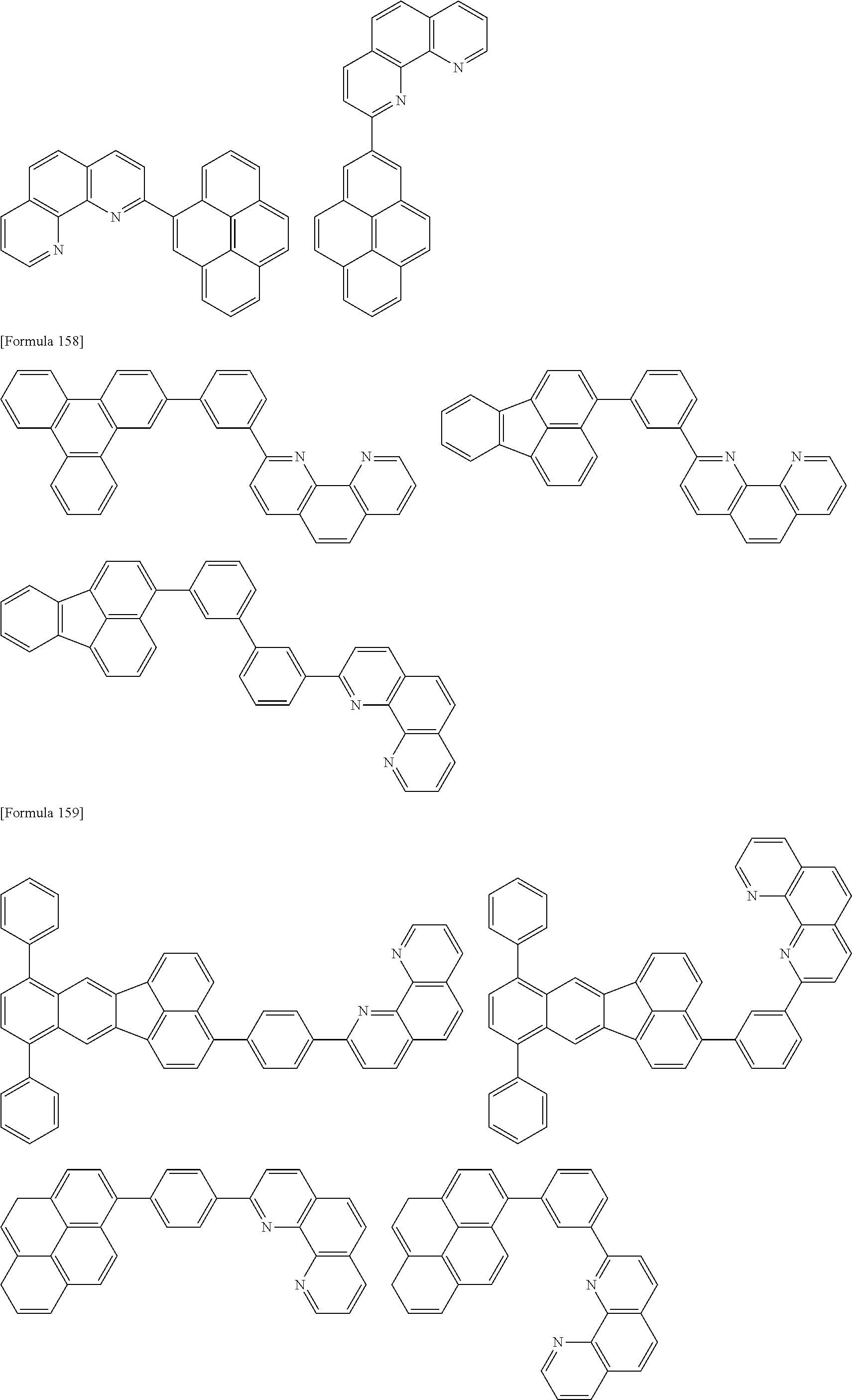

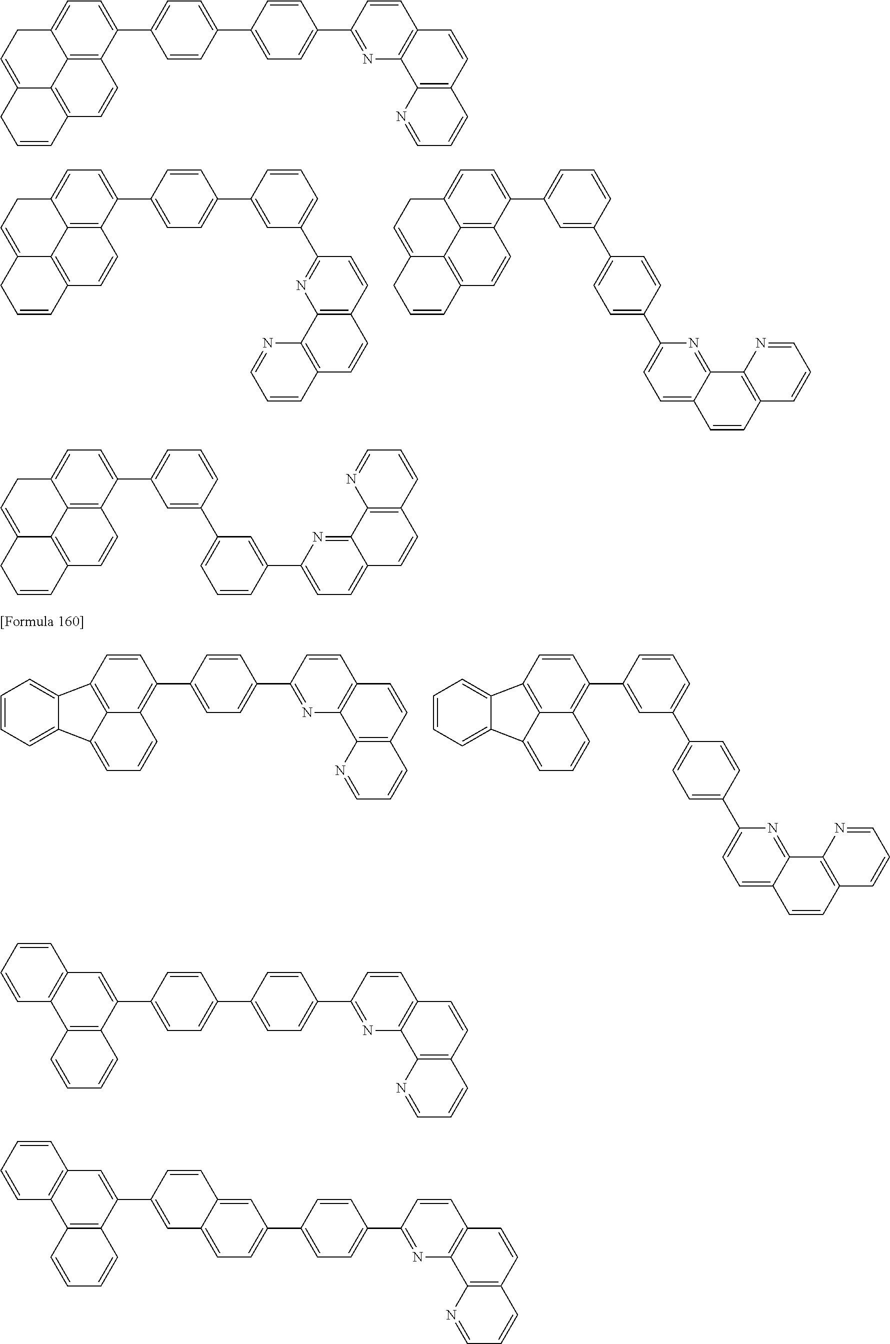

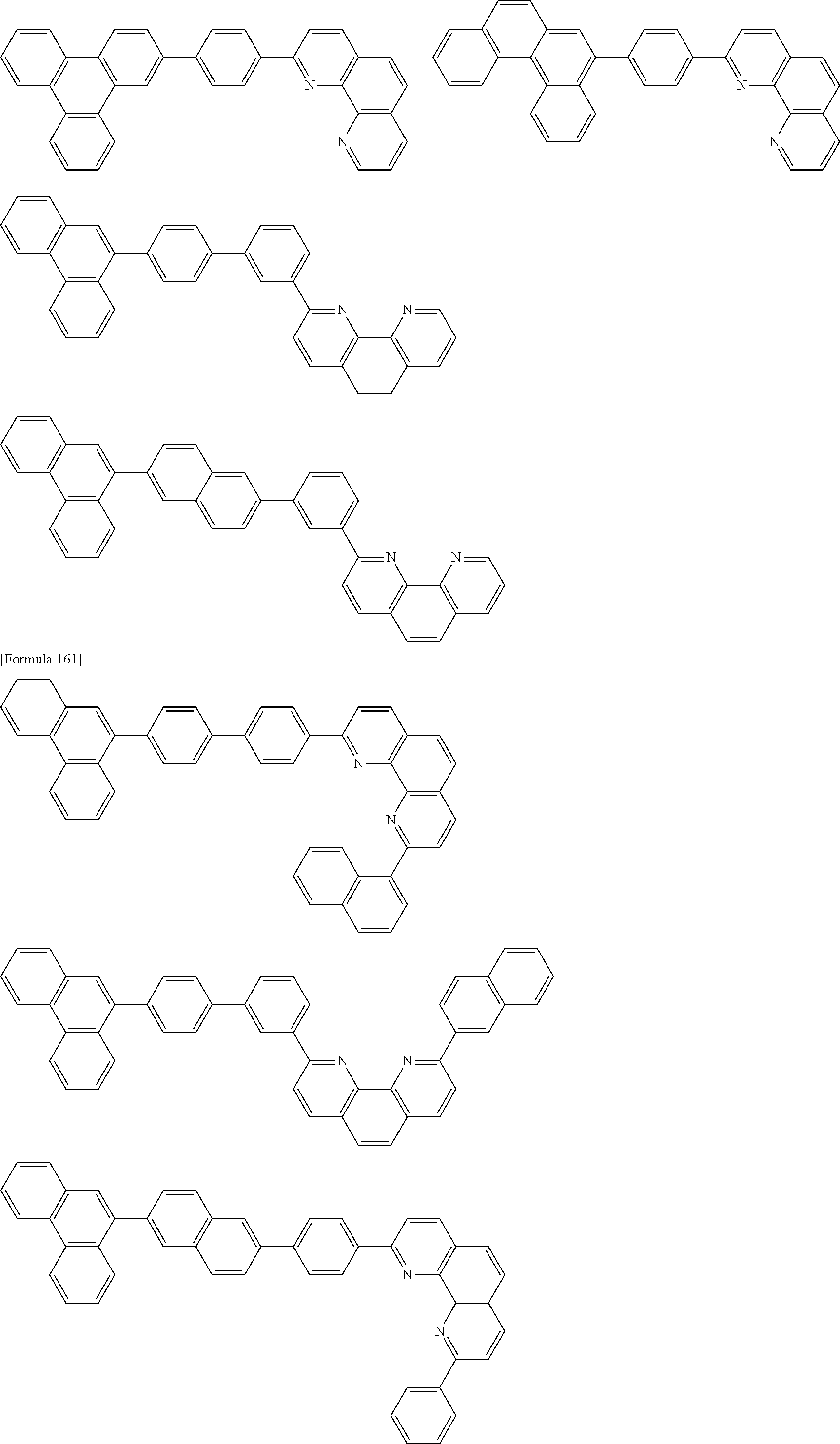

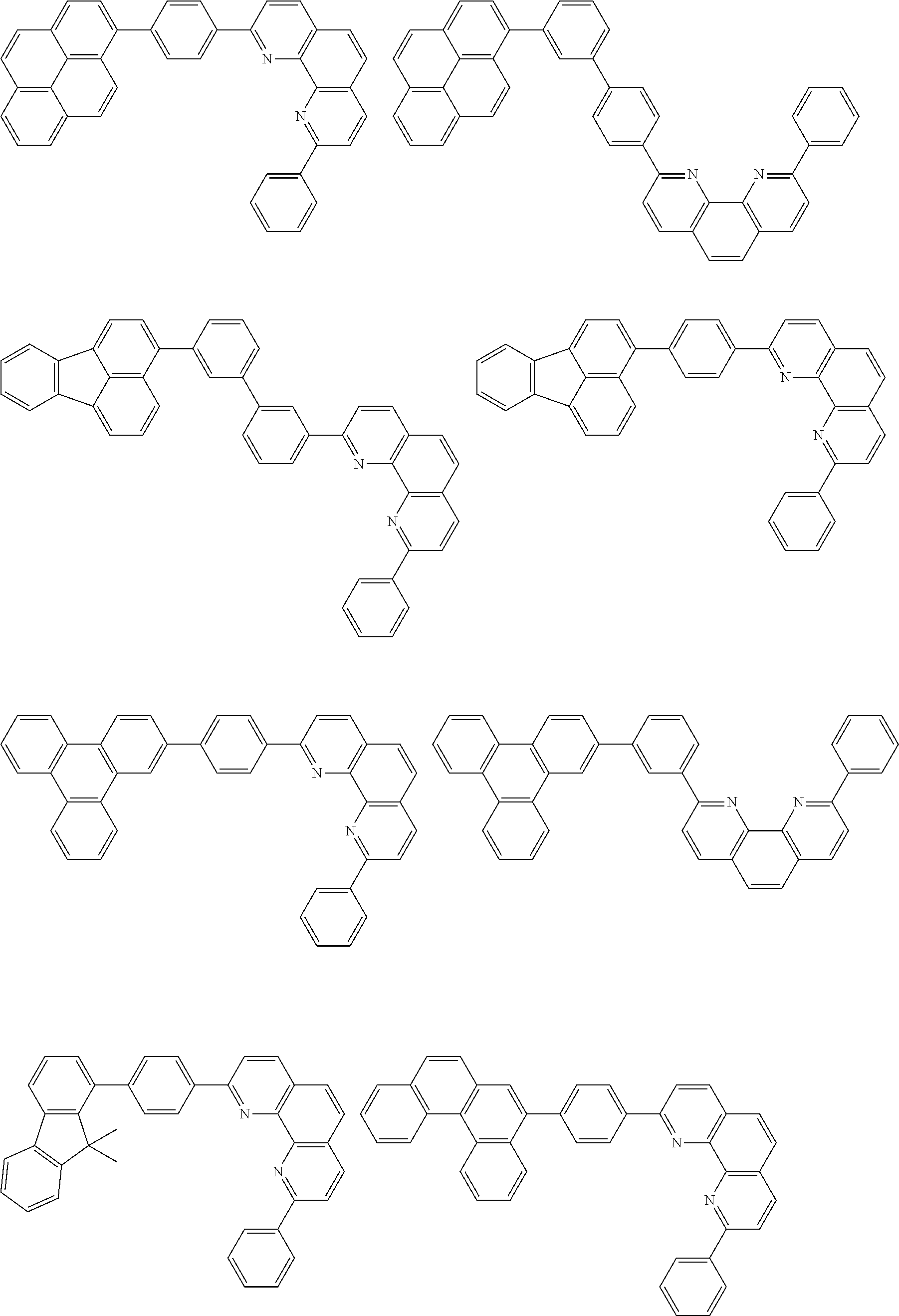

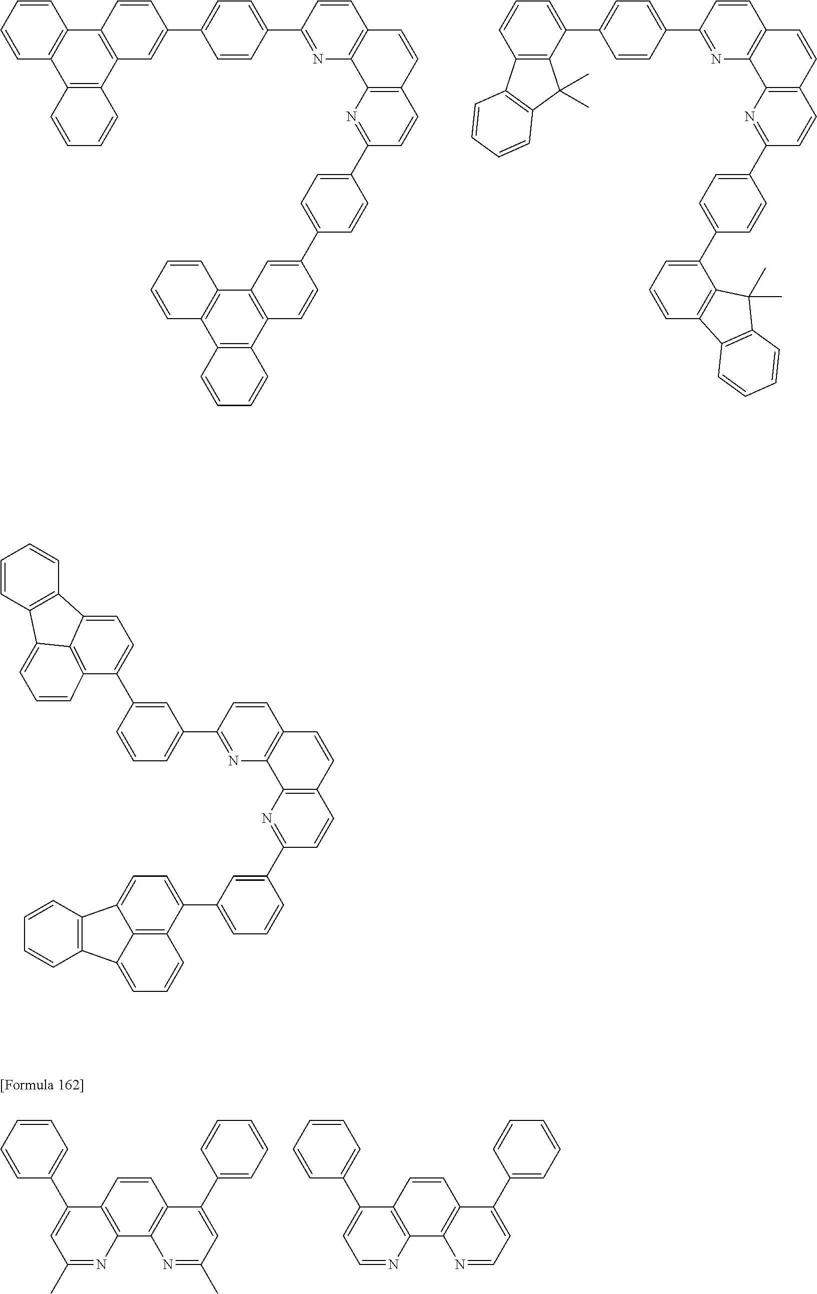

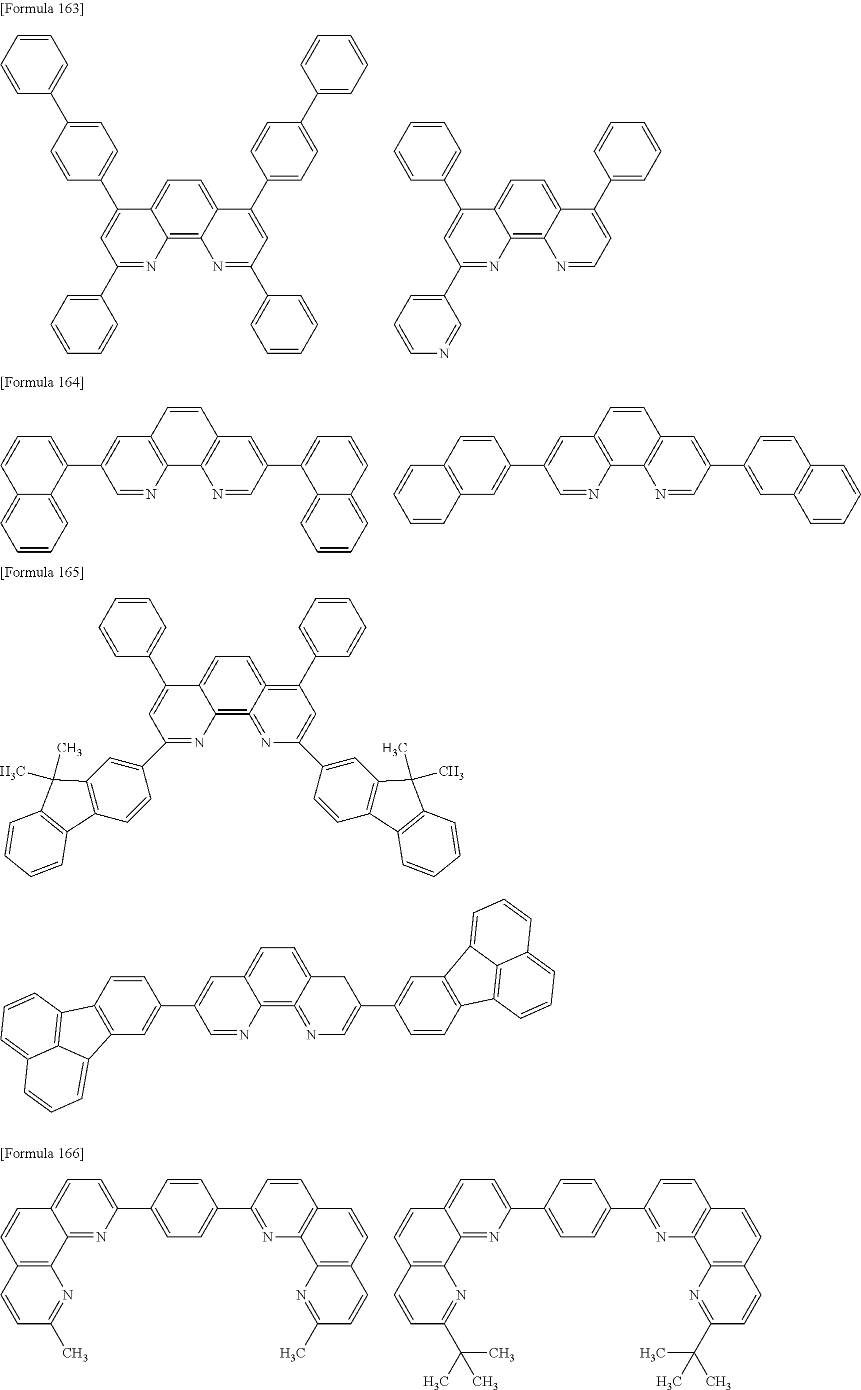

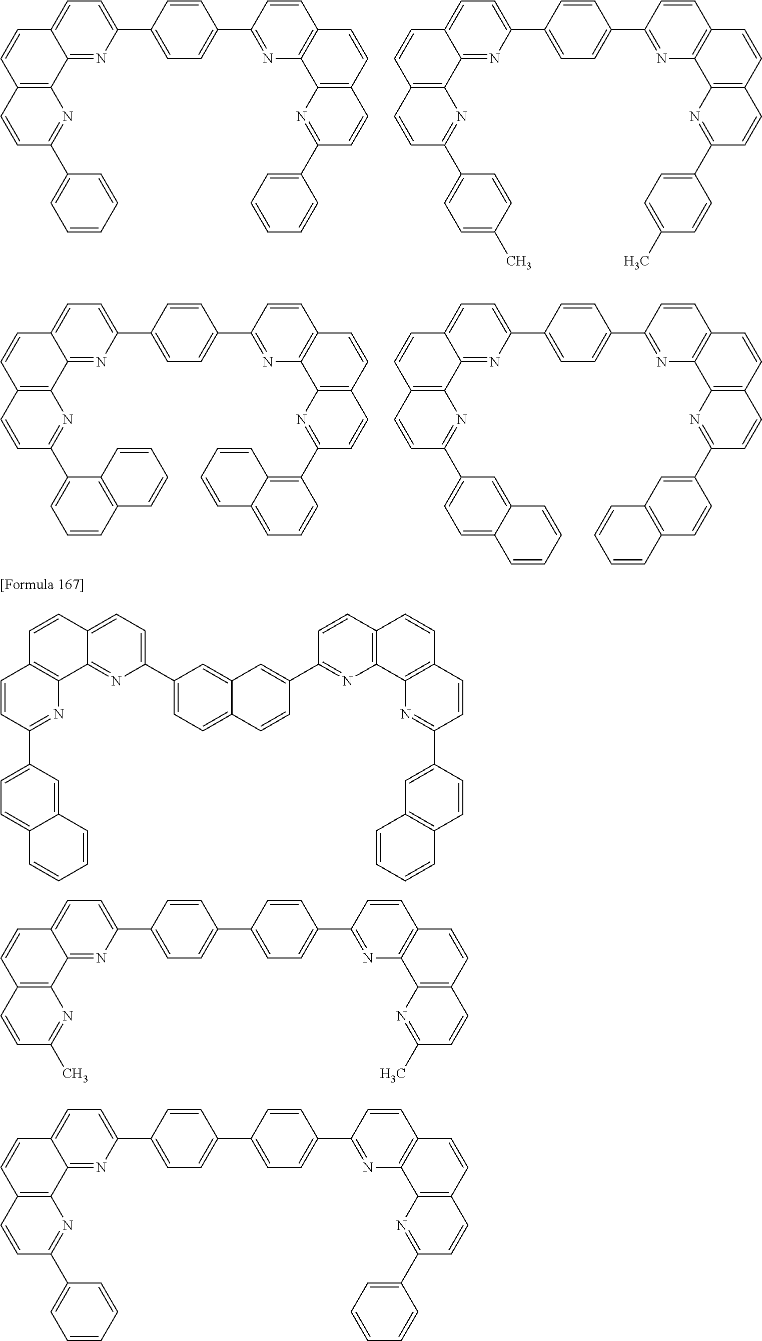

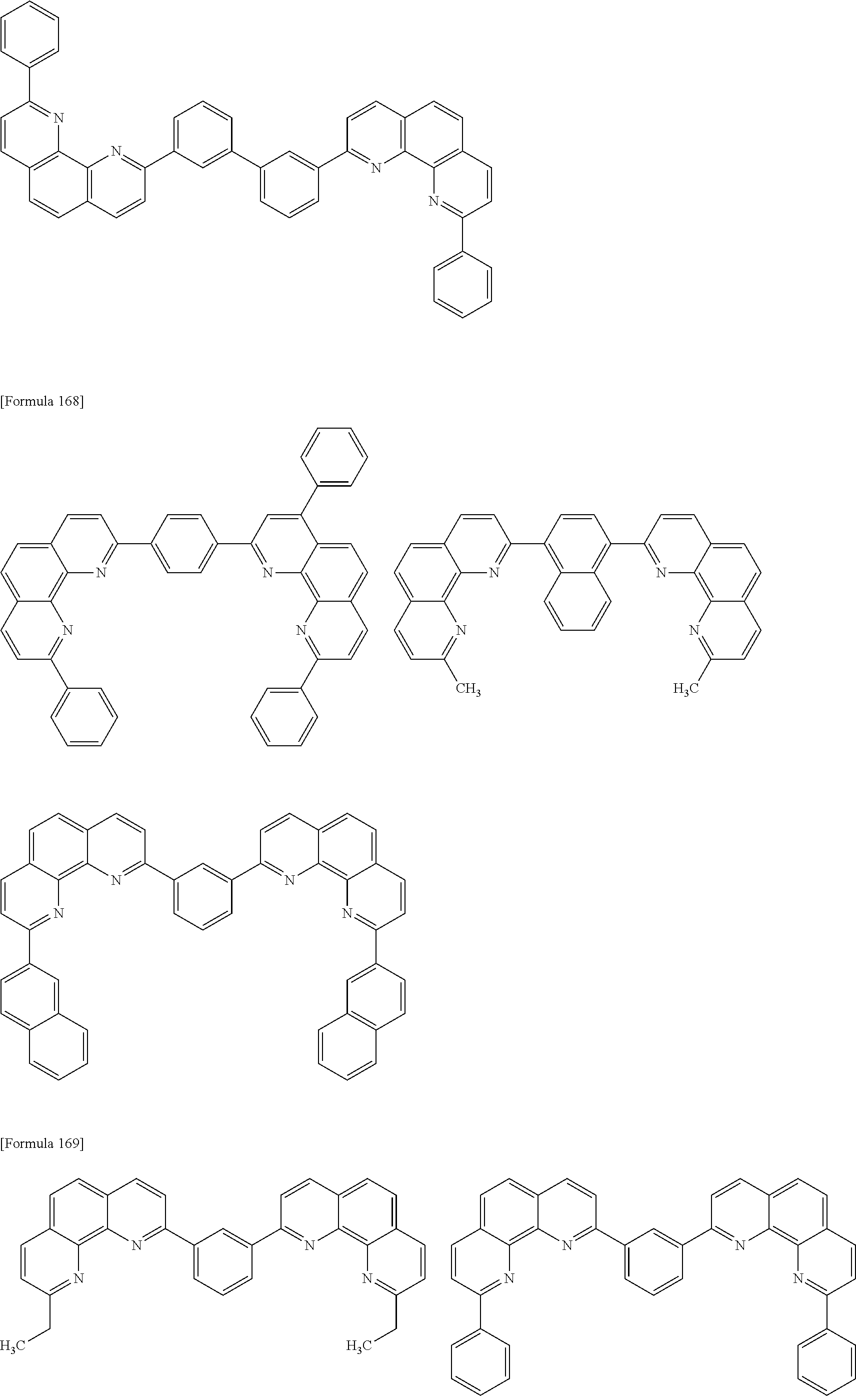

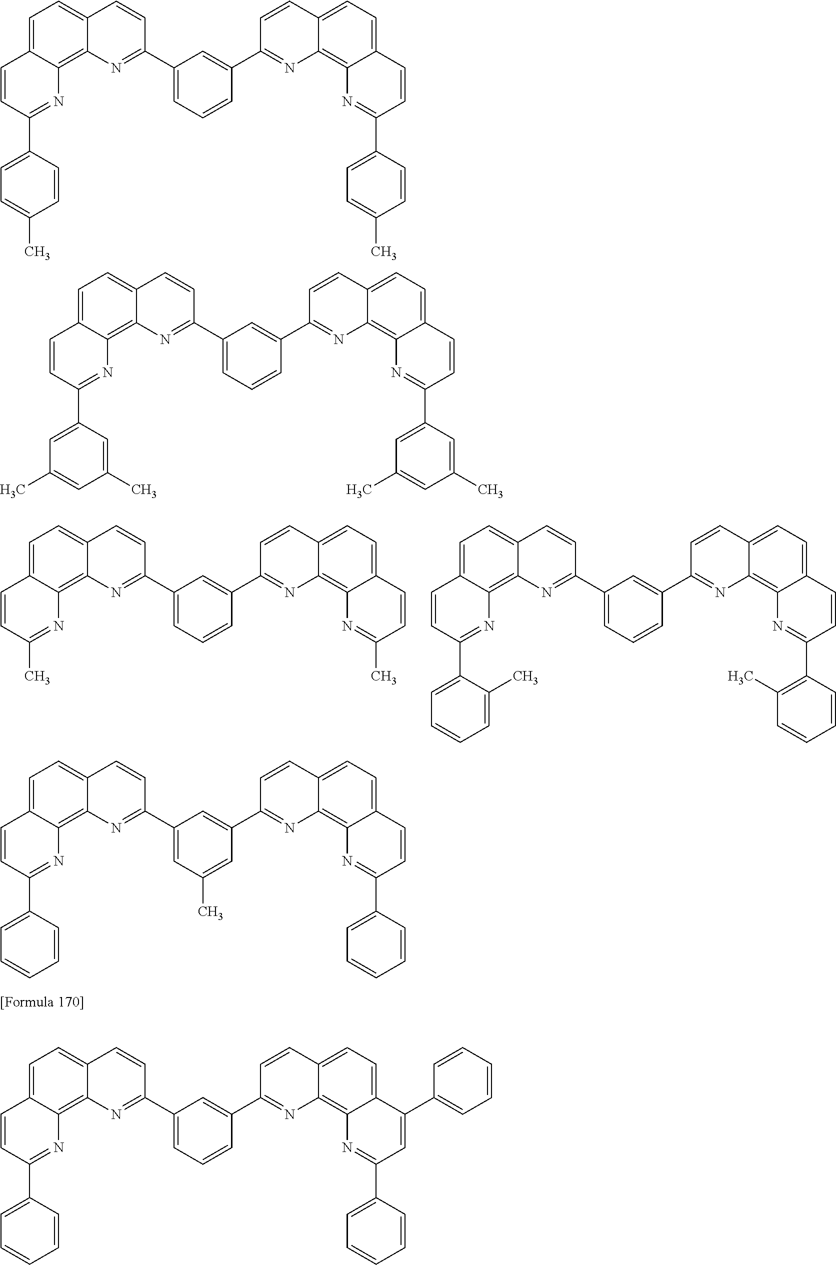

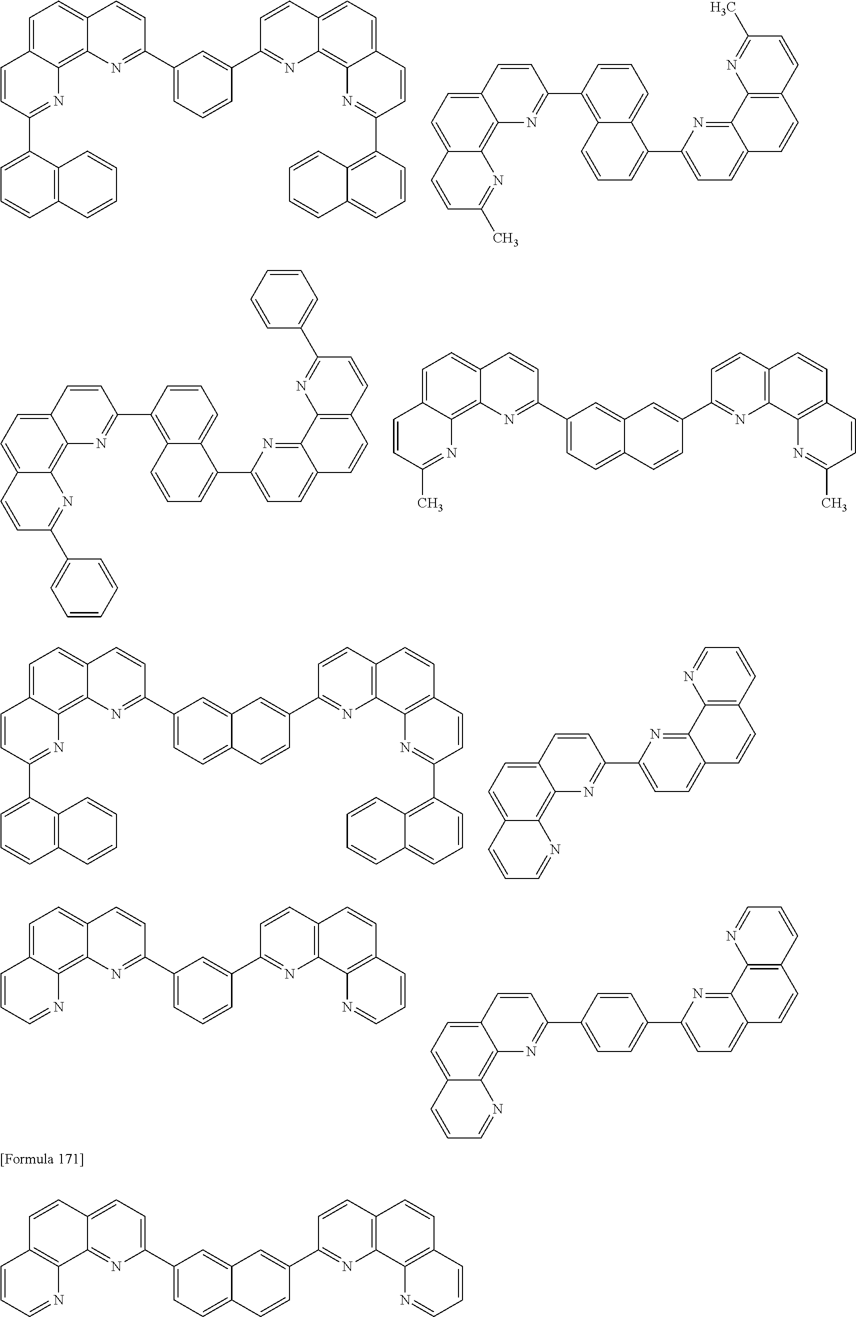

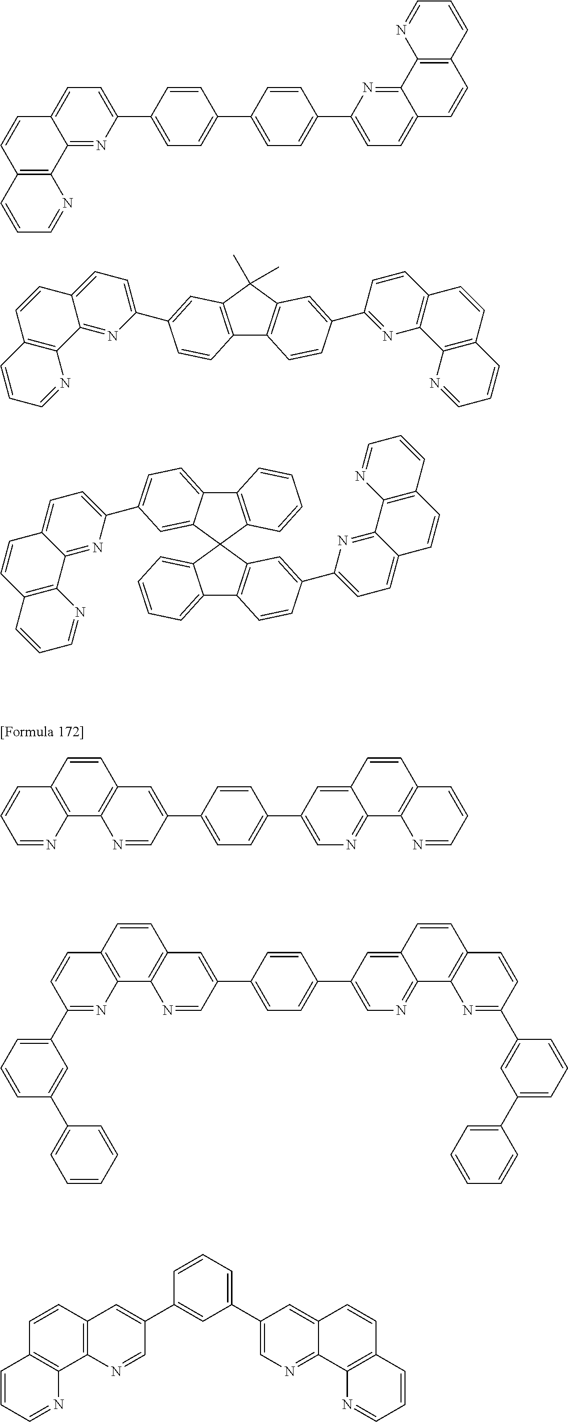 [SMILES: c1cnc2c(c1)ccc1ccc(-c3ccc(-c4ccc(-c5ccc6ccc7cccnc7c6n5)cc4)cc3)nc12 ; CC1(C)c2cc(-c3ccc4ccc5cccnc5c4n3)ccc2-c2ccc(-c3ccc4ccc5cccnc5c4n3)cc21 ; c1ccc2c(c1)-c1ccc(-c3ccc4ccc5cccnc5c4n3)cc1C21c2ccccc2-c2ccc(-c3ccc4ccc5cccnc5c4n3)cc21 ; c1cnc2c(c1)ccc1cc(-c3ccc(-c4cnc5c(ccc6cccnc65)c4)cc3)cnc12 ; c1ccc(-c2cccc(-c3ccc4ccc5cc(-c6ccc(-c7cnc8c(ccc9ccc(-c%10cccc(-c%11ccccc%11)c%10)nc98)c7)cc6)cnc5c4n3)c2)cc1 ; c1cc(-c2cnc3c(ccc4cccnc43)c2)cc(-c2cnc3c(ccc4cccnc43)c2)c1]

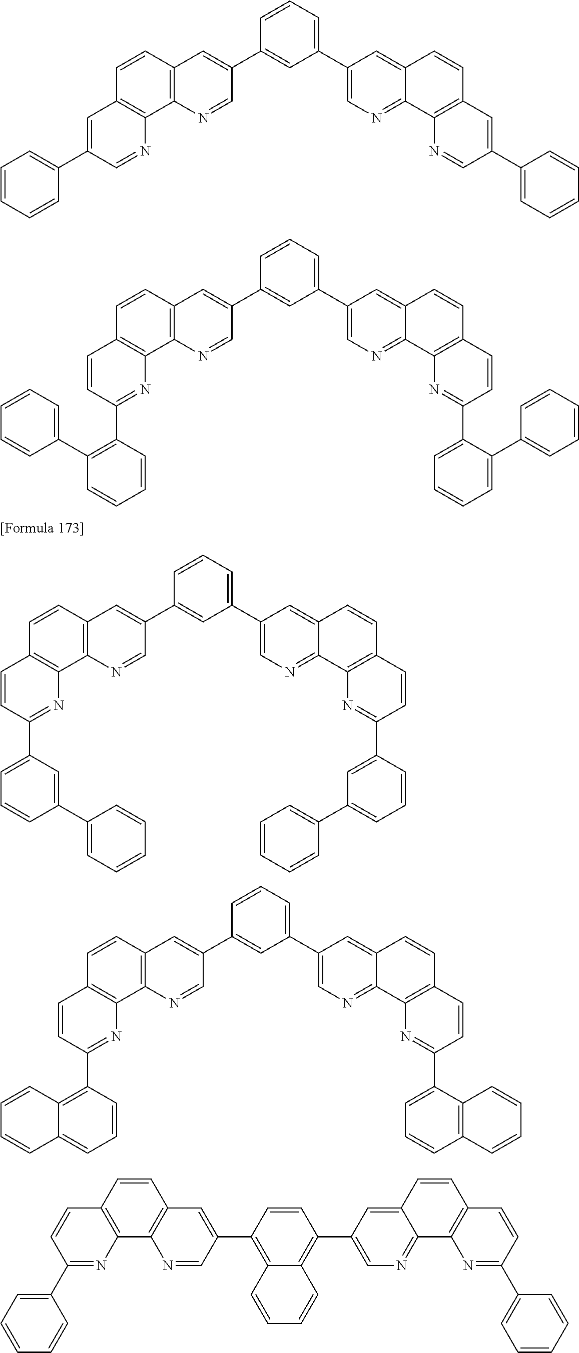

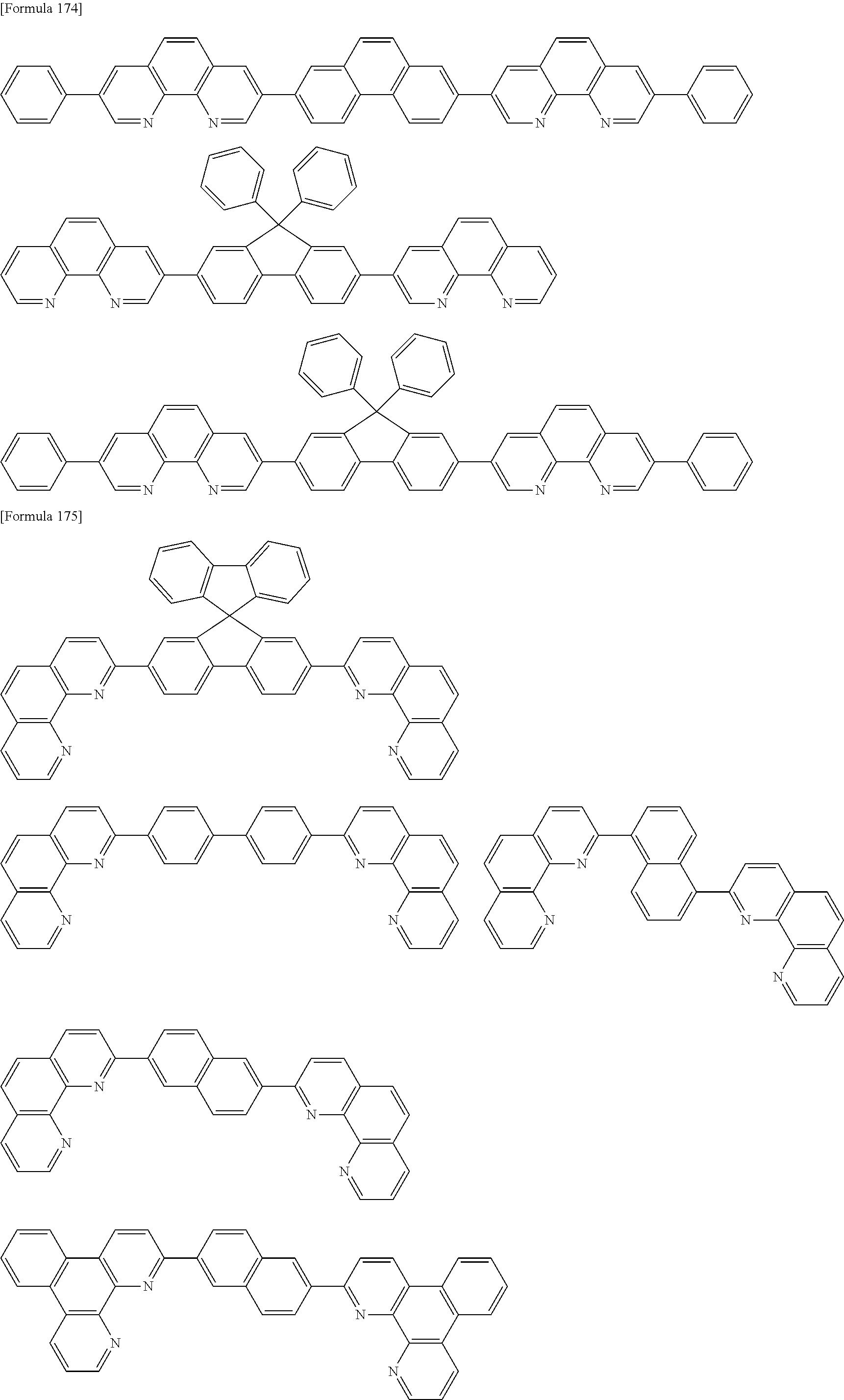

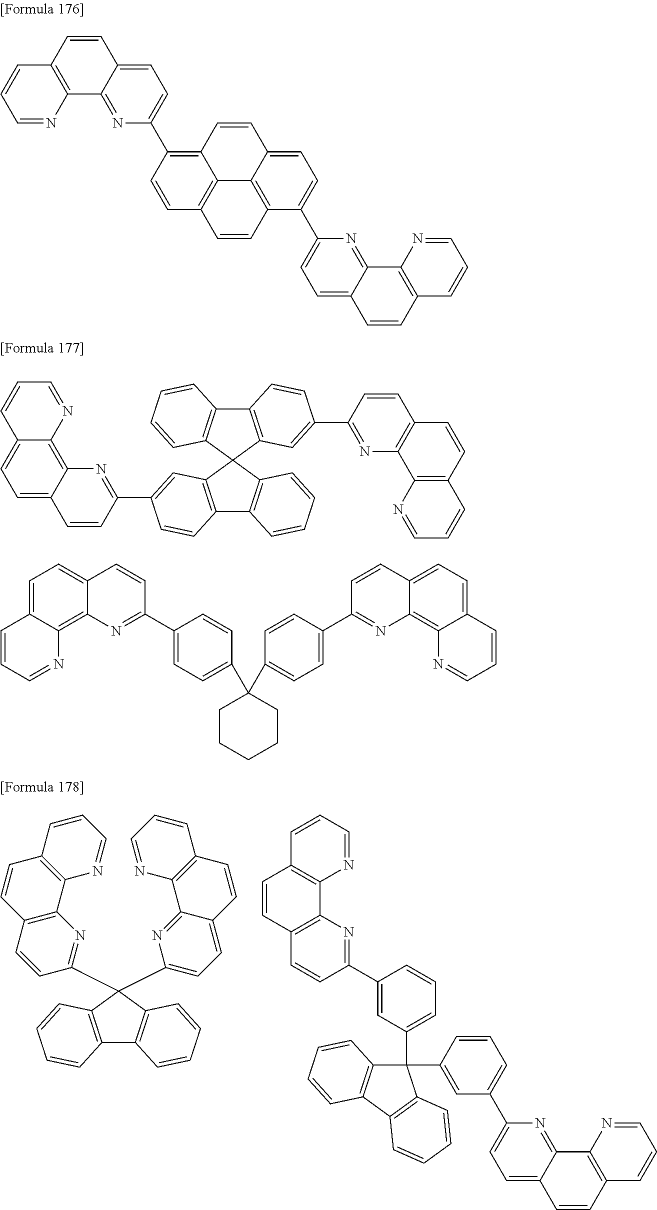

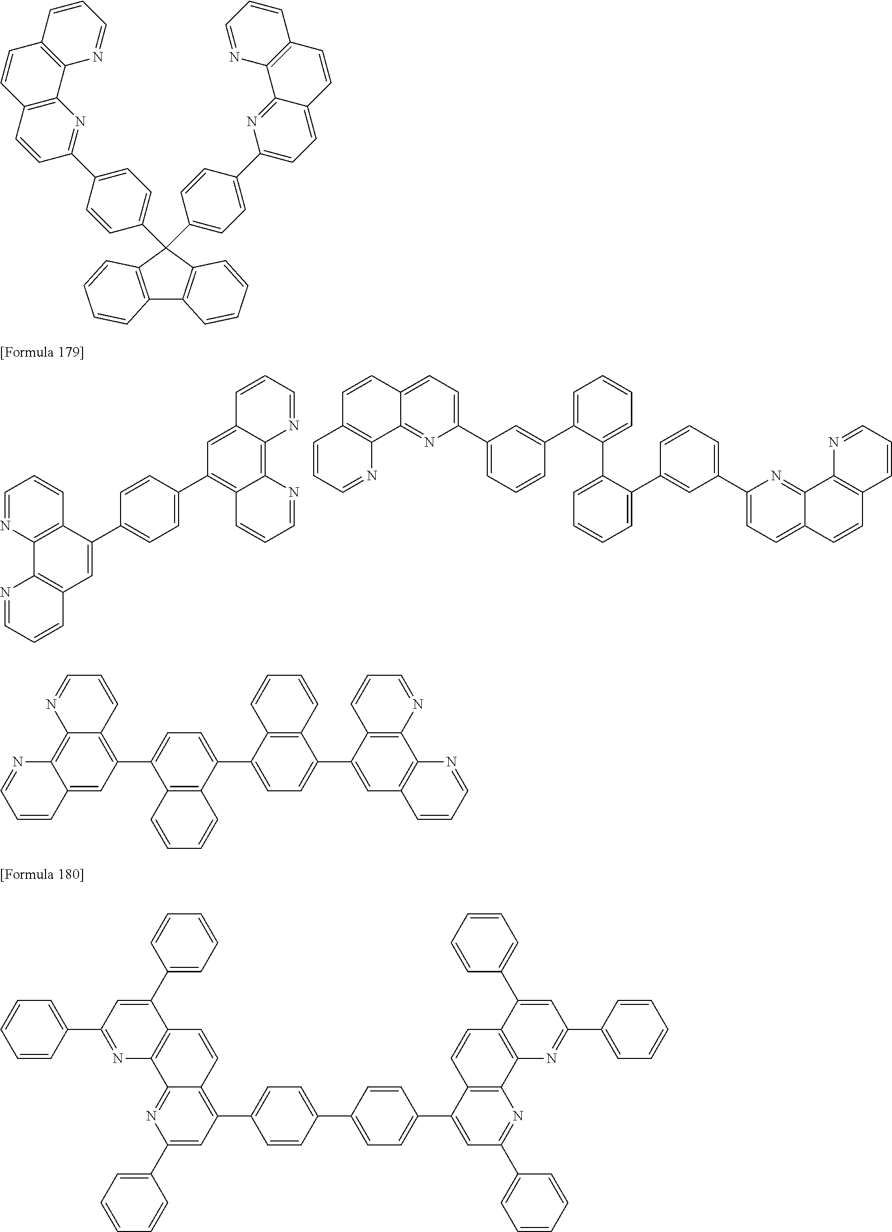

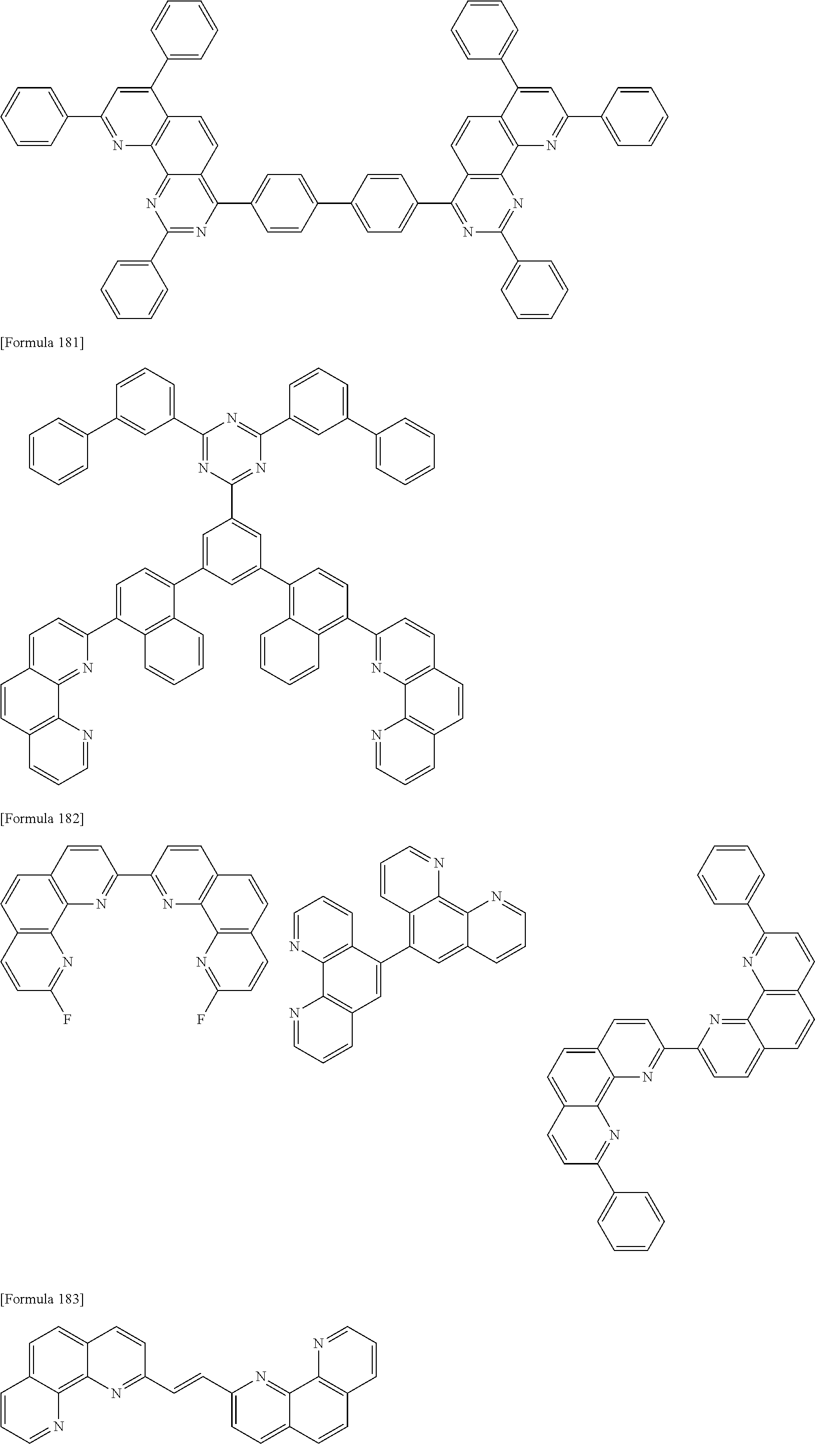

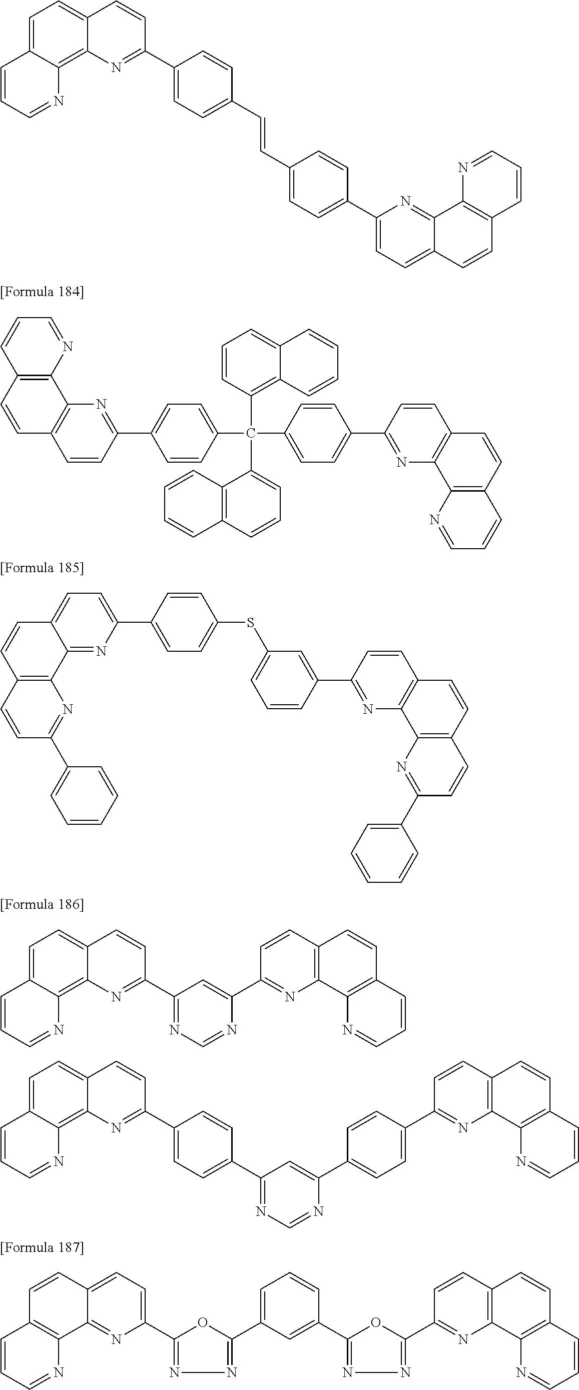

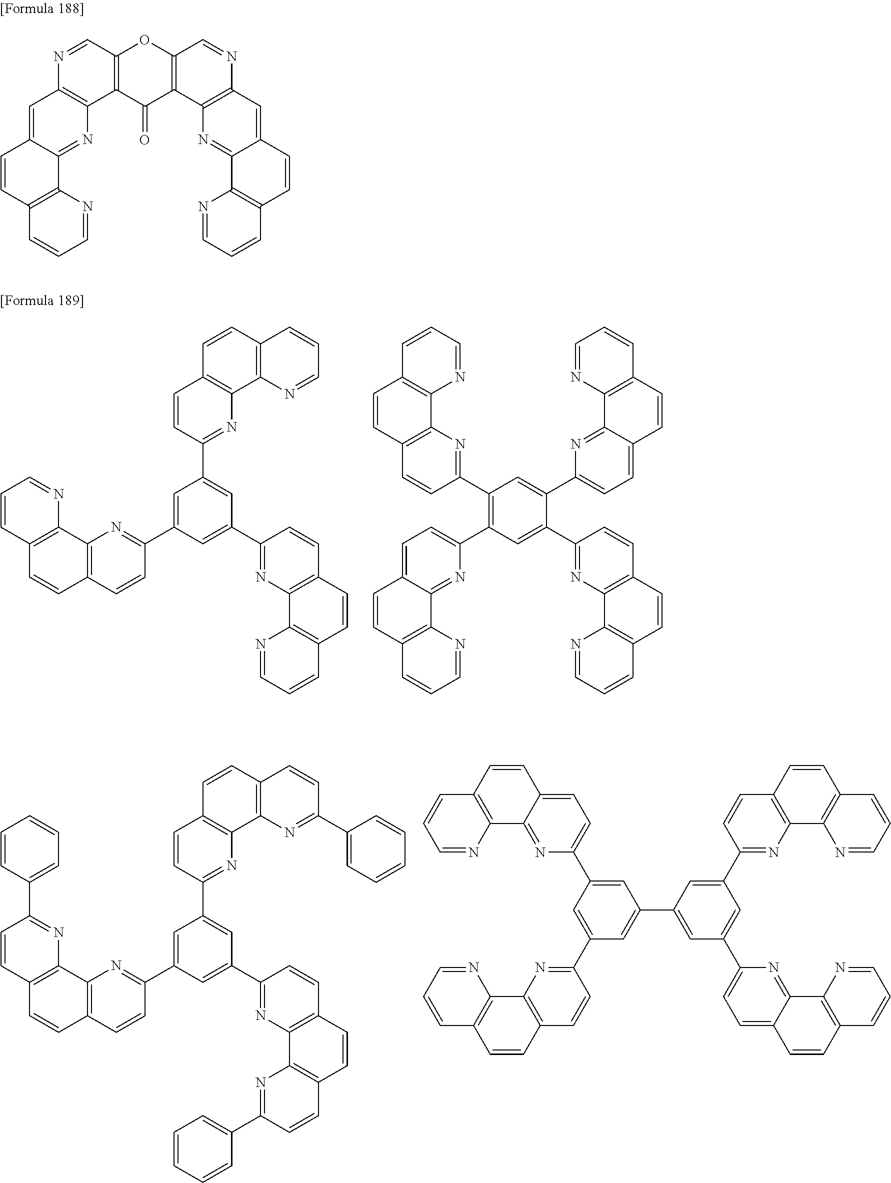

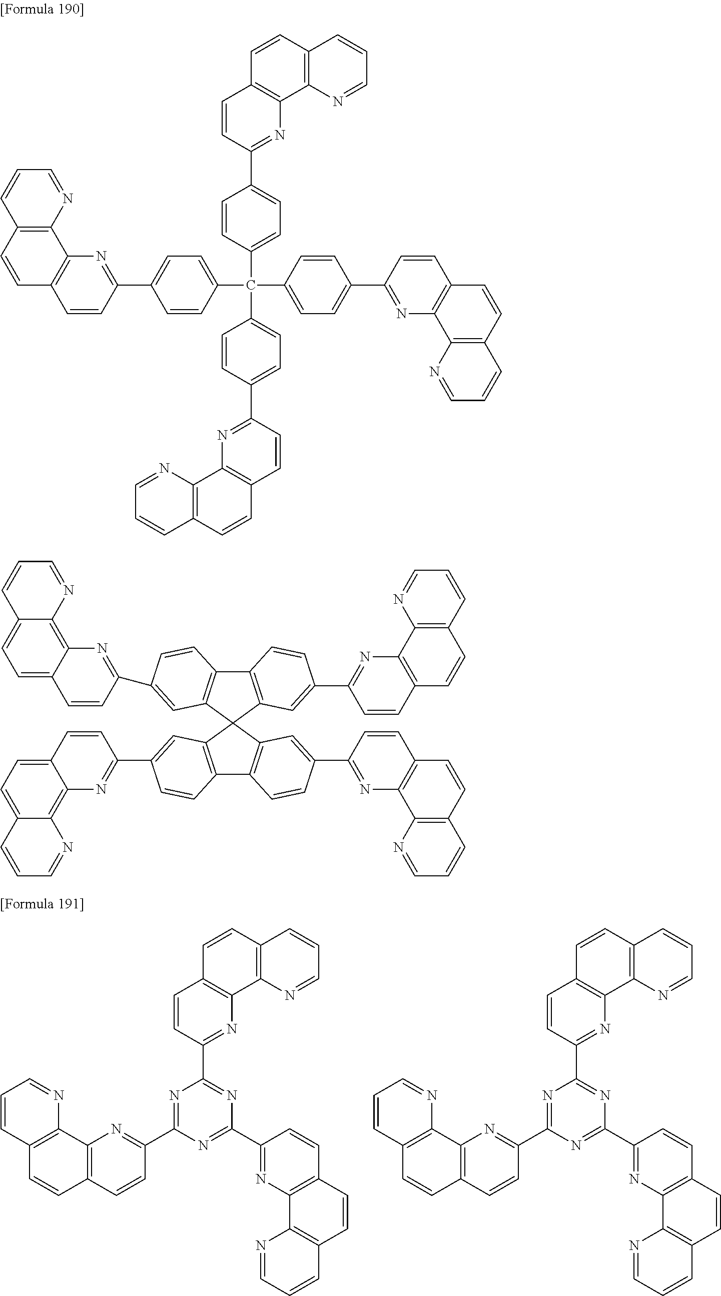 [SMILES: c1cnc2c(c1)ccc1ccc(-c3ccc(C(c4ccc(-c5ccc6ccc7cccnc7c6n5)cc4)(c4ccc(-c5ccc6ccc7cccnc7c6n5)cc4)c4ccc(-c5ccc6ccc7cccnc7c6n5)cc4)cc3)nc12 ; c1cnc2c(c1)ccc1ccc(-c3ccc4c(c3)C3(c5cc(-c6ccc7ccc8cccnc8c7n6)ccc5-4)c4cc(-c5ccc6ccc7cccnc7c6n5)ccc4-c4ccc(-c5ccc6ccc7cccnc7c6n5)cc43)nc12 ; c1cnc2c(c1)ccc1ccc(-c3nc(-c4ccc5ccc6cccnc6c5n4)nc(-c4ccc5ccc6cccnc6c5n4)n3)nc12 ; c1cnc2c(c1)ccc1ccc(-c3nc(-c4ccc5ccc6cccnc6c5n4)nc(-c4ccc5ccc6cccnc6c5n4)n3)nc12]

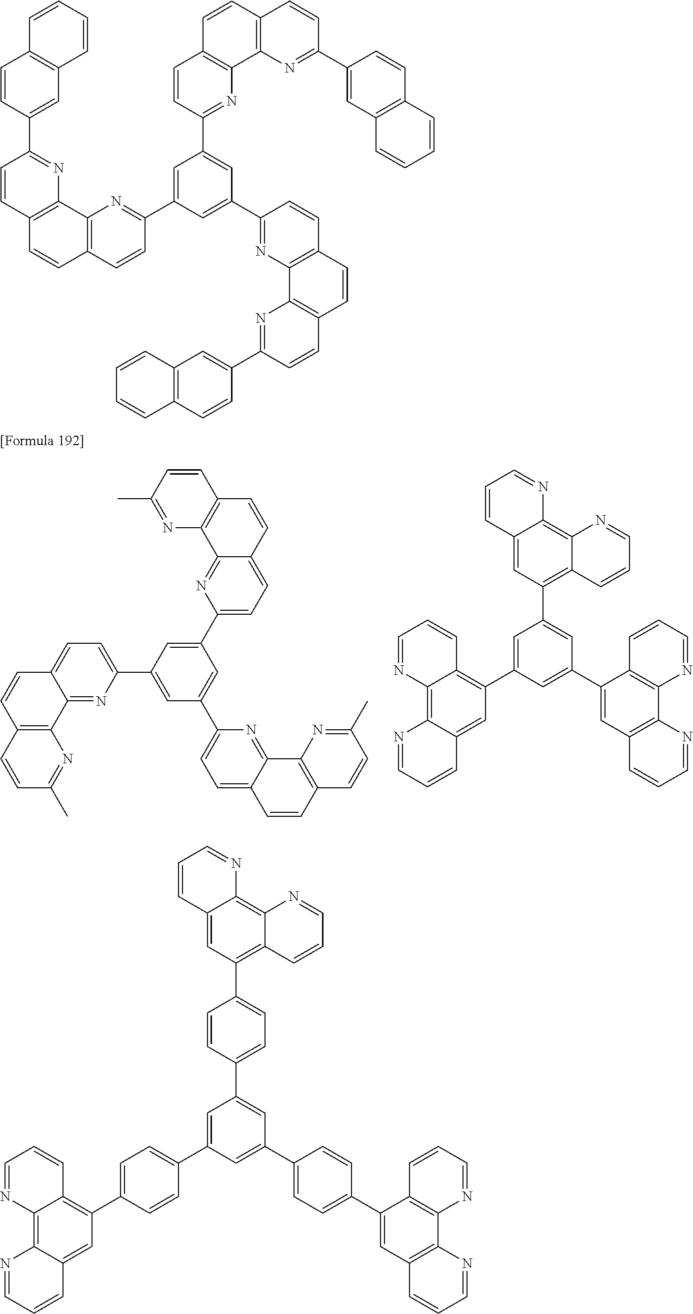

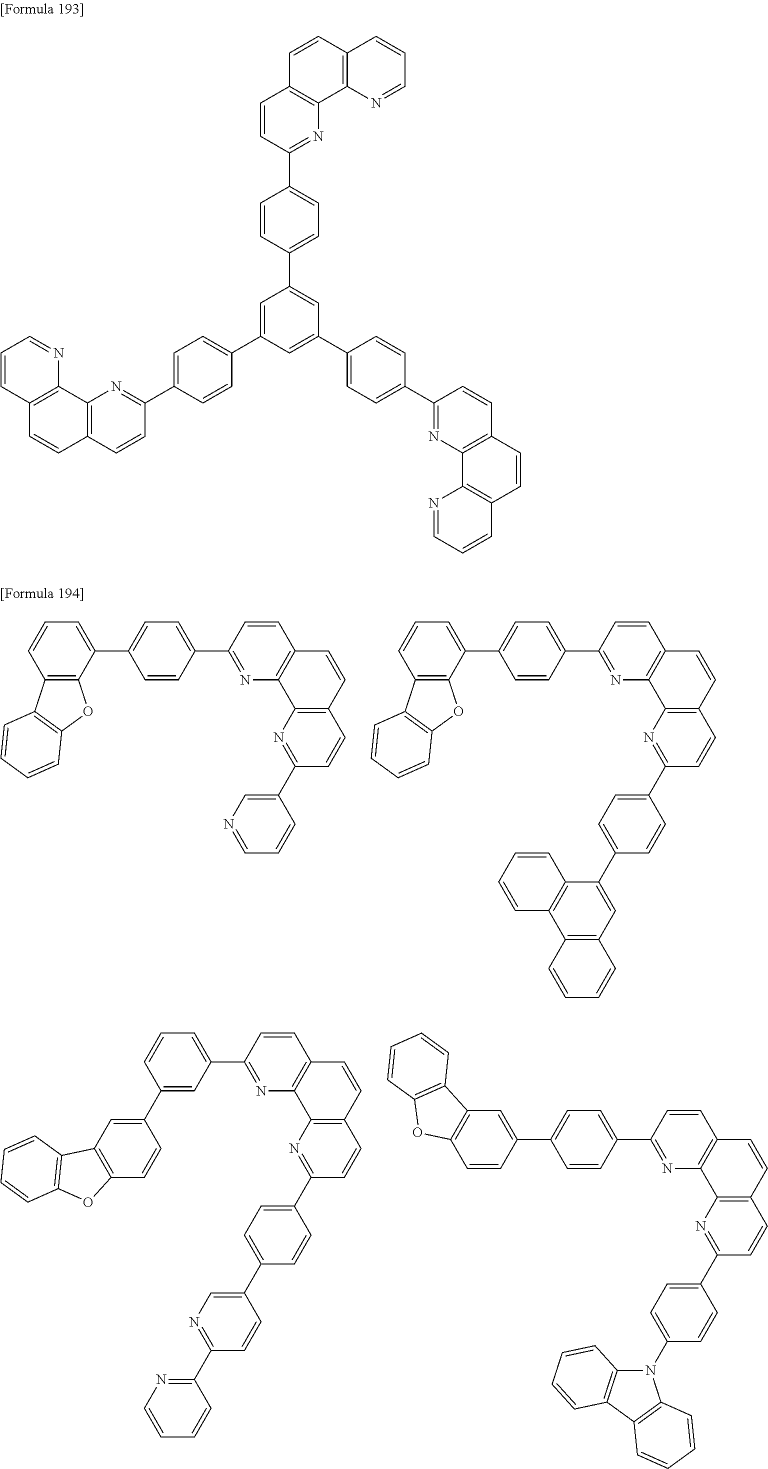

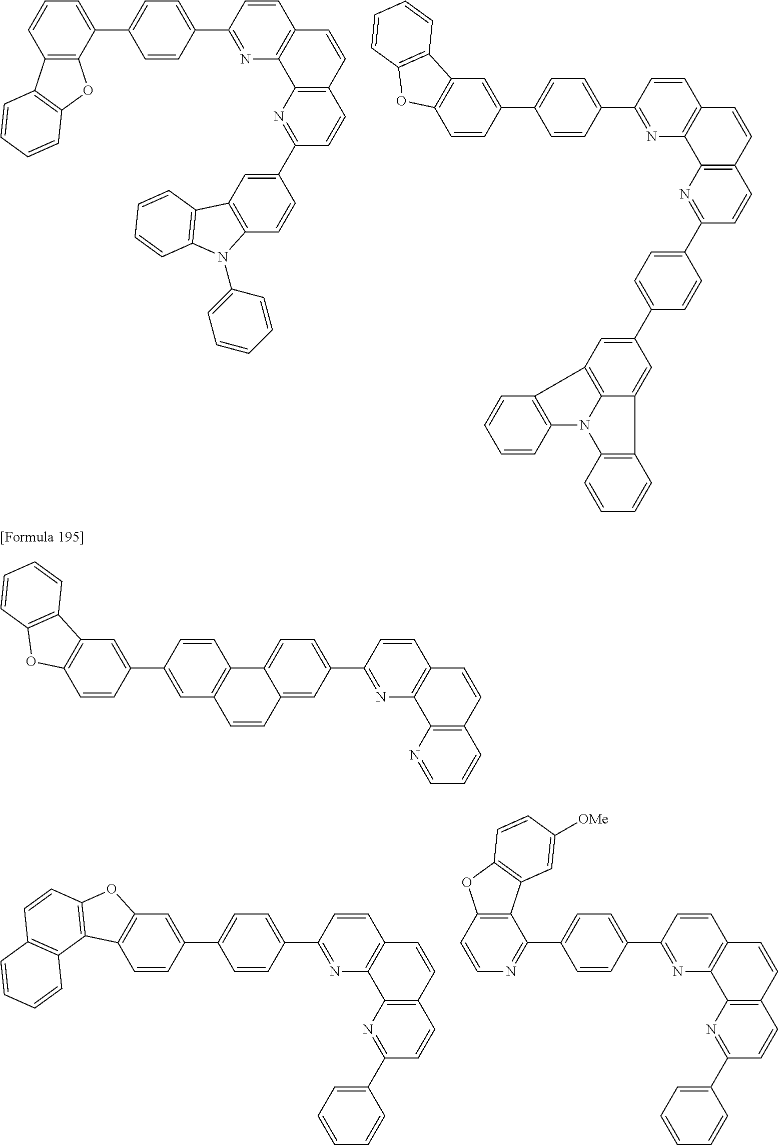

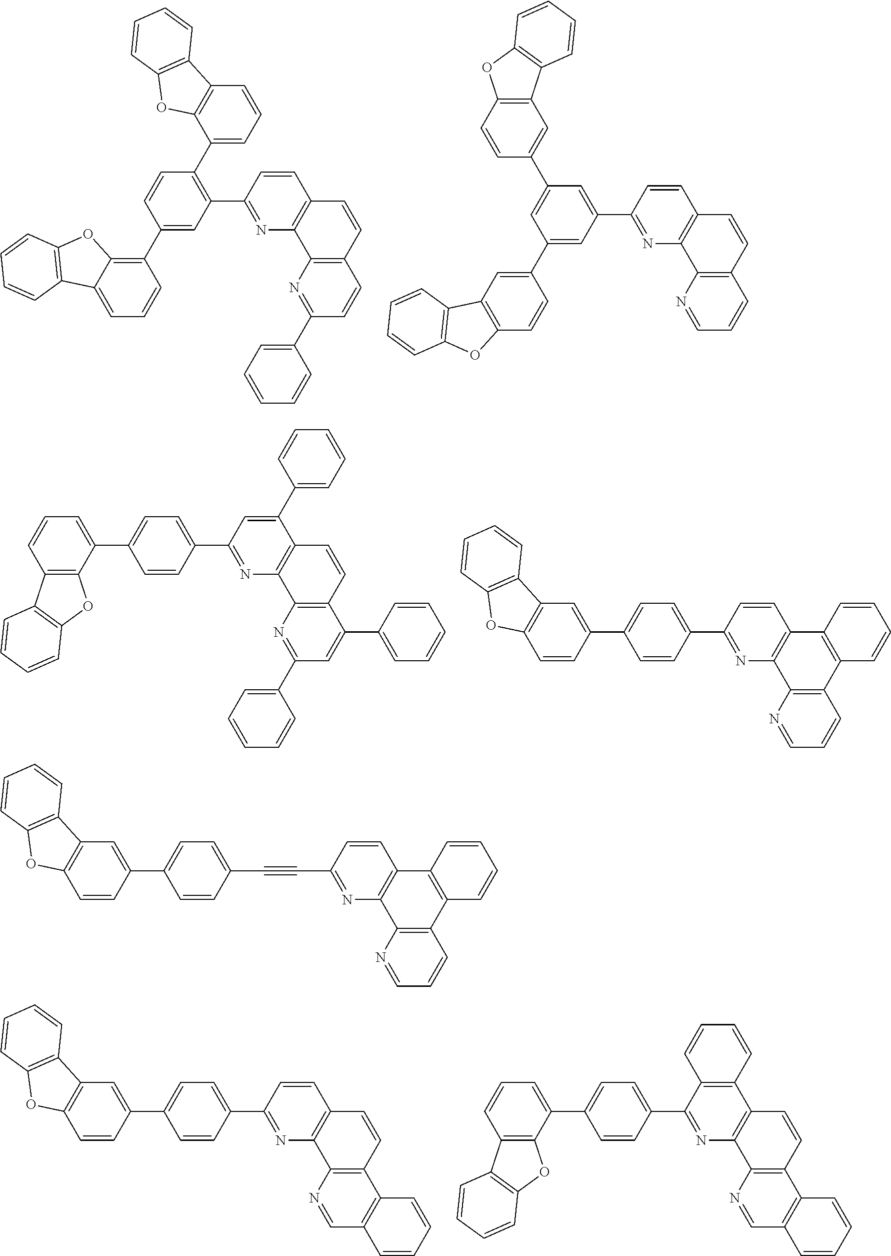

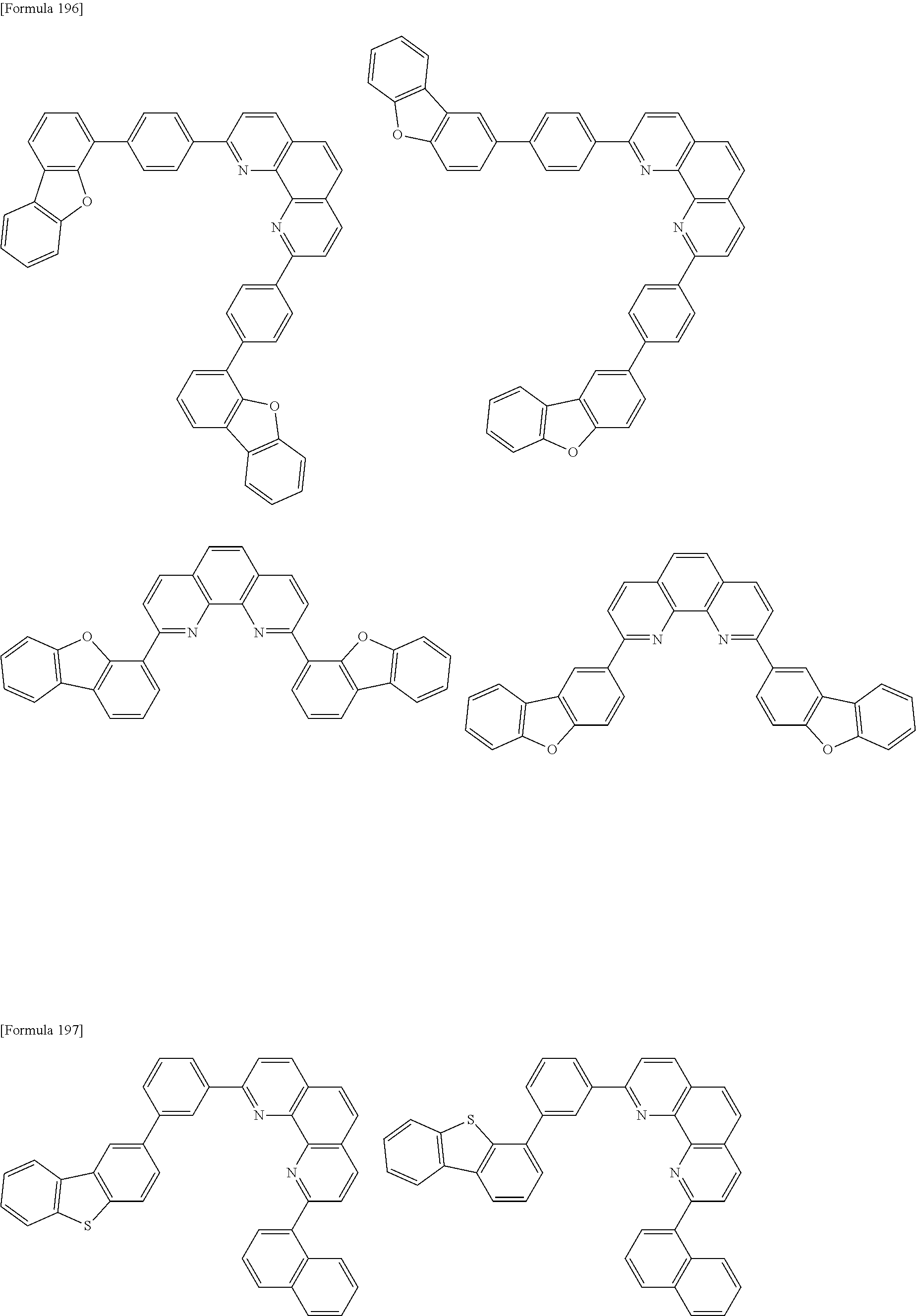

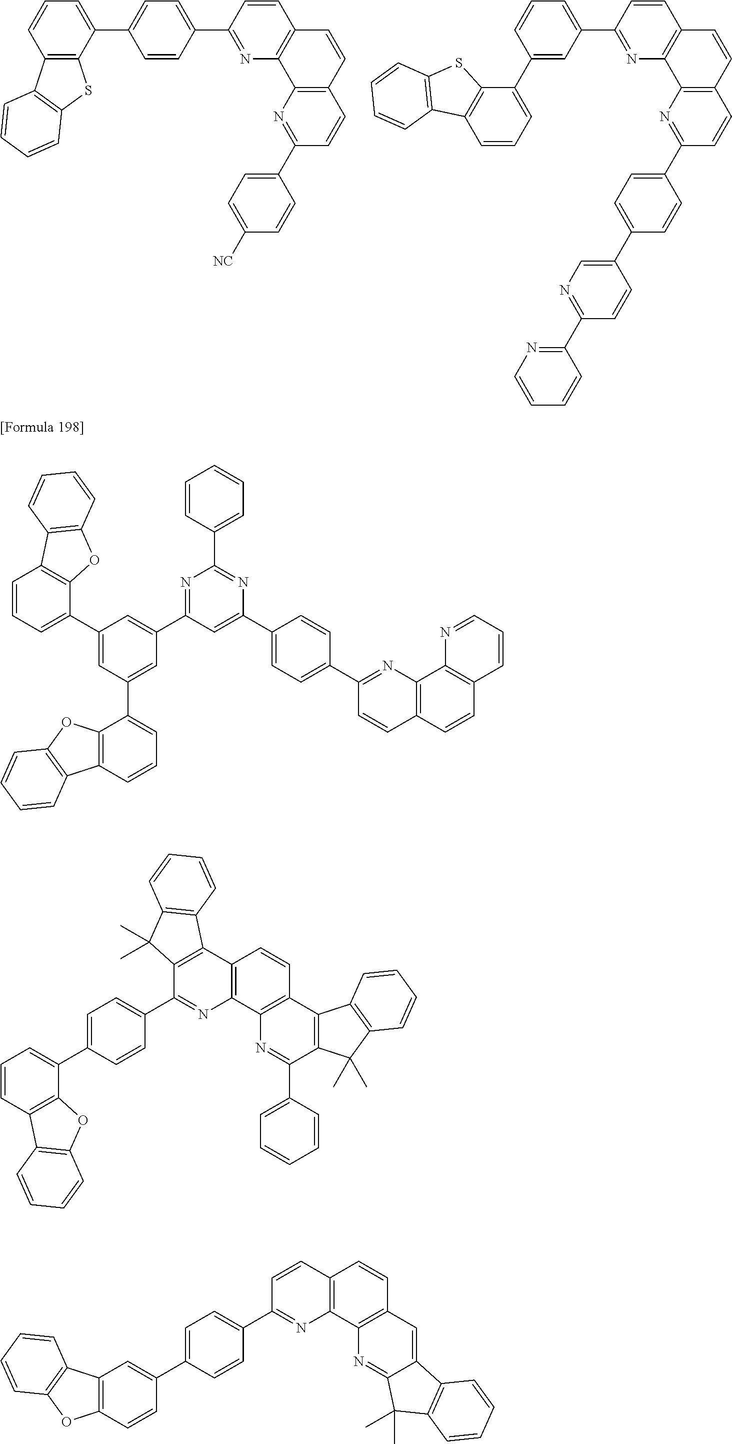

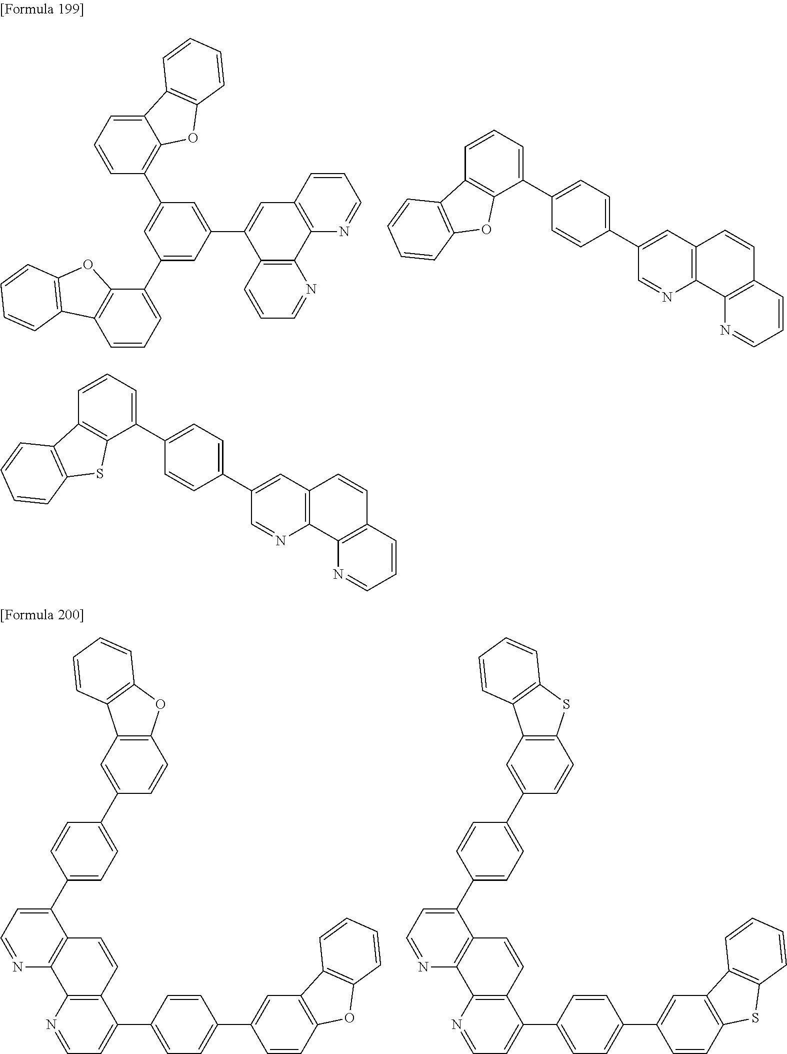

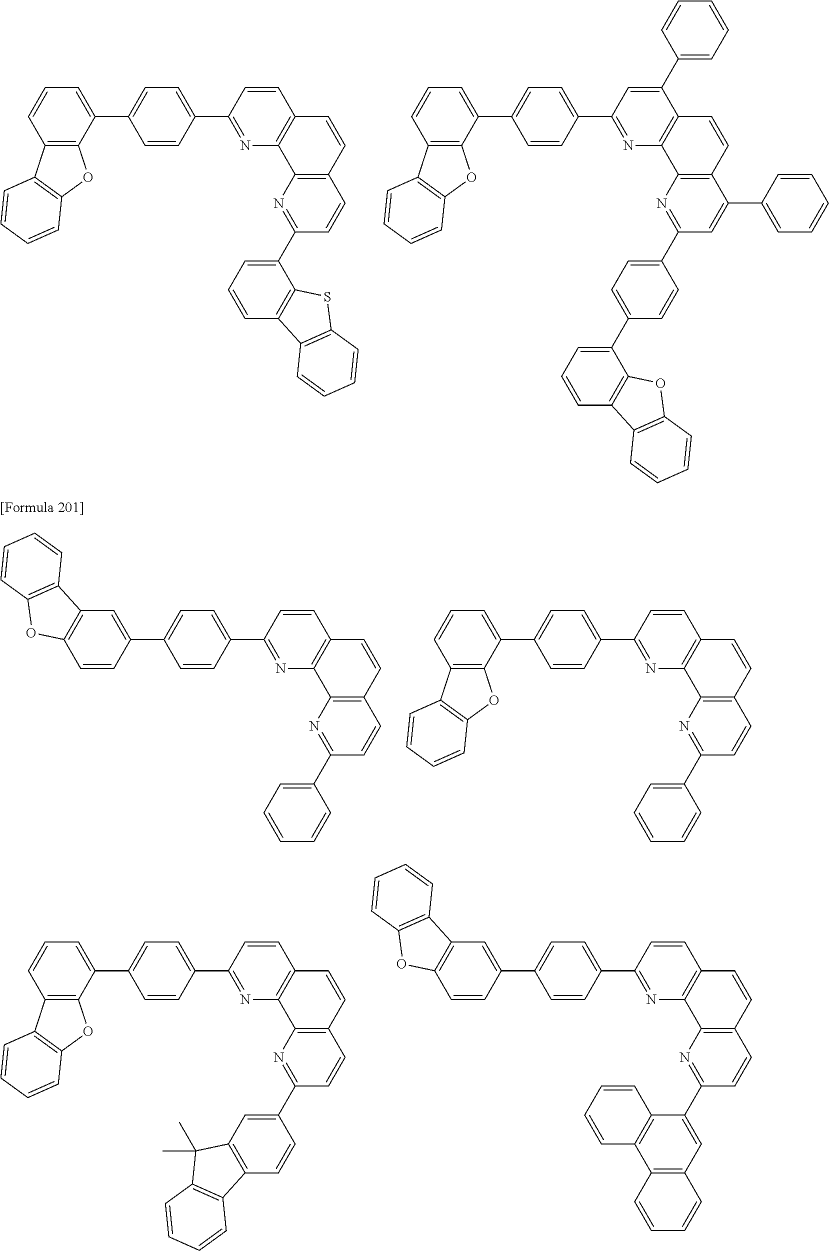

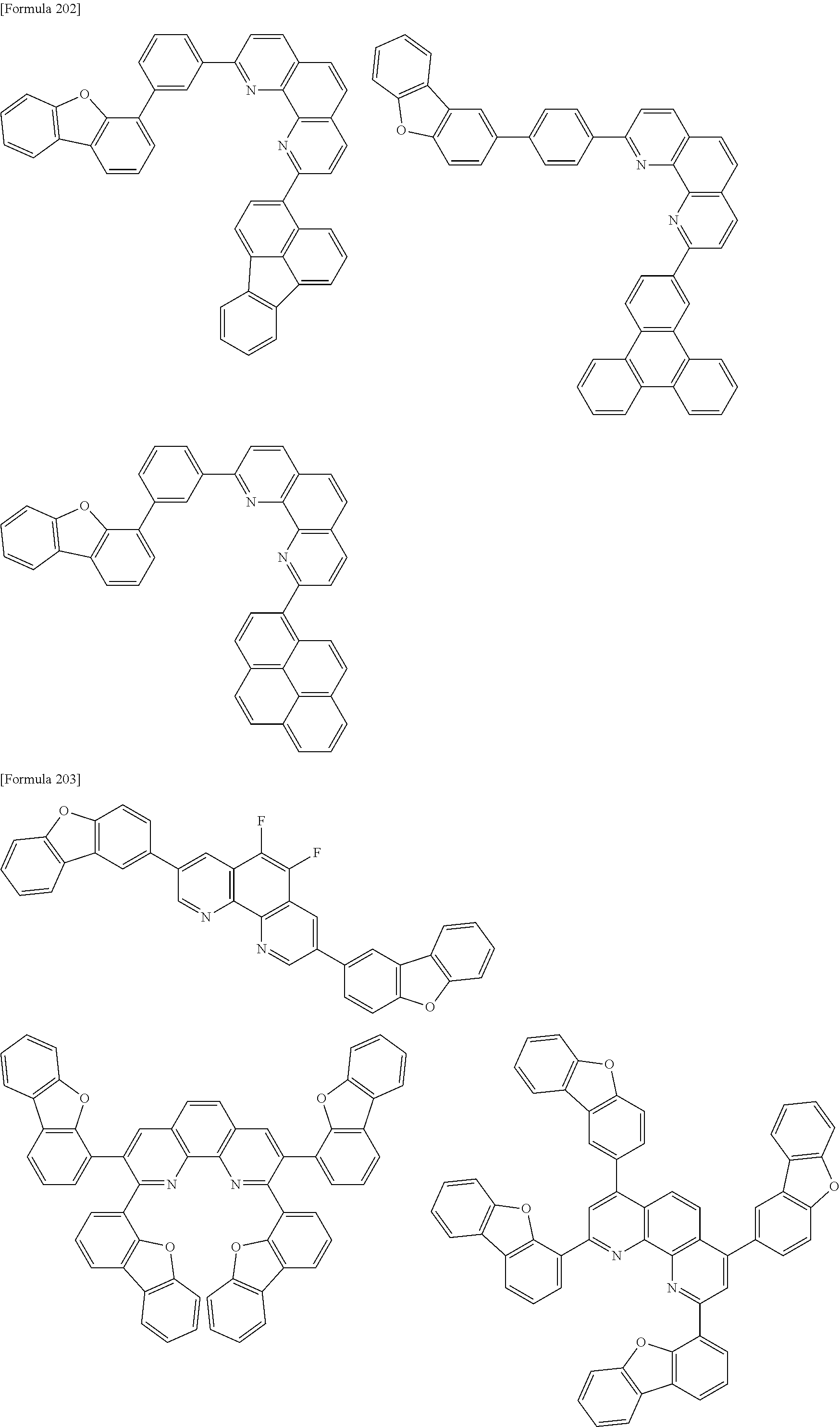

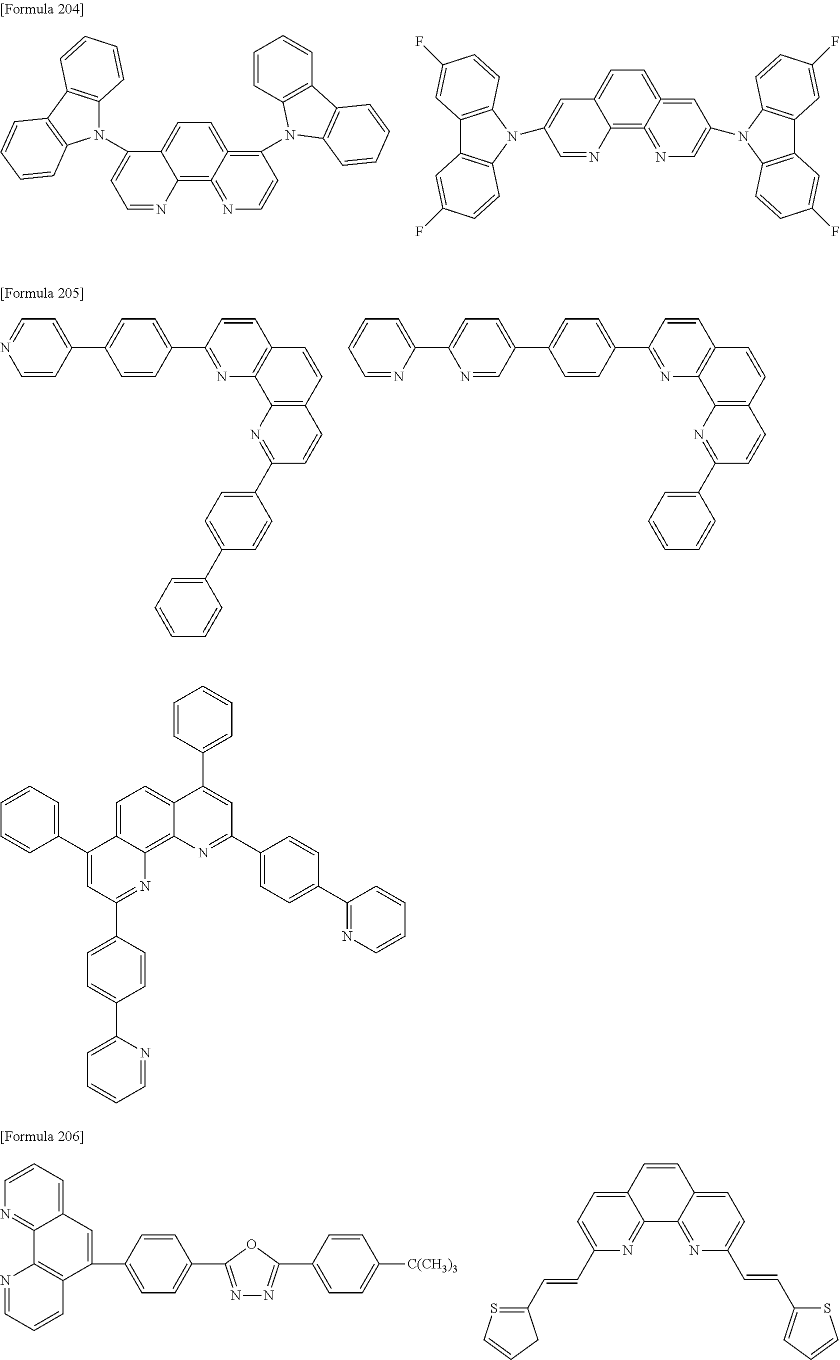

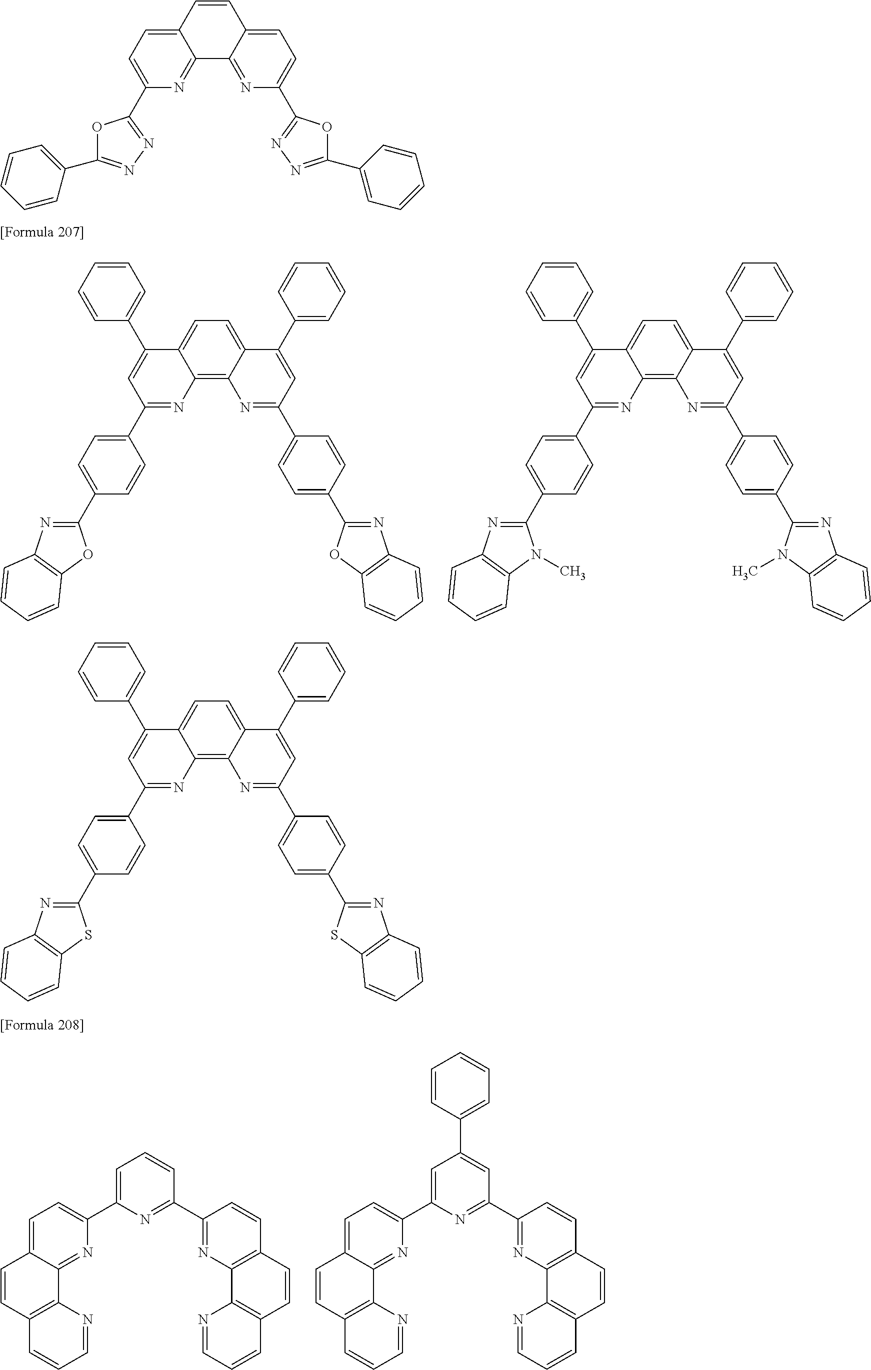

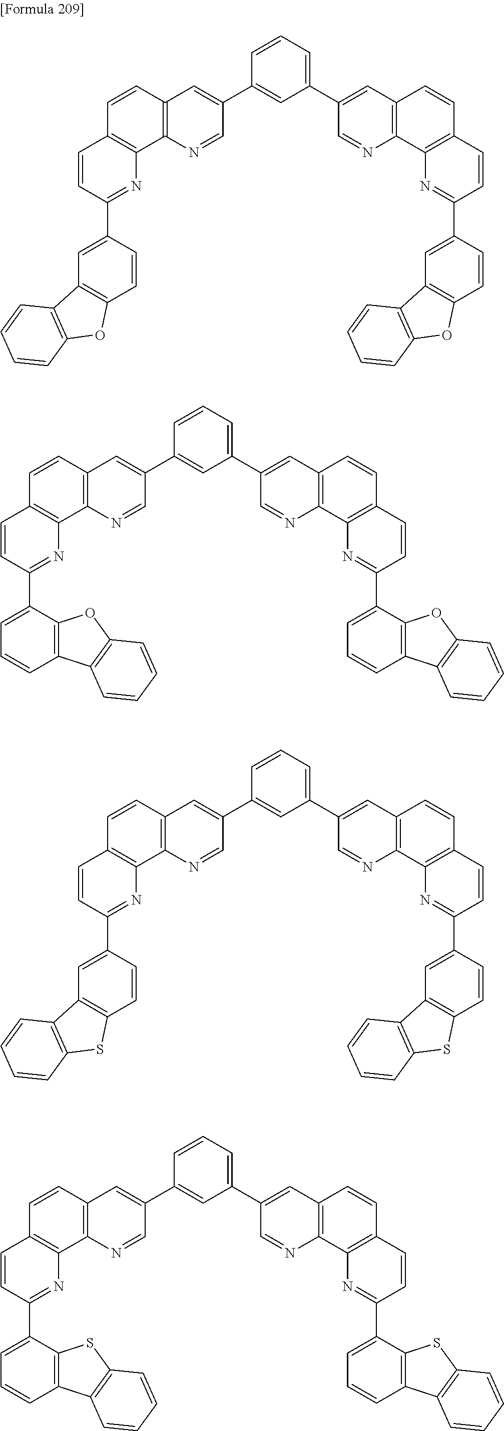 [SMILES: c1cc(-c2cnc3c(ccc4ccc(-c5ccc6oc7ccccc7c6c5)nc43)c2)cc(-c2cnc3c(ccc4ccc(-c5ccc6oc7ccccc7c6c5)nc43)c2)c1 ; c1cc(-c2cnc3c(ccc4ccc(-c5cccc6c5oc5ccccc56)nc43)c2)cc(-c2cnc3c(ccc4ccc(-c5cccc6c5oc5ccccc56)nc43)c2)c1 ; c1cc(-c2cnc3c(ccc4ccc(-c5ccc6sc7ccccc7c6c5)nc43)c2)cc(-c2cnc3c(ccc4ccc(-c5ccc6sc7ccccc7c6c5)nc43)c2)c1 ; c1cc(-c2cnc3c(ccc4ccc(-c5cccc6c5sc5ccccc56)nc43)c2)cc(-c2cnc3c(ccc4ccc(-c5cccc6c5sc5ccccc56)nc43)c2)c1]

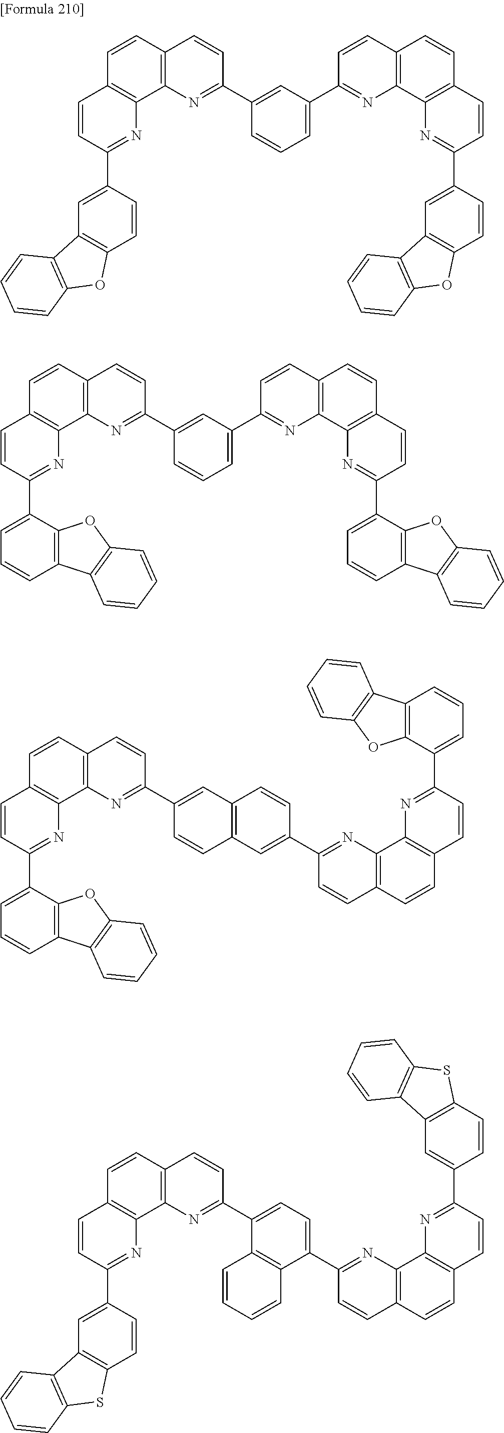 [SMILES: c1cc(-c2ccc3ccc4ccc(-c5ccc6oc7ccccc7c6c5)nc4c3n2)cc(-c2ccc3ccc4ccc(-c5ccc6oc7ccccc7c6c5)nc4c3n2)c1 ; c1cc(-c2ccc3ccc4ccc(-c5cccc6c5oc5ccccc56)nc4c3n2)cc(-c2ccc3ccc4ccc(-c5cccc6c5oc5ccccc56)nc4c3n2)c1 ; c1ccc2c(c1)oc1c(-c3ccc4ccc5ccc(-c6ccc7cc(-c8ccc9ccc%10ccc(-c%11cccc%12c%11oc%11ccccc%11%12)nc%10c9n8)ccc7c6)nc5c4n3)cccc12 ; c1ccc2c(c1)sc1ccc(-c3ccc4ccc5ccc(-c6ccc(-c7ccc8ccc9ccc(-c%10ccc%11sc%12ccccc%12c%11c%10)nc9c8n7)c7ccccc67)nc5c4n3)cc12]

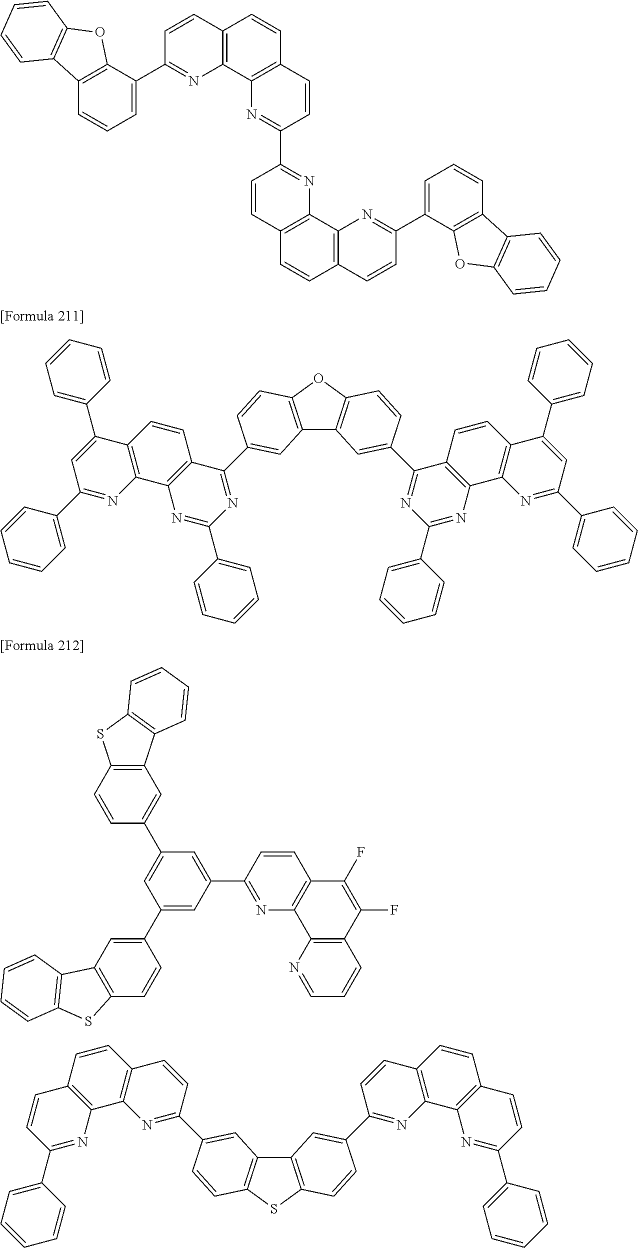

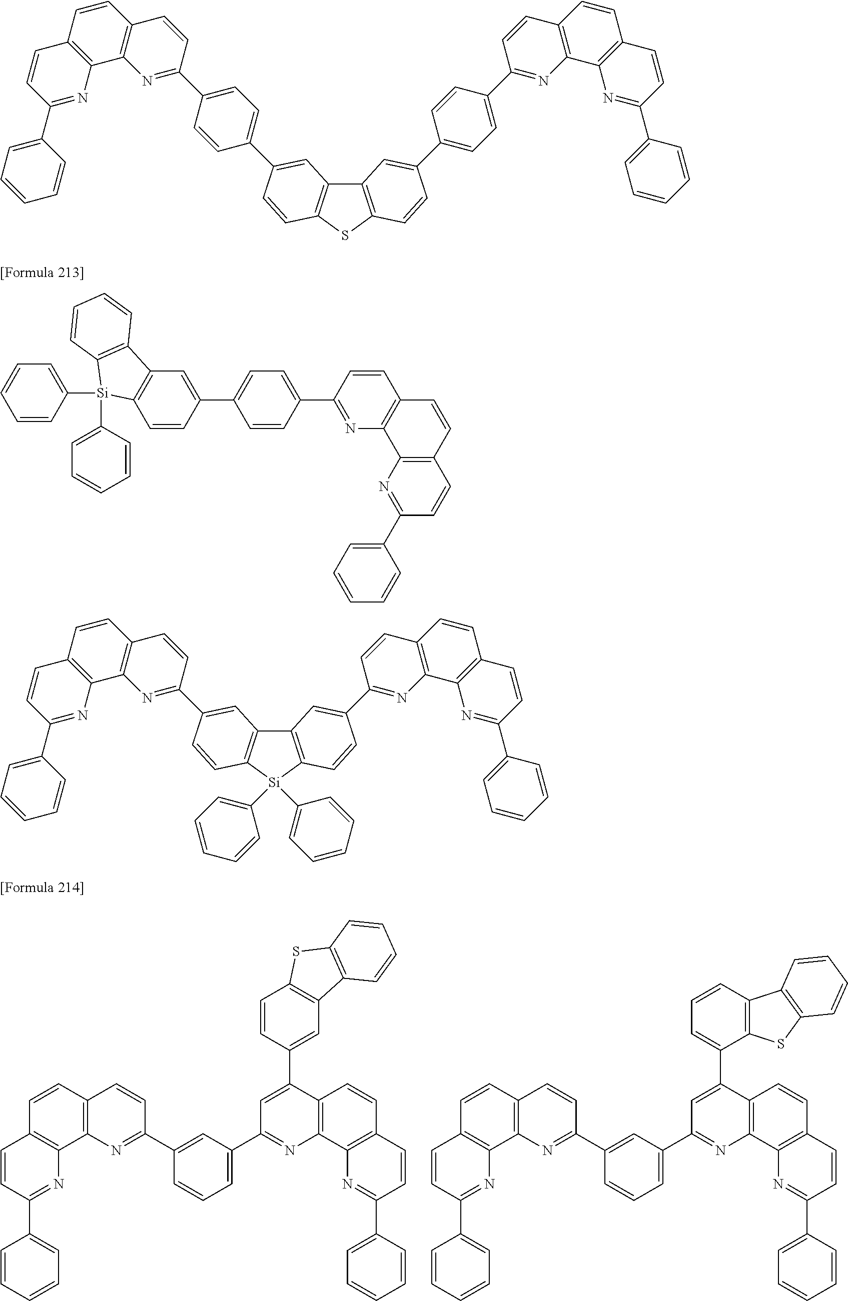

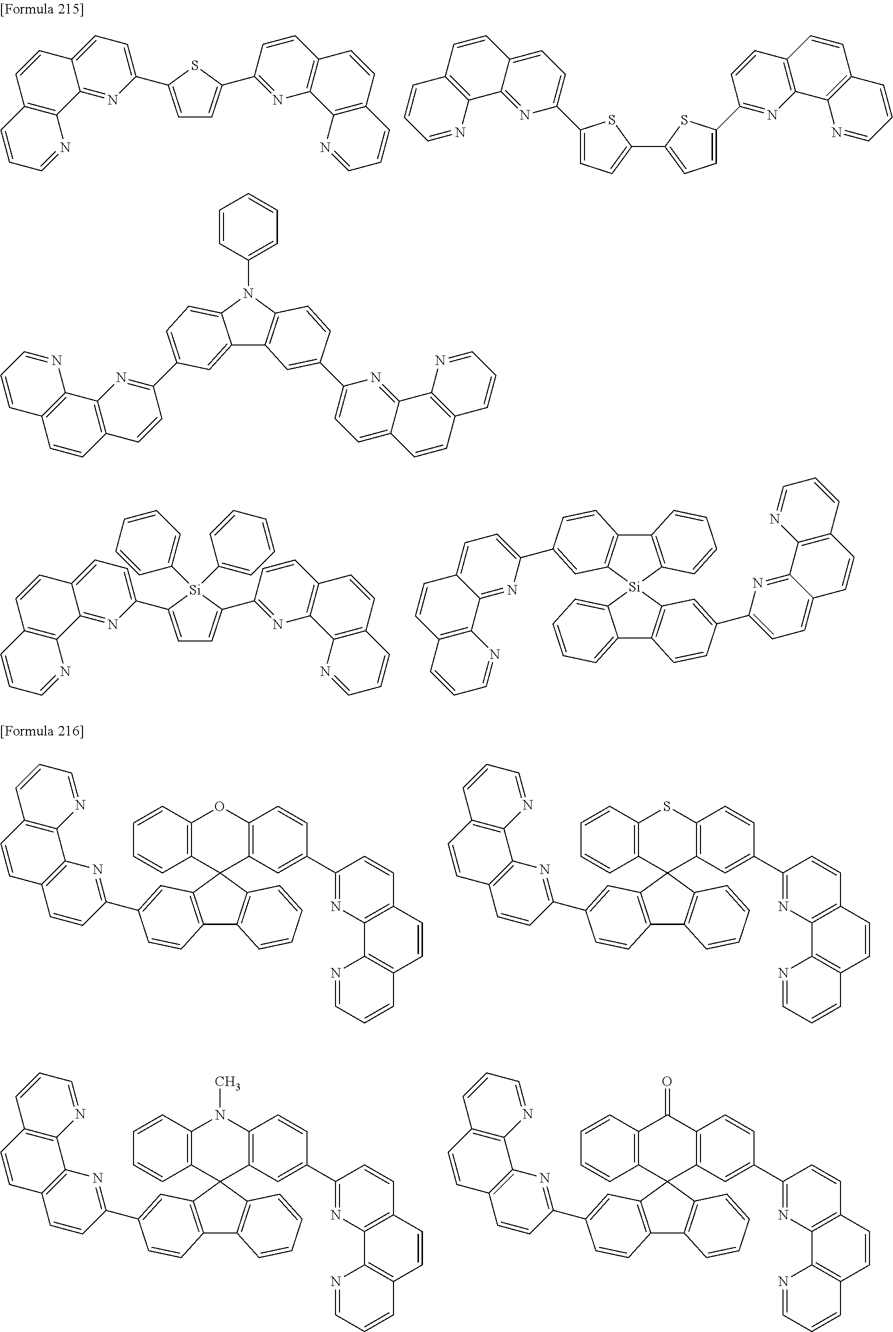

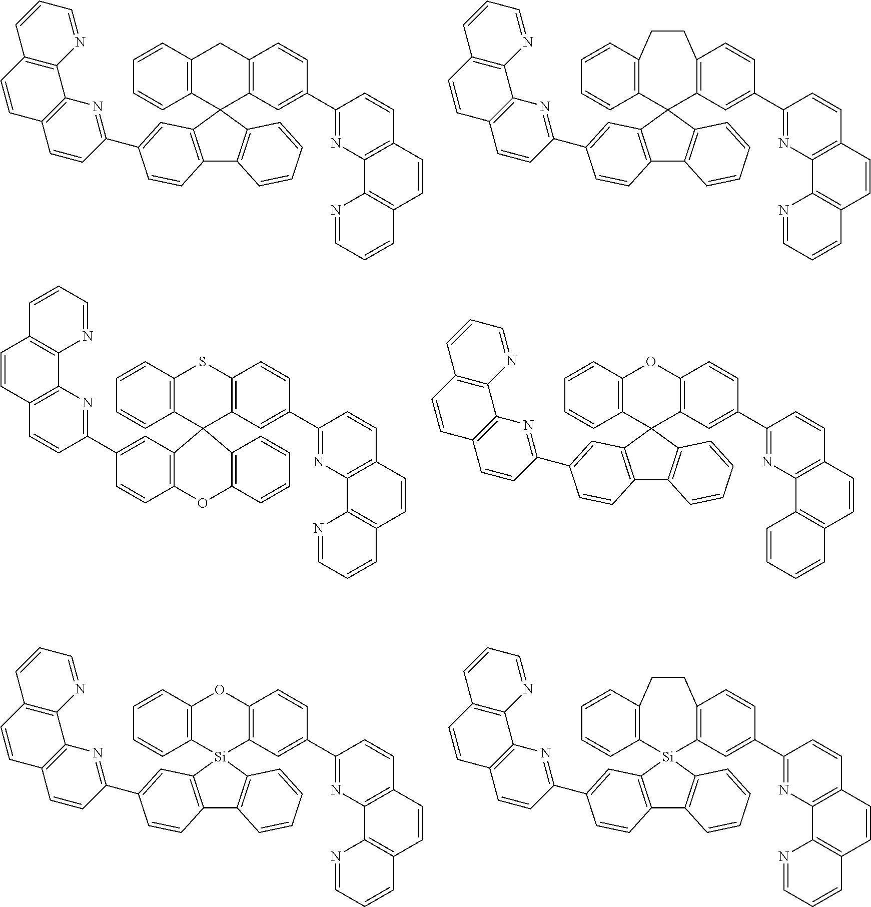

In the organic EL device 1 in the exemplary embodiment, the second electron transporting layer is preferably provided closer to the cathode than the first electron transporting layer is close to the cathode and is preferably not adjacent to the emitting layer.

It is also preferable that the second electron transporting layer contains at least one of an electron-donating dopant and an organic metal complex. A content of the electron-donating dopant or the organic metal complex in the electron transporting layer is preferably in a range of 1 mass % to 50 mass %.

The electron-donating dopant material is preferably at least one material selected from the group consisting of an alkali metal, alkali earth metal, rare earth metal, oxide of alkali metal, halide of alkali metal, oxide of alkali earth metal, halide of alkali earth metal, oxide of rare earth metal, and halide of rare earth metal.

The organic metal complex is preferably at least one selected from the group consisting of an organic metal complex including an alkali metal, organic metal complex including an alkali earth metal, and organic metal complex including a rare earth metal.

The electron-donating dopant and organic metal complex will be described in detail below.

With the organic EL device in the exemplary embodiment, a drive voltage can be decreased since the second electron transporting layer contains the compound represented by the formula (2). Moreover, since the second electron transporting layer contains the compound represented by the formula (2) and at least one of the electron-donating dopant and the organic metal complex, the electron-donating dopant and/or the organic metal complex contained in the second electron transporting layer are easily captured by a phenanthroline skeleton represented by the formula (2), so that the drive voltage is further decreased.

In the organic EL device in the exemplary embodiment, with the second electron transporting layer containing the compound represented by the formula (2), electron can be efficiently injected into the emitting layer. Moreover, a density of triplet excitons can be increased by the first electron transporting layer containing the compound represented by the formula (1) that is provided near the emitting layer, preferably adjacent to the emitting layer. The organic EL device in the exemplary embodiment includes two electron transporting layers that have respective functions for improving the electron injecting performance to the emitting layer and for trapping the triplet excitons in the emitting layer. With this arrangement, the organic EL device with a low voltage and a high efficiency are obtainable.

### Substrate

The organic EL device according to the exemplary embodiment is formed on a light-transmissive substrate. The light-transmissive plate, which supports the organic EL device, is preferably a smooth substrate that transmits 50% or more of light in a visible region of 400 nm to 700 nm. Specifically, examples of the substrate are a glass plate and a polymer plate.

### Anode and Cathode

The anode of the organic EL device is used for injecting holes into the hole injecting layer, the hole transporting layer or the emitting layer. It is effective that the anode has a work function of 4.5 eV or more. Specific examples of a material for the anode are alloys of indium-tin oxide (ITO), tin oxide (NESA), indium zinc oxide, gold, silver, platinum and copper.

The cathode is preferably formed of a material with smaller work function in order to inject electrons into the electron injecting layer, the electron transporting layer and the emitting layer. Although a material for the cathode is not particularly limited, examples of the material are indium, aluminium, magnesium, alloy of magnesium and indium, alloy of magnesium and aluminium, alloy of aluminium and lithium, alloy of aluminium, scandium and lithium, and alloy of magnesium and silver.

### Emitting Layer

The emitting layer of the organic EL device has a function for providing conditions for recombination of the electrons and the holes to emit light. The emitting layer is preferably a molecular deposit film. The molecular deposit film means a thin film formed by depositing a material compound in gas phase or a film formed by solidifying a material compound in a solution state or in liquid phase. The molecular deposit film is typically distinguished from a thin film formed by the LB (Langmuir Blodgett) method (i.e., molecular accumulation film) by differences in aggregation structures and higher order structures and functional differences arising therefrom.

### Dopant Material

The dopant material is selected from a known fluorescent material that emits fluorescence or a known phosphorescent material that emits phosphorescence.

### Host Material

As a host material, any host material applicable to the organic EL device is used. Examples of the host material include an amine derivative, azine derivative and fused polycyclic aromatic derivative.

Examples of the amine derivative are a monoamine compound, diamine compound, triamine compound, tetramine compound and amine compound substituted with a carbazole group.

Examples of the azine derivative include a monoazine derivative, diazine derivative and triazine derivative.

The fused polycyclic aromatic derivative is preferably a fused polycyclic aryl having no heterocyclic skeleton. Examples of the fused polycyclic aromatic derivative include the fused polycyclic aryl such as naphthalene, anthracene, phenanthrene, chrysene, fluoranthene and triphenylene, or derivatives thereof.

### Hole Injecting-Transporting Layer

The hole injecting-transporting layer helps injection of holes to the emitting layer and transports the holes to an emitting region. The hole injecting-transporting layer exhibits a large hole mobility and a small ionization energy.

A material for forming the hole injecting layer and the hole transporting layer is preferably a material for transporting the holes to the emitting layer at a lower electric field intensity. For instance, an aromatic amine compound is preferably used. A material for the hole injecting layer is preferably a porphyrin compound, an aromatic tertiary amine compound or a styryl amine compound, particularly preferably the aromatic tertiary amine compound such as hexacyanohexaazatriphenylene (HAT).

### Electron Injecting-Transporting Layer

The electron injection/transport layer helps injection of the electron to the luminescent layer and has a high electron mobility. The electron injecting layer is provided for adjusting energy level, by which, for instance, sudden changes of the energy level can be reduced.

In this exemplary embodiment, the electron injecting-transporting layer includes the first electron transporting layer and the second electron transporting layer. The first electron transporting layer contains the compound represented by the formula (1) and the second electron transporting layer contains the compound represented by the formula (2). In addition, an electron injecting layer may be provided and another electron transporting layer may be provided. When the electron injecting layer is provided, the first electron transporting layer, second electron transporting layer, and electron injecting layer may be laminated from the anode in this order. The electron injecting layer preferably contains a nitrogen-containing cyclic derivative as a main component. The electron injecting layer may serve as the electron transporting layer. Note that “as a main component” means that the nitrogen-containing cyclic derivative is contained in the electron injecting layer at a content of 50 mass % or more.

In the organic EL device in the exemplary embodiment, when three or more electron transporting layers are provided, it is preferable that the first electron transporting layer is provided near the emitting layer and the second electron transporting layer is provided closer to the cathode than the first electron transporting layer. In this arrangement, the first electron transporting layer is preferably near the second electron transporting layer, more preferably adjacent to the second electron transporting layer.

Moreover, the second electron transporting layer may contain an alkali metal as described above, or alternatively, a layer adjacent to a side of the second electron transporting layer closer to the cathode may contain an alkali metal. In addition to the alkali metal, the second electron transporting layer may contain an electron transporting material described below.

The electron transporting material for forming the electron transporting layer or the electron injecting layer is preferably an aromatic heterocyclic compound having at least one heteroatom in the molecule, particularly preferably a nitrogen-containing cyclic derivative. The nitrogen-containing cyclic derivative is preferably an aromatic ring having a nitrogen-containing six-membered or five-membered ring skeleton, or a fused aromatic cyclic compound having a nitrogen-containing six-membered or five-membered ring skeleton.

### Electron-Donating Dopant and Organic Metal Complex

In the organic EL device in the exemplary embodiment, it is also preferable that the second electron transporting layer and the layer adjacent to the second electron transporting layer and closer to the cathode contain at least one of the electron-donating dopant and the organic metal complex. With this arrangement, a decrease in the voltage applied on the organic EL device is enhanced. The electron-donating dopant may be at least one selected from an alkali metal, an alkali metal compound, an alkaline-earth metal, an alkaline-earth metal compound, a rare-earth metal, a rare-earth metal compound and the like.

The organic metal complex may be at least one compound selected from an organic metal complex including an alkali metal, an organic metal complex including an alkali earth metal, and an organic metal complex including rare-earth metal.

Examples of the alkali metal are lithium (Li) (work function: 2.93 eV), sodium (Na) (work function: 2.36 eV), potassium (K) (work function: 2.28 eV), rubidium (Rb) (work function: 2.16 eV) and cesium (Cs) (work function: 1.95 eV), among which a substance having a work function of 2.9 eV or less is particularly preferable. Among the above, the alkali metal is preferably K, Rb or Cs, more preferably Rb or Cs, the most preferably Cs.

Examples of the alkali earth metal are calcium (Ca) (work function: 2.9 eV), strontium (Sr) (work function: 2.0 to 2.5 eV), and barium (Ba) (work function: 2.52 eV), among which a substance having a work function of 2.9 eV or less is particularly preferable.

Examples of the rare-earth metal are scandium (Sc), yttrium (Y), cerium (Ce), terbium (Tb) and ytterbium (Yb), among which a substance having a work function of 2.9 eV or less is particularly preferable.

Since the above preferred metals have particularly high reducibility, addition of a relatively small amount of the metals to an electron injecting zone can enhance luminance intensity and lifetime of the organic EL device.

Examples of the alkali metal compound are an alkali oxide such as lithium oxide (Li₂O), cesium oxide (Cs₂O) and potassium oxide (K₂O), and an alkali halogenide such as sodium fluoride (NaF), cesium fluoride (CsF) and potassium fluoride (KF), among which lithium fluoride (LiF), lithium oxide (Li₂O) and sodium fluoride (NaF) are preferable.

Examples of the alkali earth metal compound are barium oxide (BaO), strontium oxide (SrO), calcium oxide (CaO) and a mixture thereof, i.e., BaₓSr₁₋ₓO (0<x<1), BaₓCa₁₋ₓ O (0<x<1), among which BaO, SrO and CaO are preferable.

Examples of the rare-earth metal compound are ytterbium fluoride (YbF₃), scandium fluoride (ScF₃), scandium oxide (ScO₃), yttrium oxide (Y₂O₃), cerium oxide (Ce₂O₃), gadolinium fluoride (GdF₃) and terbium fluoride (TbF₃), among which YbF₃, ScF₃ and TbF₃ are preferable.

The organic metal complex is not particularly limited, as long as the organic metal complex contains at least one of an alkali metal ion, alkali earth metal ion and rare-earth metal ion, as a metal ion. The ligand for each of the complexes is preferably quinolinol, benzoquinolinol, acridinol, phenanthridinol, hydroxyphenyl oxazole, hydroxyphenyl thiazole, hydroxydiaryl oxadiazole, hydroxydiaryl thiadiazole, hydroxyphenyl pyridine, hydroxyphenyl benzoimidazole, hydroxybenzo triazole, hydroxy fluborane, bipyridyl, phenanthroline, phthalocyanine, porphyrin, cyclopentadiene, p-diketones, azomethines, or a derivative thereof, but the ligand is not limited thereto.

Among the electron-donating dopant and the organic metal complex, lithium (Li) or lithium fluoride (LiF) is preferable. When at least one of the electron-donating dopant and the organic metal complex is contained in the second electron transporting layer, lithium (Li) is particularly preferable. When at least one of the electron-donating dopant and the organic metal complex is contained in the layer adjacent to the second electron transporting layer near the cathode, lithium fluoride (LiF) is particularly preferable.

A preferable method for adding the electron-donating dopant and the organic metal complex includes dispersing at least one of the electron-donating dopant and the organic metal complex in the electron transporting layer while co-depositing at least one of the electron-donating dopant and the organic metal complex with the compound represented by the formula (1) by resistance heating deposition. A dispersion concentration is shown by a film thickness ratio of the compound represented by the formula (1) to the electron-donating dopant and/or the organic metal complex being of 1000:1 to 1:1000, preferably 100:1 to 1:1.

When at least one of the electron-donating dopant and the organic metal complex forms a layer, after forming a layer of the compound represented by the formula (2), at least one of the electron-donating dopant and the organic metal complex is singularly deposited on the compound layer by resistance heating deposition to preferably form a 0.1 nm- to 15 nm-thick layer.

When at least one of the electron-donating dopant and the organic metal complex forms an island pattern, after forming an island pattern of the compound represented by the formula (2), at least one of the electron-donating dopant and the organic metal complex is singularly deposited on the compound layer by resistance heating deposition to preferably form a 0.05 nm- to 1 nm-thick island pattern.

A ratio of the compound represented by the formula (2) to at least one of the electron-donating dopant and the organic metal complex in the organic EL device according to the exemplary embodiment is preferably shown by a film thickness ratio of the main component to the electron-donating dopant and/or the organic metal complex being 100:1 to 1:1, more preferably 50:1 to 4:1.

### Manufacturing Method of Each Layer of Organic EL Device

A method of forming each of the layers in the organic EL device according to the exemplary embodiment is not particularly limited. Conventionally-known methods such as vacuum deposition and spin coating may be employed for forming the layers. The organic layer, which is used in the organic EL device of the exemplary embodiment, may be formed by a known method such as vacuum deposition, molecular beam epitaxy (MBE (Molecular Beam Epitaxy) method) or coating methods using a solution such as a dipping, spin coating, casting, bar coating, and roll coating.

### Film Thickness of Each Layer of Organic EL Device

A film thickness of the emitting layer is preferably in a range of 5 nm to 50 nm, more preferably in a range of 7 nm to 50 nm and most preferably in a range of 10 nm to 50 nm. By forming the emitting layer at the film thickness of 5 nm or more, the emitting layer is easily formable and chromaticity is easily adjustable. By forming the emitting layer at the film thickness of 50 nm or less, an increase in the drive voltage is suppressible.

Although the film thickness of each of other organic layers is not specifically limited, the film thickness is typically preferably in a range of several nm to 1 μm. When the film thickness is provided in the above range, defects such as pin holes caused by an excessively thinned film can be avoided while an increase in the drive voltage caused by an excessively thickened film can be suppressed.

### Electronic Device

The organic EL device of the invention is suitably applicable to an electronic device such as: an organic EL panel module; a display of a television, a mobile phone, a personal computer and the like; and an emitting unit of an illuminator or a vehicle light.

### Second Exemplary Embodiment

Next, a second exemplary embodiment is described below.

In the description of the second exemplary embodiment, the same components as those in the first exemplary embodiment are denoted by the same reference signs and names to simplify or omit an explanation of the components. In the second exemplary embodiment, the same materials and compounds as described in the first exemplary embodiment are usable.

An organic EL device according to the second exemplary embodiment is a so-called tandem-type device including a charge generating layer (as an intermediate layer) and at least two emitting units. In addition to charges injected from a pair of electrodes, charges supplied from the charge generating layer are injected into the emitting units. Accordingly, by providing the charge generating layer, luminous efficiency (current efficiency) relative to injected current is improved.

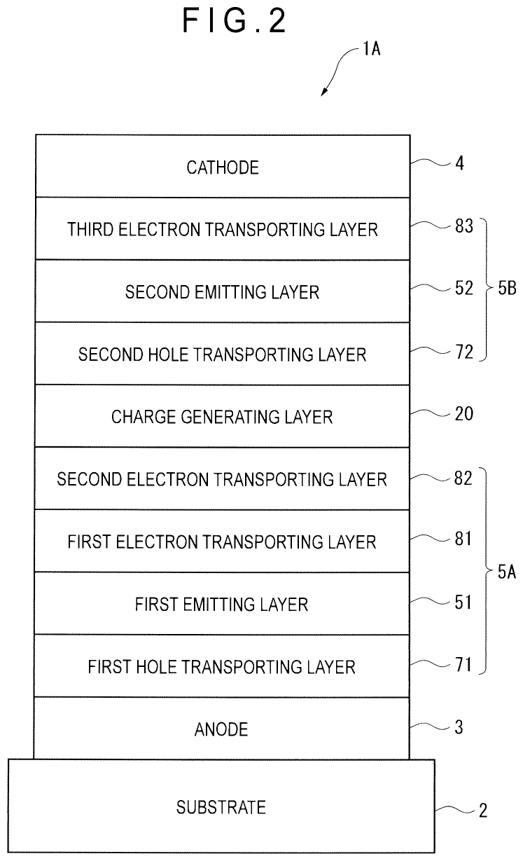

As shown in FIG. 2, an organic EL device 1A according to the second exemplary embodiment is provided by laminating an anode 3, a first emitting unit 5A, a charge generating layer 20, a second emitting unit 5B and a cathode 4 on a substrate 2 in this sequence.

The first emitting unit 5A is provided by laminating a first hole transporting layer 71, first emitting layer 51, a first electron transporting layer 81 and a second transporting layer 82 from the anode 3 in this order.

The second emitting unit 5B is provided by laminating a second hole transporting layer 72, second emitting layer 52, and a third electron transporting layer 83 from the charge generating layer 20 in this order.

The charge generating layer 20 is a layer in which charges are generated when an electrical field is applied to the organic EL device 1A. The charge generating layer 20 injects electrons into the second electron transporting layer 82 and injects holes into the second hole transporting layer 72.

As a material for the charge generating layer 20, a known material such as a material described in U.S. Pat. No. 7,358,661 is usable. Specific examples of the material include oxides, nitrides, iodides and borides of metals such as In, Sn, Zn, Ti, Zr, Hf, V, Mo, Cu, Ga, Sr, La and Ru. The second electron transporting layer 82 near an interface with the charge generating layer is preferably doped with a donor (e.g., the above-described alkali metal) in order that the third emitting layer 51 can easily accept electrons from the charge generating layer 20.

The second hole transporting layer 72 and the third electron transporting layer 83 are respectively the same as the hole transporting layer 7 and the first electron transporting layer 81 in the first exemplary embodiment.

Since the organic EL device 1A is a so-called tandem-type device, the drive voltage can be reduced and durability can also be improved.

### Modifications of Embodiment(s)

It should be noted that the invention is not limited to the above exemplary embodiment but may include any modification and improvement as long as such modification and improvement are compatible with the invention.

Although a single layer of the emitting layer is provided in the first exemplary embodiment, the emitting layer is not limited to a single layer, but may be provided by laminating a plurality of emitting layers. When the organic EL device includes the plurality of emitting layers, the plurality of emitting layers may be each independently a fluorescent emitting layer or a phosphorescent emitting layer.

It is also preferable that the organic EL device contains at least one of the electron-donating dopant and the organic metal complex in an interfacial region between the cathode and the organic layer. With this arrangement, the organic EL device can emit light with enhanced luminance intensity and have a longer lifetime. Examples of the electron-donating dopant and the organic metal complex are the same as described above.

The electron-donating dopant and the organic metal complex are added to preferably form a layer or an island pattern in the interfacial region. The layer of the electron-donating dopant or the island pattern of the organic metal complex is preferably formed by: depositing at least one of the electron-donating dopant and the organic metal complex while simultaneously depositing an organic substance that is an emitting material or an electron-injecting material for forming the interfacial region, by resistance heating deposition; and dispersing at least one of the electron-donating dopant and an organic metal complex reduction-causing dopant in the organic substance.

In the invention, it is also preferable that the emitting layer contains an assistance material for assisting injection of charges. When the emitting layer is formed of a host material that exhibits a wide energy gap, a difference in ionization potential (Ip) between the host material and the hole injecting/transporting layer etc. becomes so large that injection of the holes into the emitting layer becomes difficult, which may cause a rise in a driving voltage required for providing sufficient luminance. In the above instance, introducing a hole-injectable or hole-transportable assistance substance for assisting injection of charges in the emitting layer can contribute to facilitation of the injection of the holes into the emitting layer and to reduction of the driving voltage.

As the assistance substance for assisting the injection of charges, for instance, a general hole injecting material, a general hole transporting material or the like can be used.

Specific examples of the material for assisting the injection of charges are a triazole derivative, oxadiazole derivative, imidazoles derivative, polyarylalkane derivative, pyrazoline derivative, pyrazolone derivative, phenylenediamine derivative, arylamine derivative, amino-substituted chalcone derivative, oxazole derivative, fluorenone derivative, hydrazone derivative, stilbene derivative, silazane derivative, polysilane copolymer, aniline copolymer, and conductive polymer oligomer (particularly, a thiophene oligomer).

The hole injecting material is exemplified by the above. The hole injecting material is preferably a porphyrin compound, aromatic tertiary amine compound and styryl amine compound, particularly preferably aromatic tertiary amine compound.

In addition, 4,4′-bis(N-(1-naphthyl)-N-phenylamino)biphenyl (hereinafter, abbreviated as NPD) having two fused aromatic rings in a molecule, or 4,4′,4″-tris(N-(3-methylphenyl)-N-phenylamino)triphenylamine (hereinafter, abbreviated as MTDATA) in which three triphenylamine units are bonded in a starburst form as disclosed and the like may also be used.

Moreover, a hexaazatriphenylene derivative and the like may be also preferably used as the hole injecting material.

Alternatively, inorganic compounds such as p-type Si and p-type SiC may also be used as the hole-injecting material.

### EXAMPLES

Next, the invention will be described in further detail by exemplifying Example(s) and Comparative(s). However, the invention is not limited by the description of Example(s).

### Synthesis of Compound(s)

### Synthesis Example 1: Synthesis of Compound 4

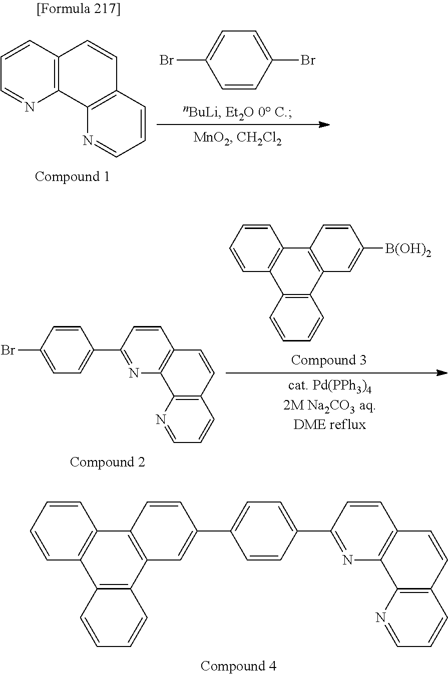 [SMILES: c1cnc2c(c1)ccc1cccnc12 ; Brc1ccc(Br)cc1 ; Brc1ccc(-c2ccc3ccc4cccnc4c3n2)cc1 ; OB(O)c1ccc2c3ccccc3c3ccccc3c2c1 ; c1cnc2c(c1)ccc1ccc(-c3ccc(-c4ccc5c6ccccc6c6ccccc6c5c4)cc3)nc12]

### (1-1) Synthesis of Compound 2

Under an argon gas atmosphere, 1,4-dibromobenzene (50 g, 211 mmol) was dissolved in diethylether (350 mL). After the obtained solution was cooled to 0 degrees C., n-butyllithium (2.69 M hexane solution) (72 mL, 194 mmol) was dropped into the cooled solution for 30 minutes. The obtained mixture solution was further stirred for 30 minutes. The prepared p-bromophenyl lithium was dropped at 0 degrees C. for 45 minutes in a suspension liquid in which 1,10-phenanthroline (15 g, 85 mmol) was suspended in diethylether (350 mL). The obtained mixture solution was further stirred for five hours. After the completion of the reaction, water was dropped at 0 degrees C. for 30 minutes in the obtained reaction solution. The reaction solution was extracted with dichloromethane. A solvent except for 200 mL of dichloromethane was distilled under reduced pressure. Manganese dioxide (150 g) was added to the obtained solution and stirred at the room temperature for 4.5 hours. Subsequently, magnesium sulfate was added to the reaction solution. The obtained deposit was subjected to filtration and a solvent was distilled under reduced pressure. A residue was purified by silica-gel column chromatography (dichloromethane/hexane/methanol). The obtained solid was washed with methanol and dried under reduced pressure, so that a compound 2 (23 g, a yield of 81%) was obtained as a white solid. By FD-MS (Field Desorption Mass Spectrometry) analysis, the obtained compound was identified to be the compound 2.

### (1-2) Synthesis of Compound 4

The compound 2 (5.0 g, 15 mmol) and a compound 3 (4.8 g, 18 mmol) were suspended in 1,2-dimethoxyethane (200 mL), to which tetrakis(triphenylphosphine)palladium(0) (0.9 g, 0.75 mmol) and an aqueous solution of 2M sodium carbonate (30 mL) were added. The obtained solution was heated to reflux for seven hours. After the completion of the reaction, water was added to the reaction solution and the reaction solution was filtrated to obtain a solid. The obtained solid was washed with water and methanol and dried under reduced pressure. The obtained crude product was purified by silica-gel column chromatography (dichloromethane). The obtained solid was washed with methanol and dried under reduced pressure, so that a compound 4 (5.4 g, a yield of 75%) was obtained as a light yellow solid. By FD-MS (Field Desorption Mass Spectrometry) analysis, the obtained compound was identified to be the compound 4.

### Synthesis Example 2: Synthesis of Compound 6

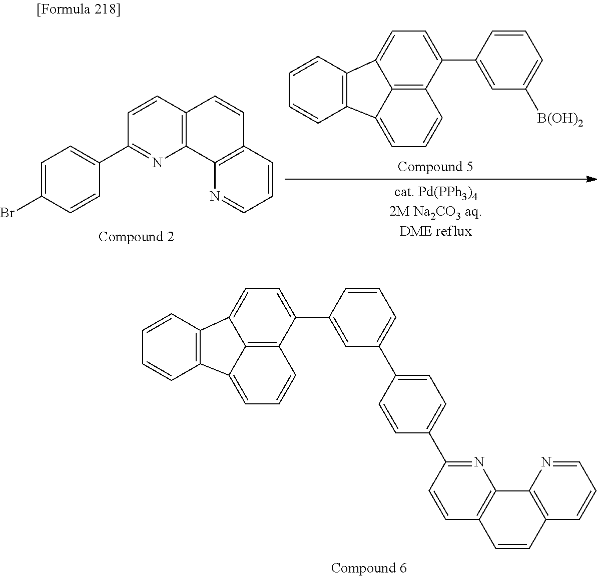

### (2-1) Synthesis of Compound 6

A compound 6 (6.6 g, a yield of 83%) was obtained as a light-yellow solid according to the same method as in (1-2) synthesis of the compound 4, except for using the compound 5 (5.8 g, 18 mmol) in place of the compound 3. By FD-MS (Field Desorption Mass Spectrometry) analysis, the obtained compound was identified to be the compound 6.

### Synthesis Example 3. Synthesis of Compound 8

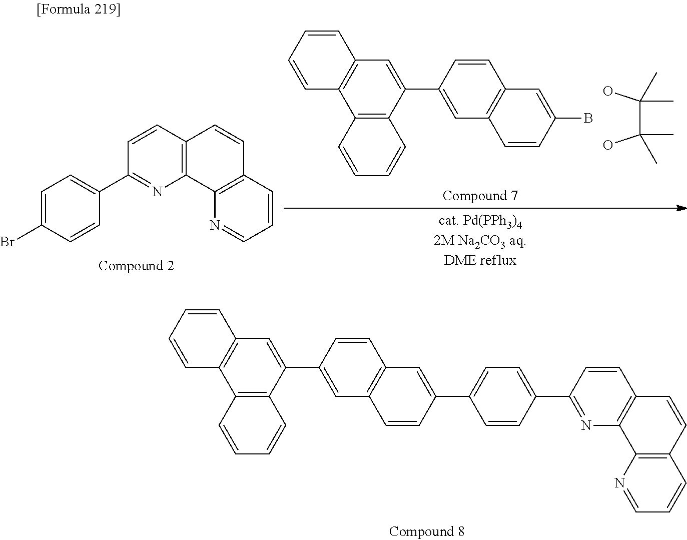

### (3-1) Synthesis of Compound 8

A compound 8 (5.3 g a yield of 64%) was obtained as a white solid according to the same method as in (1-2) synthesis of the compound 4, except for using the compound 7 (7.7 g, 18 mmol) in place of the compound 3. By FD-MS (Field Desorption Mass Spectrometry) analysis, the obtained compound was identified to be the compound 8.

### Synthesis Example 4: Synthesis of Compound 11

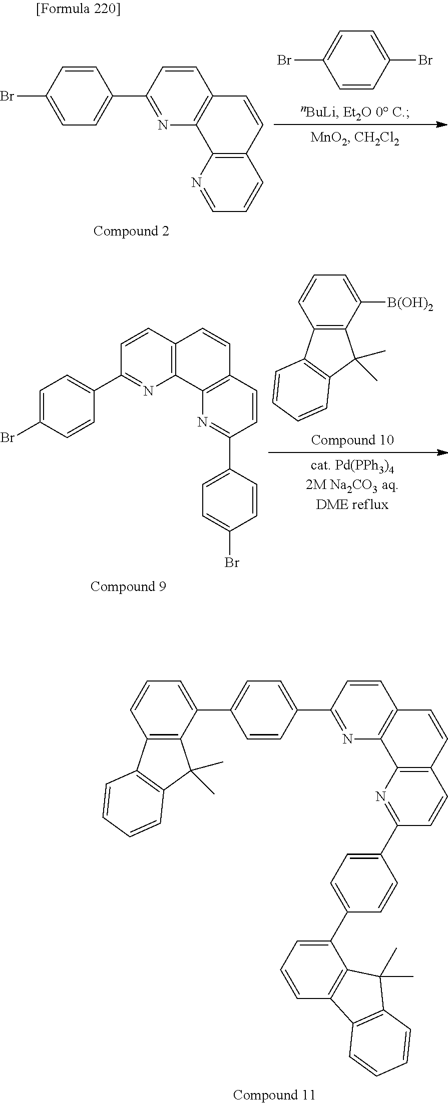

### (4-1) Synthesis of Compound 9

A compound 9 (18 g, a yield of 55%) was obtained as a white solid according to the same method as in (1-1) synthesis of the compound 4, except for using the compound 2 (23 g, 68 mmol) in place of the compound 1. By FD-MS (Field Desorption Mass Spectrometry) analysis, the obtained compound was identified to be the compound 9.

### (4-2) Synthesis of Compound 11

A compound 11 (5.7 g, a yield of 78%) was obtained as a light-yellow solid according to the same method as in (1-2) synthesis of the compound 4, except for using the compound 9 (5.0 g, 10 mmol) in place of the compound 2 and using the compound 10 (5.3 g, 22 mmol) in place of the compound 3. By FD-MS (Field Desorption Mass Spectrometry) analysis, the obtained compound was identified to be the compound 11.

### Synthesis Example 5: Synthesis of Compound 14

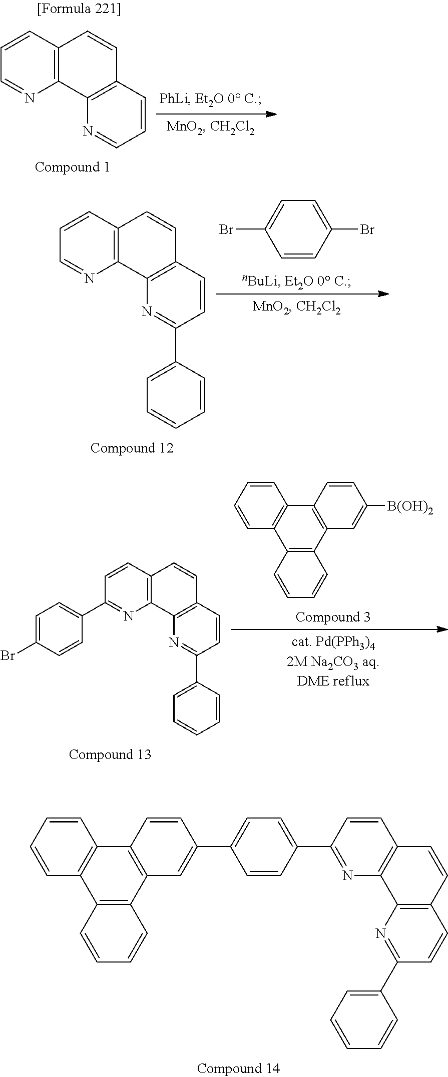 [SMILES: c1cnc2c(c1)ccc1cccnc12 ; c1ccc(-c2ccc3ccc4cccnc4c3n2)cc1 ; Brc1ccc(Br)cc1 ; Brc1ccc(-c2ccc3ccc4ccc(-c5ccccc5)nc4c3n2)cc1 ; OB(O)c1ccc2c3ccccc3c3ccccc3c2c1 ; c1ccc(-c2ccc3ccc4ccc(-c5ccc(-c6ccc7c8ccccc8c8ccccc8c7c6)cc5)nc4c3n2)cc1]

### (5-1) Synthesis of Compound 12

A compound 12 (24 g, a yield of 83%) was obtained as a yellow solid in the same manner as in the (1-1) synthesis of the compound 4, except for using phenyllithium (1.6M butylether solution)(139 mL, 222 mmol) in place of p-bromophenyllithium, the phenyllithium being used at 2 molar equivalent to 1,10-phenanthroline. By FD-MS (Field Desorption Mass Spectrometry) analysis, the obtained compound was identified to be the compound 12.

### (5-2) Synthesis of Compound 13

A compound 13 (12 g, a yield of 76%) was obtained as a yellow solid according to the same method as in (1-1) synthesis of the compound 4, except for using the compound 12 (10 g, 39 mmol) in place of the compound 1. By FD-MS (Field Desorption Mass Spectrometry) analysis, the obtained compound was identified to be the compound 13.

### (5-3) Synthesis of Compound 14

A compound 14 (4.9 g, a yield of 72%) was obtained as a light-yellow solid according to the same method as in (1-2) synthesis of the compound 4, except for using the compound 13 (5.0 g, 12 mmol) in place of the compound 2. By FD-MS (Field Desorption Mass Spectrometry) analysis, the obtained compound was identified to be the compound 14.

### Manufacture of Organic EL Device

### Example 1

A glass substrate (size: 25 mm×75 mm×0.7 mm thick, manufactured by Geomatec Co., Ltd.) having an ITO transparent electrode (anode) was ultrasonic-cleaned in isopropyl alcohol for five minutes, and then UV/ozone-cleaned for 30 minutes.

After the glass substrate having the transparent electrode line was cleaned, the glass substrate was attached to a substrate holder of a vacuum evaporation apparatus. Initially, a compound HAT below was deposited on a surface of the glass substrate so as to cover the transparent electrode line, thereby forming a 5 nm thick film of the compound HAT. The HAT film serves as the hole injecting layer. Subsequently, a compound HT-1 below was deposited on the HAT film to form a 95 nm thick HT-1 film. The HT-1 film serves as the hole transporting layer.

Then, a compound BH-1 (host material) below and a compound BD-1 (dopant material) below were co-deposited on the HT-1 film at a film thickness ratio at which the compound BD-1 accounts for 5 mass %, so that a 25 nm thick organic layer was formed. The organic layer serves as the emitting layer.

A compound ET-11 below was deposited on the emitting layer to form a 20 nm thick ET-11 film. The ET-11 film serves as the first electron transporting layer. A compound ET-21 and lithium (Li) were deposited on the ET-11 film at a film thickness ratio at which Li accounts for 4 mass %, so that a 5 nm thick ET-21 was formed. The ET-21 film serves as the second electron transporting layer. Metal (Al) was deposited on the electron transporting layer to form an 80 nm thick metal cathode. Thus, the organic EL device was manufactured.

Compounds used for manufacturing the organic EL device will be shown below.

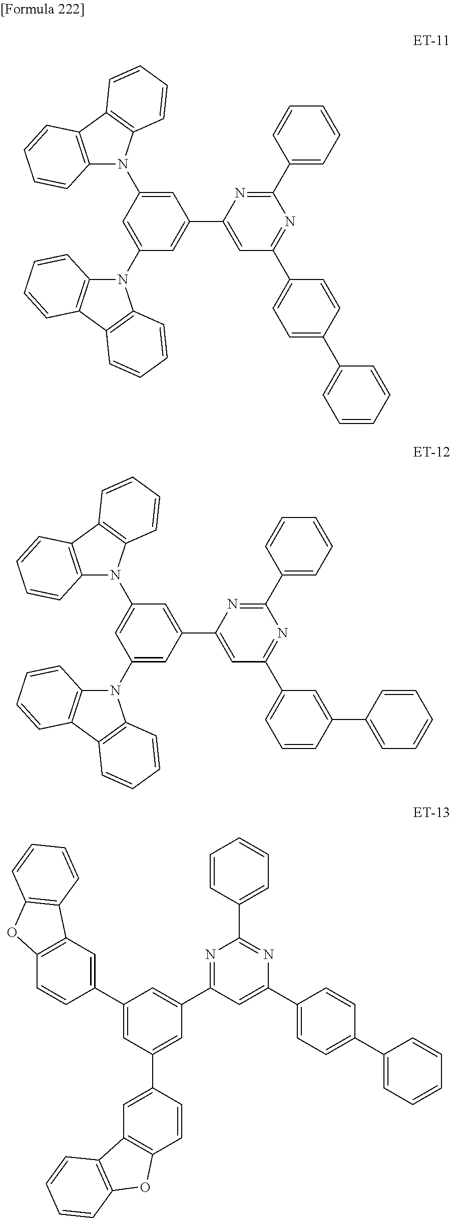 [SMILES: c1ccc(-c2ccc(-c3cc(-c4cc(-n5c6ccccc6c6ccccc65)cc(-n5c6ccccc6c6ccccc65)c4)nc(-c4ccccc4)n3)cc2)cc1 ; c1ccc(-c2cccc(-c3cc(-c4cc(-n5c6ccccc6c6ccccc65)cc(-n5c6ccccc6c6ccccc65)c4)nc(-c4ccccc4)n3)c2)cc1 ; c1ccc(-c2ccc(-c3cc(-c4cc(-c5ccc6oc7ccccc7c6c5)cc(-c5ccc6oc7ccccc7c6c5)c4)nc(-c4ccccc4)n3)cc2)cc1]

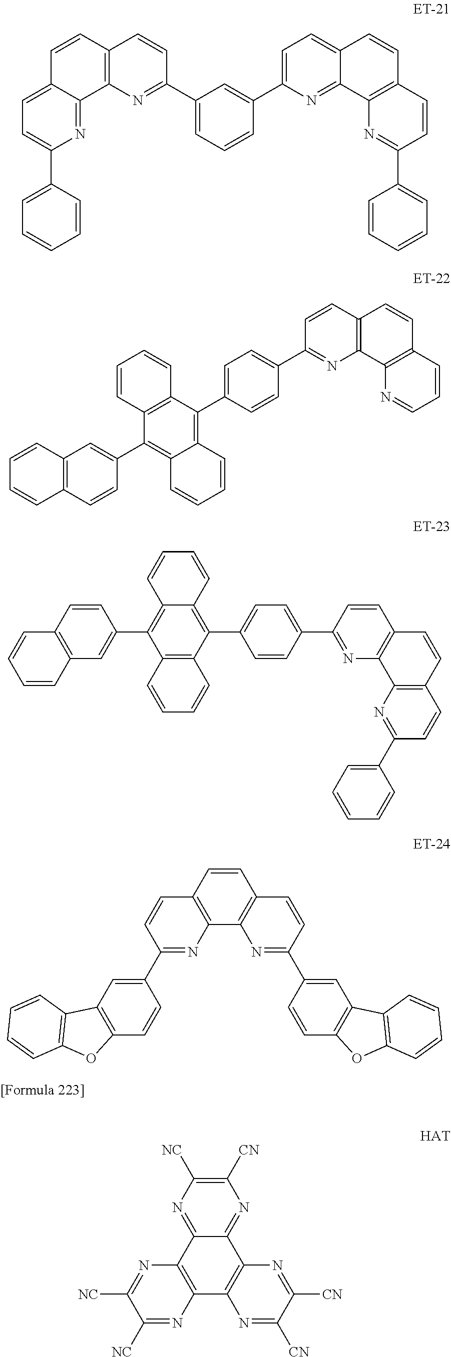 [SMILES: c1ccc(-c2ccc3ccc4ccc(-c5cccc(-c6ccc7ccc8ccc(-c9ccccc9)nc8c7n6)c5)nc4c3n2)cc1 ; c1ccc2cc(-c3c4ccccc4c(-c4ccc(-c5ccc6ccc7cccnc7c6n5)cc4)c4ccccc34)ccc2c1 ; c1ccc(-c2ccc3ccc4ccc(-c5ccc(-c6c7ccccc7c(-c7ccc8ccccc8c7)c7ccccc67)cc5)nc4c3n2)cc1 ; c1ccc2c(c1)oc1ccc(-c3ccc4ccc5ccc(-c6ccc7oc8ccccc8c7c6)nc5c4n3)cc12 ; N#Cc1nc2c3nc(C#N)c(C#N)nc3c3nc(C#N)c(C#N)nc3c2nc1C#N]

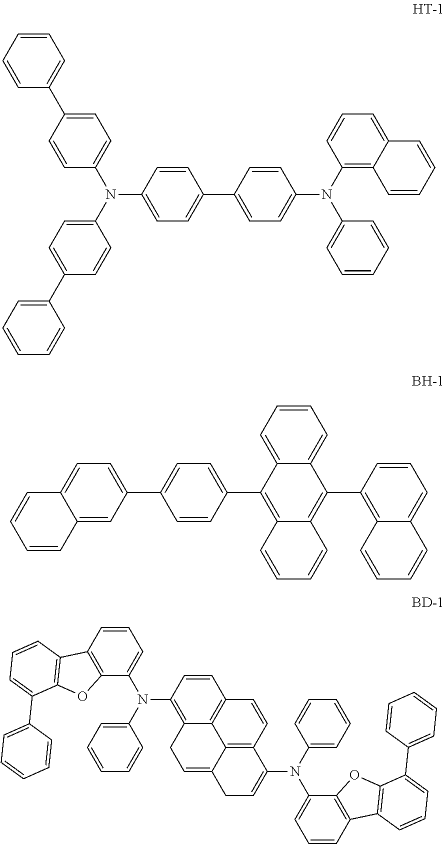

### Examples 2 to 11

In Examples 2 to 11, the organic EL devices were formed in the same manner as in Example 1 except that at least one of the materials for the first electron transporting layer and the second electron transporting layer in Example 1 was replaced by the compounds shown in Table 1.

### Comparative 1

The organic EL device in Comparative 1 was manufactured in the same manner as in Example 1 except that the material for the second electron transporting layer was replaced by the compound ET-11.

TABLE 1

| | First Electron | Second Electron |
|---|---|---|
| | Transporting Layer | Transporting Layer |
| Example 1 | ET-11 | ET-21 |
| Example 2 | ET-11 | ET-22 |
| Example 3 | ET-11 | ET-23 |
| Example 4 | ET-11 | ET-24 |
| Example 5 | ET-12 | ET-21 |
| Example 6 | ET-12 | ET-22 |
| Example 7 | ET-12 | ET-23 |
| Example 8 | ET-12 | ET-24 |
| Example 9 | ET-13 | ET-21 |
| Example 10 | ET-13 | ET-22 |
| Example 11 | ET-13 | ET-23 |
| Comparative 1 | ET-11 | ET-11 |

### Evaluation of Organic EL Devices

Voltage was applied on the manufactured organic EL devices such that a current density was 10 mA/cm², where a voltage (V) was measured. The external quantum efficiency EQE was calculated as follows.

Table 2 shows the voltage and the external quantum efficiency EQE of Comparative 1 and Examples 1 to 11.

### External Quantum Efficiency EQE

Voltage was applied on each of the organic EL devices such that a current density was 10 mA/cm², where spectral radiance spectrum was measured by the above spectroradiometer and the external quantum efficiency EQE (unit: %) was calculated based on the obtained spectral radiance spectrum, assuming that the spectrum was provided under a Lambertian radiation.

TABLE 2

| | V (V) | EQE (%) |
|---|---|---|
| Example 1 | 3.95 | 6.76 |
| Example 2 | 3.87 | 6.83 |
| Example 3 | 3.90 | 7.34 |
| Example 4 | 3.81 | 7.00 |
| Example 5 | 3.66 | 8.07 |
| Example 6 | 3.96 | 8.08 |
| Example 7 | 3.68 | 7.65 |
| Example 8 | 3.99 | 7.58 |
| Example 9 | 3.56 | 8.38 |
| Example 10 | 3.57 | 9.13 |
| Example 11 | 3.85 | 8.71 |
| Comparative 1 | 7.33 | 2.66 |

1. An organic electroluminescence device comprising:
an anode;
a cathode opposite the anode; and
an organic layer interposed between the anode and the cathode, wherein
the organic layer comprises: an emitting layer; a first electron transporting layer interposed between the emitting layer and the cathode; and a second electron transporting layer interposed between the first electron transporting layer and the cathode,
the first electron transporting layer comprises a compound represented by a formula (1) below, and
the second electron transporting layer comprises a compound represented by a formula (2) below, 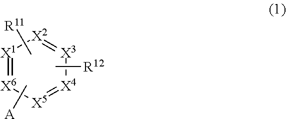
where:
X¹ to X⁶ each independently represent a nitrogen atom, CR, CA, CR¹¹ or CR¹²; at least one of X¹ to X⁶ is a nitrogen atom;
R is each independently selected from the group consisting of a hydrogen atom, a halogen atom, a cyano group, a nitro group, a hydroxyl group, a carboxyl group, a sulfonyl group, a mercapto group, a substituted or unsubstituted boryl group, a substituted or unsubstituted phosphino group, a substituted or unsubstituted acyl group, a substituted or unsubstituted amino group, a substituted or unsubstituted silyl group, a substituted or unsubstituted alkyl group having 1 to 30 carbon atoms, a substituted or unsubstituted alkenyl group having 2 to 30 carbon atoms, a substituted or unsubstituted alkynyl group having 2 to 30 carbon atoms, a substituted or unsubstituted aralkyl group having 6 to 30 carbon atoms, a substituted or unsubstituted alkoxy group having 1 to 30 carbon atoms, a substituted or unsubstituted aryloxy group having 6 to 40 ring carbon atoms, a substituted or unsubstituted heteroaryloxy group having 5 to 40 ring carbon atoms, a substituted or unsubstituted alkylthio group having 1 to 30 carbon atoms, a substituted or unsubstituted arylthio group having 6 to 40 ring carbon atoms, a substituted or unsubstituted heteroarylthio group having 5 to 40 ring carbon atoms, a substituted or unsubstituted alkoxycarbonyl group having 2 to 30 carbon atoms, a substituted or unsubstituted aryloxycarbonyl group having 6 to 40 ring carbon atoms, a substituted or unsubstituted heteroaryloxycarbonyl group having 5 to 40 ring carbon atoms, a substituted or unsubstituted aromatic hydrocarbon group having 6 to 40 ring carbon atoms, and a substituted or unsubstituted heterocyclic group having 5 to 40 ring atoms;
A is represented by a formula (11) below;
R¹¹ and R¹² are a hydrogen atom, a substituted or unsubstituted aromatic hydrocarbon group having 6 to 40 ring carbon atoms, a substituted or unsubstituted heterocyclic group having 5 to 40 ring atoms, or a group represented by the formula (11) below;
when at least one of X¹ to X⁶ is a nitrogen atom and a triphenylenyl group is present in any one of A, R¹¹ and R¹² in the formula (1), the total number of the triphenylenyl group contained in A, R¹¹ and R¹² is not 1; when two or three of X¹ to X⁶ are nitrogen atoms and the triphenylenyl group is present in any one of A, R¹¹ and R¹² in the formula (1), the total number of the triphenylenyl group contained in A, R¹¹ and R¹² is neither 1 nor 2; and
A, R¹¹ and R¹² are respectively bonded to carbon atoms of any ones of X¹ to X⁶, 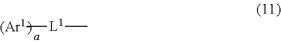
where: a is an integer of 1 to 5; L¹ is a single bond or a linking group; the linking group in L¹ is a substituted or unsubstituted polyvalent linear, branched or cyclic aliphatic hydrocarbon group having 1 to 30 carbon atoms, a substituted or unsubstituted polyvalent amino group, a substituted or unsubstituted polyvalent aromatic hydrocarbon group having 6 to 40 ring carbon atoms, a substituted or unsubstituted polyvalent heterocyclic group having 5 to 40 ring atoms, or a polyvalent multiple linking group provided by bonding two or three groups selected from the aromatic hydrocarbon group and the heterocyclic group;
in the multiple linking group, the aromatic hydrocarbon group and the heterocyclic group forming the multiple linking group are mutually the same or different, and the aromatic hydrocarbon group and the heterocyclic group which are adjacent to each other are optionally mutually bonded to form a ring;
Ar¹ is a substituted or unsubstituted aromatic hydrocarbon group having 6 to 40 ring carbon atoms or a substituted or unsubstituted heterocyclic group having 5 to 40 ring atoms; when a is from 2 to 5, Ar¹ are mutually the same or different; and
Ar¹ and L¹ are bonded to each other to form a cyclic structure or are not bonded, 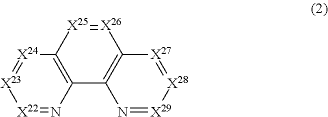
where: X²² to X²⁹ each independently represent a nitrogen atom, CR²¹, or a carbon atom to be bonded to a group represented by a formula (21) below;
at least one of X²² to X²⁹ is a carbon atom to be bonded to a group represented by a formula (21) below; when a plurality of ones of X²² to X²⁹ are carbon atoms to be bonded to the group represented by the formula (21) below, a plurality of the group represented by the formula (21) are the same or different;
R²¹ represents the same as R of the formula (1); and
R²¹ of adjacent CR²¹ in X²² to X²⁹ are bonded to each other to form a cyclic structure or are not bonded, 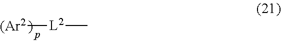
where: p is an integer of 1 to 5;
Ar² is a substituted or unsubstituted aromatic hydrocarbon group having 6 to 40 ring carbon atoms or a substituted or unsubstituted heterocyclic group having 5 to 40 ring atoms, and when p is from 2 to 5, a plurality of Ar² are mutually the same or different;
L² is a single bond or a linking group;
the linking group in L² is a substituted or unsubstituted polyvalent linear, branched or cyclic aliphatic hydrocarbon group having 1 to 30 carbon atoms, a substituted or unsubstituted polyvalent aromatic hydrocarbon group having 6 to 40 ring carbon atoms, a substituted or unsubstituted polyvalent heterocyclic group having 5 to 40 ring atoms, or a polyvalent multiple linking group provided by bonding two or three groups selected from the aromatic hydrocarbon group and the heterocyclic group;
in the multiple linking group, the aromatic hydrocarbon group and the heterocyclic group forming the multiple linking group are mutually the same or different, and the aromatic hydrocarbon group and the heterocyclic group which are adjacent to each other are optionally mutually bonded to form a ring;
the heterocyclic group having 5 to 30 ring atoms in Ar² of the formula (21) comprises a substituted or unsubstituted group derived from the compound represented by the formula (2); the compound represented by the formula (2) contains 6 or less of Ar² that is a substituted or unsubstituted group derived from the compound represented by the formula (2);
adjacent Ar² and L² are bonded to each other to form a cyclic structure or are not bonded; and
L² bonded to any one of carbon atoms of X²² to X²⁹ of the formula (2) and a carbon atom or R²¹ of CR²¹ in one of X²² to X²⁹ adjacent to the carbon atom bonded to L² are further bonded to each other to form a ring, or are not bonded.

2. The organic electroluminescence device according to claim 1, wherein Ar¹ of the formula (11) is represented by a formula (12) below, 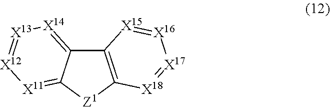
where: X¹¹ to X¹⁸ are each independently a nitrogen atom or CR¹³;
Z is an oxygen atom, sulfur atom, NR¹⁴, CR¹⁵R¹⁶, or SiR¹⁷R¹⁸;
R¹³ to R¹⁸ represent the same as R of the formula (1);
one of R¹³ to R¹⁸ is a single bond to be bonded to L¹ of the formula (11);
when the formula (12) includes a plurality of R¹³, the plurality of R¹³ are mutually the same or different;
the adjacent R¹³ in CR¹³ are optionally bonded together to form a saturated or unsaturated ring; when Z¹ is one of NR¹⁴, CR¹⁵R¹⁶ and SiR¹⁷R¹⁸ and at least one of X¹¹ and X¹⁸ is CR¹³, one of R¹⁴ to R¹⁸ is optionally bonded to R¹³ to form a ring, or alternatively, Z¹ is optionally directly bonded to R¹³ to form a ring; and
the rest of R¹³ to R¹⁸ except for a single bond to be bonded to L¹ are optionally further bonded to L¹ of the formula (11) to form a saturated or unsaturated ring.

3. The organic electroluminescence device according to claim 2, wherein, in the formula (12), X¹³ or X¹⁶ is CR¹³, and R¹³ is a single bond to be bonded to L¹.

4. The organic electroluminescence device according to claim 2, wherein, in the formula (12), X¹¹ or X¹⁸ is CR¹³, and R¹³ is a single bond to be bonded to L¹.

5. The organic electroluminescence device according to claim 2, wherein, in the formula (12), Z¹ is NR¹⁴, and X¹¹ to X¹⁸ are CR¹³.

6. The organic electroluminescence device according to claim 2, wherein, in the formula (12), Z¹ is NR¹⁴, X¹¹ to X¹⁸ are CR¹³, and R¹⁴ is a single bond to be bonded to L¹ of the formula (11).

7. The organic electroluminescence device according to claim 1, wherein
Ar¹ is a substituted or unsubstituted fused aromatic hydrocarbon group having 8 to 20 ring carbon atoms in the formula (11).

8. The organic electroluminescence device according to claim 1, wherein
Ar¹ of the formula (11) is a group represented by a formula (13) below, 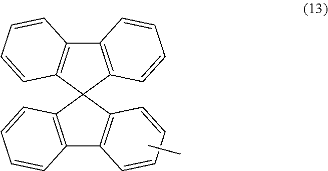
where: a bond represents a single bond between one of carbon atoms and L¹ of the formula (11).

9. The organic electroluminescence device according to claim 1, wherein Ar¹ of the formula (11) is a group represented by a formula (14) below, 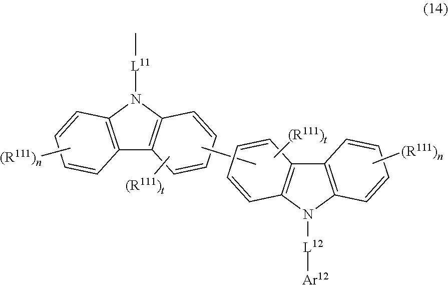
where: t is an integer of 1 to 3;
n is an integer of 1 to 4;
R¹¹¹ represents the same as R of the formula (1) and a plurality of R¹ are mutually the same or different;
L¹¹ and L¹² represent the same as L¹ of the formula (11);
Ar¹² is a substituted or unsubstituted aromatic hydrocarbon group having 6 to 40 ring carbon atoms or a substituted or unsubstituted heterocyclic group having 5 to 40 ring atoms;
L¹¹ is a single bond to be bonded to L¹ of the formula (11); the plurality of R¹¹¹ are bonded to any ones of carbon atoms of the carbazolyl group; and
two carbazolyl groups are mutually bonded at any one of the respective positions 1 to 4.

10. The organic electroluminescence device according to claim 1, wherein a is an integer of 1 to 3 in the formula (11).

11. The organic electroluminescence device according to claim 1, wherein a is 1 or 2 in the formula (11).

12. The organic electroluminescence device according to claim 1, wherein
a is 1 and L¹ is a linking group in the formula (11), and
the linking group in L¹ is a substituted or unsubstituted divalent aromatic hydrocarbon group having 6 to 40 ring carbon atoms or a substituted or unsubstituted divalent heterocyclic group having 5 to 40 ring atoms.

13. The organic electroluminescence device according to claim 1, wherein
a is 2 and L¹ is a linking group in the formula (11), and
the linking group in L¹ is a substituted or unsubstituted trivalent aromatic hydrocarbon group having 6 to 40 ring carbon atoms or a substituted or unsubstituted trivalent heterocyclic group having 5 to 40 ring atoms.

14. The organic electroluminescence device according to claim 1, wherein
L¹ of the formula (11) is a divalent or trivalent residue derived from one of benzene, biphenyl, terphenyl, naphthalene and phenanthrene.

15. The organic electroluminescence device according to claim 1, wherein
two or three of X¹ to X⁶ are nitrogen atoms in the formula (1).

16. The organic electroluminescence device according to claim 1, wherein
two or three of X¹, X³ and X⁵ are nitrogen atoms in the formula (1).

17. The organic electroluminescence device according to claim 1, wherein
R¹¹ or R¹² of the formula (1) is a group selected from groups represented by formulae (1a) to (1p) below,

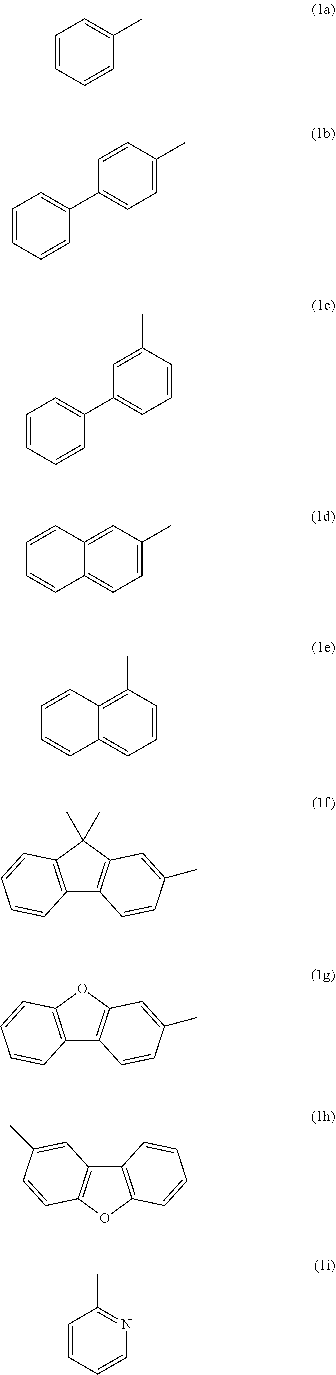

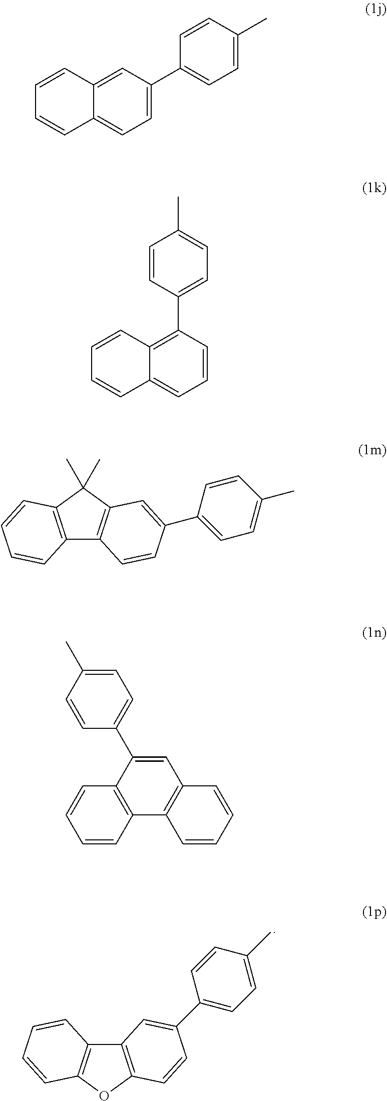

18. The organic electroluminescence device according to claim 17, wherein
in the formula (1), X¹ and X³ are nitrogen atoms, X⁴ is CR¹², and R¹² is a group selected from the groups represented by the formulae (1a) to (1p).

19. The organic electroluminescence device according to claim 1, wherein
X²² or X²⁹ of the formula (2) is a carbon atom to be bonded to the group represented by the formula (21).

20. The organic electroluminescence device according to claim 1, wherein
Ar² is a substituted or unsubstituted fused aromatic hydrocarbon group having 8 to 20 ring carbon atoms in the formula (21).

21. The organic electroluminescence device according to claim 1, wherein
Ar² is a substituted or unsubstituted anthryl group in the formula (21).

22. The organic electroluminescence device according to claim 1, wherein
Ar² is a substituted or unsubstituted heterocyclic group having 5 to 40 ring carbon atoms in the formula (21).

23. The organic electroluminescence device according to claim 1, wherein
Ar² is a substituted or unsubstituted group derived from the compound of the formula (2).

24. The organic electroluminescence device according to claim 1, wherein
Ar² of the formula (21) is represented by a formula (22) below, 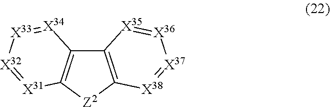
where: X³¹ to X³⁸ are each independently a nitrogen atom or CR²³;
Z² is an oxygen atom, sulfur atom, NR²⁴, CR²⁵R²⁶, or SiR²⁷R²⁸;
R²³ to R²⁸ represent the same as R of the formula (1);
one of R²³ to R²⁸ is a single bond to be bonded to L² of the formula (21);
when the formula (22) includes a plurality of R²³, the plurality of R²³ are mutually the same or different;
R²³ in adjacent CR²³ are optionally bonded together to form a saturated or unsaturated ring; when Z² is one of NR²⁴, CR²⁵R²⁶ and SiR²⁷R²⁸ and at least one of X²¹ and X²⁸ is CR²³, one of R²⁴ to R²⁸ is optionally bonded to R²³ to form a ring, or alternatively, Z² is optionally directly bonded to R²³ to form a ring; and
the rest of R²³ to R²⁸ except for a single bond to be bonded to L² are optionally further bonded to L² of the formula (21) to form a saturated or unsaturated ring.

25. The organic electroluminescence device according to claim 1, wherein the group of the formula (21) is represented by a formula (23) below, 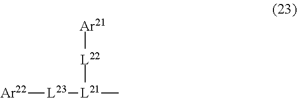
where: L²¹ is a linking group and the linking group in L²¹ is a substituted or unsubstituted trivalent linear, branched or cyclic aliphatic hydrocarbon group having 1 to 30 carbon atoms, a substituted or unsubstituted trivalent aromatic hydrocarbon group having 6 to 40 ring carbon atoms, or a substituted or unsubstituted trivalent heterocyclic group having 5 to 40 ring atoms;
L²² and L²³ are each independently a single bond or a linking group and the linking group in L²² and L²³ is a substituted or unsubstituted divalent linear, branched or cyclic aliphatic hydrocarbon group having 1 to 30 carbon atoms, a substituted or unsubstituted divalent aromatic hydrocarbon group having 6 to 40 ring carbon atoms, or a substituted or unsubstituted divalent heterocyclic group having 5 to 40 ring atoms;
Ar²¹ and Ar²² each independently represent the same as Ar² of the formula (2); and
the heterocyclic group having 5 to 30 ring atoms in Ar²¹ comprises a substituted or unsubstituted group derived from the compound represented by the formula (2).

26. The organic electroluminescence device according to claim 1, wherein
the rest of X²² to X²⁹ except for the carbon atoms bonded to the group represented by the formula (21) are CR²¹ in the formula (2).

27. The organic electroluminescence device according to claim 2, wherein
the group of the formula (12) is selected from groups represented by formulae (12A-1) to (12A-3) and (12B-1) to (12B-6) below, 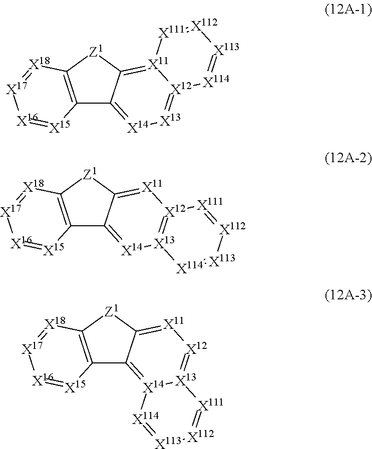
where: Z¹ and X¹¹ to X¹⁸ represent the same as Z¹ and X¹¹ to X¹⁸ in the formula (12) and X¹¹¹ to X¹¹⁴ represent the same as X¹¹ to X¹⁸ in the formula (12), 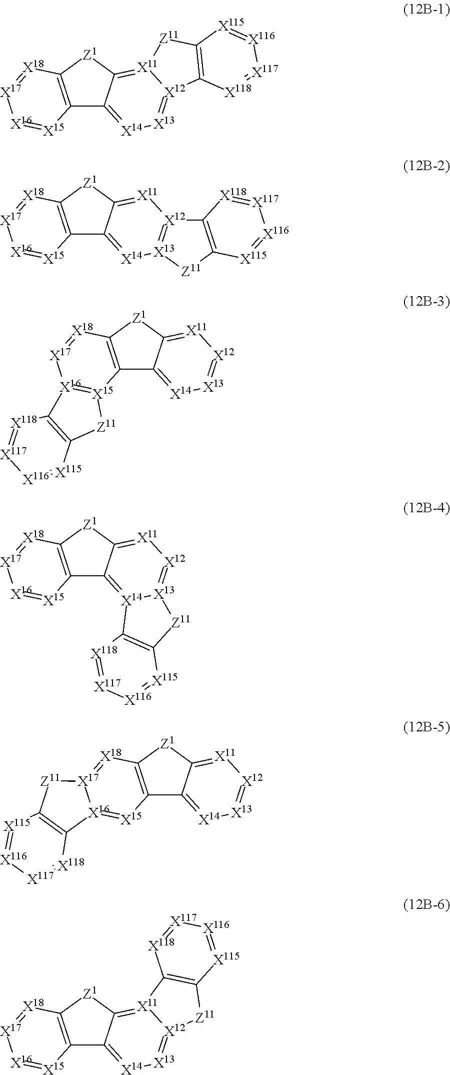
where: Z¹ and X¹¹ to X¹⁸ represent the same as Z¹ and X¹¹ to X¹⁸ in the formula (12), Z¹¹ represents the same as Z¹ in the formula (12), and X¹¹⁵ to X¹¹⁸ represent the same as X¹¹ to X¹⁸ in the formula (12).

28. The organic electroluminescence device according to claim 1, wherein
the compound of the formula (1) is represented by a formula (1-1) below, 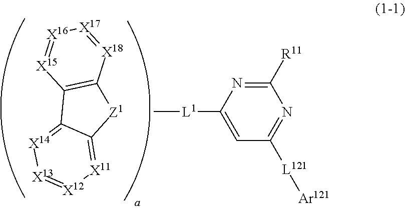
where: R¹¹ represents the same as R¹¹ of the formula (1);
L¹ and L¹²¹ represent the same as L¹ of the formula (11);
a represents the same as a in the formula (11);
Ar¹²¹ represents the same as Ar¹ in the formula (11);
Z¹ and X¹¹ to X¹⁸ respectively represent the same as Z¹ and X¹¹ to X¹⁸ in the formula (12); and
L¹ is bonded to one of Z¹ and X¹¹ to X¹⁸.

29. The organic electroluminescence device according to claim 1, wherein
the compound of the formula (1) is represented by a formula (1-2) below, 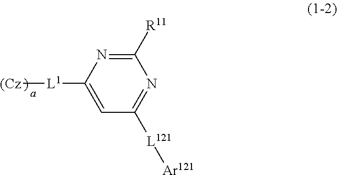
where: R¹¹ represents the same as R¹¹ of the formula (1);
L¹ and L¹²¹ represent the same as L¹ of the formula (11);
a represents the same as a of the formula (11);
Ar¹²¹ represents the same as Ar¹ in the formula (11); and
Cz is a group represented by a formula (1-21) below, 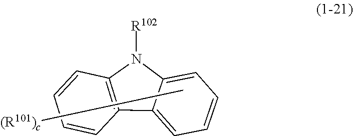
R¹⁰¹ each independently represents the same as R¹³ of the formula (12) and R¹⁰² represents the same as R¹⁴ of the formula (12);
c is an integer of 1 to 8; when c is 2 or more, a plurality of R¹⁰¹ are mutually the same or different;
at least one of R¹⁰¹ and R¹⁰² is a single bond to be bonded to L¹ in the formula (1-2); and
R¹⁰¹ is bonded to any one of carbon atoms of the carbazolyl group.

30. The organic electroluminescence device according to claim 1, wherein
the compound of the formula (1) is represented by a formula (1-3) below, 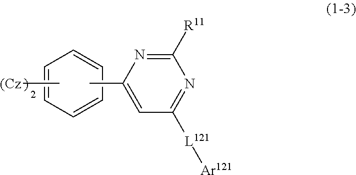
where: R¹¹, L¹²¹, Ar¹²¹ and Cz respectively represent the same as R¹¹, L¹²¹, Ar¹²¹ and Cz of the formula (1-2); and
Cz and a pyrimidine ring are bonded to any ones of carbon atoms of a benzene ring.

31. The organic electroluminescence device according to claim 1, wherein
the compound of the formula (1) is represented by a formula (1-4) below, 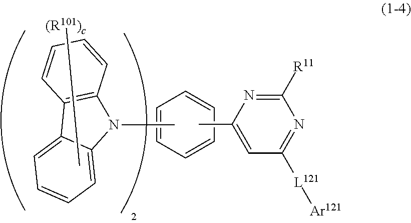
where: R¹¹, L¹²¹, Ar¹²¹ and Cz respectively represent the same as R¹¹, L¹²¹, Ar¹²¹ and Cz of the formula (1-2);
R¹⁰¹ and c respectively represent the same as R¹⁰¹ and c of the formula (1-21);
a carbazolyl group and a pyrimidine ring are bonded to any ones of carbon atoms of a benzene ring; and
R¹⁰¹ is bonded to any one of carbon atoms of the carbazolyl group.

32. The organic electroluminescence device according to claim 1, wherein
the group of the formula (21) is represented by a formula (23-1) below, 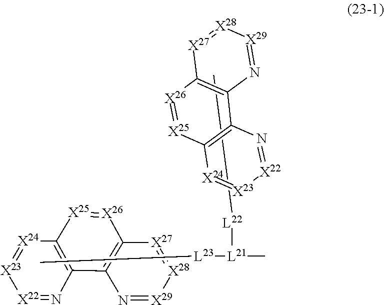
where: X²² to X²⁹ represent the same as X²² to X²⁹ of the formula (2);
L²¹, L²² and L²³ represent the same as L²¹, L²² and L²³ of the formula (23);
a plurality of each of X²² to X²⁹ are mutually the same or different; and
L²² and L²³ are respectively bonded to any ones of X²² to X²⁹.

33. The organic electroluminescence device according to claim 1, wherein
the compound of the formula (2) is represented by a formula (2-2A) or (2-2B) below, 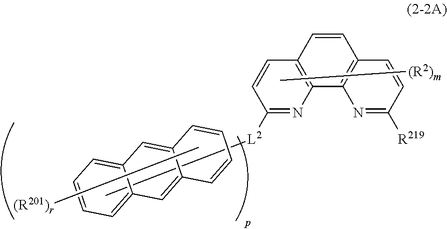
where: L² and p respectively represent the same as L² and p in the formula (21);
m is an integer of 1 to 6 and r is an integer of 1 to 9;
R², R²⁰¹ and R²¹⁹ represent the same as R²¹ of the formula (2); a plurality of each of R² and R²⁰¹ are mutually the same or different;
R² is bonded to any one(s) of carbon atoms at positions 3 to 8 of a phenanthroline ring; and
L² and R²⁰¹ are bonded to any ones of carbon atoms at positions 1 to 10 of an anthracene ring, 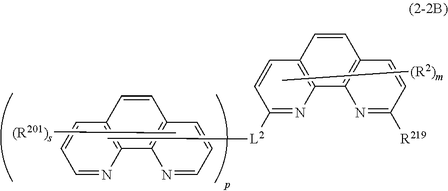
where: L² and p respectively represent the same as L² and p in the formula (21);
m is an integer of 1 to 6 and s is an integer of 1 to 7;
R², R²⁰¹ and R²¹⁹ represent the same as R²¹ of the formula (2); a plurality of each of R² and R²⁰¹ are mutually the same or different;
R² is bonded to any one(s) of carbon atoms at positions 3 to 8 of a phenanthroline ring; and
L² and R²⁰¹ are bonded to any ones of carbon atoms at positions 3 to 8 of the phenanthroline ring.

34. The organic electroluminescence device according to claim 1, wherein
the compound of the formula (2) is represented by one of formulae (2-3A1), (2-3A2) and (2-3B) below, 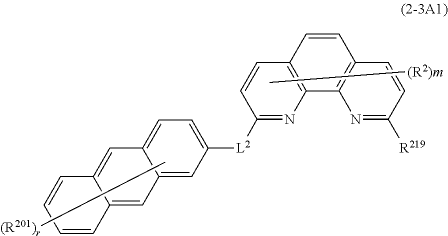
where; L² represents the same as L² of the formula (21);
m is an integer of 1 to 6 and r is an integer of 1 to 9;
R², R²⁰¹ and R²¹⁹ represent the same as R²¹ of the formula (2); a plurality of each of R² and R²⁰¹ are mutually the same or different;
R² is bonded to any one(s) of carbon atoms at positions 3 to 8 of a phenanthroline ring; and
R²⁰¹ is bonded to any one(s) of carbon atoms at positions 1, 3 and 10 of an anthracene ring, 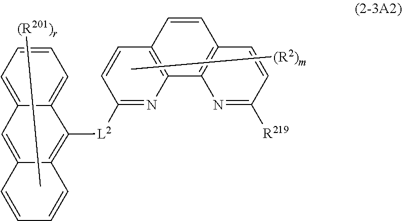
where; L² represents the same as L² of the formula (21);
m is an integer of 1 to 6 and r is an integer of 1 to 9;
R², R²⁰¹ and R²¹⁹ represent the same as R²¹ of the formula (2); a plurality of each of R² and R²⁰¹ are mutually the same or different;
R² is bonded to any one(s) of carbon atoms at positions 3 to 8 of a phenanthroline ring; and
R²⁰¹ is bonded to any one(s) of carbon atoms at positions 1 to 8 and 10 of an anthracene ring, 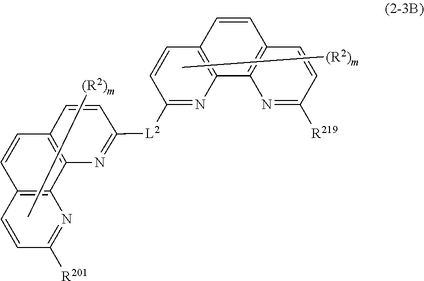
where: L², R², R²⁰¹, R²¹⁹ and m respectively represent the same as L², R², R²⁰¹, R²¹⁹ and m of the formula (2-2B); and
R² is bonded to any one(s) of carbon atoms at positions 3 to 8 of the phenanthroline ring.

35. The organic electroluminescence device according to claim 1, wherein
the compound of the formula (2) is represented by one of formulae (2-2C) to (2-2G) below, 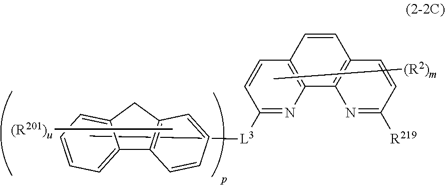
where: L³ is not a single bond but a linking group and the linking group in L³ is a substituted or unsubstituted polyvalent linear, branched or cyclic aliphatic hydrocarbon group having 1 to 30 carbon atoms, a substituted or unsubstituted polyvalent amino group, a substituted or unsubstituted polyvalent aromatic hydrocarbon group having 6 to 40 ring carbon atoms, a substituted or unsubstituted polyvalent heterocyclic group having 5 to 40 ring atoms, or a polyvalent multiple linking group provided by bonding two or three groups selected from the aromatic hydrocarbon group and the heterocyclic group;
in the multiple linking group, the aromatic hydrocarbon group and the heterocyclic group forming the multiple linking group are mutually the same or different, and the aromatic hydrocarbon group and the heterocyclic group which are adjacent to each other are optionally mutually bonded to form a ring;
p represents the same as p of the formula (21);
m is an integer of 1 to 6 and u is an integer of 1 to 9;
R², R²⁰¹ and R²¹⁹ represent the same as R²¹ of the formula (2); a plurality of each of R² and R²⁰¹ are mutually the same or different;
R² is bonded to any one(s) of carbon atoms at positions 3 to 8 of a phenanthroline ring; R²⁰¹ and L² are bonded to any ones of carbon atoms at positions 1 to 9 of a fluorene ring; and
when u is 2 or more, two R²⁰¹ are optionally bonded to the position 9 of the fluorene ring, 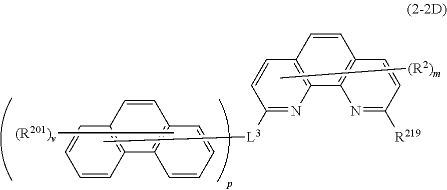
where: L³ represents the same as L³ of the formula (2-2C); p represents the same as p of the formula (21);
m is an integer of 1 to 6 and v is an integer of 1 to 9;
R², R²⁰¹ and R²¹⁹ represent the same as R²¹ of the formula (2); a plurality of each of R² and R²⁰¹ are mutually the same or different;
R² is bonded to any one(s) of carbon atoms at positions 3 to 8 of a phenanthroline ring; and
R²⁰¹ and L² are bonded to any ones of carbon atoms at positions 1 to 10 of the phenanthrene ring, 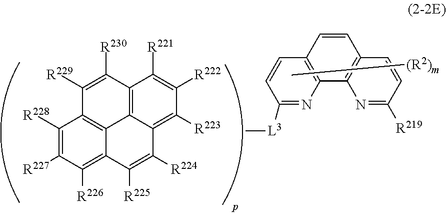
where: L³ represents the same as L³ of the formula (2-2C); p represents the same as p of the formula (21); m is an integer of 1 to 6;
R², R²¹⁹ and R²²¹ to R²³⁰ represent the same as R²¹ of the formula (2); one of R²²¹ to R²³⁰ is a single bond to be bonded to L²; a plurality of R² are mutually the same or different; and
R² is bonded to any one(s) of carbon atoms at positions 3 to 8 of a phenanthroline ring, 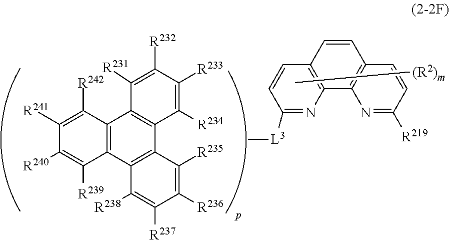
where: L³ represents the same as L³ of the formula (2-2C); p represents the same as p of the formula (21);
m is an integer of 1 to 6;
R², R²¹⁹ and R²³¹ to R²⁴² represent the same as R²¹ of the formula (2); one of R²³¹ to R²⁴² is a single bond to be bonded to L²; a plurality of R² are mutually the same or different; and
R² is bonded to any one(s) of carbon atoms at positions 3 to 8 of a phenanthroline ring, 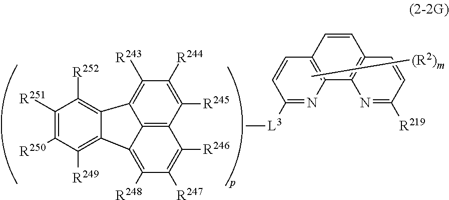
where: L³ represents the same as L³ of the formula (2-2C); p represents the same as p of the formula (21);
m is an integer of 1 to 6;
R², R²¹⁹ and R²⁴³ to R²⁵² represent the same as R²¹ of the formula (2); one of R²³¹ to R²⁴² is a single bond to be bonded to L²; a plurality of R² are mutually the same or different; and
R² is bonded to any one(s) of carbon atoms at positions 3 to 8 of a phenanthroline ring.

36. The organic electroluminescence device according to claim 1, wherein
the first electron transporting layer is adjacent to the emitting layer.

37. The organic electroluminescence device according to claim 1, wherein
the second electron transporting layer comprises at least one of an electron-donating dopant and an organic metal complex.

38. An electronic device comprising the organic electroluminescence device according to claim 1.
